(19) United States
(12) Patent Application Publication
(10) Pub. No.: US 2024/0198310 A1
(43) Pub. Date: Jun. 20, 2024

(54) Library Preparation Systems and Associated Methods

(71) Applicant: ILLUMINA, INC., San Diego, CA (US)

(72) Inventors: Emily Parker, Encinitas, CA (US); Bryan Crane, San Diego, CA (US); Peter Thomas, Poway, CA (US); Megan Blanchard, Poway, CA (US); James Osmus, San Diego, CA (US); Bradley Drews, Poway, CA (US); Richard Lemoine, San Diego, CA (US); Gregory Holst, San Diego, CA (US)

(21) Appl. No.: 18/540,851

(22) Filed: Dec. 14, 2023

Related U.S. Application Data
(60) Provisional application No. 63/433,389, filed on Dec. 16, 2022.

(51) Int. Cl. B01J 19/00

(52) U.S. Cl. CPC B01J 19/0046; B01J 2219/00351; B01J 2219/005

(57) ABSTRACT

Library preparation systems and methods are disclosed. In an implementation, a modular bay for preparing a library of samples for sequencing, the modular bay comprising a contact dispenser and a working area comprising a working plate receptacle adapted to receive a working plate, a thermocycler, a magnet, and a drawer. The drawer comprising a consumables area adapted to receive a sample plate adapted to contain a sample, and a plurality of consumables for interacting with the sample in the working plate. The contact dispenser is linearly movable in a longitudinal direction between the consumables area and the working area, such that the contact dispenser is configured to (i) move the sample from the sample plate in the consumables area to the working plate in the working area, and (ii) move the plurality of consumables between the consumables area and the working plate in the working area.

### RELATED APPLICATION

This application claims the benefit of and priority to U.S. Provisional Patent Application No. 63/433,389, filed Dec. 16, 2022, the content of which is incorporated by reference herein in its entirety and for all purposes.

### BACKGROUND

DNA libraries may be prepared to allow samples to be sequenced.

### SUMMARY

Shortcomings of the prior art can be overcome and benefits as described later in this disclosure can be achieved through the provision of library preparation systems and methods. Various implementations of the apparatus and methods are described below, and the apparatus and methods, including and excluding the additional implementations enumerated below, in any combination (provided these combinations are not inconsistent), may overcome these shortcomings and achieve the benefits described herein.

In a first implementation, a modular system for preparing a library of samples for sequencing, the modular system includes a first assay bay, a common bay, and a mover. The first assay bay includes a first contact dispenser; a first working area, and a first drawer. The first working area includes a working plate receptacle adapted to receive a working plate, a thermocycler, and a magnet. The first drawer includes a consumables area adapted to receive a sample plate adapted to contain a sample, and a plurality of consumables for interacting with the sample in the working plate. The common bay includes an analyzer area includes an imaging system. The mover is operatively coupled to the first assay bay and the common bay. The first contact dispenser is linearly movable in a first direction between the consumables area and the first working area, such that the first contact dispenser is configured to (i) move the sample from the sample plate in the consumables area to the working plate in the first working area, and (ii) move the plurality of consumables between the consumables area and the working plate in the first working area. The mover is movable in the first direction and a second direction perpendicular to the first direction between the first assay bay and the common bay, such that the mover is configured to move the working plate from the first working area to the analyzer area for analysis by the imaging system.

In a second implementation, a modular bay for preparing a library of samples for sequencing is disclosed, the modular bay includes a contact dispenser and a working area includes a working plate receptacle adapted to receive a working plate, a thermocycler, and a magnet, and a drawer. The drawer includes a consumables area adapted to receive a sample plate adapted to contain a sample, and a plurality of consumables for interacting with the sample in the working plate. The contact dispenser is linearly movable in a longitudinal direction between the consumables area and the working area, such that the contact dispenser is configured to (i) move the sample from the sample plate in the consumables area to the working plate in the working area, and (ii) move the plurality of consumables between the consumables area and the working plate in the working area.

In a third implementation, an apparatus includes a system having a consumables area including a consumables receptacle, a mover, a contact dispenser, a stage to move the contact dispenser, a plate receptacle, a magnet, a thermocycler, and an analyzer area includes an imaging system. The consumables receptacle is to receive a tip tray including a first tip and a second tip, a first plate having a well containing a sample, a second plate having a well, an index tray having a well containing indexes, and a bead tray having a well containing beads.

In a fourth implementation, an apparatus is provided that includes a library preparation system and a sequencing system fluidly coupled to the library preparation system.

In a fifth implementation, an apparatus is provided that includes a system including a bay, a second working area, a second contact dispenser, a second stage, and a mover. The bay includes a consumables area and a working area. The consumable area includes a consumables receptacle, a first contact dispenser, and a stage to move the first contact dispenser. The working area includes a first plate receptacle, a magnet, and a thermocycler. The second working area includes a second plate receptacle and an analyzer area includes an imaging system. The second stage to move the second contact dispenser relative to the first bay and the second working area.

In a sixth implementation, an apparatus is provided that includes a system having a consumables area, a mover, a working area, a magnet, a thermocycler, an analyzer area, and a reagent reservoir receptacle to receive a reagent reservoir. The consumables area includes a consumables receptacle to receive a tip tray including a first tip and a second tip, a first plate having a well containing a sample, a second plate having a well, an index tray having a well containing indexes, and a bead tray having a well containing beads. The working area includes a contact dispenser, a non-contact dispenser, and a stage including a first plate receptacle, a second plate receptacle, and a third plate receptacle. The analyzer area includes a substrate and an imaging system. The mover is to move the tip tray, the first plate, the second plate, and the index tray between the consumables area and the working area. The stage is to align the first plate receptacle, the second plate receptacle, and the third plate receptacle relative to the contact dispenser and the non-contact dispenser. The non-contact dispenser is to be fluidly coupled to the reagent reservoir.

In a seventh implementation, an apparatus is provided that includes a system having a consumables area, a mover, a working area, a magnet, a thermocycler, an analyzer area, and a reagent reservoir receptacle to receive a reagent reservoir. The consumables area includes a consumables receptacle to receive a tip tray including a first tip and a second tip, a first plate having a well containing a sample, a second plate having a well, an index tray having a well containing indexes, and a bead tray having a well containing beads. The working area includes a contact dispenser, a non-contact dispenser, and a stage including a first plate receptacle, a second plate receptacle, and a third plate receptacle. The analyzer area includes a substrate and an imaging system. The mover is to move the tip tray, the first plate, the second plate, and the index tray between the consumables area and the working area. The stage is to align the first plate receptacle, the second plate receptacle, and the third plate receptacle relative to the contact dispenser and the non-contact dispenser.

In an eighth implementation, a modular system for preparing a library of samples for sequencing is provided that includes a first assay bay, a common bay, and a mover, the first assay bay includes a first contact dispenser; a first working area includes a working plate receptacle; a thermocycler; and a magnet; and a first drawer. The first drawer includes a consumables area adapted to receive a sample plate adapted to contain a sample, and a plurality of consumables for interacting with the sample. The common bay includes an analyzer area including an imaging system. The mover is operatively coupled to the first assay bay and the common bay. The first contact dispenser is linearly movable in a first direction between the consumables area and the first working area, such that the first contact dispenser is configured to (i) move the sample plate in the consumables area to the working plate in the first working area, and (ii) move the plurality of consumables between the consumables area and the sample plate in the first working area. The mover is movable in the first direction and a second direction perpendicular to the first direction between the first assay bay and the common bay, such that the mover is configured to move the sample plate from the first working area to the analyzer area for analysis by the imaging system.

In a ninth implementation, a modular bay for preparing a library of samples for sequencing, the modular bay includes a contact dispenser and a working area includes a working plate receptacle; a thermocycler; and a magnet; and a drawer. The drawer includes a consumables area adapted to receive a sample plate adapted to contain a sample, and a plurality of consumables for interacting with the sample. The contact dispenser is linearly movable in a longitudinal direction between the consumables area and the working area, such that the contact dispenser is configured to (i) move the sample plate in the consumables area to the working plate in the working area, and (ii) move the plurality of consumables.

In a tenth implementation, an apparatus includes a pipette assembly including a body, a guide, a bar, a plurality of pipettes, a plurality of gaskets, and a pipette cam assembly. The body includes a base defining a plurality of pipette apertures. The guide includes a plurality of protrusions that define pipette apertures that align with the pipette apertures of the base. The bar includes a plurality of apertures through which the protrusions of the guide extend. The plurality of pipettes is coupled to the body and extend through the pipette apertures of the body and the guide. The pipettes each having an end including a flange. The plurality of gaskets are positioned between corresponding flanges of the pipettes and the protrusions. The pipette cam assembly is to move the body away from the guide and move the flanges of the pipettes toward the protrusions to compress the gaskets and to move body toward the guide and move the flanges of the pipettes away from the protrusions to relax the gaskets.

In an eleventh implementation, an apparatus includes a thermocycler a base, a plate receptacle, and a cover assembly. The base includes a stop wall and the plate receptacle is on the base. The cover assembly is movably coupled to the base. The cover assembly has a sled, a cover, a cover follower, and a cam assembly. The sled includes a front wall and a rear wall and a receptacle defined between the front wall and the rear wall. The cover is positioned within the receptacle of the sled. The cover follower is positioned within the receptacle of the sled and movably coupled to the cover. The cam assembly is to cause the cover to move toward the base to cover the plate receptacle and to cause the cover follower to move toward the base. The plate receptacle is to receive a plate having a well and the thermocycler is to adjust a temperature of a sample within the well of the plate.

In a twelve implementation, an apparatus includes a drawer includes a platform. The drawer includes a plate receptacle, a small liquid reagent well plate receptacle, a large liquid reagent well plate receptacle, a dry well plate receptable, and a waste reservoir. The plate receptacle is coupled to the platform. The small liquid reagent well plate receptacle is coupled to the platform and includes a base, a first end wall, and a second end wall. The first end wall is coupled to the base and has an inward extending lip that forms a first groove with the base. The second end wall is coupled to the base and has an inward extending lip that forms a second groove with the base and includes a key. The large liquid reagent well plate receptacle is coupled to the platform and has a base, a first end wall, and a second end wall. The first end wall has an inward extending lip that forms a first groove with the base of the large liquid reagent well plate receptacle. The second end wall is coupled to the base and has an inward extending lip that forms a second groove with the base of the large liquid reagent well plate receptacle and includes a key. The dry well plate receptable positioned on the platform and defining a waste reservoir compartment. The waste reservoir having a wider portion including an inlet and a narrow portion that extends from the wider portion and is positioned within the waste reservoir compartment.

In a thirteenth implementation, an apparatus includes a reagent well plate and a plurality of reagent wells. The reagent well plate includes a first end wall includes a male snap-fit component, a second end wall includes a male snap-fit component, and a panel coupled to and extending between the first end wall and the second end wall. The panel defining a plurality of reagent well receptacles and including a top surface. The reagent wells include an end having an annular collar. The reagent wells positioned within the reagent well receptacles and the annular collars engaging the top surface.

In a fourteenth implementation, an apparatus includes a well plate including a rectangular wall, a panel, and a plurality of reagent wells. The rectangular wall includes end walls and side walls. The end walls each include a cutout and a recess that form a handle that extends between the side walls. The panel is coupled to the rectangular wall. The panel defines a plurality of reagent well receptacles and including a top surface. The end walls and the side walls extending outwardly from the panel. The reagent wells include an end having an annular collar. The reagent wells positioned within the reagent well receptacles and the annular collars engaging the top surface.

In a fifteenth implementation, an apparatus includes a consumables area, a plurality of assay bays, a common bay, and a cross-bay gantry. The consumables area to carry a plurality of well plates. Each of the well plates include a rectangular wall include a cutout. The assay bays each include an assay bay plate receptacle, a pipette assembly, a thermocycler and a magnet to perform amplification processes and cleanup processes associated with preparing a library of samples for sequencing. The common bay includes a common bay plate receptacle and an imaging system to perform quantification processes associated with preparing the library of samples for sequencing. The cross-bay gantry includes a gripper movable between the consumables area, the assay bays, and the common bay. The gripper includes arms including inward extending extensions that are movable toward or away from one another. The extensions of the gripper are positionable in the cutout of a corresponding well plate to move the well plate between any of the consumables bay, the assay bay plate receptacle, and the common bay plate receptacle.

In a sixteenth implementation, an apparatus includes a library preparation system including a consumables area, a plurality of assay bays, a common bay, a cross-bay gantry, and a sample sipper assembly. The consumables area to carry a plurality of well plates. The assay bays each include an assay bay plate receptacle, a pipette assembly, a thermocycler and a magnet to perform amplification processes and cleanup processes associated with preparing a library of samples for sequencing. The common bay includes a common bay plate receptacle and an imaging system to perform quantification processes associated with preparing the library of samples for sequencing. The cross-bay gantry includes a gripper movable between the consumables area, the assay bays, and the common bay. The sample sipper assembly includes a plurality of sippers and an actuator to move the sippers relative to a plate receptacle of the library preparation system. The sample sipper assembly associated with transferring the library of samples to a sequencing system.

In a seventeenth implementation, an apparatus includes a plurality of assay bays, a common bay, and a cross-bay gantry. The assay bays each include an assay bay plate receptacle, a pipette assembly, a thermocycler and a magnet to perform amplification processes and cleanup processes associated with preparing a library of samples for sequencing. The pipette assembly including a body, a guide, a bar, a plurality of pipettes, a plurality of gaskets, and a pipette cam assembly. The body includes a base defining a plurality of pipette apertures. The guide includes a plurality of protrusions that define pipette apertures that align with the pipette apertures of the base. The bar includes a plurality of apertures through which the protrusions of the guide extend. The pipettes coupled to the body and extending through the pipette apertures of the body and the guide. The pipettes each having an end including a flange. The gaskets positioned between corresponding flanges of the pipettes and the protrusions. The pipette cam assembly to move the body away from the guide and move the flanges of the pipettes toward the protrusions to compress the gaskets and to move the body toward the guide and move the flanges of the pipettes away from the protrusions to relax the gaskets. The common bay includes a common bay plate receptacle and an imaging system to perform quantification processes associated with preparing the library of samples for sequencing. The cross-bay gantry includes a gripper movable between the assay bays and the common bay.

In an eighteenth implementation, an apparatus is provided that includes a plurality of assay bays each include an assay bay plate receptacle, a pipette assembly, a thermocycler and a magnet to perform amplification processes and cleanup processes associated with preparing a library of samples for sequencing, the thermocycler includes a base including a stop wall; a plate receptacle on the base; and a cover assembly movably coupled to the base, the cover assembly includes a sled includes a front wall and a rear wall and a receptacle defined between the front wall and the rear wall; a cover positioned within the receptacle of the sled; a cover follower positioned within the receptacle of the sled and movably coupled to the cover; and a cam assembly to cause the cover to move toward the base to cover the plate receptacle and to cause the cover follower to move toward the base, wherein the plate receptacle is to receive a plate having a well and the thermocycler is to adjust a temperature of a sample within the well of the plate, a common bay includes a common bay plate receptacle and an imaging system to perform quantification processes associated with preparing the library of samples for sequencing; and a cross-bay gantry including a gripper movable between the assay bays and the common bay.

In a nineteenth implementation, an apparatus is provided that includes a plurality of assay bays each include a drawer including a platform, an assay bay plate receptacle, a pipette assembly, a thermocycler and a magnet to perform amplification processes and cleanup processes associated with preparing a library of samples for sequencing, the drawer includes a plate receptacle coupled to the platform; a small liquid reagent well plate receptacle coupled to the platform and includes a base; a first end wall having an inward extending lip that forms a first groove with the base; a second end wall coupled to the base and having an inward extending lip that forms a second groove and includes a key; and a large liquid reagent well plate receptacle coupled to the platform and includes a base; a first end wall having an inward extending lip that forms a first groove with the base of the large liquid reagent well plate receptacle; a second end wall coupled to the base and having an inward extending lip that forms a second groove with the base of the large liquid reagent well plate receptacle and includes a key; a dry well plate receptable positioned on the platform and defining a waste reservoir compartment; a waste reservoir having a wider portion including an inlet and a narrow portion that extends from the wider portion and is positioned within the waste reservoir compartment; a common bay includes a common bay plate receptacle and an imaging system to perform quantification processes associated with preparing the library of samples for sequencing; and a cross-bay gantry includes a gripper movable between the assay bays and the common bay.

In a twentieth implementation, a library preparation system for preparing a library of samples for sequencing is provided that includes a working area for preparing a library of samples for genomic sequencing; a communication interface; and one or more processors communicatively coupled to the communication interface, and configured to communicate, via the communication interface, with a sequencer by transmitting or receiving information related to the library of samples.

In a twenty first implementation, a method for communicating between a library preparation system and a sequencer, the method includes preparing, by a library preparation system having a contact dispenser and a working area for causing one or more consumables to interact with a sample, a library of samples for genomic sequencing; and communicating, by one or more processors in the library preparation system via a communication interface, with a sequencer by transmitting or receiving information related to the library of samples.

In a twenty second implementation, a method includes receiving a small liquid reagent well plate by a small liquid reagent well plate receptacle on a platform of a drawer, wherein a snap-fit connection is formed when the small liquid reagent well plate receptacle receives the small liquid reagent well plate; receiving a large liquid reagent well plate by a large liquid reagent well plate receptacle coupled to the platform of the drawer, wherein a snap-fit connection is formed when the large liquid reagent well plate receptacle receives the large liquid reagent well plate; receiving a dry reagent well plate by a dry well plate receptable positioned on the platform and defining a waste reservoir compartment, wherein a waste reservoir is positioned within the waste reservoir compartment; and receiving the drawer in one of a plurality of assay bays of a library preparation system, the library preparation system includes the assay bays and a common bay, the assay bays to each perform amplification processes and cleanup processes associated with preparing a library of samples for sequencing, the common bay to perform library quantification processes, library pooling processes, denaturing processes, and diluting processes associated with preparing the library of samples for sequencing.

In a twenty third implementation, a method, includes performing amplification processes and cleanup processes associated with preparing a library of samples for sequencing on a sample contained in a well plate positioned on a plate receptacle of a thermocycler on an assay bay of a library preparation system; removing the well plate from the plate receptacle of the thermocycler using a gripper by positioning the gripper within a cutout of the well plate and moving gripper away from the plate receptacle; moving the well plate using the gripper to an common bay of the library preparation system; and performing library quantification processes associated with preparing the library of samples for sequencing at the common bay.

In a twenty forth implementation, a method includes inserting ends of pipettes of a pipette assembly of an assay bay of a library preparation system and the gaskets carried by the pipettes into pipette tips on a drawer of the assay bay; compressing the gaskets of the pipette assembly to couple the pipette tips and the pipette assembly; dispensing reagent into a well plate on a plate receptacle of the assay bay using the pipette assembly and the pipette tips; and performing amplification processes and cleanup processes at the plate receptacle.

In a twenty fifth implementation, a method includes moving a sled of a cover assembly of a thermocycler toward a stop wall of a base of the thermocycler, a cover positioned within a receptacle of the sled and a cover follower positioned within the receptacle of the sled and movably coupled to the cover; engaging the stop wall with cover bearings coupled to the cover; engaging a rear wall of the sled with cover follower bearings coupled to the cover follower; moving inner slot bearings within inner cam slots of inner cam plates to move the cover toward covering a plate receptacle, the inner slot bearings coupled to the cover and the inner cam plates coupled to the sled; and moving outer slot bearings within outer cam slots of outer cam plates to move the cover follower toward the base, the outer slot bearings coupled to the cover follower and the outer cam plates coupled to the plate.

In a twenty sixth implementation, a method includes performing amplification processes and cleanup processes on samples in well plates at a plurality of assay bays, each assay bay includes an assay bay plate receptacle, a pipette assembly, a thermocycler and a magnet to perform; moving the samples from the assay bays to a common bay using a cross-bay gantry; performing library quantification processes on the samples at the common bay; and archiving the library of samples.

In a twenty seventh implementation, a method includes performing amplification processes and cleanup processes on samples in well plates at a plurality of assay bays, each assay bay includes an assay bay plate receptacle, a pipette assembly, a thermocycler and a magnet to perform; moving the samples from the assay bays to a common bay using a cross-bay gantry; and performing library quantification processes and library pooling processes on the samples at the common bay.

In a twenty eighth implementation, a method includes performing amplification processes and cleanup processes on samples in well plates at a plurality of assay bays, each assay bay includes an assay bay plate receptacle, a pipette assembly, a thermocycler and a magnet is to perform amplification processes and cleanup processes; moving the samples from the assay bays to a common bay using a cross-bay gantry; and preparing a sequence ready pool of samples at the common bay.

In a twenty ninth implementation, a method includes performing amplification processes and cleanup processes on samples in well plates at a plurality of assay bays, each assay bay includes an assay bay plate receptacle, a pipette assembly, a thermocycler and a magnet; moving the samples from the assay bays to a common bay using a cross-bay gantry; preparing a sequence ready pool of samples at the common bay; and transferring the sequence ready pool of samples to a sequencer.

In a thirtieth implementation, an apparatus includes a library preparation system includes an assay bay to perform amplification processes and cleanup processes; and a common bay to perform quantification process.

In a thirty first implementation, an apparatus includes a library preparation system includes a sample sipper assembly includes sippers to be coupled to a sample cartridge; and a sequencing instrument includes a flow cell interface to be coupled to a flow cell having a plurality of channels; a central valve and an auxiliary waste fluidic line coupled to the central valve and to be coupled to a waste reservoir, the central valve coupled to the flow cell interface and movable between a first position fluidically connecting an inlet of the plurality of channels to the auxiliary waste fluidic line and a second position fluidically connecting a reagent reservoir and the plurality of channels; and a sample loading assembly positioned between the flow cell interface and the sample sipper assembly of the library preparation system, the sample loading assembly includes a body carrying a plurality of sample valves and defining a plurality of sample ports and a plurality of flow cell ports, each sample port coupled to a corresponding sipper of the sample sipper assembly via a sample fluidic line, wherein sample sipper assembly of the library preparation system is positioned downstream of the flow cell interface of the sequencing instrument.

In a thirty second implementation, an apparatus is provided that includes a library preparation system, includes a sample sipper assembly including sippers to be coupled to a sample cartridge a sequencing instrument, includes one or more valves adapted to be coupled to corresponding reagent reservoirs; a flow cell interface adapted to be coupled to a flow cell; a pump adapted to load a channel of the flow cell with the sample of interest via the flow cell interface associated with an outlet of the flow cell and a corresponding sipper of the sample sipper assembly.

In a thirty third implementation, a method includes moving a first sample valve of one or more sample valves to a first position to fluidically couple a first sipper of a sipper manifold assembly of a library preparation system with a first pump of a sequencing instrument; drawing a first sample of interest through the first sipper of the sipper manifold assembly from the library preparation system toward the first pump of the sequencing instrument; moving the first sample valve to a second position to fluidically couple the first pump and a channel of a flow cell coupled to a flow cell interface of the sequencing instrument; and pumping the first sample of interest into the first channel of the flow cell through an outlet of the first channel.

In a thirty fourth implementation, a method, includes priming fluidic lines and a sample sipper assembly of a library preparation system with lead buffer using a pump manifold assembly of a sequencing instrument; drawing samples of interest into the fluidic lines and the sequencing instrument from the library preparation system using the sample sipper assembly and the pump manifold assembly; drawing lag buffer into the fluidic lines and behind the samples of interest and the sequencing instrument from the library preparation system using the sample sipper assembly and the pump manifold assembly; and urging the lag buffer, the samples of interest, and the lead buffer toward channels of a flow cell positioned on a flow cell interface of the sequencing instrument.

In further accordance with the foregoing the first-thirty fourth implementations, an apparatus and/or method may further include or comprise any one or more of the following:

In an implementation, the first contact dispenser is not movable in the second direction.

In another implementation, the first contact dispenser and the mover are both movable in a third direction perpendicular to the first and second directions.

In another implementation, the common bay includes a pooling area, and wherein the mover is configured to move between the analyzer area and the pooling area.

In another implementation, the first contact dispenser includes a first contact head configured to hold tips.

In another implementation, the first drawer is linearly movable in the first direction relative to the first working area between a loading position and an operating position, wherein when the first drawer is in the loading position, the first drawer is spaced a first distance from the first working area, and when the first drawer is in the operating position, the first drawer is spaced a second distance from the first working area, the second distance being less than the first distance.

In another implementation, the apparatus includes a movable stage coupled to the first contact dispenser for linearly moving the first contact dispenser in the first direction and the third direction.

In another implementation, the apparatus includes a first actuator operatively coupled to the movable stage and to the magnet, the first actuator configured to actuate movement of the movable stage and to move the magnet relative to the working plate receptacle.

In another implementation, the apparatus includes a gantry system, wherein the mover is movable along the gantry system.

In another implementation, the apparatus includes a second actuator configured to actuate movement of the mover in the first and second directions.

In another implementation, the apparatus includes a door that is movable to enclose the thermocycler and the working plate receptacle.

In another implementation, the thermocycler is aligned with the working plate receptacle in the first direction.

In another implementation, the thermocycler is configured to amplify the sample within the working plate.

In another implementation, the consumables area is adapted to receive a lid for the working plate, and wherein the first contact dispenser is configured to move the lid from the consumables area and to place the lid on the working plate.

In another implementation, the plurality of consumables include a tip tray includes a first reusable tip and a second reusable tip, a second working plate, an index tray adapted to contain indexes, a bead tray adapted to contain beads, and a reagent reservoir adapted to contain a reagent.

In another implementation, the first contact dispenser is configured to move the sample from the sample plate in the consumables area to the working plate in the first working area using the first reusable tip and to move one or more indexes from the index tray in the consumables area to the working plate using the second reusable tip.

In another implementation, the first contact dispenser is configured to aspirate the indexes from the index tray in the consumables area and to dispense the indexes in the working plate in the first working area.

In another implementation, the first contact dispenser is configured to aspirate the beads from the bead tray in the consumables area and to dispense the beads in the working plate in the first working area.

In another implementation, the magnet is movable toward the working plate receptacle to draw the beads in the working plate toward the magnet, and wherein the first contact dispenser is configured to aspirate a first reagent from the reagent reservoir in the consumables area and to dispense the first reagent in the working plate.

In another implementation, the first contact dispenser is configured to aspirate the sample and the first reagent from the working plate, the mover is configured to move the working plate to the consumables area and to move the second working plate in the consumables area to the working plate receptacle in the first working area, and the first contact dispenser is configured to dispense the sample and the first reagent in the second working plate.

In another implementation, the mover is configured to move the second working plate from the first working area to the analyzer area.

In another implementation, the imaging system is configured to obtain image data of the portion of the first reagent and the sample to determine a concentration of the sample.

In another implementation, the apparatus includes a second assay bay disposed in parallel with the first assay bay, the second assay bay includes a second contact dispenser; a second working area includes a working plate receptacle adapted to receive a working plate; a thermocycler; and a magnet; and a second drawer. The second drawer includes a second consumables area adapted to receive a sample plate adapted to contain a sample, and a plurality of consumables for interacting with the sample in the working plate in the second working area, wherein the mover is operatively coupled to the second assay bay, wherein the second contact dispenser is linearly movable in the first direction between the consumables area and the second working area, such that the second contact dispenser is configured to (i) move the sample from the sample plate in the second consumables area to the working plate in the second working area, and (ii) move the plurality of consumables between the second consumables area and the working plate in the second working area, and wherein the mover is movable in the first direction and the second direction between the second assay bay and the common bay, such that the mover is configured to move the working plate from the second working area to the analyzer area for analysis by the imaging system.

In another implementation, the second workflow is performed simultaneously with the first workflow.

In another implementation, the apparatus includes a sequencer; and a plurality of fluidic lines fluidly coupling the common bay and the sequencer, such that the prepared samples automatically flow from the common bay to the sequencer.

In another implementation, the common bay includes a sipper assembly having a plurality of sippers. The sequencer includes a plurality of flowcells, and wherein the plurality of fluidic lines fluidly couple the plurality of sippers with the plurality of flowcells.

In another implementation, the contact dispenser includes a contact head configured to hold tips.

In another implementation, the drawer is linearly movable in the longitudinal direction relative to the working area between a loading position and an operating position, wherein when the drawer is in the loading position, the drawer is spaced a first distance from the working area, and when the drawer is in the operating position, the drawer is spaced a second distance from the working area, the second distance being less than the first distance.

In another implementation, the contact dispenser is not movable in a lateral direction perpendicular to the longitudinal direction.

In another implementation, the apparatus includes a movable stage operatively coupled to the contact dispenser for linearly moving the contact dispenser in the longitudinal direction.

In another implementation, the apparatus includes an actuator configured to actuate movement of the movable stage in the longitudinal direction.

In another implementation, the apparatus includes a door that is movable to enclose the thermocycler and the working plate receptacle.

In another implementation, the thermocycler is aligned with the working plate receptacle in the longitudinal direction.

In another implementation, the thermocycler is configured to amplify the sample within the working plate.

In another implementation, the consumables area is adapted to receive a lid for the working plate, and wherein the contact dispenser is configured to move the lid from the consumables area and to place the lid on the working plate.

In another implementation, the plurality of consumables include a tip tray including a first reusable tip and a second reusable tip, a second working plate, an index tray adapted to contain indexes, a bead tray adapted to contain beads, and a reagent reservoir adapted to contain a reagent.

In another implementation, the contact dispenser is configured to move the sample from the sample plate in the consumables area to the working plate in the working area using the first reusable tip and to move one or more indexes from the index tray in the consumables area to the working plate using the second reusable tip.

In another implementation, the contact dispenser is configured to aspirate the indexes from the index tray in the consumables area and to dispense the indexes in the working plate in the working area.

In another implementation, the contact dispenser is configured to aspirate the beads from the bead tray in the consumables area and to dispense the beads in the working plate in the working area.

In another implementation, the magnet is movable toward the working plate receptacle to draw the beads in the working plate toward the magnet, and wherein the contact dispenser is configured to aspirate a first reagent from the reagent reservoir in the consumables area and to dispense the first reagent in the working plate.

In another implementation, the contact dispenser is configured to aspirate the sample and the first reagent from the working plate, the mover is configured to move the working plate to the consumables area and to move the second working plate in the consumables area to the working plate receptacle in the working area, and the contact dispenser is configured to dispense the sample and the first reagent in the second working plate.

In another implementation, the apparatus includes an actuator to move the magnet relative to the plate receptacle.

In another implementation, the thermocycler is aligned with the plate receptacle.

In another implementation, the mover is to move the first plate from the consumables area to the plate receptacle.

In another implementation, the stage is to align the contact dispenser with the tip tray and the contact dispenser is to couple with the first tip from the tip tray, wherein the stage is to align the contact dispenser with the index tray and the contact dispenser is to aspirate the indexes from the index tray, wherein the stage is to align the contact dispenser with the first plate, and wherein the contact dispenser is to dispense the indexes into the well of the first plate.

In another implementation, the thermocycler is to amplify the sample within the well of the first plate.

In another implementation, the consumables area further includes a lid and wherein the mover is to move the lid from the consumables area and to place the lid on the first plate to cover the well of the first plate with the lid.

In another implementation, the mover is to move the lid from the first plate to the consumables area.

In another implementation, the stage is to align the contact dispenser with the bead tray and the contact dispenser is to aspirate the beads from the bead tray, wherein the stage is to align the contact dispenser with the first plate, and wherein the contact dispenser is to dispense the beads into the well of the first plate.

In another implementation, the stage is to align the contact dispenser with the first plate and the contact dispenser is to dispense a first reagent into the well of the first plate.

In another implementation, the stage is to align the contact dispenser with the tip tray and the contact dispenser is to place the first tip in the tip tray and the contact dispenser to couple with the second tip from the tip tray.

In another implementation, the actuator is to move the magnet toward the plate receptacle to draw the beads toward the magnet and wherein the stage is to align the contact dispenser with the first plate to allow the contact dispenser to aspirate the first reagent from the well.

In another implementation, the system includes a waste and wherein the contact dispenser is to dispense the first reagent into the waste.

In another implementation, the stage is to align the contact dispenser with the first plate and the contact dispenser is to dispense a second reagent into the well of the first plate.

In another implementation, the actuator is to move the magnet toward the plate receptacle to draw the beads toward the magnet, wherein the stage is to align the contact dispenser with the first plate to allow the contact dispenser to aspirate the second reagent and the sample from the well of the first plate, and wherein the stage is to align the contact dispenser with the second plate to allow the contact dispenser to dispense the second reagent and the sample into the well of the second plate.

In another implementation, the imaging system is to obtain image data of a portion of the second reagent and the sample and the system to determine a concentration of the sample.

In another implementation, the apparatus includes a second contact dispenser.

In another implementation, the stage is to align the second plate with the second contact dispenser and wherein the second contact dispenser is to dispense a diluent into the well of the second plate to dilute the sample based on the concentration of the sample determined.

In another implementation, the system includes a first working area includes the contact dispenser, the stage to move the contact dispenser, the plate receptacle, the magnet, and the thermocycler.

In another implementation, the system includes a second working area includes the mover, a second contact dispenser, a plate receptacle, and the analyzer includes the imaging system.

In another implementation, the system includes a loading area.

In another implementation, the loading area includes a sipper assembly.

In another implementation, the sipper assembly includes a sample sipper assembly.

In another implementation, the loading area includes a plate receptacle.

In another implementation, the loading area includes a stage to move the plate receptacle relative to the sipper assembly.

In another implementation, the apparatus includes a second system.

In another implementation, the second system is fluidly coupled to the system.

In another implementation, the second system includes a sequencing instrument.

In another implementation, the apparatus further includes an actuator to move the magnet relative to the second plate receptacle.

In another implementation, the apparatus further includes a door that is movable to enclose the reagent reservoir receptacle.

In another implementation, the apparatus further includes a gas source fluidly coupled to the regent reservoir.

In another implementation, the apparatus further including a valve to control a flow of gas from the gas source to the reagent reservoir receptacle.

In another implementation, the gas includes nitrogen.

In another implementation, the thermocycler is aligned with the second plate receptacle.

In another implementation, the thermocycler is carried by the stage.

In another implementation, the mover is to move the tip tray from the consumables area to the first plate receptacle, the mover is to move the first plate from the consumables area to the second plate receptacle, and the mover is to move the index tray from the consumables area to the third plate receptacle.

In another implementation, the stage is to align the contact dispenser with the tip tray and the contact dispenser is to couple with the first tip from the tip tray, the stage is to align the contact dispenser with the index tray and the contact dispenser is to aspirate the indexes from the index tray, the stage is to align the contact dispenser with the first plate, and the contact dispenser is to dispense the indexes into the well of the first plate.

In another implementation, the thermocycler is to amplify the sample within the well of the first plate.

In another implementation, the consumables area further includes a lid and the mover is to move the lid from the consumables area and to place the lid on the first plate to cover the well of the first plate with the lid.

In another implementation, the mover is to move the lid from the first plate to the consumables area.

In another implementation, the mover is to move the index tray from the third receptacle to the consumables area.

In another implementation, the mover is to move the bead tray from the consumables area to the third plate receptacle.

In another implementation, the stage is to align the contact dispenser with the bead tray and the contact dispenser is to aspirate the beads from the bead tray. The stage is to align the contact dispenser with the first plate, and the contact dispenser is to dispense the beads into the well of the first plate.

In another implementation, the stage is to align the non-contact dispenser with the first plate and the non-contact dispenser is to dispense a first reagent from the reagent reservoir into the well of the first plate.

In another implementation, the stage is to align the contact dispenser with the tip tray and the contact dispenser is to place the first tip in the tip tray. The contact dispenser to couple with the second tip from the tip tray.

In another implementation, the actuator is to move the magnet toward the second plate receptacle to draw the beads toward the magnet and the stage is to align the contact dispenser with the first plate to allow the contact dispenser to aspirate the first reagent from the well.

In another implementation, the system includes a waste and the contact dispenser is to dispense the first reagent into the waste.

In another implementation, the mover is to move the bead tray from the third plate receptacle to the consumables area.

In another implementation, the stage is to align the non-contact dispenser with the first plate and the non-contact dispenser is to dispense a second reagent from the reagent reservoir into the well of the first plate.

In another implementation, the mover is to move a second plate from the consumables area to the third plate receptacle.

In another implementation, the actuator is to move the magnet toward the second plate receptacle to draw the beads toward the magnet, the stage is to align the contact dispenser with the first plate to allow the contact dispenser to aspirate the second reagent and the sample from the well of the first plate, and the stage is to align the contact dispenser with the second plate to allow the contact dispenser to dispense the second reagent and the sample into the well of the second plate.

In another implementation, the stage is to align the contact dispenser with the tip tray and the contact dispenser is to place the second tip in the tip tray and to couple with the first tip from the tip tray.

In another implementation, the substrate of the analyzer area is carried by the stage and the substrate includes a pair of plates between which a gap is defined.

In another implementation, the substrate includes an inlet and an outlet in fluid communication with the gap.

In another implementation, the substrate further includes a seal positioned between the pair of plates and defines a channel between the inlet and the outlet.

In another implementation, the apparatus further including a waste reservoir fluidly coupled to the outlet of the substrate.

In another implementation, the stage is to align the contact dispenser with the second plate and the contact dispenser is to aspirate a portion of the second reagent and the sample from the well of the second plate and the stage is to align the contact dispenser with the inlet of the substrate to allow the contact dispenser to dispense the portion of the second reagent and the sample into the inlet of the substrate.

In another implementation, the imaging system is to obtain image data of the portion of the second reagent and the sample and the system to determine a concentration of the sample.

In another implementation, the stage is to align the second plate with the non-contact dispenser and the non-contact dispenser is to dispense a diluent into the well of the second plate to dilute the sample based on the concentration of the sample determined.

In another implementation, the thermocycler is positioned beneath the second plate receptacle.

In another implementation, the second plate receptacle includes a thermal block defining well receptacles and the thermocycler is positioned beneath the well receptacles.

In another implementation, the apparatus further includes a heat sink coupled to the thermocycler.

In another implementation, the apparatus includes a lid and an actuator, the actuator to move the lid relative to the plate receptacle to cover the plate receptacle.

In another implementation, the gaskets being compressed when the end of the pipettes are positioned within a pipette tip enables the gasket to form a coupling with the pipette tip.

In another implementation, the gaskets being relaxed when the end of the pipettes are positioned within a pipette tip enables the pipette tip to not be coupled with the pipette.

In another implementation, the guide includes outward facing channels and wherein the bar includes a first arm and a second arm that extend within the outward facing channels of the guide.

In another implementation, the bar includes a first arm and a second arm and wherein the pipette cam assembly includes guide bearings coupled to the guide; bar bearings coupled to the corresponding first arm and the second arm; a cam shaft including inner lobes and outer lobes, the inner lobes to engage the guide bearings and the outer lobes to engage the bar bearings.

In another implementation, the inner lobes of the cam shaft engaging the guide bearings move the body away from the guide and move the flanges of the pipettes toward the protrusions to compress the gaskets.

In another implementation, the inner lobes of the cam shaft engaging the guide bearings is a second position enable the guide to move toward the bar and move the flanges of the pipettes away from the protrusions to relax the gaskets.

In another implementation, the outer lobes of the cam shaft engaging the bar bearings move the bar toward the flanges of the pipettes to enable the bar to engage pipette tips carried by the pipettes and urge the pipette tips to be released from the pipette assembly.

In another implementation, one of the bar bearings face one of the guide bearings.

In another implementation, the apparatus includes bar springs positioned between the bar and the guide to urge the bar bearings toward the corresponding outer lobe.

In another implementation, the apparatus includes a guide spring positioned between the body and the guide to urge the body away from the guide.

In another implementation, the body includes a first side and a second side and wherein the cam shaft includes a first cam shaft portion and a second cam shaft portion, the first cam shaft portion coupled to the first side of the body and the second cam shaft portion coupled to the second side of the body.

In another implementation, the first cam shaft portion is spaced from the second cam shaft portion.

In another implementation, the apparatus includes a motor and a gear set, the gear set coupled to the cam shaft.

In another implementation, movement of the motor rotates the cam shaft.

In another implementation, the gear set includes first and second gears, first and second pinions, and a shaft coupling the first and second pinions.

In another implementation, the body includes a first side and a second side and wherein the first gear is coupled to the first side of the body and the inner lobe and the outer lobe on the first side of the body and the second gear is coupled to the second side of the body and the inner lobe and the outer lobe on the second side of the body.

In another implementation, the first side and the second side of the body define shaft apertures and the shaft is rotationally coupled within the shaft apertures.

In another implementation, the shaft is spaced from the pipettes.

In another implementation, the body includes a first side and a second side, and a wall defining a receptacle, the pipettes positioned within the receptacle.

In another implementation, the pipettes each include a barrel, the apparatus includes a plurality of pistons positioned and movable within the corresponding barrels.

In another implementation, the apparatus includes an actuator coupled to the pistons to move the pistons between a retracted position and an extended position within the barrels.

In another implementation, the actuator includes a ball screw.

In another implementation, the actuator includes a pair of linear rails and a lift, the lift coupled to the pistons and the linear rails and movable by the ball screw.

In another implementation, the lift is C-shaped and has ends. the apparatus includes carriages coupled to the corresponding ends of the lift and coupled to the linear rails.

In another implementation, the cam assembly includes inner cam plates, outer cam plates, cover bearings, inner slot bearings, cover follower bearings, and outer slot bearings. The inner cam plates are coupled to the sled and each defining an inner cam slot. The outer cam plates coupled to the base and each defining an outer cam slot. The cover bearings coupled to the cover and positioned to engage the stop wall. The inner slot bearings coupled to the cover and movably positioned within the inner cam slot. The cover follower bearings coupled to the cover follower and positioned to engage the rear wall of the sled. The outer slot bearings coupled to the cover follower and movably positioned within the outer cam slot.

In another implementation, the cover bearings are to engage the stop wall and cause the inner slot bearings to move within the inner cam slot and the cover bearings to move along the stop wall to move the cover toward the base to cover the plate receptacle.

In another implementation, the cover follower bearings are to engage the rear wall and cause the outer slot bearings to move within the outer cam slot and the cover follower bearings to move along the rear wall to move the cover follower toward the base.

In another implementation, the apparatus includes springs biasing the cover away from the cover follower.

In another implementation, the apparatus includes guide rods movably coupling the cover and the cover follower.

In another implementation, the cover includes blind bores and the cover follower includes through bores, the guide rods positioned within the corresponding blind bores of the cover and through bores of the cover follower.

In another implementation, the springs surround corresponding guide rods.

In another implementation, the cover defines cover bearing receptacles in which the cover bearings are positioned.

In another implementation, the cover follower defines cover follower bearing receptacles in which the cover follower bearings are positioned.

In another implementation, the apparatus includes linear rails coupled to the base and carriages coupled to the rear wall and coupled to the linear rails.

In another implementation, the stop wall includes extensions between which an opening is defined, the front wall sized to pass between the extensions.

In another implementation, the cover bearings are to engage the extension.

In another implementation, the apparatus includes a magnet and an actuator, wherein the actuator is to move the magnet relative to the plate receptacle.

In another implementation, the apparatus includes a central well plate support wall extending from the base and positioned between the first end wall and the second end wall of the small liquid reagent well plate receptacle.

In another implementation, the apparatus includes a central well plate support wall extending from the base and positioned between the first end wall and the second end wall of the large liquid reagent well plate receptacle.

In another implementation, the small liquid reagent well plate receptacle is positioned between the plate receptacle and the large liquid reagent well plate receptacle.

In another implementation, the large liquid reagent well plate receptacle is positioned between the small liquid reagent well plate receptacle and the dry well plate receptacle.

In another implementation, the dry well plate receptacle is positioned between the large liquid reagent well plate receptacle and the wider portion of the waste reservoir including the inlet.

In another implementation, the inlet includes a rectangular inlet to receive waste associated with a multiple-tip pipette.

In another implementation, the drawer further includes a tip receptacle.

In another implementation, the tip receptacle is positioned between the large liquid reagent well plate receptacle and the dry well plate receptacle.

In another implementation, the first end wall includes a keying notch.

In another implementation, the apparatus includes an impermeable barrier coupled to the ends of the reagent wells.

In another implementation, the apparatus includes a machine-readable code coupled to the panel.

In another implementation, the reagent well plate includes a small liquid reagent well plate.

In another implementation, the first end wall and the second end wall extend outwardly from the panel.

In another implementation, the panel is concave.

In another implementation, the panel is substantially flat when the first end wall and the second end wall are coupled to a reagent well plate receptacle.

In another implementation, each of the reagent wells includes a second annular collar longitudinally spaced from the annular collar. The panel positioned between the annular collar and the second annular collar of the corresponding reagent wells.

In another implementation, the panel includes a plurality of second reagent well receptacles having a different size than the reagent well receptacles. The second reagent well receptacles positioned between the second end wall and the reagent well receptacles.

In another implementation, the apparatus includes bulk reagent wells to be positioned within the second reagent well receptacles.

In another implementation, the apparatus includes an L-tab coupled to each of the bulk reagent wells.

In another implementation, the L-tab includes a first leg coupled to the bulk reagent well and a second leg extending from the first leg at an angle that corresponds to an angle of the second end wall.

In another implementation, the L-tab is positioned within a dimensional envelope of the reagent well plate.

In another implementation, the second end wall includes a first wall section and a second wall section that are coupled to form a recess.

In another implementation, the L-tab is positioned within a dimensional envelope of the recess.

In another implementation, the first end wall includes a pair of first end wall portions and a pair of the male snap-fit components. Each first end wall portion including one of the male snap-fit components.

In another implementation, the second end wall includes a pair of second end wall portions and a pair of the male snap-fit components. Each second end wall portion including one of the male snap-fit components.

In another implementation, the apparatus includes a plurality of impermeable barriers. Each reagent well covered by one of the impermeable barriers.

In another implementation, each of the reagent wells include a second annular collar longitudinally spaced from the annular collar and a snap-fit connection is formed between the plate, the annular collar, and the second annular collar.

In another implementation, the male snap fit components include a tapered tab.

In another implementation, the cutout and the recess of an adjacent plate form an opening.

In another implementation, the end walls include the handle form a dog-bone shape.

In another implementation, the rectangular wall and the panel form a step.

In another implementation, the rectangular wall includes an end that forms an opening that is sized to receive the step of an adjacent well plate.

In another implementation, the panel includes a plurality of rows of the reagent well receptacles.

In another implementation, the panel includes a row of the reagent well receptacles.

In another implementation, the apparatus includes a lid including a lid rectangular wall and a lid panel. The lid rectangular wall includes lid end walls and lid side walls. The lid end walls each include a lid cutout that forms a lid handle that extend between the lid side walls.

In another implementation, the lid cutout and an adjacent plate form an opening.

In another implementation, the lid rectangular wall and the lid panel form a lid step.

In another implementation, the lid rectangular wall includes an end that forms a lid opening that is sized to receive the lid step of an adjacent lid.

In another implementation, the rectangular wall and the panel form a step and wherein the lid opening is sized to receive the step of an adjacent well plate.

In another implementation, the apparatus includes fluidic lines fluidly coupling the sippers of the sample sipper assembly and the sequencing instrument.

In another implementation, the apparatus includes a loading area including the sample sipper assembly and the plate receptacle.

In another implementation, the loading area includes a stage to move the plate receptacle relative to the sample sipper assembly.

In another implementation, the gaskets being compressed when the end of the pipettes are positioned within a pipette tip enables the gasket to form a coupling with the pipette tip.

In another implementation, the gaskets being relaxed when the end of the pipettes are positioned within a pipette tip enables the pipette tip to not be coupled with the pipette.

In another implementation, the guide includes outward facing channels and wherein the bar includes a first arm and a second arm that extend within the outward facing channels of the guide.

In another implementation, the bar includes a first arm and a second arm and wherein the pipette cam assembly includes guide bearings coupled to the guide; bar bearings coupled to the corresponding first arm and the second arm; a cam shaft includes inner lobes and outer lobes, the inner lobes to engage the guide bearings and the outer lobes to engage the bar bearings.

In another implementation, the inner lobes of the cam shaft engaging the guide bearings move the body away from the guide and move the flanges of the pipettes toward the protrusions to compress the gaskets.

In another implementation, the inner lobes of the cam shaft engaging the guide bearings in a second position enable the guide to move toward the bar and move the flanges of the pipettes away from the protrusions to relax the gaskets.

In another implementation, the outer lobes of the cam shaft engaging the bar bearings move the bar toward the flanges of the pipettes to enable the bar to engage pipette tips carried by the pipettes and urge the pipette tips to be released from the pipette assembly.

In another implementation, one of the bar bearings face one of the guide bearings.

In another implementation, the apparatus includes bar springs positioned between the bar and the guide to urge the bar bearings toward the corresponding outer lobe.

In another implementation, the apparatus includes a guide spring positioned between the body and the guide to urge the body away from the guide.

In another implementation, the body includes a first side and a second side and wherein the cam shaft includes a first cam shaft portion and a second cam shaft portion. The first cam shaft portion coupled to the first side of the body and the second cam shaft portion coupled to the second side of the body.

In another implementation, the first cam shaft portion is spaced from the second cam shaft portion.

In another implementation, the apparatus includes a motor and a gear set, the gear set coupled to the cam shaft.

In another implementation, movement of the motor rotates the cam shaft.

In another implementation, the gear set includes first and second gears, first and second pinions, and a shaft coupling the first and second pinions.

In another implementation, the body includes a first side and a second side and wherein the first gear is coupled to the first side of the body and the inner lobe and the outer lobe on the first side of the body and the second gear is coupled to the second side of the body and the inner lobe and the outer lobe on the second side of the body.

In another implementation, the first side and the second side of the body define shaft apertures and the shaft is rotationally coupled within the shaft apertures.

In another implementation, the shaft is spaced from the pipettes.

In another implementation, the body includes a first side and a second side, and a wall defining a receptacle, the pipettes positioned within the receptacle.

In another implementation, the pipettes each include a barrel. the apparatus includes a plurality of pistons positioned and movable within the corresponding barrels.

In another implementation, the apparatus includes an actuator coupled to the pistons to move the pistons between a retracted position and an extended position within the barrels.

In another implementation, the actuator includes a ball screw.

In another implementation, the actuator includes a pair of linear rails and a lift, the lift coupled to the pistons and the linear rails and movable by the ball screw.

In another implementation, the lift is C-shaped and has ends. the apparatus includes carriages coupled to the corresponding ends of the lift and coupled to the linear rails.

In another implementation, the apparatus includes a sample sipper assembly includes a plurality of sippers and an actuator to move the sippers relative to a well plate, the sample sipper assembly associated with transferring the library of samples to a sequencing system.

In another implementation, the cam assembly includes inner cam plates coupled to the sled and each defining an inner cam slot; outer cam plates coupled to the base and each defining an outer cam slot; cover bearings coupled to the cover and positioned to engage the stop wall; inner slot bearings coupled to the cover and movably positioned within the inner cam slot; cover follower bearings coupled to the cover follower and positioned to engage the rear wall of the sled; and outer slot bearings coupled to the cover follower and movably positioned within the outer cam slot.

In another implementation, the cover bearings are to engage the stop wall and cause the inner slot bearings to move within the inner cam slot and the cover bearings to move along the stop wall to move the cover toward the base to cover the plate receptacle.

In another implementation, the cover follower bearings are to engage the rear wall and cause the outer slot bearings to move within the outer cam slot and the cover follower bearings to move along the rear wall to move the cover follower toward the base.

In another implementation, the apparatus includes springs biasing the cover away from the cover follower.

In another implementation, the apparatus includes guide rods movably coupling the cover and the cover follower.

In another implementation, the cover includes blind bores and the cover follower includes through bores, the guide rods positioned within the corresponding blind bores of the cover and through bores of the cover follower.

In another implementation, the springs surround corresponding guide rods.

In another implementation, the cover defines cover bearing receptacles in which the cover bearings are positioned.

In another implementation, the cover follower defines cover follower bearing receptacles in which the cover follower bearings are positioned.

In another implementation, the apparatus includes linear rails coupled to the base and carriages coupled to the rear wall and coupled to the linear rails.

In another implementation, the stop wall includes extensions between which an opening is defined, the front wall sized to pass between the extensions.

In another implementation, the cover bearings are to engage the extension.

In another implementation, the apparatus includes an actuator. The actuator is to move the magnet relative to the plate receptacle.

In another implementation, the apparatus includes a central well plate support wall extending from the base and positioned between the first end wall and the second end wall of the small liquid reagent well plate receptacle.

In another implementation, the apparatus includes a central well plate support wall extending from the base and positioned between the first end wall and the second end wall of the large liquid reagent well plate receptacle

In another implementation, wherein the small liquid reagent well plate receptacle is positioned between the plate receptacle and the large liquid reagent well plate receptacle.

In another implementation, wherein the large liquid reagent well plate receptacle is positioned between the small liquid reagent well plate receptacle and the dry well plate receptacle.

In another implementation, wherein the dry well plate receptacle is positioned between the large liquid reagent well plate receptacle and the wider portion of the waste reservoir including the inlet.

In another implementation, wherein the inlet includes a rectangular inlet to receive waste associated with a multiple-tip pipette.

In another implementation, wherein the drawer further includes a tip receptacle.

In another implementation, wherein the tip receptacle is positioned between the large liquid reagent well plate receptacle and the dry well plate receptacle.

In another implementation, wherein to communicate with the sequencer, the one or more processors are configured to: transmit, to the sequencer via the communication interface, identification information for the library of samples.

In another implementation, to communicate with the sequencer, the one or more processors are configured to: transmit, to the sequencer via the communication interface, one or more run parameters for sequencing the library of samples.

In another implementation, the one or more processors are configured to: transmit, to the sequencer via the communication interface, an indication of a preparation status of the library of samples.

In another implementation, to communicate with the sequencer, the one or more processors are configured to: transmit, to the sequencer via the communication interface, an instruction indicating a particular lane of a flow cell for the sequencer to sequence a particular sample of the library of samples.

In another implementation, to communicate with the sequencer, the one or more processors are configured to: receive, via the communication interface, status information from the sequencer.

In another implementation, to communicate with the sequencer, the one or more processors are configured to: receive, via the communication interface, an indication from the sequencer that the sequency is ready to receive the library of samples.

In another implementation, the library preparation system includes a fluidic line configured to be coupled to the library preparation system and the sequencer. The library preparation system transmits the library of samples to the sequencer via the fluidic line in response to receiving the indication that the sequencer is ready to receive the library of samples.

In another implementation, the working area includes a working plate; and a thermocycler.

In another implementation, the library preparation system includes a contact dispenser; and a drawer including a consumables area adapted to receive a sample plate adapted to contain a sample, and a plurality of consumables for interacting with the sample. The contact dispenser is configured to (i) move the sample plate in the consumables area to the working plate in the working area, and (ii) move the plurality of consumables.

In another implementation, the communication interface includes a wired communication link attached to the library preparation system and the sequencer.

In another implementation, communicating with the sequencer includes transmitting, by the one or more processors to the sequencer via the communication interface, identification information for the library of samples.

In another implementation, communicating with the sequencer includes transmitting, by the one or more processors to the sequencer via the communication interface, one or more run parameters for sequencing the library of samples.

In another implementation, communicating with the sequencer includes transmitting, by the one or more processors to the sequencer via the communication interface, an indication of a preparation status of the library of samples.

In another implementation, communicating with the sequencer includes transmitting, by the one or more processors to the sequencer via the communication interface, an instruction indicating a particular lane of a flow cell for the sequencer to sequence a particular sample of the library of samples.

In another implementation, communicating with the sequencer includes receiving, at the one or more processors via the communication interface, status information from the sequencer.

In another implementation, communicating with the sequencer includes receiving, at the one or more processors via the communication interface, an indication from the sequencer that the sequency is ready to receive the library of samples.

In another implementation, the method includes transmitting, by the library preparation system via a fluidic line coupled to the library preparation system and the sequencer, the library of samples to the sequencer in response to receiving the indication that the sequency is ready to receive the library of samples.

In another implementation, the communication interface includes a wired communication link attached to the library preparation system and the sequencer.

In another implementation, each assay bay includes an assay bay plate receptacle, a pipette assembly, a thermocycler and a magnet to perform the amplification processes and cleanup processes associated with preparing the library of samples for sequencing.

In another implementation, the small liquid reagent well plate includes a first end wall including a male snap-fit component, a second end wall including a male snap-fit component, a panel coupled to and extending between the first end wall and the second end wall, and a plurality of reagent wells positioned within the reagent well receptacles.

In another implementation, the small liquid reagent well plate receptacle includes a base, a first end wall and having an inward extending lip that forms a first groove with the base, and a second end wall coupled to the base and having an inward extending lip that forms a second groove with the base and including a key.

In another implementation, receiving the small liquid reagent well plate by the small liquid reagent well plate receptacle on the platform of the drawer includes a keying notch of the small liquid reagent well plate receiving a key of the small liquid reagent well plate receptacle.

In another implementation, the large liquid reagent well plate includes a first end wall including a male snap-fit component, a second end wall coupled to the base and including a male snap-fit component, a panel coupled to and extending between the first end wall and the second end wall, and a plurality of reagent wells positioned within the reagent well receptacles.

In another implementation, the large liquid reagent well plate receptacle includes a base, a first end wall coupled to the base and having an inward extending lip that forms a first groove with the base, and a second end wall coupled to the base and having an inward extending lip that forms a second groove with the base and including a key.

In another implementation, receiving the large liquid reagent well plate by the large liquid reagent well plate receptacle on the platform of the drawer includes a keying notch of the large liquid reagent well plate receiving a key of the large liquid reagent well plate receptacle.

In another implementation, receiving the small liquid reagent well plate by the small liquid reagent well plate receptacle on the platform of the drawer includes supporting a panel of the small liquid reagent well plate using a central well plate support wall extending from the base of the small liquid reagent well plate receptacle and positioned between a first end wall and a second end wall of the small liquid reagent well plate receptacle.

In another implementation, supporting the panel of the small liquid reagent well plate includes supporting the panel using a plurality of the central well plate support walls of the small liquid reagent well plate receptacle.

In another implementation, the panel of the small liquid reagent well plate being supported by the central well plate of the small liquid reagent well plate receptacle enables the panel of the small liquid reagent well plate to be substantially flat.

In another implementation, a coupling between the small liquid reagent well plate and the small liquid reagent well plate receptacle and enables the panel of the small liquid reagent well plate to be substantially flat.

In another implementation, receiving the large liquid reagent well plate by the large liquid reagent well plate receptacle on the platform of the drawer includes supporting a panel of the large liquid reagent well plate using a central well plate support wall extending from the base of the small liquid reagent well plate receptacle and positioned between a first end wall and a second end wall of the small liquid reagent well plate receptacle.

In another implementation, supporting the panel of the large liquid reagent well plate includes supporting the panel using a plurality of the central well plate support walls of the large liquid reagent well plate receptacle.

In another implementation, the panel of the large liquid reagent well plate being supported by the central well plate support of the large liquid reagent well plate receptacle enables the panel of the large liquid reagent well plate to be substantially flat.

In another implementation, a coupling between the large liquid reagent well plate and the large liquid reagent well plate receptacle enables the panel of the large liquid reagent well plate to be substantially flat.

In another implementation, the large liquid reagent well plate includes reagent well receptacles and second reagent well receptacles, the second reagent well receptacles having a different size than the reagent well receptacles, bulk reagent wells positioned within the second reagent well receptacles.

In another implementation, the method includes performing library pooling processes at the common bay.

In another implementation, the method includes performing at least one of denaturing processes or diluting processes at the common bay.

In another implementation, the well plate includes a rectangular wall including end walls and side walls, the end walls each include the cutout and a recess that form a handle that extends between the side walls; a panel coupled to and extending between the rectangular wall, the panel defining a plurality of reagent well receptacles and including a top surface, the end walls and the side walls extending outwardly from the panel; and a plurality of reagent wells including an end having an annular collar, the reagent wells positioned within the reagent well receptacles and the annular collars engaging the top surface.

In another implementation, the method includes removing a lid from the well plate at the plate receptacle using the gripper.

In another implementation, removing the lid includes positioning the gripper in a lid cutout that that forms a lid handle that extends between lid side walls of the lid and moving the lid away from the well plate.

In another implementation, the method includes moving a well plate from a consumables area to the assay bay using the gripper.

In another implementation, moving the well plate includes positioning the gripper in a cutout of the well plate that forms a handle of the well plate and moving the well plate away from the consumables area.

In another implementation, the method includes moving the well plate from the consumables area includes moving the well plate from a stack of well plate at the consumables area.

In another implementation, compressing the gaskets includes moving a body of the pipette assembly away from a guide of the pipette assembly and moving flanges of the pipettes toward protrusions of the guide to compress the gaskets.

In another implementation, moving the body of the pipette assembly away from the guide of the pipette assembly and moving the flanges of the pipettes toward protrusions of the guide to compress the gaskets includes using a pipette cam assembly.

In another implementation, the method includes moving a body of the pipette assembly away from a guide of the pipette assembly and moving flanges of the pipettes toward protrusions of the guide to compress the gaskets by engaging inner lobes of a cam shaft of a pipette cam assembly of the pipette assembly with guide bearings of the pipette assembly.

In another implementation, the method includes relaxing the gaskets to enable the pipette tips to be uncoupled from the pipette assembly.

In another implementation, relaxing the gaskets includes moving a body of the pipette assembly toward a guide of the pipette assembly and moving flanges of the pipettes away from protrusions of the guide to relax the gaskets.

In another implementation, moving the guide of the pipette assembly toward the bar of the pipette assembly and moving the flanges of the pipettes away from the protrusions of the guide to relax the gasket includes using a pipette cam assembly.

In another implementation, the method includes enabling flanges of the pipettes to move away from protrusions of a guide of the pipette assembly to relax the gaskets by positioning inner lobes of a cam shaft of a pipette cam assembly of the pipette assembly in a second position relative to guide bearings of the pipette assembly to enable the body to move toward a guide of the pipette assembly and enable the flanges of the pipettes to move away from the protrusions of the guide to relax the gaskets.

In another implementation, the method includes releasing pipette tips from the pipette assembly.

In another implementation, releasing the pipette tips from the pipette assemblies includes moving the bar toward the flanges of the pipettes and engaging the pipette tips with the bar and urging the pipette tips to be released from the pipette assembly.

In another implementation, the method includes moving pistons within barrels of the pipettes between a retracted position and an extended position using an actuator.

In another implementation, the method includes performing amplification processes on a sample in a well plate positioned on the plate receptacle.

In another implementation, the method includes performing cleanup processes on the sample in the well plate positioned on the plate receptacle.

In another implementation, performing clean up processes includes moving a magnet toward the well plate on the plate receptacle.

In another implementation, the cover bearings engaging the stop wall causes the inner slot bearings to move within the inner cam slot and the cover bearings to move along the stop wall to move the cover toward the base to cover the plate receptacle.

In another implementation, the cover follower bearings engaging the rear wall causes the outer slot bearings to move within the outer cam slot and the cover follower bearings to move along the rear wall to move the cover follower toward the base.

In another implementation, the method includes biasing the cover away from the cover follower.

In another implementation, archiving the library of samples includes sealing the samples.

In another implementation, archiving the library of samples includes freezing the samples.

In another implementation, the library pooling processes includes preparing an equimolar pool using the samples based on the quantification values determined by the library quantification processes.

In another implementation, the method includes archiving a remaining portion the library of samples.

In another implementation, archiving the library of samples includes sealing the samples.

In another implementation, archiving the library of samples includes freezing the samples.

In another implementation, the preparing the sequence ready pool of samples at the common bay includes performing library quantification processes and library pooling processes on the samples.

In another implementation, the preparing the sequence ready pool of samples at the common bay includes performing denaturing processes on the samples.

In another implementation, the preparing the sequence ready pool of samples at the common bay includes performing diluting processes on the samples.

In another implementation, transferring the sequence ready pool of samples to the sequencer includes transferring the library of samples to a sequencing system using a sample sipper assembly.

In another implementation, the preparing the sequence ready pool of samples at the common bay includes performing library quantification processes and library pooling processes on the samples.

In another implementation, the preparing the sequence ready pool of samples at the common bay includes performing denaturing processes on the samples.

In another implementation, the preparing the sequence ready pool of samples at the common bay includes performing diluting processes on the samples.

In another implementation, each flow cell port is coupled to a corresponding port of the flow cell interface and associated with one of the plurality of channels of the flow cell via a flow cell fluidic line.

In another implementation, the sample valves are movable to fluidically couple a sipper of the library preparation system and sample port of the sequencing instrument and a corresponding outlet of a channel of the plurality of channels of the flow cell.

In another implementation, the sample valves are movable to fluidically decouple a sipper of the library preparation system and sample port of the sequencing instrument and a corresponding outlet of a channel of the plurality of channels of the flow cell.

In another implementation, the sample valves are operable to individually load each channel of the plurality of channels of the flow cell.

In another implementation, the sequencing instrument includes a plurality of pumps and wherein the body of the sample loading assembly further defines a plurality of pump ports, each pump port being coupled to one of the pumps of the plurality of In another implementation, each sample valve is operable to fluidly couple a sipper of the sample sipper assembly of the library preparation system and a corresponding pump of the plurality of pumps of the sequencing instrument and to fluidly couple a pump of the plurality of pumps and a corresponding channel of the plurality of channels of the flow cell.

In another implementation, the pumps are operable to individually control fluid flow for each channel of the plurality of channels of the flow cell.

In another implementation, the outlets of the plurality of channels are fluidly couplable to a waste reservoir.

In another implementation, the sequencing instrument includes a pump manifold assembly including a plurality of pumps and wherein the pump manifold assembly is to fluidly couple the outlets of the plurality of channels to the waste reservoir.

In another implementation, the sequencing instrument includes a pump manifold assembly includes the pumps and a cache. The sequencing instrument includes a bypass valve and a bypass fluidic line coupling the bypass valve and the cache.

In another implementation, the sequencing instrument further includes a shared line valve, a plurality of dedicated reagent fluidic lines, and a shared reagent fluidic line, the shared reagent fluidic line coupling the shared line valve and the central valve and adapted to flow one or more reagents to the flow cell, each dedicated reagent fluidic line coupling the bypass fluidic line and the central valve and adapted to flow one or more reagents toward the flow cell.

In another implementation, the pump manifold assembly carries a plurality of pump valves and a cache valve and includes a plurality of pump-channel fluidic lines, a plurality of pump fluidic lines, a shared fluidic line, a cache fluidic line, and a primary waste fluidic line, the cache fluidic line being coupled to and between the cache and the cache valve, each pump valve being coupled to a corresponding pump-channel fluidic line, a corresponding pump fluidic line, and the shared fluidic line, the cache valve being coupled to the cache fluidic line, the primary waste fluidic line, and the shared fluidic line.

In another implementation, the pump valves and the pumps are operable to individually control fluid flow for each channel of the plurality of channels of the flow cell and the pump valves, the cache valve, and the pumps are operable to control fluid flow between the bypass fluidic line and the shared fluidic line.

In another implementation, the pump valves, the cache valve, and the pumps are operable to control fluid flow between the shared fluidic line and the primary waste fluidic line.

In another implementation, the sequencing instrument includes a pump manifold assembly having a plurality of pumps including the pump and a plurality of pump valves, wherein each pump and a corresponding pump valve are operable to individually control the flow of the sample of interest between each sipper of the sample sipper assembly of the library preparation system and a corresponding channel of the flow cell.

In another implementation, the sequencing instrument includes a sample loading assembly having a plurality of sample valves, wherein each sample valve is operable to individually load each channel of the plurality of channels of the flow cell with the sample of interest.

In another implementation, the apparatus includes a flow cell assembly including the flow cell having a plurality of channels and a flow cell manifold, wherein the flow cell manifold includes an inlet, a plurality of fluidic lines, and a plurality of outlets, wherein each outlet of the flow cell manifold is coupled to a corresponding channel of the flow cell.

In another implementation, the method includes; moving a second sample valve of the one or more sample valves to a first position to fluidically couple a second sipper of the sipper manifold assembly of the library preparation system with a second pump of the sequencing instrument; drawing a second sample of interest through the second sipper of the sipper manifold assembly from the library preparation system toward the second pump of the sequencing instrument; moving the second sample valve to a second position to fluidically couple the second pump and a second channel of the flow cell coupled to the flow cell interface of the sequencing instrument; and pumping the second sample of interest into the second channel of the flow cell through an outlet of the second channel.

In another implementation, the method includes fluidically coupling a reagent reservoir with an inlet of the channel of the flow cell.

In another implementation, pumping the first sample of interest from the first sample reservoir into the channel of the flow cell includes moving the first sample of interest from a sample cartridge at the library preparation system using the sipper of the sipper manifold assembly to a corresponding sample port of a sample loading assembly of the sequencing instrument, out of an associated pump port of the sample loading assembly, and into a pump-channel fluidic line of a pump manifold assembly of the sequencing instrument, and moving the first sample of interest from the pump-channel fluidic line, through the associated pump port, and through a corresponding flow cell port of the sample loading assembly, each flow cell port being coupled to a corresponding port of the flow cell interface and associated with one of the channels of the plurality of channels of the flow cell.

In another implementation, moving the first sample valve of the one or more sample valves to the first position includes fluidically coupling a sample port of a sample loading assembly of the sequencing instrument and the sipper of the sample sipper assembly and a corresponding pump of the sequencing instrument and wherein moving the first sample valve of the one or more sample valves to the second position includes fluidically coupling the corresponding pump and the channel of the plurality of channels of the flow cell.

In another implementation, the method includes operating one or more of a plurality of pumps of the sequencing instrument to individually control fluid flow for each channel of the plurality of channels of the flow cell.

In another implementation, the method includes flowing the first sample of interest out of the first channel of the flow cell and into an auxiliary waste fluidic line of the sequencing instrument, the auxiliary waste fluidic line being upstream of the flow cell and fluidically coupled to a central valve and a waste reservoir of the sequencing instrument.

In another implementation, an inlet of the first channel is fluidically connected to a waste reservoir via a central valve when the central valve is in a first position, the sequencing instrument including the waste reservoir and the central valve.

In another implementation, the method includes moving the central valve to a second position to fluidically couple a reagent reservoir with the channel and a second channel of the flow cell; and pumping a first volume of reagent through the first channel and into the waste reservoir.

In another implementation, further comprising flowing the lag buffer into the channels of the flow cell before the samples of interest.

In another implementation, the method includes drawing an air bubble into the fluidic lines between the lead buffer and the samples of interest.

In another implementation, the method includes drawing an air bubble into the fluidic lines between the lag buffer and the samples of interest.

In another implementation, the method includes flowing a disinfectant through the fluidic lines.

In another implementation, the disinfectant includes bleach.

In another implementation, the lead buffer and the lag buffer comprise buffer.

It should be appreciated that all combinations of the foregoing concepts and additional concepts discussed in greater detail below (provided such concepts are not mutually inconsistent) are contemplated as being part of the subject matter disclosed herein and/or may be combined to achieve the particular benefits of a particular aspect described herein. In particular, all combinations of claimed subject matter appearing at the end of this disclosure are contemplated as being part of the subject matter disclosed herein.

### BRIEF DESCRIPTION OF THE DRAWINGS

FIG. 1 illustrates a schematic diagram of an implementation of a system in accordance with the teachings of this disclosure.

FIG. 2 is a schematic implementation of another system that can be used to implement the system of FIG. 1.

FIG. 3 is an isometric view of implementation of another system that can be used to implement the system of FIG. 1.

FIG. 4 is a detailed isometric view of the system of FIG. 3 showing the lid removed from one of the working areas and the lid being shown in another one of the working areas.

FIG. 5 is another detailed isometric view of the system of FIG. 3.

FIG. 6 is another detailed isometric view of the system of FIG. 3 showing the first working area, the actuator, the lid, and the contact dispenser.

FIG. 7 is another detailed isometric view of the system of FIG. 3 showing two of the first working areas and two of the contact dispensers.

FIG. 8 is a detailed isometric view of the system of FIG. 3 showing the first working area and the first plate receptacle.

FIG. 9 is a detailed isometric view of the system of FIG. 3 showing the second working area and a portion of one of the first working areas.

FIG. 10 is a detailed isometric view of the system of FIG. 3 showing the loading area including the sipper assembly and the stage that moves the plate receptacle relative to the sipper assembly.

FIG. 11 is a detailed isometric view of the system of FIG. 3 showing the loading area including the sipper assembly with the housing of the sipper assembly removed to show the sippers.

FIG. 12 is a front view of another implementation of a system that can be used to implement the system of FIG. 1.

FIG. 13 is a top plan view of the system of FIG. 12.

FIG. 14 is an isometric view of one of the consumables areas and one of the first working areas of the system of FIG. 12.

FIG. 15 is an isometric view of one of the consumables areas implemented by a drawer shown in the extended position.

FIG. 16 is top plan view one of the consumables areas and one of the first working areas of the system of FIG. 12.

FIG. 17 is top plan view one of the consumables areas of the system of FIG. 12.

FIG. 18 is an isometric view of the contact dispenser, the mover, and the stage of the system of FIG. 12.

FIG. 19 is a top plan view of the second working area and the loading area of the system of FIG. 12.

FIG. 20 is an isometric view of an implementation of another system that can be used to implement the system of FIG. 1.

FIG. 21 is a detailed isometric view of the consumables area, the first working area, and the second working area of the system of FIG. 20.

FIG. 22 is a detailed isometric view of the consumables area, the first working area, the second working area, and the loading area of the system of FIG. 20.

FIG. 23 illustrates a schematic diagram of an implementation of another system in accordance with the teachings of this disclosure.

FIG. 24A illustrates a schematic diagram of an implementation of another system in accordance with the teachings of this disclosure.

FIG. 24B is a schematic illustration of an implementation of a portion of the pump manifold assembly for use with the system of FIG. 24A.

FIGS. 24C-24F show the process of loading one or more sample of interest from a library preparation system and loading those samples of interest into a flow cell of a sequencing instrument.

FIG. 25 illustrates a schematic implementation of a sipper assembly of a first system and a second system carrying a plurality of flow cells.

FIG. 26 is a schematic implementation of another system that can be used to implement the system of FIG. 1.

FIGS. 27-30 are workflows that can be performed using the teachings of this disclosure.

FIG. 31 illustrates a schematic implementation of a sipper assembly of a first system and a second system carrying a plurality of flow cells.

FIG. 32 is an of another system that can be used to implement the system of FIG. 1.

FIG. 33 is a plan view of the system of FIG. 32.

FIG. 34 is a front view of the system of FIG. 32.

FIG. 35 is an isometric view of one of the assay bays of the system 3300 of FIG. 32.

FIG. 36 is a top view of the assay bay of FIG. 35 including the pipette assembly, the thermocycler, the magnet, and the drawer that are used to perform amplification processes and cleanup processes associated with preparing a library of samples for sequencing.

FIG. 37 is an isometric view of a portion of the example pipette assembly of FIG. 36.

FIG. 38 is an isometric view of the pipette assembly of FIG. 37 with the guide 3348 removed.

FIG. 39 is a cross-sectional front view of the pipette assembly of FIG. 38.

FIG. 40 is a rear isometric view of the pipette assembly of FIG. 38.

FIG. 41 is a front view of the pipette assembly of FIG. 38 including a printed circuit board assembly.

FIG. 42 is a side view of the pipette assembly of FIG. 38 showing the end of the pipette inserted into a pipette tip.

FIG. 43 is a side view of the pipette assembly of FIG. 38 showing the cam shaft rotated 90 relative to the position of the cam shaft of FIG. 42.

FIG. 44 is a side view of the pipette assembly of FIG. 38 showing the cam shaft rotated 90 relative to the position of the cam shaft of FIG. 43.

FIG. 45 is a side view of the pipette assembly of FIG. 38 showing the cam shaft rotated 90 relative to the position of the cam shaft of FIG. 44.

FIG. 46 is an isometric view of the thermocycler of one of the assay bays of FIG. 32.

FIG. 47 is an expanded isometric view of the thermocycler of FIG. 46.

FIG. 48 is an isometric view of the thermocycler of FIG. 46 with the inner cam plates, the outer cam plates, the sled, and the springs omitted.

FIG. 49 is a side view of the thermocycler with the cover assembly in the rear position and not covering the plate.

FIG. 50 is a side view of the thermocycler with the cover assembly in the forward and lowered position covering the plate.

FIG. 51 is an isometric view of the drawer of one of the assay bays of FIG. 32.

FIG. 52 is an isometric view of the drawer of FIG. 51 with the consumables removed but including the dry well plate receptacle and the waste reservoir.

FIG. 53 is an isometric view of the drawer of FIG. 51 with the consumables and the dry well plate receptacle removed.

FIG. 54 is a top view of the drawer of FIG. 51 with the consumables, the dry well plate receptacle, and the waste reservoir included.

FIG. 55 is a side view of the drawer of FIG. 51 with the consumables, the dry well plate receptacle, and the waste reservoir included.

FIG. 56 is an isometric view of a reagent well plate including a first end wall, a second end wall, and a panel that be used with the system of FIG. 32.

FIG. 57 is a side view of the reagent well plate of FIG. 56.

FIG. 58 is an isometric view of one of the reagent wells of the reagent well plate of FIG. 56.

FIG. 59 is an isometric view of another reagent well plate including a first end wall, a second end wall, and a panel.

FIG. 60 is a side view of the reagent well plate of FIG. 59.

FIG. 61 is a side view of the bulk reagent well of FIG. 60.

FIG. 62 is an isometric view of the bulk reagent well of FIG. 60.

FIG. 63 is an isometric view of another reagent well plate including a first end wall, a second end wall, and a panel that be used with the system of FIG. 32.

FIG. 64 is a side view of the reagent well plate of FIG. 63.

FIG. 65 is an isometric view of the reagent well of the reagent well plate of FIG. 63.

FIG. 66 is an isometric view of a well plate that can be used with the system of FIG. 32.

FIG. 67 is an isometric view of a stack of well plates of FIG. 66.

FIG. 68 is a cross-sectional end view of a stack of the well plates of FIG. 66.

FIG. 69 is a top view of the well plate of FIG. 66.

FIG. 70 is a side view of the well plate of FIG. 66.

FIG. 71 is an isometric view of a stack of other well plates that can be used with the system of FIG. 32.

FIG. 72 is a bottom isometric view of the well plate of FIG. 71.

FIG. 73 is a cross-sectional end view of a stack of the well plates of FIG. 71.

FIG. 74 is an isometric view of a stack of lids that can be used to cover the well plates of FIG. 66.

FIG. 75 is an isometric view of a lid that can be used to cover the well plates of FIG. 71.

FIG. 76 is top plan view of an example sample cartridge including a plurality of wells that can be used with any of the disclosed implementations.

FIG. 77 is top plan view of an example reagent cartridge including a plurality of wells that can be used with any of the disclosed implementations.

FIG. 78 is a plan view of an example system that be used to implement the system of FIG. 1.

FIG. 79 is a front isometric view of an implementation of the system of FIG. 78.

FIG. 80 is a rear isometric view of an implementation of the system of FIG. 78.

FIG. 81 is an isometric view of one of the assay bays of the system 5050 of FIG. 79.

FIG. 82 is an isometric view of the assay bay of FIG. 81 with the drawer partially removed.

FIG. 83 is an isometric view of an example implementation of an assay bay including an alternative pipette assembly that can be used to implement system of FIG. 1 and/or the system of FIG. 32.

FIG. 84 is a side view of the drawer of the assay bay of FIG. 83.

FIG. 85 is a plan view of an example system that be used to implement the system of FIG. 1.

### DETAILED DESCRIPTION

Although the following text discloses a detailed description of implementations of methods, apparatuses and/or articles of manufacture, it should be understood that the legal scope of the property right is defined by the words of the claims set forth at the end of this patent. Accordingly, the following detailed description is to be construed as examples only and does not describe every possible implementation, as describing every possible implementation would be impractical, if not impossible. Numerous alternative implementations could be implemented, using either current technology or technology developed after the filing date of this patent. It is envisioned that such alternative implementations would still fall within the scope of the claims.

At least one aspect of this disclosure is related to systems that automate library preparation processes and use less consumables, thereby reducing a footprint of the systems and an amount of solid waste produced. The systems disclosed also use a single well plate for many operations and use lower working/regent volumes as a result. The systems store consumables and reagents for multiple runs and allow variable batch processing such that a single sample can be processed without consuming reagents associated with a 24 sample kit, for example.

The disclosed implementations enable workflows from extracted material through library preparation and/or enable the ability to automate assays of varying complexity. The disclosed implementations enable the ability to run multiple assay workflows in parallel and/or the ability to perform staggered starts. The disclosed implementations enable the ability to run variable batch sizes and/or provide low touchpoint, walkaway consumable loading. The disclosed implementations enable architecture extensibility (e.g., 24, 48 samples) and/or onboard library quantification and/or onboard library pooling, denaturation and/or dilution. The disclosed implementations enable the ability to transfer libraries to the sequencer and/or management of cross-contamination. The disclosed implementations enable minimized plastic waste and/or minimal increase in cost per sample versus manual library prep workflows, as an example. The disclosed implementations enable no or marginal increase to assay runtime versus manual library prep workflows and/or enable no or limited change in assay performance versus manual library prep workflows. The disclosed implementations enable high sample success rate and/or instrument uptime and/or provide a small instrument footprints. The disclosed implementations enable digital system integration (e.g., LIMS) and/or operates in standard lab.

The disclosed implementations enable the integration of extraction and/or enable the integration with a sequencer. The disclosed implementations enable architecture extensibility (e.g., 96 samples) and/or provide multiple “Assay Bays” & single “Common Bay.” Any number of “Assay Bays” may be included such as twelve assay bays. In one example, the disclosed implementations enable greater than about 90% of assay automation to be achieved within individual Assay Bays. Other percentages equal to or less than about 90% of the assay automation may be achieved within individual Assay Bays, however. In one example, the disclosed implementations enable up to twelve samples to be processed within each Assay Bay. The disclosed implementations can process a number of samples greater than or less than twelve samples, however. The disclosed implementations enable the system to be configured as a 2-Assay Bay (24 samples), as a 4-Assay Bay (48 samples), etc., although other bay numbers may be used. The disclosed implementations enable the common bay to be leveraged to quant, pool, denature, and/or dilute samples from multiple Assay Bays. In one example, the disclosed implementations enable different assays to be run in different Assay Bays in parallel. The disclosed implementations enable Assay Bays to be accessed and/or started at different times (staggered starts). The disclosed implementations enable integrated thermal magnet stations in each Assay Bay to reduce the footprint and increase workflow control. The disclosed implementations use pipet mixing in some examples and do not include a shaker. The Assay Bays and/or the Common bay may include a reagent chiller in some implementations.

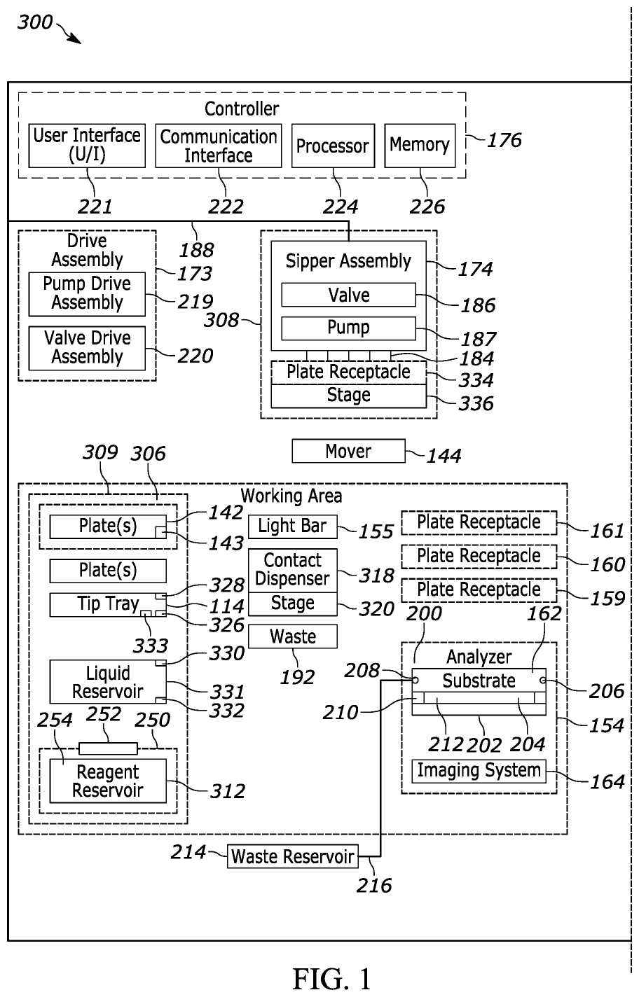

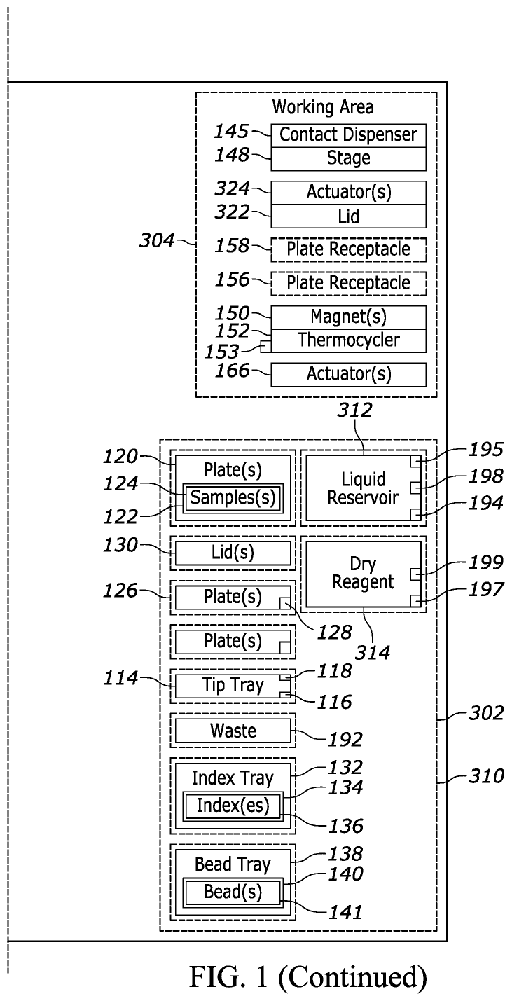

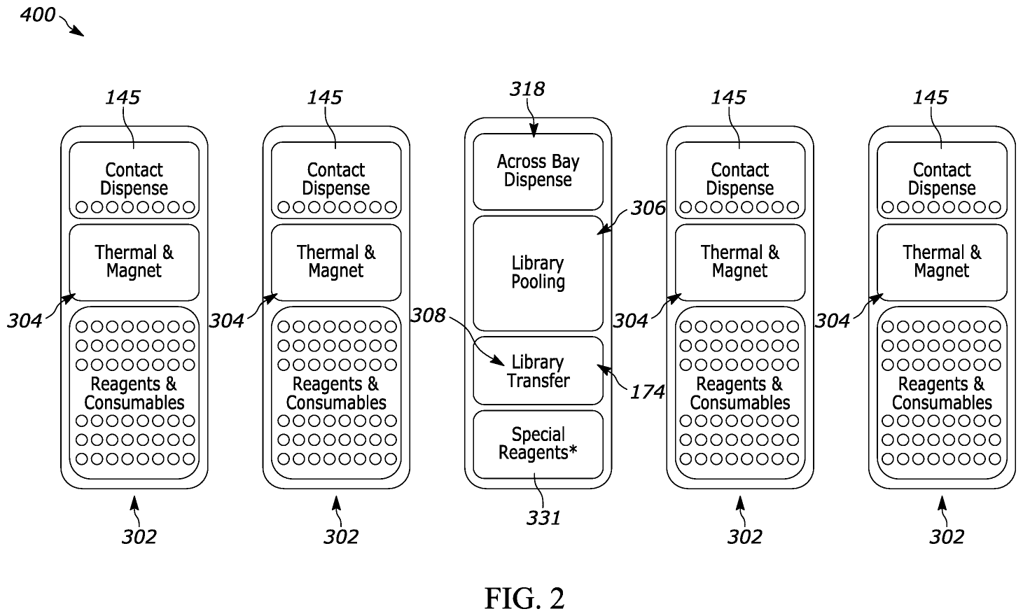

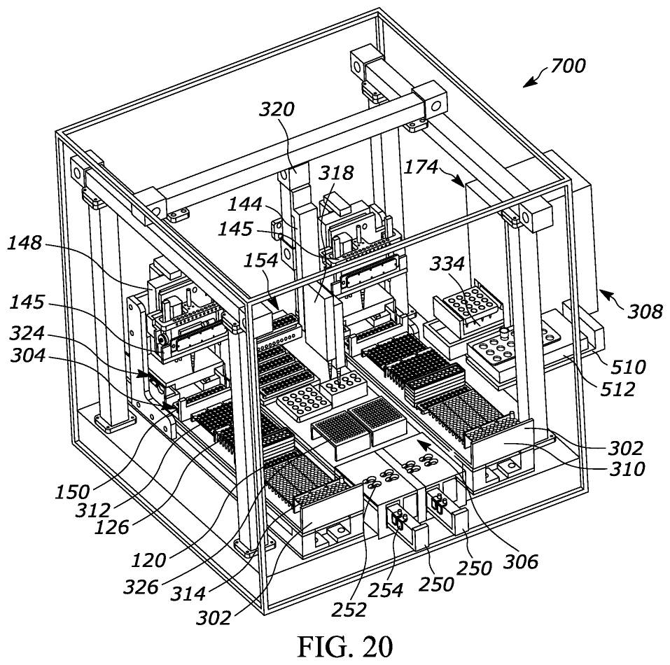

FIG. 1 illustrates a schematic diagram of an implementation of a system 300 in accordance with the teachings of this disclosure. The system 300 can be used to automatically, easily, and efficiently prepare DNA libraries for sequencing applications, for example. The system 300 includes a consumables area 302, a first working area 304, a second working area 306, and a loading area 308. The second working area 306 also includes a consumables area 309 in the implementation shown. The consumables area 302 and the first working area 304 may be referred to as a first bay (e.g., a first assay bay) and the second working area 306 may be referred to as a common bay. The system 300 may include any number of consumables areas 302 and a corresponding number of first working areas 304. The system 300 may include four consumables areas 302 and four first working areas 304 as shown in FIGS. 2-4 or the system 300 may include two consumables areas 302 and two first working areas 304 as shown in FIGS. 20 and/or 32 as examples. Other numbers of working areas may be used. If more than one consumables area 302/first working area 304 are included, the system 300 can perform a corresponding number of workflows at the same time and/or at different times. One workflow (e.g., one assay) may be performed at one of the first working areas 304 and another workflow (e.g., a second assay) can be performed at another one of the first working areas 304 as an example.

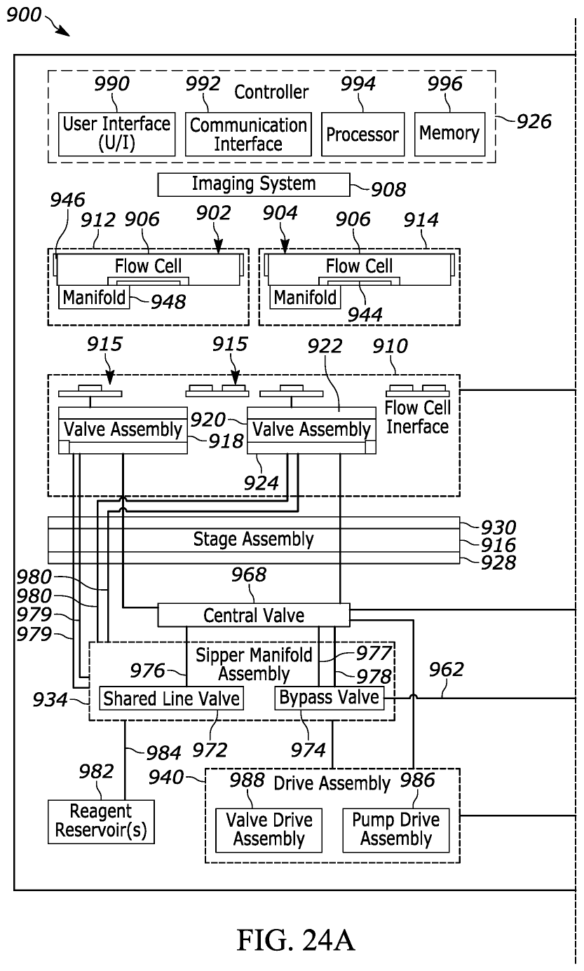

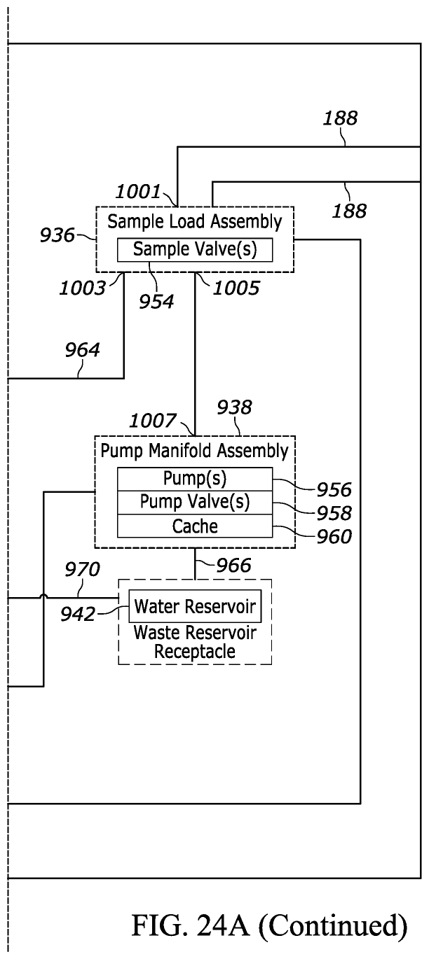

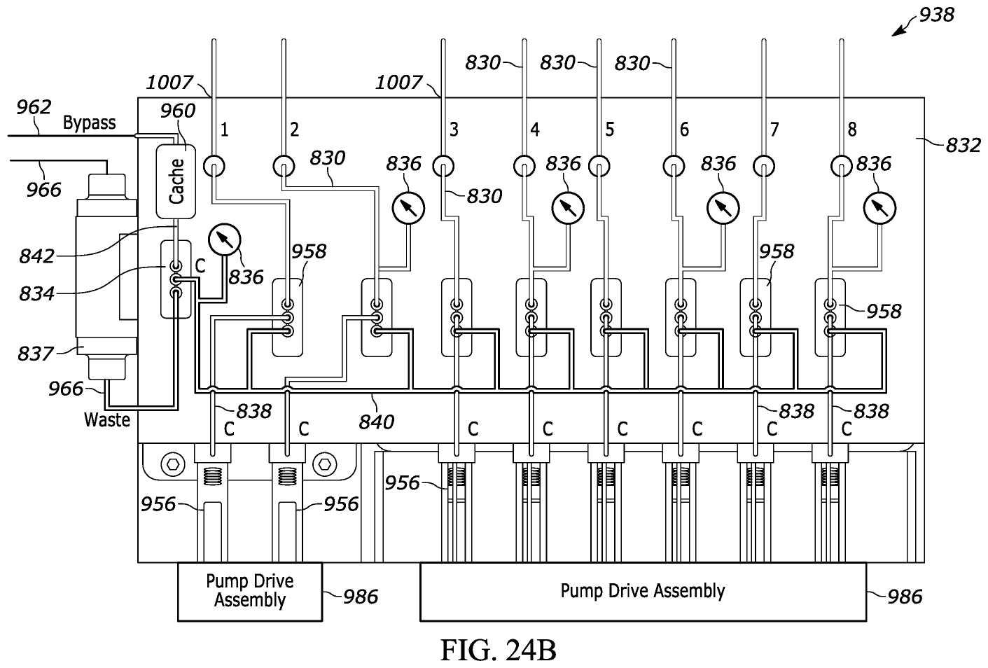

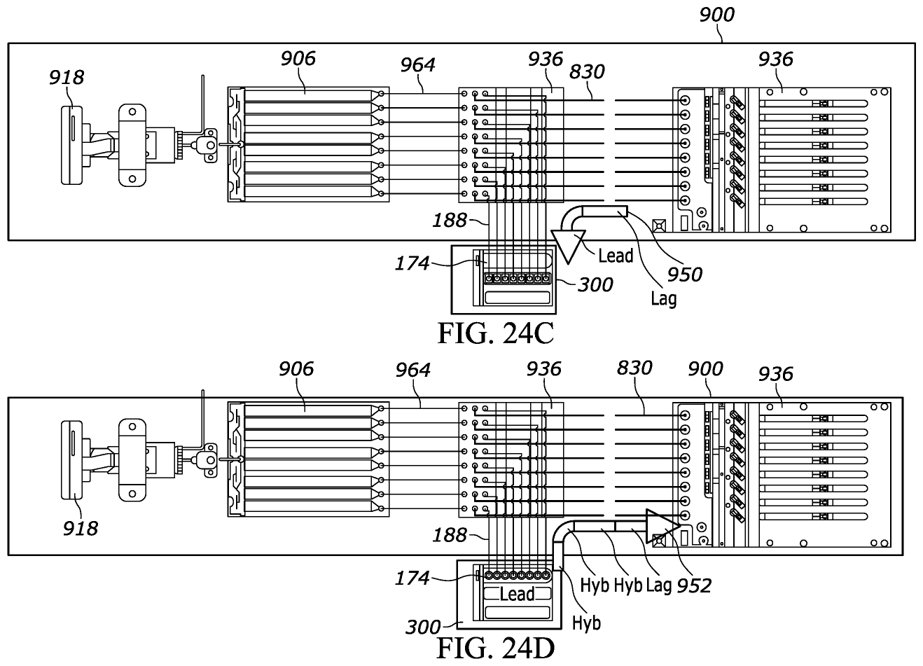

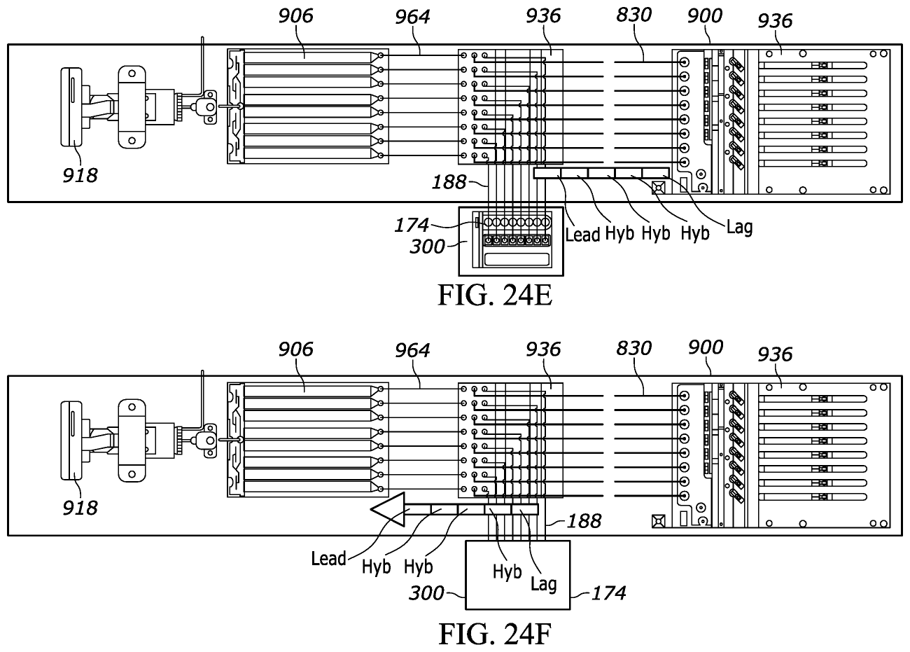

The system 300 may perform DNA library preparation workflows that include amplification processes, cleanup processes, quantification processes, library normalization processes, pooling processes, denaturing processes, and/or diluting processes in some implementations. The loading area 308 may be associated with loading and/or transferring a prepared sample to a system such as a sequencing system and/or a next generation sequencing system (see, FIG. 24A). The first working area 304 may be associated with amplification processes and cleanup processes and the second working area 306 may be associated with quantification processes, library normalization processes, pooling processes, denaturing processes, and/or diluting processes.

The system 300 may perform different workflows. The workflows may include whole genome sequencing (WGS) workflows, DNA & RNA enrichment workflows, methylation workflows, split-pool amplicon workflows, amplicon workflows, exome sequencing workflows, ChIP-seq workflows, Methyl-Seq workflows, metagenomic, mate-pair workflows, single-cell workflows, cDNA workflows, ligation workflows, adapter ligation workflows, tagmentation workflows, multiplexing workflows, and/or long-read workflows, as examples. The DNA library preparation workflow can be performed on any number of samples such as between one sample and twenty four samples. The system 300 thus allows for variable batch processing.

The consumable area(s) 302 and/or 309 may be used to load and store reagents and consumables needed for a library prep. Process, including, disposable tips, wet or dry assay specific reagents, wet or dry bulk reagents, and reaction plates and wells. The consumables area 302 includes a consumables receptacle 310 that is shown receiving a tip tray 114 having a first tip 116 and a second tip 118, a first plate 120 having a well 122 containing a sample 124, and a second plate 126 having a well 128. The consumables receptacle 310 may be a drawer that can be pulled out from the system 300 and loaded with the consumables 114, 116, 118, 120, 126. The consumables receptacle 310 is also shown having a lid 130, an index tray 132 having a well 134 containing indexes 136, a bead tray 138 having a well 140 containing beads 141, a liquid reservoir 312, and a dry reagent reservoir 314. One or more of these reagents 136 and/or 141 may be lyophilized and included with the dry reagent reservoir 314. The second working area 306 may also have a tip tray 114 and a third plate 142 having a well 143.

The first plate 120, the second plate, 126, the third plate 142, the index tray 132, and/or the bead tray 138 may be a stack of the corresponding plates 120 and/or 126 and/or trays 132 and/or 138. The first plate 120, the second plate, 126, and the third plate 142 may be stacked in some implementations while the index tray 132 and/or the bead tray 138 may not be stacked. Other approaches may prove suitable. The tip tray 114 may have a plurality of the first tips 116, a plurality of the second tips 118, and/or one or more tips that are different sizes from the first tips 116 and/or the second tips 118. The tips in the tip tray 114 may be reusable for at least multiple portions of a workflow, as will be discussed in greater detail below. While the tip tray 114 is mentioned having the first tip 116 and the second tip 118, the tip tray 114 can have any number of tips such as 24 tips. The plates 120, 126, 142 may have any number of wells, however.

While the plates 120 and 126 are mentioned having a single well 122, 128, the plates 120, 126 may have a plurality of wells such as 24 wells. The plates 120, 126 may include a 2×12 array of wells that allow for side access for all wells in some implementations. The plates 120, 126 being implemented by a 2×12 array allows a fill level/bubbles to be inspected in every well with side-view computer vision, increases heat transfer in heating (PCR) operations, and opportunities in magnetic pull down operations.

The system 300 includes a mover 144 and the first working area 304 includes a contact dispenser 145, a stage 148, a magnet 150, and a thermocycler 152. The contact dispenser 318 may be included in the first working area 304 in some implementations. The contact dispenser 145 may be movable to aspirate/dispense liquid to the consumables area 302 and/or to the first working area 304.

The stage 148 may be an x-z stage, such that the stage 148 is movable in the x and z directions (but not in the y direction). The stage 148 and the contact dispenser 148 may be movable to aspirate and/or dispense fluid between and above the consumables area 302 and the first working area 304 as a result. The contact dispenser 145 may, for example, move linearly in the x direction, which thereby reduces the risk of cross-contamination (between different samples) and allows some or all of the tips employed in the system 300 to be reusable for at least part of the processes performed by the system 300. The stage 148 may be implemented differently, however.

The second working area 306 includes an analyzer area 154, and the system 300 also includes a contact dispenser 318 and a stage 320 in the implementation shown. The stage 320 may be referred to as a cross-bay gantry. The contact dispenser 318 may additionally or alternatively be implemented by a non-contact dispenser for aspirating/dispensing throughout the system 300. The dispenser 318 and the stage 320 can operate in the first working area 304 and the second working area 306.

The contact dispenser 318 may be movable to aspirate/dispense liquid to the consumables area 302, to the first working area 304, and/or to the second working area 306. The contact dispenser 318 may carry two tips (or two sets of tips) in some implementations, where one of the tips can hold a first volume of fluid and the other one of the tips can hold a second volume of fluid. The contact dispenser 318 may include two contact dispensers, where each dispenser carries one of tips. The two contact dispensers may be independently movable relative to one another. The first volume may be about 50 microliters and the second volume may be about 500 microliters.

The stage 320 may be an x-y-z stage, such that the mover 144 is movable in the x, y, and z directions. The stage 320 and the contact dispenser 318 may be movable to aspirate and/or dispense fluid between and above the consumables area 302, the first working area 304, and/or the second working area 306 as a result.

The mover 144 may include a robotic arm and/or include grippers. The stage 320 may carry the mover 144 and the contact dispenser 318 in some implementations.

The second working area 306 may also include a light bar 155 that may be used to degrade oligonucleotides. The first working area 304 may additionally or alternatively include the light bar 155 in some implementations. The light bar 155 may be a high power ultraviolet light (UV) light bar that is regularly used throughout a workflow to repeatedly degrade oligonucleotides to deter cross contamination in some implementations.

The first working area 304 has a first plate receptacle 156 and a second plate receptacle 158, the second working area 306 has a third plate receptacle 159, a fourth plate receptacle 160, and a fifth plate receptacle 161, and the analyzer area 154 includes a substrate 162 and an imaging system 164 in the implementation shown. The analyzer area 154 may be implemented differently, however, to perform quantification processes in other implementations. The analyzer area 154 may the substrate 162 that is implemented by a well plate in which a portion of the sample and a dye are dispensed as an example. The imaging system 164 may image the portion of the sample in the well plate to determine a concentration of the sample.

The first plate receptacle 156 may include a thermal block defining well receptacles and the thermocycler 152 can be positioned beneath the well receptacles. The thermocycler 152 may be positioned beneath the first plate receptacle 156 in some implementations. A heat sink 153 is shown coupled to the thermocycler 152. The heat sink 153 may be omitted, however.

The plate receptacles 156, 158, 159, 160, 161 may be referred to as plate stations. The imaging system 164 may be a fluorescent imaging system, a fluorescence spectrophotometer including an objective lens and/or a solid-state imaging device. The solid-state imaging device may include a charge coupled device (CCD) and/or a complementary metal oxide semiconductor (CMOS). While five plate receptacles 156, 158, 159, 160, 161 are shown, any number of plate receptacles may be included such as six plate receptacles.

The second working area 306 also includes a reagent receptacle 250 having an access opening 252. A reagent reservoir 254 is shown received within the reagent receptacle 250. The first working area 304 may additionally or alternatively include a reagent receptacle 250 having an access opening 252. The reagent receptacle 250 may be refrigerated and may be a drawer that can be pulled out from the system 300 and loaded with the reagent reservoir 254. The reagent reservoir 254 may be accessed through the access opening 252 by the contact dispenser 318 to aspirate reagent from the reagent reservoir 254, for example.

The loading area 308 includes a sipper assembly 174 in the implementation shown. The sipper assembly 174 may be referred to as a sample sipper manifold assembly or a sample sipper assembly. The sipper assembly 174 may include sippers 184. Any number of sippers 184 may be included such as between two sippers 184 and sixteen sippers 184 as an example. The sipper assembly 174 may be coupled to a corresponding number of the flow cells of another system (see FIG. 24A, for example) via sippers 184. The sipper assembly 174 includes a plurality of ports in some implementations where each port of the sipper assembly 174 may receive one of the sippers 184. The sippers 184 may be referred to as fluidic lines. The sipper assembly 174 also includes a valve 186 that may be selectively actuated to control the flow of fluid through a fluidic line 188. The fluidic line 188 may be referred to as a sample sipper assembly. The sipper assembly 174 also includes a pump 187 to selectively flow the prepared sample from a well 128, 143 through the sipper 184, through the fluidic line 188, and out of the system 300 to another system (see, for example, FIG. 24A). The other system may be used to perform an analysis on one or more samples of interest. The sample may include one or more DNA clusters that are linearized to form a single stranded DNA (sstDNA). The other system may be sequencing system and/or a next generation sequencing system, as an example.

The valve 186 may be implemented by a rotary valve, a pinch valve, a flat valve, a solenoid valve, a check valve, a piezo valve, etc. Other fluid control devices may prove suitable. The pump 187 may be implemented by a syringe pump, a peristaltic pump, and/or a diaphragm pump. Other types of fluid transfer devices may be used, however. The controller 176 is electrically and/or communicatively coupled to components of the system 300 to perform various functions as disclosed herein. The sipper assembly 174 may alternatively be omitted.

The actuator 166 can move the magnet 150 between an upward position where the magnet 150 affects any plate positioned on the first plate receptacle 156 and a downward position where the magnet 150 does not affect any plate positioned on the first plate receptacle 156. The magnet 150 being moved relative to the first plate receptacle 156 and any plate 120, 126, 142 positioned on the first plate receptacle 156 allows less area on the first working area 304 to be consumed. The magnet 150 can moreover be moved with relatively higher confidence as compared to an alternative approach to moving one of the plates 120, 126, 142 filled with samples to a separate magnet station. The magnet 150 may be implemented by a halbach array configuration to strengthen and focus the corresponding magnetic fields.

The mover 144 moves the first plate 120 from the consumables area 302 to the first plate receptacle 156. Different wells 122 of the first plate 120 may contain different samples 124. The samples 124 may be a biological sample derived from a human, animal, plant, bacteria, virus, or fungi. Other sources of obtaining the biological samples may prove suitable. The mover 144 may include a gantry having grippers that can pick-and-place objects such as the plates 120, 126 and/or the trays 132, 138 between different areas 302, 304, 306, 308 of the system 300. The mover 144 may be implemented in different ways, however.

The stage 148 aligns the contact dispenser 145 with the tip tray 114 and the contact dispenser 145 couples with the first tip 116 from the tip tray 114. While the contact dispenser 145 is mentioned coupling with one first tip 116, the contact dispenser 145 may couple with a number of the first tips 116 that corresponds to the number of the wells 122 in the first plate 120 and/or the number of the wells 122 in the first plate 120 containing the sample 124. The first tip 116 may be a smaller pipette tip that is used to move smaller fluid volumes and the second tip 118 may be a larger pipette tip that is used to move larger fluid volumes, for example. Each of the first tip 116 and/or the second tip 118 may be exposed to a single sample during a workflow reducing the likelihood of cross-contamination and reducing the need to obtain a new tip after each operation. In some library preparation workflows, for example, each of the first tip 116 and/or the second tip 118 may be used through an entire workflow. The contact dispenser 145 may couple with and/or use different ones of the tips 116, 118 depending on the workflow and/or the processes within a workflow that the system 300 is implementing.

In one example workflow, the stage 148 aligns the contact dispenser 145 with the index tray 132 and the contact dispenser 145 aspirates the indexes 136 from the index tray 132 using the first tip 116. The stage 148 can then align the contact dispenser 145 with the first plate 120 and the contact dispenser 145 dispenses the indexes 13 into the well 122 of the first plate 120. The mover 144 moves the lid 130 from the consumables area 302 and places the lid 130 on the first plate 120 to cover the well 122 of the first plate 120 with the lid 130. The first working area 304 also includes a lid 322 and an actuator 324. The lid 322 may be referred to as a cover and/or a door. The actuator 324 may move the lid 322 in operation relative to the plate receptacle 156 to cover the plate receptacle 156 during amplification processes, for example. The lid 322 may thus be positioned to enclose the first plate 120 during the amplification processes. The lid 130 and/or the lid 322 may enclose the first plate 120 during the amplification processes.

The thermocycler 152 is aligned with the first plate receptacle 156 and the thermocycler 152 amplifies the sample 124 within the well 122 of the first plate 120. The thermocycler 152 and/or the magnet 150 can act on a plate 120, 126 received at the first plate receptacle 156 in the implementation shown. The thermocycler 152 may alternatively be spaced from the magnet 150.

The actuator 324 can move the lid 322 off of the first plate 120 and/or the mover 144 can move the lid 130 from the first plate 120 to the consumables area 302 after the amplification processes are complete. The thermocycler 152 may include the lid 322 that covers the first plate 120 and a separate consumables lid may be positioned on top of the first plate 120 between the first plate 120 and the lid 322, for example. The consumables area 302 includes a waste 192 that can receive used consumables such as, for example, the lid 130. The lid 130 may alternatively be reused, however. The waste 192 may be a waste tray having an absorbent material to absorb liquid waste.

The system 300 may perform cleanup processes after the amplification processes are performed. The stage 148 aligns the contact dispenser 145 with the bead tray 138 and the contact dispenser 145 aspirates the beads 141 from the bead tray 138. The contact dispenser 145 may aspirate the beads 141 using the same first tip 116 used to aspirate the indexes 136. The contact dispenser 145 may alternatively use another one of the first tips 116 or one of the second tips 118 to aspirate the beads 141.

The stage 148 aligns the contact dispenser 145 with the first plate 120 and the contact dispenser 145 dispenses the beads 141 into the well 140 of the first plate 120 as part of the cleanup process. The stage 148 aligns the contact dispenser 145 with the liquid reservoir 312 and the contact dispenser 145 aspirates first reagent 194 from the liquid reservoir 312. The stage 148 then aligns the contact dispenser 145 with the first plate 120 and the contact dispenser 145 dispenses the first reagent 194 into the well 122 of the first plate 120. The contact dispenser 145 may alternatively aspirate hydrating liquid 195 from the liquid reservoir 312 and then dispense the hydrating liquid 195 into dried reagent 197 contained within the dry reagent reservoir 314 to rehydrate the dried reagent 197 and form the first reagent 194. The contact dispenser 145 may pipette mix the dried reagent 197 and the hydrating liquid 195. The first reagent 194 may be a bead buffer and the sample 124 may bind to the beads 141 in the presence of the bead buffer. The contact dispenser 145 may be able to jet dispense with adequate liquid velocity to enable jet mixing in some implementations. The system 300 may also include a shaker to enable mixing.

The stage 148 aligns the contact dispenser 145 with the tip tray 114 and the contact dispenser 145 places the first tip 116 in the tip tray 116 and the contact dispenser 145 then couples with the second tip 118 from the tip tray 114. While the contact dispenser 145 is mentioned coupling with one second tip 118, the contact dispenser 145 may couple with a number of the second tips 118 that corresponds to the number of the wells 122 in the first plate 120 and/or the number of the wells 122 in the first plate 120 containing the sample 124.

The actuator 166 moves the magnet 150 toward the plate receptacle 156 and the magnet 150 draws the beads 141 toward the magnet 150. The beads 141 and the sample 124 bound to the beads 141 may be positioned toward the bottom of the well 122 of the first plate 120 or on a side(s) of the well 122. The tips 116 and/or 118 may easily access the well 122 if the beads 141 are on the side of the well 122. The magnet 150 may cause the beads 141 to be in any position within the well 122, however.

The stage 148 aligns the contact dispenser 145 with the first plate 120 and the contact dispenser 145 aspirates the first reagent 194 from the well 122 of the first plate 120. The contact dispenser 145 may dispense the first reagent 194 aspirated from the well 122 of the first plate 120 into the waste 192.

The stage 148 aligns the contact dispenser 145 with the liquid reservoir 312 and the contact dispenser 145 aspirates second reagent 198 from the liquid reservoir 312 and the stage 148 then aligns the contact dispenser 145 with the first plate 120 and the contact dispenser 145 dispenses the second reagent 198 into the well 122 of the first plate 120. The contact dispenser 145 may alternatively aspirate hydrating liquid 195 from the liquid reservoir 312 and then dispense the hydrating liquid 195 into a dried reagent 199 contained within the dry reagent reservoir 314 to rehydrate the dried reagent 199 and form the second reagent 198. The second reagent 198 may be an elution buffer that releases the sample 124 from being bound to the beads 141 and, specifically, releases DNA associated with the sample 124 from being bound to the beads 141.

The mover 144 moves the second plate 126 from the consumables area 302 to the second plate receptacle 158. The system 300 can use the second plate 126 for a transfer operation. The second plate 126 may alternatively remain in the consumables area 302 during the transfer operation. The actuator 166 moves the magnet 150 toward the second plate receptacle 158 to draw the beads 141 toward the magnet 150 and, thus, provide a substantially bead-free eluate solution comprising the second reagent 198 and the sample 124 within the well 122.

The stage 148 aligns the contact dispenser 145 with the first plate 120 and the contact dispenser 145 aspirates the second reagent 198 and the sample 124 from the well 122 of the first plate 120 using the second tip 118, for example. The stage 148 aligns the contact dispenser 145 with the second plate 126 and the contact dispenser 145 dispenses the second reagent 198 and the sample 124 into the well 128 of the second plate 126. The second plate 126 may alternatively be positioned in the consumables area 302 when the contact dispenser 145 dispenses the second reagent 198 and the sample 124 into the well 128 of the second plate 126. The second plate receptacle 158 may be omitted from the second working area 306 in such implementations.

The system 300 may perform the quantification processes after the cleanup processes are performed. The mover 144 moves the second plate 126 from the first working area 304 to the plate receptacle 159 of the second working area 306 to initiate the quantification processes in some implementations. The mover 144 may alternatively move the second plate 126 from the consumables area 302 to the plate receptacle 159 of the second working area 306 to initiate the quantification processes in implementations when the second plate 126 remains in the consumables area 302 during the transfer operations. The stage 320 aligns the contact dispenser 318 with the tip tray 114 of the second working area 306 in some implementations and the contact dispenser 145 couples with a tip 326 from the tip tray 114.

The substrate 162 may be a plate having a well. The substrate may be a consumable that is disposed of after use. The imaging system 164 may be spaced from the substrate 162 and coupled to a portion of the system 300 such as a frame of the system 300. The imaging system 164 may alternatively be carried by a stage.

The stage 320 aligns the contact dispenser 318 with the second plate 126 to perform the quantification processes and the contact dispenser 318 aspirates a portion of the second reagent 198 and the sample 124 from the well 128 of the second plate 126. The portion of the second reagent 198 and the sample 124 may be about 2 μL.

The stage 320 aligns the contact dispenser 318 with the substrate 162 and the contact dispenser 318 dispenses the portion of the second reagent 198 and the sample 124 into a well of the substrate 162 as an example. A dye may also be dispensed into the well of the substrate 162 by the contact dispenser 318. The imaging system 164 obtains image data of the portion of the second reagent 198 and the sample 124 within the well of the substrate 162. The imaging system 164 and/or the system 300 uses the image data to determine a concentration of the sample 124. The mover 144 may move the substrate 162 to the waste 192 of the consumables area 309 of the second working area 306.

The substrate 162 may alternatively be implemented by a pair of plates 200, 202 between which a gap 204 is defined. The substrate 162 in such an implementation includes an inlet 206 and an outlet 208 in fluid communication with the gap 204 and a seal 210 positioned between the pair of plates 200, 202. The plates 200, 202 and the seal 210 define a channel 212 between the inlet 206 and the outlet 208. A waste reservoir 214 may be fluidly coupled to the outlet 208 of the substrate 162 by a fluidic line 216.

In the alternative implementation of the substrate 162, the stage 320 aligns the contact dispenser 318 with the inlet 206 of the substrate 162 and the contact dispenser 318 dispenses the portion of the second reagent 198 and the sample 124 into the inlet 206 of the substrate 162. The portion of the second reagent 198 and the sample 124 may flow and/or be positioned between the inlet 206 and the outlet 208 in this implementation and the imaging system 164 obtains image data of the portion of the second reagent 198 and the sample 124. The imaging system 164 and/or the system 300 uses the image data to determine a concentration of the sample 124. Negative pressure, oil, and/or another substance may be used to urge the portion of the second reagent 198 and the sample 124 between the inlet 206 and the outlet 208. The first plate 200 may alternatively be hingably coupled or removably coupled to the second plate 202 to allow the contact dispenser 318 to dispense the portion of the second reagent 198 and the sample 124 onto the second plate 202 prior to the first plate 200 being positioned overtop of the second plate 202. The system 300 may perform quantification processes in different ways, however.

The system 300 may perform the normalization processes after the quantification processes are performed. The stage 320 aligns the contact dispenser 318 to initiate the normalization processes in some implementations. The contact dispenser 318 aspirates a diluent 330 from a liquid reservoir 331 of the second working area 306. The stage 320 then aligns the contact dispenser 318 with the second plate 126 and the contact dispenser 318 dispenses the diluent 330 into the well 128 of the second plate 126 to dilute the sample 124 based on the concentration of the sample determined. The sample 124 within the well 128 of the second plate 126 will have a concentration within a threshold value after the diluent 330 is added to the well 128 as a result. The diluent 330 may be a buffer.

The system 300 may perform the pooling processes after the quantification processes are performed. The stage 320 aligns the contact dispenser 318 with the tip tray 114 of the second working area 306 and the contact dispenser 318 places the tip 326 in the tip tray 114 and the contact dispenser 318 then couples with another tip 328 from the tip tray 114 to initiate the pooling processes in some implementations. The mover 144 moves a plate 142 from the second working area 306 to the plate receptacle 160 of the second working area 306. The stage 320 aligns the contact dispenser 318 with the second plate 126 and the contact dispenser 318 aspirates the sample 124 from the well 128 of the second plate 126. The stage 320 then aligns the contact dispenser 318 with the third plate 142 and the contact dispenser 318 dispenses the sample 124 into the well 143 of the third plate 142. Additional samples from other wells of the second plate 126 may be deposited into the well 143 of the third plate 142 in a similar manner to combine a plurality of normalized samples together. A single tip can be used for the pooling processes. The contact dispenser 318 may pipette from final archive library well directly to pool and, thus, unique tips per sample may be used.

The system 300 may perform the denaturing processes after the pooling processes are performed (though in some implementations, denaturing need not be performed). The stage 320 aligns the contact dispenser 318 with the tip tray 114 of the second working area 306 and the contact dispenser 318 places the tip 328 in the tip tray 114 and the contact dispenser 318 then couples with another tip 333 from the tip tray 114 to initiate the denaturing processes in some implementations. The contact dispenser 1318 may use the same tip 328 used during the pooling processes in some implementations, however. The stage 320 aligns the contact dispenser 318 with the liquid reservoir 331 and the contact dispenser 318 aspirates reagent 332 from the liquid reservoir 331. The stage 320 then aligns the contact dispenser 318 with the third plate 142 and the contact dispenser 318 dispenses the reagent 332 into the well 143 of the third plate 142 to denature the pooled and normalized samples. The reagent 332 may be Sodium hydroxide (NaOH). Other denaturing processes may prove suitable. For example, Formamide or an equivalent may be used during the denaturing processes.

The system 300 may dilute the pooled and denatured samples after the pooling and the denaturing processes are performed. The contact dispenser 318 aspirates a diluent 330 from a liquid reservoir 331 of the second working area 306. The stage 320 then aligns the contact dispenser 318 with the third plate 142 and the contact dispenser 318 dispenses the diluent 330 into the well 128 of the third plate 142 to dilute the sample 124 based on the concentration of the sample based on specifications of the sequencing system (e.g., the system 900) into which the sample is to be loaded. The pooled samples 124 within the well 143 of the third plate 142 will have a concentration within a threshold value after the diluent 330 is added to the well 143 as a result. The diluent 330 may be a buffer.

The system 300 may perform the loading processes after the denaturing processes and/or after the diluting processes are performed. The mover 144 moves the third plate 142 from the second working area 306 to a plate receptacle 334 of the loading area 308. The loading area 308 is shown including a stage 336 that can be used to move the plate receptacle 334 relative to the sipper assembly 174. The sipper assembly 174 flows the denatured samples from the well 128, 143 through the corresponding sipper(s) 184, through the fluidic line(s) 188, and out of the system 300 to another system for sequencing (see, for example, FIG. 24A).

While the above example discloses loading samples to another system 300, the system 300 may provide a plurality of options. For example, the system 300 may perform library quantification processes on the samples at the second working area 306 and the library of samples may be archived. The second working area 306 may be referred to as a common bay. The quantification values may be associated and/or tracked with each library. The library of samples may be archived by sealing the samples and/or freezing the samples.

As another alternative, the system 300 may perform library quantification processes and library pooling processes on the samples at the second working area 306. The library pooling processes may include preparing an equimolar pool using the samples based on the quantification values determined by the library quantification processes. The quantification values may be used to determine what volume of each sample should be used to create an equimolar pool, for example. The system 300 may pipette different volumes of each sample into the pool based on the quantification value to create an equimolar pool. The process may also include archiving a remaining portion the library of samples.

Another alternative example, the system 300 may prepare a sequence ready pool of samples at the second working area 306. The sequence ready pool of samples may be prepared by performing library quantification processes and library pooling processes on the samples. The sequence ready pool of samples may be prepared by performing denaturing processes on the samples and/or performing diluting processes on the samples as examples. The reagents used for denaturing and/or diluting may be based on the sequencer type to be used. The final sequencing-ready concentration of the pool may be based on a threshold loading concentration for the application (library type) and/or the flow cell.

Another alternative example, the system 300 may prepare a sequence ready pool of samples at the second working area 306 and the sequence ready pool of the library of samples may be transferred to another system such as a sequencer. The system 300 and the other system (e.g., a sequencer) may communicate and coordinate transfer of pools through a fluidic line for sequencing, for example.

The system 300 also includes a drive assembly 173. The drive assembly 173 includes a pump drive assembly 219 and a valve drive assembly 220. The pump drive assembly 219 may be adapted to interface with the pump 187 to pump fluid from the reagent reservoir 110 to the non-contact dispenser 146. The valve drive assembly 220 may be adapted to interface with the valve 186 to control the position of the valve 186.

The controller 176 includes a user interface 221, a communication interface 222, one or more processors 224, and a memory 226 storing instructions executable by the one or more processors 224 to perform the various functionality discussed herein. The user interface 221, the communication interface 222, and the memory 226 are electrically and/or communicatively coupled to the one or more processors 224. In some implementations, the controller 176 is located in the same area as the other components of the system 300 and may be physically coupled to the other components of the system 300, for example via a wired connection. In other implementations, the controller 176 is located remotely from the other components of the system 300 and may be communicatively coupled to the other components of the system 300, for example via a wireless connection. For example, the controller 176 may be implemented on a cloud computing device.

In an implementation, the user interface 221 receives input from a user and provides information to the user associated with the operation of the system 300 (e.g., information about the analysis taking place). The user interface 221 may include a touch screen, a display, a key board, a speaker(s), a mouse, a track ball, and/or a voice recognition system. The touch screen and/or the display may display a graphical user interface (GUI).

In an implementation, the communication interface 222 enables communication between the system 300 and a remote system(s) (e.g., computers) using a network(s). The network(s) may include the Internet, an intranet, a local-area network (LAN), a wide-area network (WAN), a coaxial-cable network, a wireless network, a wired network (e.g., Ethernet), a satellite network, a digital subscriber line (DSL) network, a cellular network, a Bluetooth connection, a near field communication (NFC) connection, etc. For example, the library preparation system and the sequencer may be directly coupled to each other via a wired communication link, such as an Ethernet cable. The library preparation system may then transmit and receive communications to and from the sequencer via the wired communication link.

Some of the communications provided to the remote system may be associated with an amplification process(es), a cleanup process(es), a library normalization process(es), a pooling process(es), a denaturing process(es), and/or a loading process(es)), etc. generated or otherwise obtained by the system 300. Some of the communications provided to the system 300 may be associated with an amplification process(es), a cleanup process(es), a library normalization process(es), a pooling process(es), a denaturing process(es), and/or a loading process(es) to be executed by the system 300.

The one or more processors 224 and/or the system 300 may include one or more of a processor-based system(s) or a microprocessor-based system(s). In some implementations, the one or more processors 224 and/or the system 300 includes a reduced-instruction set computer(s) (RISC), an application specific integrated circuit(s) (ASICs), a field programmable gate array(s) (FPGAs), a field programmable logic device(s) (FPLD(s)), a logic circuit(s), and/or another logic-based device executing various functions including the ones described herein.

The memory 226 can include one or more of a hard disk drive, a flash memory, a read-only memory (ROM), erasable programmable read-only memory (EPROM), electrically erasable programmable read-only memory (EEPROM), a random-access memory (RAM), non-volatile RAM (NVRAM) memory, a compact disk (CD), a digital versatile disk (DVD), a cache, and/or any other storage device or storage disk in which information is stored for any duration (e.g., permanently, temporarily, for extended periods of time, for buffering, for caching).

The memory 226 may store instructions executable on the processors 224 that make up a library preparation system communication module. The library preparation system communication module may transmit and receive communications, via the communication interface 222 for example using an Ethernet network, to a sequencer. These communications may include information related to the library of samples. For example, the library preparation system communication module may transmit an indication of a preparation status of the library of samples to the sequencer. The preparation status may be that library preparation has been completed, and the library of samples is ready to load into the sequencer. The preparation status may also include an estimated duration until library preparation is complete.

The library preparation system communication module may also transmit identification information for each sample (e.g., a sample ID) in the library of samples to the sequencer. Moreover, for each sample, the library preparation system communication module may transmit, to the sequencer, an indication of an index attached to the sample. An index is a short piece of DNA (e.g., 6-20 bases) attached to each sample which acts as a barcode or tag to identify and/or separate the sample amongst the library of samples.

Additionally, the library preparation system communication module may transmit instructions to the sequencer indicating a particular lane of a flow cell for each sample to be sequenced in. For example, the flow cell may have 8 lanes, and the library preparation system communication module may instruct the sequencer to place samples 1-10 in lane 1, samples 11-20 in lane 2, etc.

The library preparation system communication module may also transmit one or more run parameters to the sequencer, so that the sequencer can sequence the library of samples using the received run parameters. The run parameters may include the number of cycles for each sample, or any other suitable run parameters for sequencing.

Furthermore, the library preparation system communication module may receive communications from the sequencer. For example, the library preparation system communication module may receive status information for the sequencer, such as the sequencer is ready to receive the library of samples, an estimated duration until the sequencer can reach a safe pause point in the sequencing recipe that would not cause data quality issues to any runs that are already occurring and can receive the library of samples, indications of the sequencing setup steps that have been completed, the sequencing setup steps that remain, the expected durations of the remaining sequencing setup steps, etc. The library preparation system communication module may also receive acknowledgements from the sequencer of the communications transmitted by the library preparation system. Still further, the library preparation system communication module may transmit and receive any suitable communications to and from the sequencer related to the library of samples.

The library preparation system and the sequencer may communicate back and forth to ensure the sequencer is ready to receive the library of samples when library preparation has been completed. For example, the library preparation system may transmit a communication to the sequencer indicating the preparation status of the library of samples. In response to receiving the communication, the sequencer may then transmit a communication to the library preparation system indicating an estimated duration until the sequencer can receive the library of samples. This allows the library preparation system to plan its preparation steps to ensure sample integrity.

For example, if the library preparation system estimates the library of samples will be prepared before the sequencer can receive the library of samples, the library preparation system may delay denaturing and diluting the samples until the sequencer is ready to receive the library of samples or at least until the sequencer is within a threshold time period of being ready to receive the library of samples. The library preparation system may also store the samples until the sequencer is ready to receive them.

As the library preparation system prepares the samples and the sequencer performs sequencing steps before receiving the samples, the library preparation system and the sequencer may provide updated estimates of the amount of time remaining until the respective devices are ready to load and receive the samples. This allows the devices to adjust their preparation steps accordingly so that the samples do not degrade waiting for the sequencer, and the sequencer does not waste time waiting for the samples to be ready.

Then when the library of samples is ready to load into the sequencer, and/or the sequencer is ready to receive the library of samples, the library preparation system may transmit the library of samples to the sequencer across a fluidic line configured to be coupled to the library preparation system and the sequencer, such as the fluidic line 188. For example, the library preparation system may transmit the library of samples to the sequencer across the fluidic line in response to receiving the indication from the sequencer that the sequencer is ready to receive the library of samples.

FIG. 2 is a schematic implementation of another system 400 that can be used to implement the system 300 of FIG. 1. The system 400 of FIG. 2 is similar to the system 300 of FIG. 1. Like the system 300, the system 400 of FIG. 2 includes four consumables areas 302, four first working areas 304, and one second working area 306 and one loading area 308. Each of the first working areas 304 may perform the amplification process(es) and cleanup process(es) and the second working area 306 may perform the quantification processes, the library normalization processes, the pooling processes, the denaturing processes, and/or diluting processes. The sipper assembly 174 may be used to transfer the prepared library to another system such as a sequencing instrument.

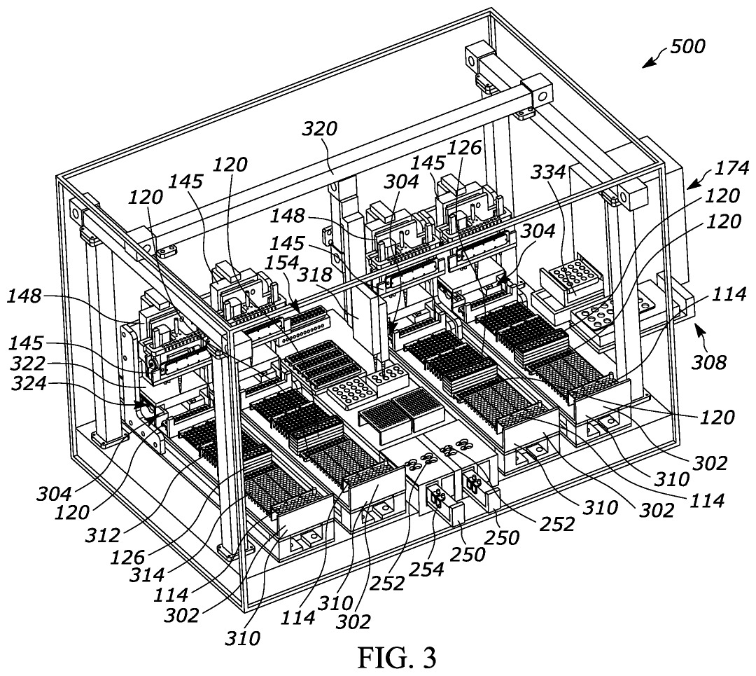

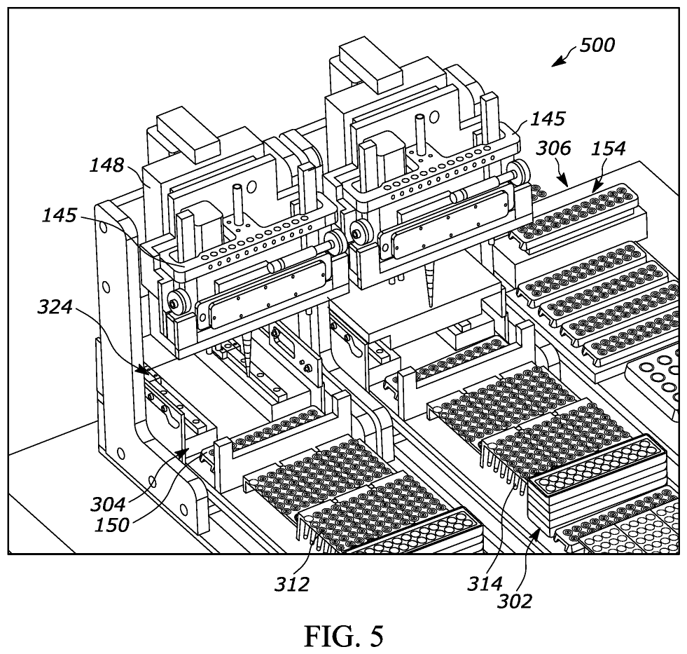

FIG. 3 is an isometric view of implementation of another system 500 that can be used to implement the system 300 of FIG. 1. The system 500 of FIG. 5 is similar to the system 400 of FIG. 2 in that the system 500 of FIG. 3 includes four consumables areas 302, four first working areas 304, one second working area 306, and one loading area 308.

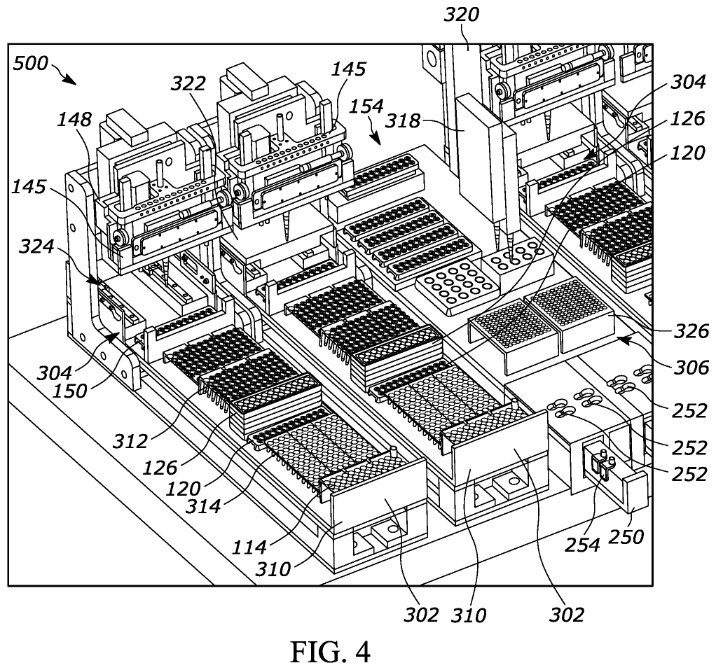

FIG. 4 is a detailed isometric view of the system 500 of FIG. 3 showing the lid 322 removed from one of the working areas 302 and the lid 322 shown in another one of the working areas 302.

FIG. 5 is another detailed isometric view of the system 500 of FIG. 3.

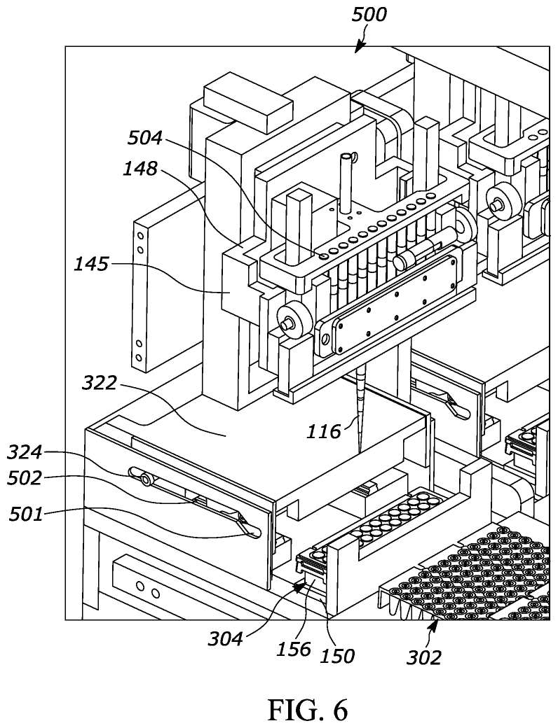

FIG. 6 is another detailed isometric view of the system 500 of FIG. 3 showing the first working area 304, the actuator 324, the lid 322, and the contact dispenser 145. The first working area 304 includes a guide 501 including a track 502 that guides the movement of the lid 322 between the retracted position shown and a position where the 322 is positioned over top of the receptacle 156. The contact dispenser 145 is shown having twelve heads 504 that receive the tips 116.

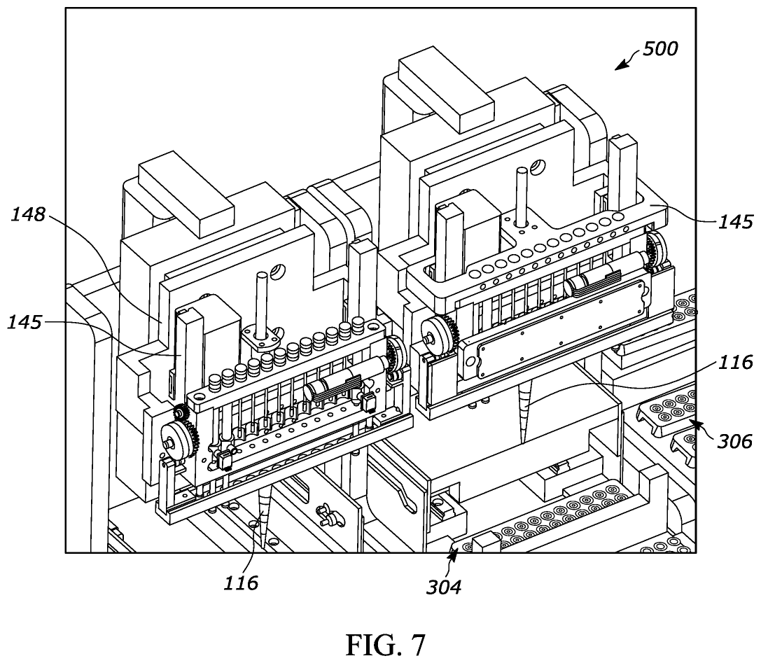

FIG. 7 is another detailed isometric view of the system 500 of FIG. 3 showing two of the first working areas 304 and two of the contact dispensers 145. A portion of the one of the contact dispensers 145 is removed to more clearly show the inner workings of the contact dispenser 145.

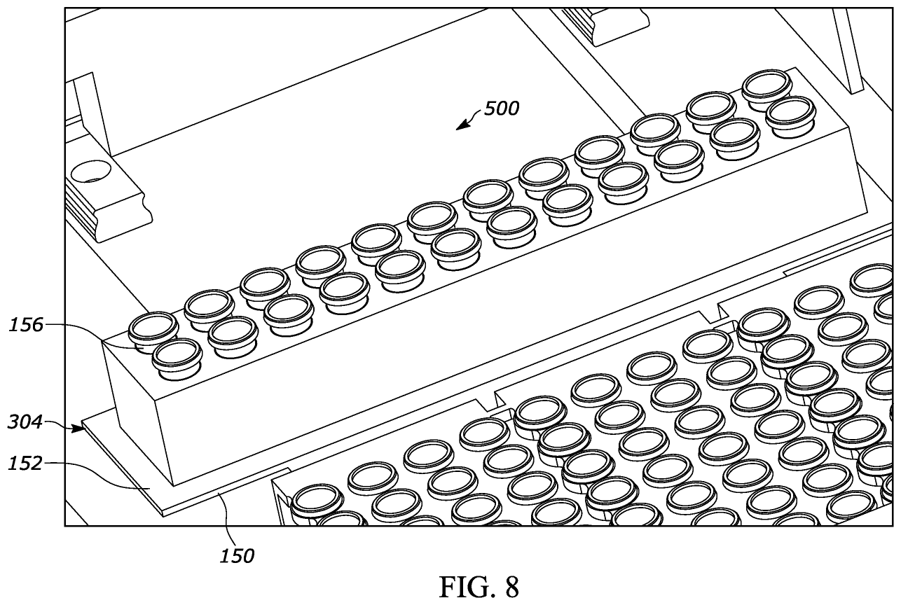

FIG. 8 is a detailed isometric view of the system 500 of FIG. 3 showing the first working area 304 and the first plate receptacle 156. The plate receptacle 156 is shown including a thermal block defining well receptacles and the thermocycler 152 is positioned beneath the well receptacles.

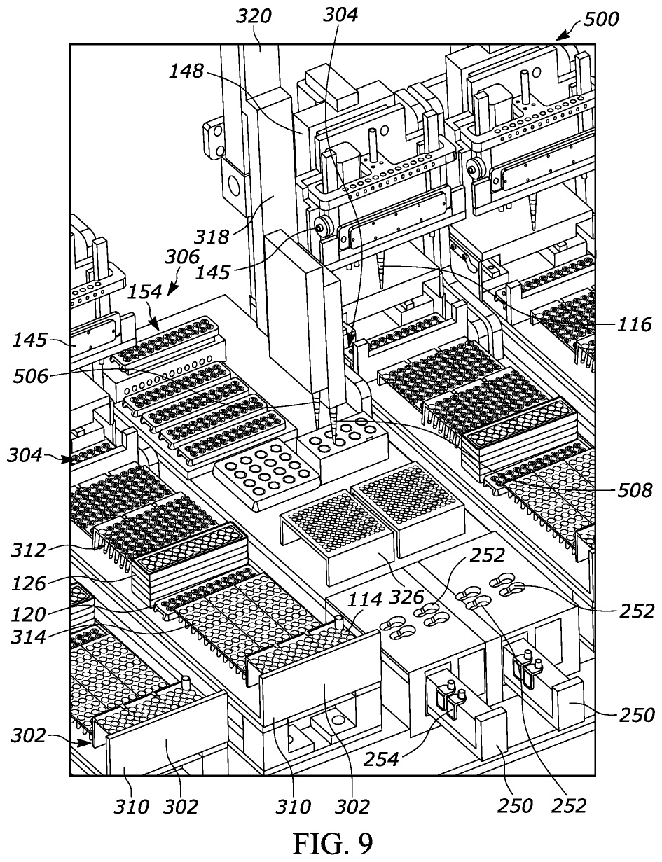

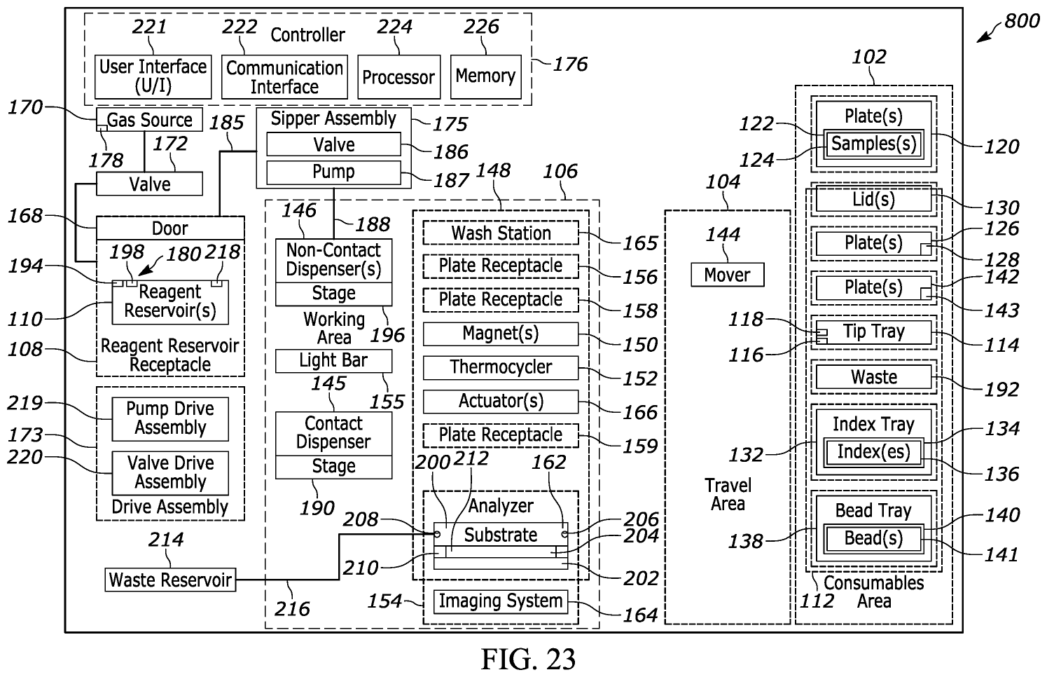

FIG. 9 is a detailed isometric view of the system 500 of FIG. 3 showing the second working area 306 and a portion of one of the first working areas 304. The second working area 306 includes the analyzer area 154 and the contact dispenser 318. The contact dispenser 318 includes a first head 506 that carries a first tip and a second head 508 that carries a second tip. The first head 506 may be used to aspirate/dispense a first volume of fluid and the second head 508 may be used to aspirate/dispense a second volume of fluid. The contact dispenser 318 may include additional heads for carrying additional tips. For example, the contact dispenser 318 may include twelve heads 506 that carry twelve tips. The contact dispenser 318 may additionally or alternatively include or carry a non-contact dispenser (see, the non-contact dispenser 146 of FIG. 23, for example).

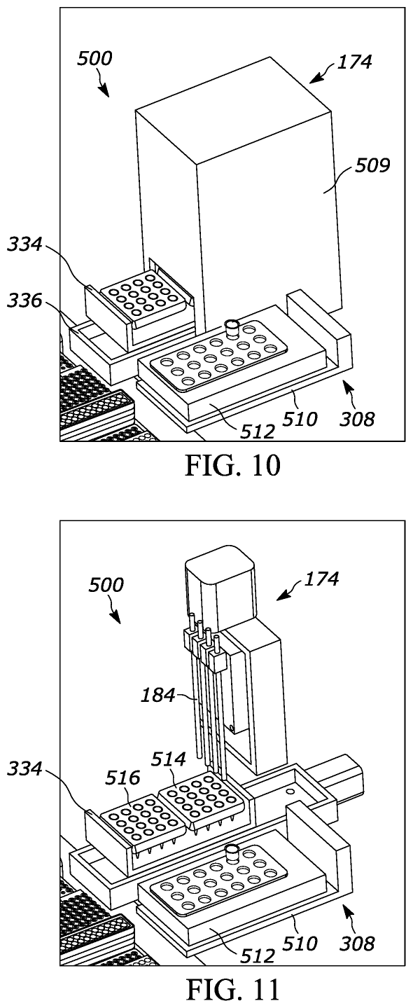

FIG. 10 is a detailed isometric view of the system 500 of FIG. 3 showing the loading area 308 including the sipper assembly 174 and the stage 336 that moves the plate receptacle 334 relative to the sipper assembly 174. The sipper assembly 174 includes a housing 509 that encloses the sippers 184. The loading area 308 also includes a reagent cartridge receptacle 510 carrying a reagent cartridge 512 in the implementation shown. The reagent cartridge 512 may be a dry reagent cartridge carrying a flow cell in some implementations. As used herein, a “flow cell” can include a device having a lid extending over a reaction structure to form a flow channel therebetween that is in communication with a plurality of reaction sites of the reaction structure, and can include a detection device that detects designated reactions that occur at or proximate to the reaction sites. The contact dispenser 318 may be used to load the prepared libraries in the reagent cartridge 512.

FIG. 11 is a detailed isometric view of the system 500 of FIG. 3 showing the loading area 308 including the sipper assembly 174 with the housing 509 of the sipper assembly 174 removed to show the sippers 184. The plate receptacle 334 is shown carrying two plates 514, 516.

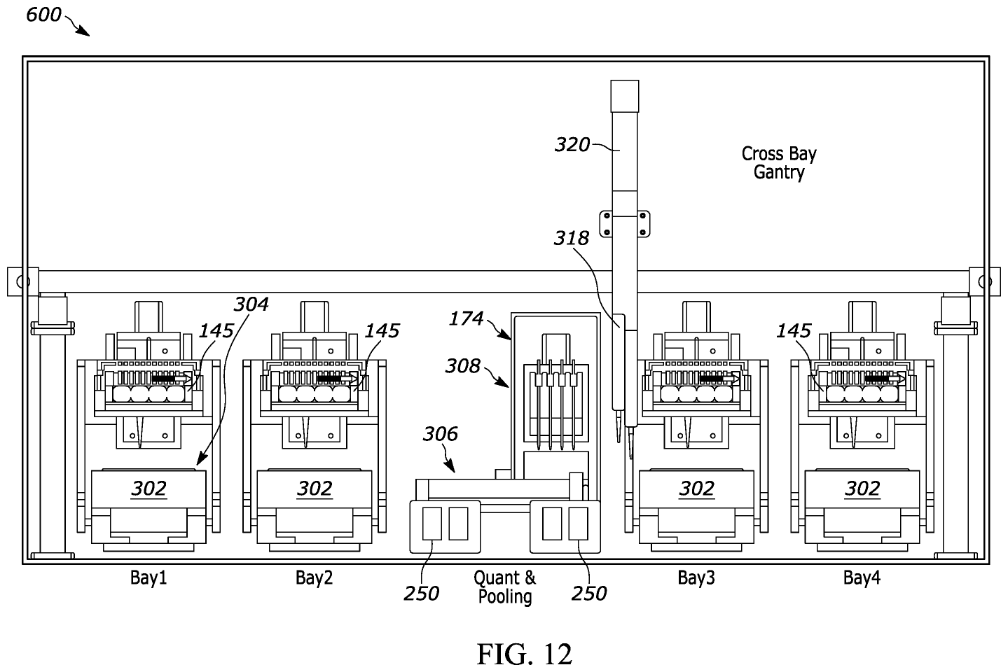

FIG. 12 is a front view of another implementation of a system 600 that can be used to implement the system 300 of FIG. 1. The system 600 of FIG. 12 is similar to the system 500 of FIG. 3 in that the system 600 of FIG. 12 includes four consumables areas 302, four first working areas 304, one second working area 306, and one loading area 308. The loading area 308 is shown adjacent and/or part of the second working area 306 in the system 600 of FIG. 12, however. The reagent cartridge receptacle 510 is also shown included with the second working area 306 in the system 600 of FIG. 12.

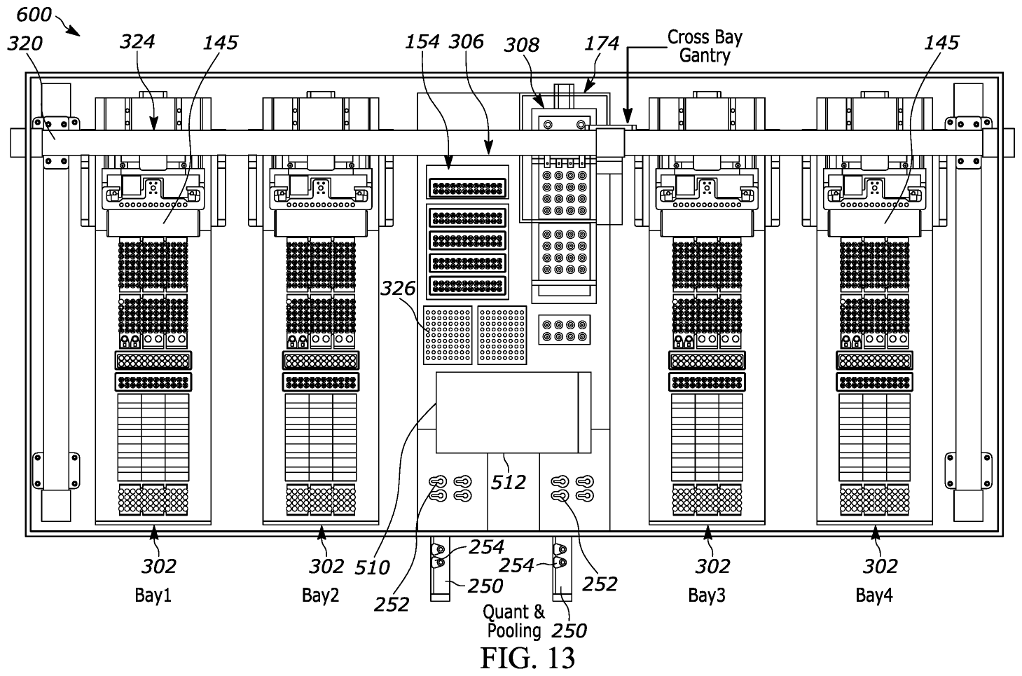

FIG. 13 is a top plan view of the system 600 of FIG. 12.

FIG. 14 is an isometric view of one of the consumables areas 302 and one of the first working areas 304 of the system 600 of FIG. 12.

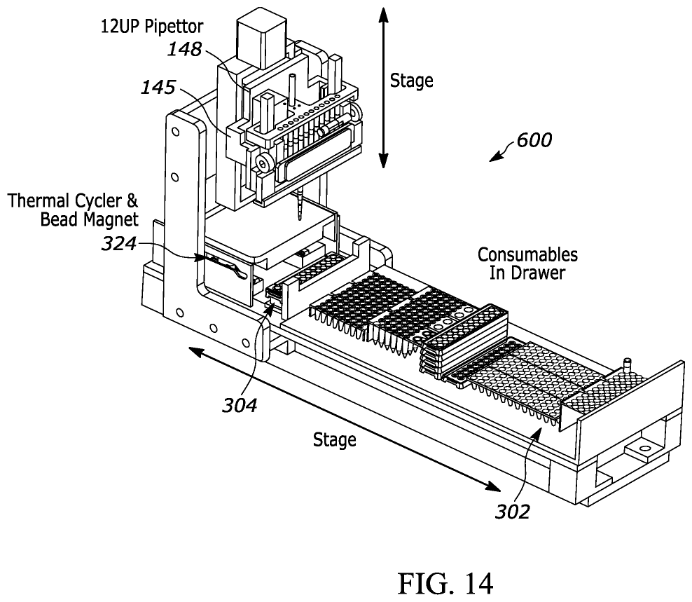

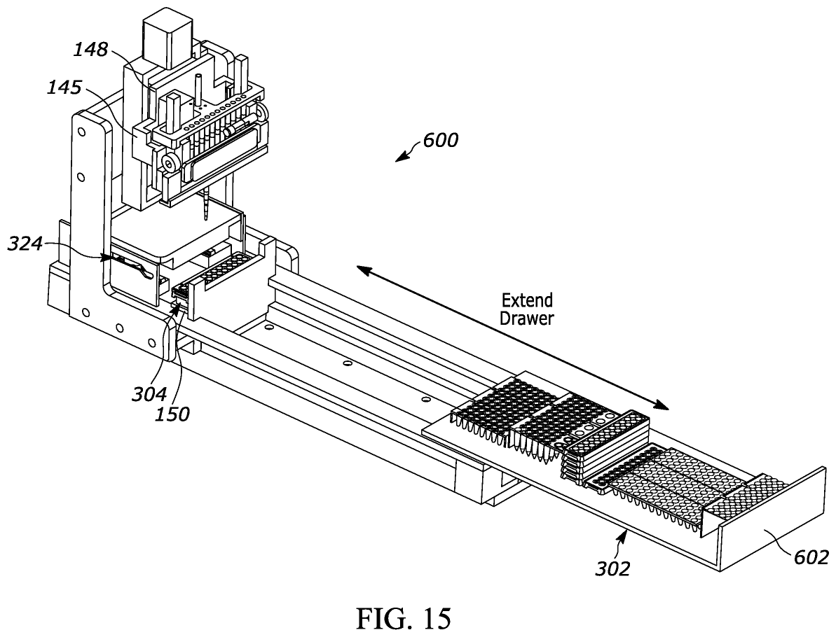

FIG. 15 is an isometric view of one of the consumables areas 302 implemented by a drawer 602 shown in the extended (or loading) position.

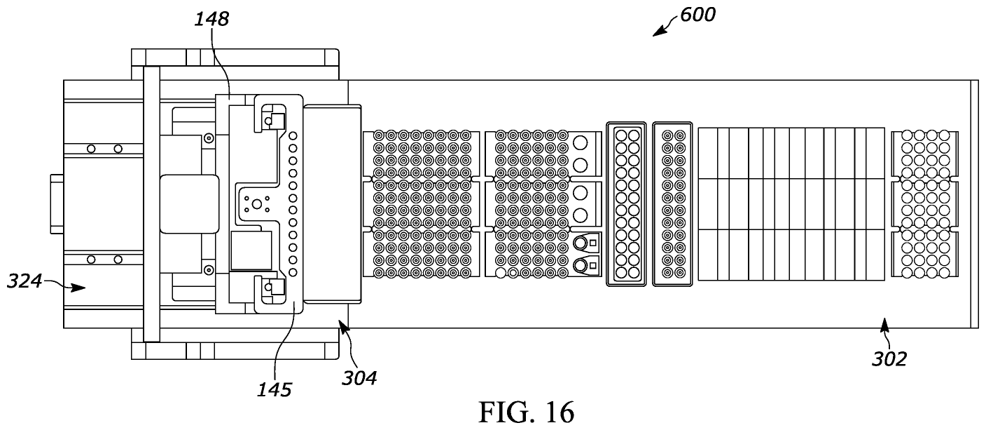

FIG. 16 is top plan view one of the consumables areas 302 and one of the first working areas 304 of the system 600 of FIG. 12.

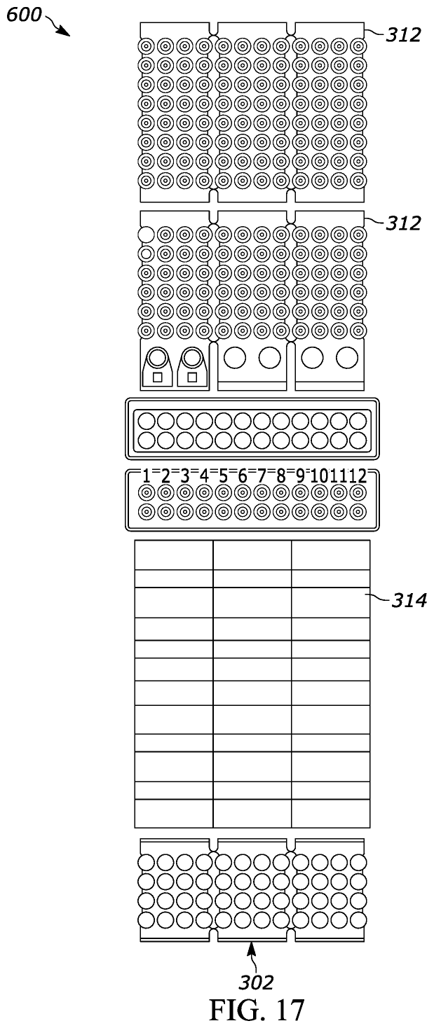

FIG. 17 is top plan view of one of the consumables areas 302 of the system 600 of FIG. 12. The consumables area 302 of FIG. 17 includes liquid reagents, bulk reagents, working plates, lyophilized reagents, and disposable tips. The lyophilized reagents may include twelve columns and twelve rows of individual reagent containers in some implementations. Any number of columns and/or rows of lyophilized reagents may be provided, however. Each column (up and down as shown in FIG. 17) may be used when processing a single sample and, thus, the same pipette tips and/or less pipette tips may be used during amplification processes and/or cleanup processes with no or less concerns of cross-contamination, for example.

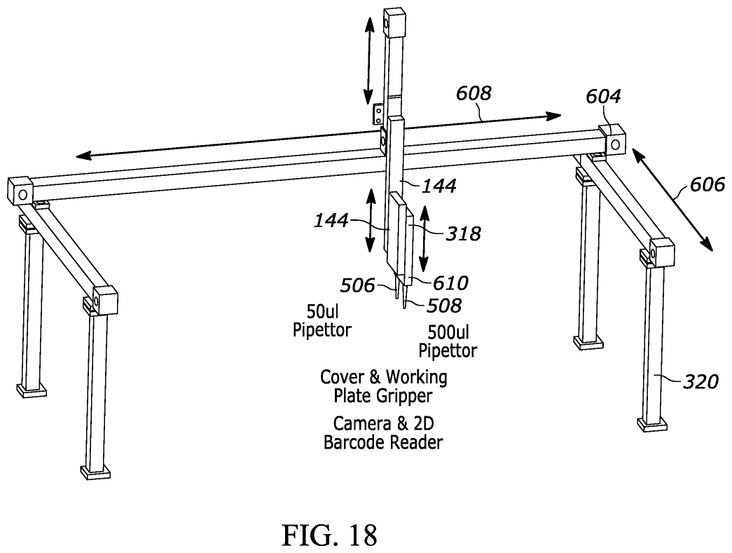

FIG. 18 is an isometric view of the contact dispenser 318, the mover 144, and the stage 320 of the system 600 of FIG. 12. The stage 320 is implemented by a gantry 604 that allows the contact dispenser 318 and the mover 144 to move in the x-direction 606, the y-direction 608, and the z-direction 610. The heads 506, 508 of the contact dispenser 318 may be independently movable in the z-direction in some implementations, thereby allowing the heads 506, 508 to be independently operable.

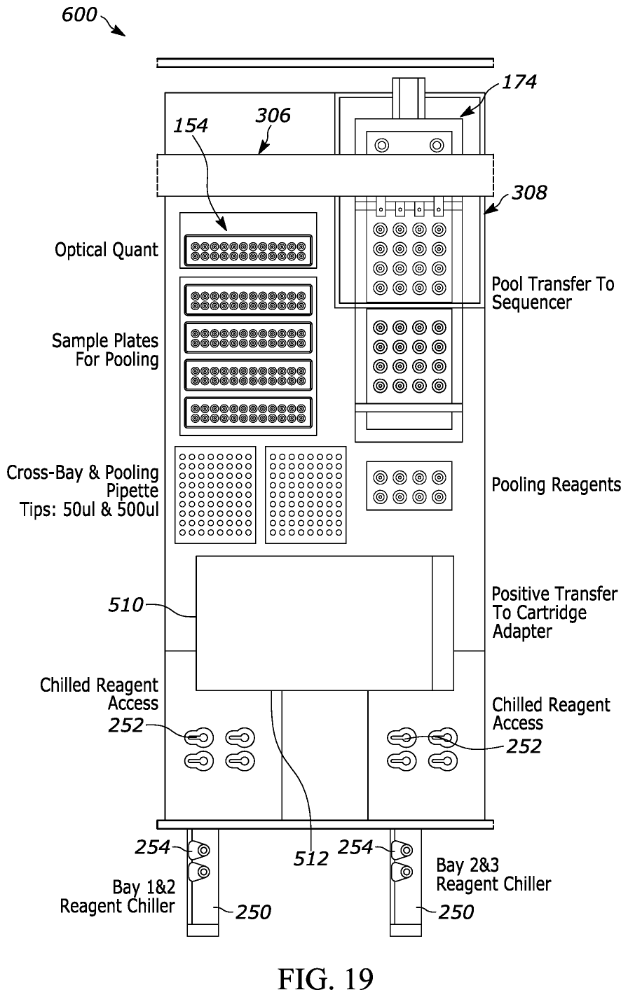

FIG. 19 is a top plan view of the second working area 306 and the loading area 308 of the system 600 of FIG. 12.

FIG. 20 is an isometric view of an implementation of another system 700 that can be used to implement the system 300 of FIG. 1. The system 700 of FIG. 20 is similar to the system 500 of FIG. 3 but the system 700 of FIG. 20 includes two consumables areas 302, two first working areas 304, one second working area 306, and one loading area 308. The loading area 308 and the reagent cartridge receptacle 510 are shown on the side of the system 700.

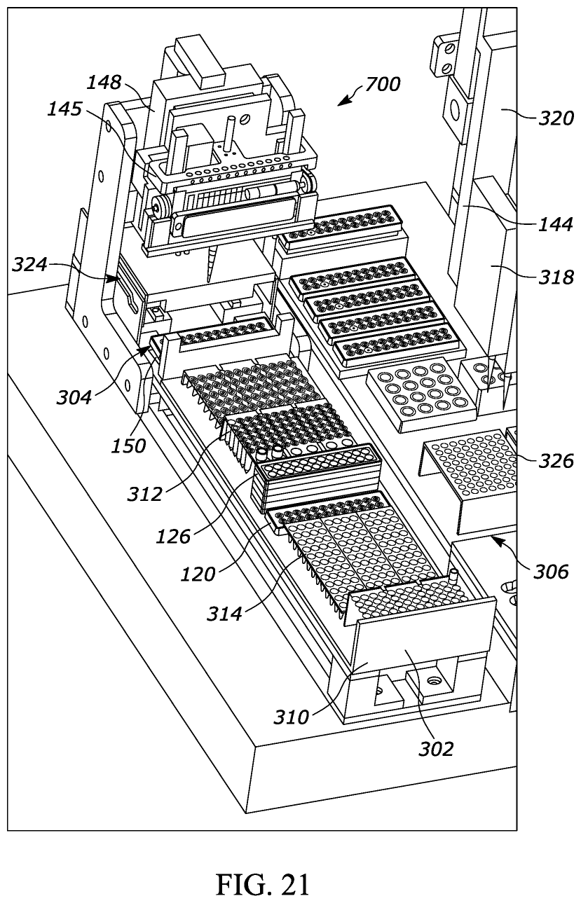

FIG. 21 is a detailed isometric view of the consumables area 302, the first working area 304, and the second working area 306 of the system 700 of FIG. 20.

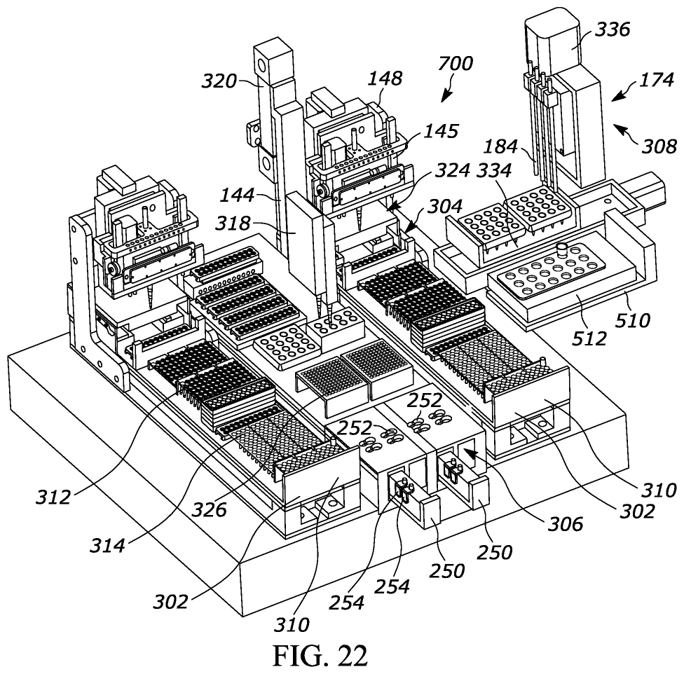

FIG. 22 is a detailed isometric view of the consumables area 302, the first working area 304, the second working area 306, and the loading area 308 of the system 700 of FIG. 20.

FIG. 23 illustrates a schematic diagram of an implementation of another system 800 in accordance with the teachings of this disclosure. The system 800 may can be used to automatically, easily, and efficiently prepare DNA libraries for sequencing applications, for example. The system 800 includes a consumables area 102, a travel area 104, a working area 106, and a reagent reservoir receptacle 108 to receive a reagent reservoir 110 in the implementation shown. The reagent reservoir receptacle 108 may alternatively be positioned above the working area 106.

The consumables area 102 includes a consumables receptacle 112 that is shown receiving a tip tray 114 having a first tip 116 and a second tip 118, a first plate 120 having a well 122 containing a sample 124, and a second plate 126 having a well 128. The consumables receptacle 112 may be a drawer that can be pulled out from the system 800 and loaded with the consumables 114, 116, 118, 120, 126. The consumables receptacle 112 is also shown receiving a lid 130, an index tray 132 having a well 134 containing indexes 136, and a bead tray 138 having a well 140 containing beads 141. The consumables area 102 may also have a third plate 142 having a well 143.

The first plate 120, the second plate, 126, the index tray 132, and/or the bead tray 138 may be a stack of the corresponding plates 120 and/or 126 and/or trays 132 and/or 138. The tip tray 116 may have a plurality of the first tips 116, a plurality of the second tips 118, and/or one or more tips that are different sizes from the first tips 116 and/or the second tips 118. While the tip tray 114 is mentioned having the first tip 116 and the second tip 118, the tip tray 114 can have any number of tips such as 24 tips. The plates 120, 126 may have any number of wells, however.

While the plates 120 and 126 are mentioned having a single well 122, 128, the plates 120, 126 may have a plurality of wells such as 24 wells. The plates 120, 126 may include a 2×12 array of wells that allow for side access for all wells in some implementations. The plates 120, 126 being implemented by a 2×12 array allows a fill level/bubbles to be inspected in every well with side-view computer vision, increased heat transfer in heating (PCR) operations, and opportunities in magnetic pull down operations.

The travel area 104 includes a mover 144 and the working area 106 includes a contact dispenser 145, a non-contact dispenser 146, a stage 148, a magnet 150, a thermocycler 152, and an analyzer area 154 in the implementation shown. The mover 144 may include a robotic arm. The non-contact dispenser 146 is fluidly coupled to the reagent reservoir 110. The non-contact dispenser 146 may alternatively aspirate reagent from the reagent reservoir 110 using tips 116 and/or 118 and dispense the reagent into the wells of the corresponding plates 120, 126. The non-contact dispenser 146 may not be fluidly coupled to the reagent reservoir 110 in such implementations. The stage 148 may be an x-y stage. While the contact dispenser 145 and the non-contact dispenser 146 are shown schematically on the side of the stage 148, the contact dispenser 145 and the non-contact dispenser 146 may be positioned above the stage 148. The working area 106 may also include a light bar 155 that may be used to degrade oligonucleotides. The light bar 155 may be a high power ultraviolet light (UV) light bar that is regularly used throughout a workflow to repeatedly degrade oligonucleotides to deter cross contamination in some implementations.

The stage 148 has a first plate receptacle 156, a second plate receptacle 158, and a third plate receptacle 159 and the analyzer area 154 includes a substrate 162 and an imaging system 164. The plate receptacles 156, 158, 159 may be referred to as plate stations. The imaging system 164 may be a fluorescent imaging system, a fluorescence spectrophotometer including an objective lens and/or a solid-state imaging device. The solid-state imaging device may include a charge coupled device (CCD) and/or a complementary metal oxide semiconductor (CMOS). The stage 148 may also include a wash station 165 that can be used to clean and/or rinse the tips 116 and/or 118 between sequential interfaces to the sample/reagent(s) to deter against reagent-to-reagent cross contamination, for example. The wash station 165 may alternatively be omitted. While the stage 148 is shown including the three plate receptacles 156, 158, 159, any number of plate receptacles may be included such as six plate receptacles.

The system 800 may perform DNA library preparation workflows that include amplification processes, cleanup processes, library normalization processes, and/or pooling processes in some implementations. The system 800 may perform workflows such as whole genome sequencing (WGS) workflows, DNA & RNA enrichment workflows, methylation workflows, split-pool amplicon workflows, and/or amplicon workflows. The DNA library preparation workflow can be performed on any number of samples such as between one sample and twenty four samples. The system 800 thus allows for variable batch processing.

The mover 144 moves the tip tray 114, the first plate 120, the second plate 126, the lid 130, and the index tray 132 in operation to perform the processes of the work flow between the consumables area 102 and the working area 106 and the stage 148 aligns the first plate receptacle 156, the second plate receptacle 158, and the third plate receptacle 159 relative to the contact dispenser 145 and the non-contact dispenser 146. The system 800 also includes an actuator 166, a door 168, a gas source 170, a valve 172, a drive assembly 173, a sipper assembly 175, and a controller 176. The gas source 170 is fluidly coupled to the regent reservoir 110. The actuator 166, the door 168, and/or the gas source 170 may alternatively be omitted.

The door 168 is movable to enclose the reagent reservoir receptacle 108 and the actuator 166 moves the magnet 150 relative to the second plate receptacle 158. The actuator 166 can move the magnet 150 between an upward position where the magnet 150 affects any plate positioned on the second plate receptacle 158 and a downward position where the magnet 150 does not affect any plate positioned on the second plate receptacle 158. The magnet 150 being moved relative to the second plate receptacle 158 and any plate 120, 126, 142 positioned on the second plate receptacle 158 allows less area on the working area 106 to be consumed. The magnet 150 can moreover be moved with relatively higher confidence as compared to an alternative approach to moving one of the plates 120, 126, 142 filled with samples to a separate magnet station. The magnet 150 may be implemented by a halbach array configuration to strengthen and focus the corresponding magnetic fields.

The valve 172 controls a flow of gas 178 from the gas source 170 to the reagent reservoir receptacle 108. The gas 178 may include nitrogen. The door 168 encloses the reagent reservoir receptacle 108 and allows the reagent reservoir 110 and reagent 180 contained within the reagent reservoir 110 to not be exposed to ambient light. The reagent reservoir receptacle 108 can be filled with the gas 178 and/or not be exposed to less ambient light and the reagent 180 contained within the reagent reservoir 110 allowing the reagent to not be exposed to atmospheric gases such as oxygen and/or to be less exposed to ambient gases. An environment such as a temperature of the reagent reservoir receptacle 108 can, thus, be temperature controlled. The reagent reservoir 110 storing the reagent 180 in a controlled temperature allows the reagent 180 to be stored on the system 800 for a threshold amount of time such as many weeks.

The sipper assembly 175 may be coupled to a corresponding number of the reagent reservoirs 110 via reagent sippers 185. The reagent reservoir 110 may contain fluid (e.g., reagent and/or another reaction component). The sipper assembly 175 includes a plurality of ports in some implementations where each port of the sipper assembly 175 may receive one of the reagent sippers 185. The reagent sippers 185 may be referred to as fluidic lines. The sipper assembly 175 also includes a valve 186 that may be selectively actuated to control the flow of fluid through a fluidic line(s) 188. The fluidic line 188 may include a plurality of fluidic lines where each fluidic line 188 is used to flow one sample, for example. The sipper assembly 175 also includes a pump 187 to selectively flow the reagent(s) 180 from the reagent reservoir 110, through the reagent sipper 185, through the fluidic line 188, and out of the non-contact dispenser 146. The pump 187 may additionally or alternatively be used to actuate valves of the non-contact dispenser 146.

The valve 186 may be implemented by a rotary valve, a pinch valve, a flat valve, a solenoid valve, a check valve, a piezo valve, etc. Other fluid control devices may prove suitable. The pump 187 may be implemented by a syringe pump, a peristaltic pump, and/or a diaphragm pump. Other types of fluid transfer devices may be used, however. The controller 176 is electrically and/or communicatively coupled to the mover 144, the thermocycler 152, the actuator 166, the imaging system 164, the contact dispenser 145, the non-contact dispenser 146, the sipper assembly 175, the valve 186, the pump 187, the door 168, and the drive assembly 173 to perform various functions as disclosed herein. The sipper assembly 175 may alternatively be omitted.

The mover 144 moves the tip tray 114 from the consumables area 102 to the first plate receptacle 156, the first plate 120 from the consumables area 102 to the second plate receptacle 158, and the index tray 132 from the consumables area 102 to the third plate receptacle 159 in some implementations to initiate a DNA library preparation workflow. Different wells 122 of the first plate 120 may contain different samples 124. The samples 124 may be a biological sample derived from a human, animal, plant, bacteria, or fungi. Other sources of obtaining the biological samples may prove suitable. The mover 144 may include a gantry having grippers that can pick-and-place objects such as the plates 120, 126 and/or the trays 132, 138 between different areas 102, 104, 106 of the system 800. The mover 144 may be implemented in different ways, however.

The stage 148 aligns the contact dispenser 145 with the tip tray 114 and the contact dispenser 145 couples with the first tip 116 from the tip tray 114. While the contact dispenser 145 is mentioned coupling with one first tip 116, the contact dispenser 145 may couple with a number of the first tips 116 that corresponds to the number of the wells 122 in the first plate 120 and/or the number of the wells 122 in the first plate 120 containing the sample 124. The first tip 116 may be a smaller pipette tip that is used to move smaller fluid volumes and the second tip 118 may be a larger pipette tip that is used to move larger fluid volumes, for example. Each of the first tip 116 and/or the second tip 118 may be exposed to a single sample during a workflow reducing the likelihood of cross-contamination. Each of the first tip 116 and/or the second tip 118 may be used through an entire workflow. The contact dispenser 145 may couple with and/or use different ones of the tips 116, 118 depending on the workflow and/or the processes within a workflow that the system 800 is implementing.

A stage 190 may be coupled to and move the contact dispenser 145 to allow the contact dispenser 145 to couple with the tips 116, 118. The stage 190 may be a z-stage. The stage 190 may alternatively be omitted. The stage 148 aligns the contact dispenser 145 with the index tray 132 and the contact dispenser 145 aspirate the indexes 136 from the index tray 132 using the first tip 116. The stage 148 can then align the contact dispenser 145 with the first plate 120 and the contact dispenser 145 dispenses the indexes 13 into the well 122 of the first plate 120. The mover 144 moves the lid 130 from the consumables area 102 and places the lid 130 on the first plate 120 to cover the well 122 of the first plate 120 with the lid 130. The lid 130 may alternatively be movably coupled to the thermocycler 152. The lid 130 may not be disposable in such implementations.

The thermocycler 152 is aligned with the second plate receptacle 158 and the thermocycler 152 amplifies the sample 124 within the well 122 of the first plate 120. The thermocycler 152 is carried by the stage 148 in the implementation shown. The thermocycler 152 and/or the magnet 150 can, thus, act on a plate 120, 126 received at the second plate receptacle 158. The thermocycler 152 may alternatively be spaced from the magnet 150 and/or not carried by the stage 148.

The mover 144 can move the lid 130 from the first plate 120 to the consumables area 102 after the amplification processes and the mover 144 moves the index tray 132 from the third plate receptacle 159 to the consumables area 102. The consumables area 102 includes a waste 192 that can receive used consumables such as, for example, the lid 130. The lid 130 may alternatively be reused, however. The waste 192 may be a waste tray having an absorbent material to absorb liquid waste.

The system 800 may perform the cleanup processes after the amplification processes are performed. The mover 144 moves the bead tray 138 from the consumables area 102 to the third plate receptacle 159 to initiate the cleanup processes in some implementations. The stage 148 aligns the contact dispenser 145 with the bead tray 138 and the contact dispenser 145 aspirates the beads 141 from the bead tray 138. The contact dispenser 145 may aspirate the beads 141 using the same first tip 116 used to aspirate the indexes 136. The contact dispenser 145 may alternatively use another one of the first tips 116 or one of the second tips 118 to aspirate the beads 141.

The stage 148 aligns the contact dispenser 145 with the first plate 120 and the contact dispenser 145 dispenses the beads 141 into the well 140 of the first plate 120 as part of the cleanup process. The stage 148 aligns the non-contact dispenser 146 with the first plate 120 and the non-contact dispenser 146 dispenses a first reagent 194 from the reagent reservoir 110 into the well 122 of the first plate 120. The first reagent 194 may be a bead buffer and the sample 124 may bind to the beads 141 in the presence of the bead buffer. The non-contact dispenser 146 may be able to jet dispense with adequate liquid velocity to enable jet mixing in some implementations. The accuracy of the non-contact dispenser 146 may allow less reagent to be used as compared to manual workflows and between about % of the reagent to about % of the reagent may be used, for example. The non-contact dispenser 146 can deliver volumes of reagent of about 1 μL with a precision error of less than about 2% coefficient of variation (CV) and an accuracy error of less than about 4% such that the total fluid volume error is less than about 10%, in some examples. The non-contact dispenser 146 may deliver about 50 μL to each well 122 of the first plate 120 in about 48 seconds (sec) for larger volume deliveries and may delivery about 5 μL to each well 122 of the first plate 120 in about 24 seconds (sec) for smaller volume deliveries.

A stage 196 may be coupled to and move the non-contact dispenser 146 to allow the non-contact dispenser 146 to dispense liquid such as the first reagent 194 into the well 122 of the first plate 120. The first reagent 194 may be a bead buffer and the sample 124 may bind to the beads 141 in the presence of the first reagent 194. The stage 196 may be a z-stage or an x-y-x stage in some implementations. The stage 196 may be an x-y-z stage in implementations when the non-contact dispenser 146 couples with a tip 116 and/or 118 and the non-contact dispenser 146 is positioned to aspirate the reagent 180 directly from the reagent reservoir 110. The stage 196 may alternatively be omitted.

The stage 148 aligns the contact dispenser 145 with the tip tray 114 and the contact dispenser 145 places the first tip 116 in the tip tray 114 and the contact dispenser 145 then couples with the second tip 118 from the tip tray 114. While the contact dispenser 145 is mentioned coupling with one second tip 118, the contact dispenser 145 may couple with a number of the second tips 118 that corresponds to the number of the wells 122 in the first plate 120 and/or the number of the wells 122 in the first plate 120 containing the sample 124.

The actuator 166 moves the magnet 150 toward the second plate receptacle 158 and the magnet 150 draws the beads 141 toward the magnet 150. The beads 141 and the sample 124 bound to the beads 141 may be positioned toward the bottom of the well 122 of the first plate 120 or on a side(s) of the well 122. The tips 116 and/or 118 may easily access the well 122 if the beads 141 are on the side of the well 122. The magnet 150 may cause the beads 141 to be in any position within the well 122, however.

The stage 148 aligns the contact dispenser 145 with the first plate 120 and the contact dispenser 145 aspirates the first reagent 194 from the well 122 of the first plate 120. The mover 144 can move the bead tray 138 from the third plate receptacle 159 to the consumables area 102. The mover 144 may move the waste 192 to the third plate receptacle 159 and the contact dispenser 145 may dispense the first reagent 194 aspirated from the well 122 of the first plate 120 into the waste 192. The mover 144 may move the waste 192 back to the consumables area 102.

The stage 148 aligns the non-contact dispenser 146 with the first plate 120 and the non-contact dispenser 146 dispenses a second reagent 198 from the reagent reservoir 110 into the well 122 of the first plate 120. The second reagent 198 may be an elution buffer that releases the sample 124 from being bound to the beads 141 and, specifically, releases DNA associated with the sample 124 from being bound to the beads 141.

The mover 144 moves the second plate 126 from the consumables area 102 to the third plate receptacle 159. The system 800 can use the second plate 126 for a transfer operation. The actuator 166 moves the magnet 150 toward the second plate receptacle 158 to draw the beads 141 toward the magnet 150 and, thus, suspend the second reagent 198 and the sample 124 within the well 122.

The stage 148 aligns the contact dispenser 145 with the first plate 120 and the contact dispenser 145 aspirates the second reagent 198 and the sample 124 from the well 122 of the first plate 120 using the second tip 118, for example. The stage 148 aligns the contact dispenser 145 with the second plate 126 and the contact dispenser 145 dispenses the second reagent 198 and the sample 124 into the well 128 of the second plate 126.

The system 800 may perform the quantification processes after the cleanup processes are performed. The stage 148 aligns the contact dispenser 145 with the tip tray 114 to initiate the quantification processes in some implementations and the contact dispenser 145 places the second tip 118 in the tip tray 114 and the contact dispenser 145 couples with the first tip 116 from the tip tray 114.

The substrate 162 of the analyzer area 154 is carried by the stage 148 in the implementation shown and the imaging system 164 is spaced from the stage 148 and coupled to a portion of the system 800 such as a frame of the system 800. The imaging system 164 may alternatively be carried by the stage 148. The substrate 162 is shown including a pair of plates 200, 202 between which a gap 204 is defined. The substrate 162 also has an inlet 206 and an outlet 208 in fluid communication with the gap 204 and a seal 210 positioned between the pair of plates 200, 202. The plates 200, 202 and the seal 210 define a channel 212 between the inlet 206 and the outlet 208. A waste reservoir 214 is fluidly coupled to the outlet 208 of the substrate 162 by a fluidic line 216.

The stage 148 align the contact dispenser 145 with the second plate 126 to perform the quantification processes and the contact dispenser 145 aspirates a portion of the second reagent 198 and the sample 124 from the well 128 of the second plate 126. The portion of the second reagent 198 and the sample 124 may be about 2 μL.

The stage 148 aligns the contact dispenser 145 with the inlet 206 of the substrate 162 and the contact dispenser 145 dispenses the portion of the second reagent 198 and the sample 124 into the inlet 206 of the substrate 162. The portion of the second reagent 198 and the sample 124 may flow and/or be positioned between the inlet 206 and the outlet 208 in this implementation and the imaging system 164 obtains image data of the portion of the second reagent 198 and the sample 124. The imaging system 164 and/or the system 800 uses the image data to determine a concentration of the sample 124. Negative pressure, oil, and/or another substance may be used to urge the portion of the second reagent 198 and the sample 124 between the inlet 206 and the outlet 208. The first plate 200 may alternatively be hingably coupled or removably coupled to the second plate 202 to allow the contact dispenser 145 to dispense the portion of the second reagent 198 and the sample 124 onto the second plate 202 prior to the first plate 200 be positioned overtop of the second plate 202.

The system 800 may perform the normalization processes after the quantification processes are performed. The stage 148 aligns the second plate 126 with the non-contact dispenser 146 to initiate the normalization processes in some implementations. The non-contact dispenser 146 dispenses a diluent 218 into the well 128 of the second plate 126 to dilute the sample 124 based on the concentration of the sample determined. The sample 124 within the well 128 of the second plate 126 will have a concentration within a threshold value after the diluent 218 is added to the well 128 as a result. The diluent 218 may be a buffer.

The system 800 may perform the pooling processes after the quantification processes are performed. The stage 148 aligns the contact dispenser 145 with the tip tray 114 and the contact dispenser 145 places the first tip 116 in the tip tray 114 and the contact dispenser 145 then couples with the second tip 118 from the tip tray 114 to initiate the pooling processes in some implementations. The mover 144 moves the tip tray 114 from the first plate receptacle 156 to the consumables area 102 and the mover 144 moves the third plate 142 to the first plate receptacle 156. The stage 148 aligns the second plate 126 with the contact dispenser 145 and the contact dispenser 145 aspirates the sample 124 from the well 128 of the second plate 126. The stage 148 then aligns the third plate 142 with the contact dispenser 145 and the contact dispenser 145 dispenses the sample 124 into the well 143 of the third plate 142. Additional samples from other wells of the second plate 126 may be deposited into the well 143 of the third plate 142 in a similar manner to combine a plurality of normalized samples together. A single tip can be used for the pooling processes.

The drive assembly 173 includes a pump drive assembly 219 and a valve drive assembly 220. The pump drive assembly 219 may be adapted to interface with the pump 187 to pump fluid from the reagent reservoir 110 to the non-contact dispenser 146. The valve drive assembly 220 may be adapted to interface with the valve 186 to control the position of the valve 186.

The controller 176 includes a user interface 221, a communication interface 222, one or more processors 224, and a memory 226 storing instructions executable by the one or more processors 224 to perform various functions including the disclosed implementations. The user interface 221, the communication interface 222, and the memory 226 are electrically and/or communicatively coupled to the one or more processors 224.

In an implementation, the user interface 221 receives input from a user and provides information to the user associated with the operation of the system 800 and/or an analysis taking place. The user interface 221 may include a touch screen, a display, a key board, a speaker(s), a mouse, a track ball, and/or a voice recognition system. The touch screen and/or the display may display a graphical user interface (GUI).

In an implementation, the communication interface 222 enables communication between the system 800 and a remote system(s) (e.g., computers) using a network(s). The network(s) may include an intranet, a local-area network (LAN), a wide-area network (WAN), the intranet, etc. Some of the communications provided to the remote system may be associated with an amplification process(es), a cleanup process(es), a library normalization process(es), and/or a pooling process(es)), etc. generated or otherwise obtained by the system 800. Some of the communications provided to the system 800 may be associated with an amplification process(es), a cleanup process(es), a library normalization process(es), and/or a pooling process(es) to be executed by the system 800.

The one or more processors 224 and/or the system 800 may include one or more of a processor-based system(s) or a microprocessor-based system(s). In some implementations, the one or more processors 224 and/or the system 800 includes a reduced-instruction set computer(s) (RISC), an application specific integrated circuit(s) (ASICs), a field programable gate array(s) (FPGAs), a field programable logic device(s) (FPLD(s)), a logic circuit(s), and/or another logic-based device executing various functions including the ones described herein. F

The memory 226 can include one or more of a hard disk drive, a flash memory, a read-only memory (ROM), erasable programable read-only memory (EPROM), electrically erasable programable read-only memory (EEPROM), a random-access memory (RAM), non-volatile RAM (NVRAM) memory, a compact disk (CD), a digital versatile disk (DVD), a cache, and/or any other storage device or storage disk in which information is stored for any duration (e.g., permanently, temporarily, for extended periods of time, for buffering, for caching).

FIG. 24A illustrates a schematic diagram of an implementation of another system 900 in accordance with the teachings of this disclosure. The system 900 may be a sequencing system and/or a next generation sequencing (NGS) system. The system 900 can be used to perform an analysis on one or more samples of interest. The sample may include one or more DNA clusters that have been linearized to form a single stranded DNA (sstDNA). In the implementation shown, the system 900 is adapted to receive a pair of flow cell assemblies 902, 904 including corresponding flow cells 906 and includes, in part, an imaging system 908 and a flow cell interface 910 having flow cell receptacles 912, 914 that support the corresponding flow cell assemblies 902, 904. The flow cell interface 910 may be associated with and/or referred to as a flow cell deck structure. The system 900 also includes a stage assembly 916, a pair or reagent selector valve assemblies 918, 920 that each include a reagent selector valve 922 and a valve drive assembly 924, and a controller 926. The reagent selector valve assemblies 918, 920 may be referred to as mini-valve assemblies. The controller 926 is electrically and/or communicatively coupled to the imaging system 908, reagent selector valve assemblies 918, 920, and to the stage assembly 916 and is adapted to cause the imaging system 908, reagent selector valve assemblies 918, 920 and the stage assembly 916 to perform various functions as disclosed herein.

In this implementation, the reagent selector valve assemblies 918, 920 are carried by the flow cell interface 910 and are positioned immediately adjacent to the corresponding flow cell assembly 902, 103. The proximity between the reagent selector valve assemblies 918, 920 and the corresponding flow cell assemblies 902, 103 allows for reagent consumption reduction, dead volume reduction within, for example, fluidic lines, carry over reduction, switching times reduction, and/or time-to-time results. The stage assembly 916 includes an x-motor and ball screw 928 that moves the flow cell interface 910 in the x-direction relative to the imaging system 908 and a y-motor and ball screw 930 that moves the flow cell interface 910 in the y-direction relative to the imaging system 908.

Referring still to the system 900 of FIG. 24A, in the implementation shown, the system 900 also includes a sipper assembly 934, a sample loading assembly 936, a pump manifold assembly 938, a drive assembly 940, and a waste reservoir 942. The controller 926 is electrically and/or communicatively coupled to the sipper assembly 934, the sample loading assembly 936, the pump manifold assembly 938, and the drive assembly 940 and is adapted to cause the sipper assembly 934, the sample loading assembly 936, the pump manifold assembly 938, and the drive assembly 940 to perform various functions as disclosed herein. The sample loading assembly 936 may include sample ports 1001 and flow cell ports 1003.

The controller 926 may also be electrically and/or communicatively coupled to the controller 176 of another system (e.g., the system 300, 400, 500, 600, 700, 800). The sample loading assembly 936 may be referred to as a sample manifold loading assembly.

Referring to the flow cells 906, in the implementation shown, each of the flow cells 906 includes a plurality of channels 944, each having a first channel opening positioned at a first end of the flow cell 906 and a second channel opening positioned at a second end of the flow cell 906. Depending on the direction of flow through the channels 944, either of the channel openings may act as an inlet or an outlet. While the flow cells 906 are shown including two channels 944 in FIG. 24A, any number of channels 944 may be included (e.g., 1, 2, 6, 8).

Each of the flow cell assemblies 902, 904 also includes a flow cell frame 946 and a flow cell manifold 948 coupled to the first end of the corresponding flow cell 906. As used herein, a “flow cell” (also referred to as a flowcell) can include a device having a lid extending over a reaction structure to form a flow channel therebetween that is in communication with a plurality of reaction sites of the reaction structure. Some flow cells may also include a detection device that detects designated reactions that occur at or proximate to the reaction sites. As shown, the flow cell 906, the flow cell manifold 948, and/or any associated gaskets used to establish a fluidic connection between the flow cell 906 and the system 900 are coupled or otherwise carried by the flow cell frame 946. While the flow cell frame 946 is shown included with the flow cell assemblies 902, 904 of FIG. 24A, the flow cell frame 946 may be omitted. As such, the flow cell 906 and the associated flow cell manifold 948 and/or gaskets may be used with the system 900 without the flow cell frame 946.

In some implementations, components of the system 900 that are shown once and being coupled to both of the flow cells 906 can be duplicated such that each flow cell 906 has its own corresponding components. Each flow cell 906, for example, may be associated with a separate sample loading assembly 936, pump manifold assembly 938, etc. In other implementations, the system 900 may include a single flow cell 906 and corresponding components.

The system 900 is fluidly coupled to another system (e.g., the system 300, 400, 500, 600, 700, 800) by the fluidic lines 188 in the implementation shown. The fluidic line 188 flows one or more samples of interest (e.g., an analyte, a library) to the sample manifold assembly 936 in the implementation shown. While two fluidic lines 188 are shown, any number of fluidic lines 188 may be included.

The sample loading assembly 936 includes one or more sample valves 954 and the pump manifold assembly 938 includes one or more pumps 956, one or more pump valves 958, and a cache 960. One or more of the valves 954, 958 may be implemented by a rotary valve, a pinch valve, a flat valve, a solenoid valve, a check valve, a piezo valve, a two-way valve, a three-way valve, an electrically-actuated valve, a pneumatically-actuated valve, and combinations thereof. However, different types of fluid control devices may be used. One or more of the pumps 956 may be implemented by a syringe pump, a peristaltic pump, and/or a diaphragm pump. However, other types of fluid transfer devices may be used. The cache 960 may be a serpentine cache and may temporarily store one or more reaction components during, for example, bypass manipulations of the system 900 of FIG. 24A. While the cache 960 is shown being included in the pump manifold assembly 938, in another implementation, the cache 960 may be located in a different location. For example, the cache 960 may be included in the sipper assembly 934 or in another manifold downstream of a bypass fluidic line 962.

The sample loading assembly 936 and the pump manifold assembly 938 flow one or more samples of interest from the other system (e.g., the system 300, 400, 500, 600, 700, 800) through a fluidic line 964 toward the flow cell assembly 902, 904.

The fluidic line 964 may referred to as a flow cell fluidic line. In some implementations, the sample manifold assembly 936 can individually load/address each channel 944 of the flow cell 906 with a sample of interest. The process of loading the channels 944 of the flow cell 906 with a sample of interest may occur automatically using the system 900 of FIG. 24A.

The sample loading assembly 936 may draw the one or more samples of interest through the sample fluidic lines 188 and through the sample ports 1001. The sample loading assembly 936 may then flow the one or more samples of interest through the sample ports 1003, the flow cell fluidic lines 964, and toward the flow cell assemblies 902, 904. Each sample fluidic line 188 and each flow cell fluidic line 964 may be associated with one channel of the flow cell assembly 902, 904.

As shown in the system 900 of FIG. 24A, the sample loading assembly 936 are positioned downstream of the flow cell assemblies 902, 904. Thus, the sample loading manifold assembly 936 may load a sample of interest into the flow cell 906 from the rear of the flow cell 906. Loading a sample of interest from the rear of the flow cell 906 may be referred to as “back loading.” Back loading the sample of interest into the flow cell 906 may reduce contamination. In the implementation shown, the sample loading assembly 936 is coupled between the flow cell assemblies 902, 904 and the pump manifold assembly 938.

To draw a sample of interest from the other system (e.g., the system 300, 400, 500, 600, 700, 800) and toward the pump manifold assembly 938, the sample valves 954, the pump valves 958, and/or the pumps 956 may be selectively actuated to urge the sample of interest toward the pump manifold assembly 938. The fluidic line 188 may include a plurality of sample fluidic lines that are coupled between the sippers 184 of the sipper assembly 174 and are selectively fluidically accessible via the corresponding sample valve 954. Thus, each sample can be selectively isolated from other samples using the corresponding fluidic lines and/or sample valves 954.

To individually flow the sample of interest toward a corresponding channel of one of the flow cells 906 and away from the pump manifold assembly 938, the sample valves 954, the pump valves 958, and/or the pumps 956 can be selectively actuated to urge the sample of interest toward the flow cell assembly 902 and into the respective channels 944 of the corresponding flow cell 906. In some implementations, each channel 944 of the flow cell 906 receives the sample of interest. In other implementations, one or more of the channels 944 of the flow cell(s) 906 selectively receives the sample of interest and others of the channels 944 of the flow cell(s) 906 do not receive the sample of interest. The channels 944 of the flow cell(s) 906 that may not receive the sample of interest may receive a wash buffer instead, for example.

The drive assembly 940 interfaces with the sipper assembly 934 and the pump manifold assembly 938 to flow one or more reagents that interact with the sample within the corresponding flow cell 906. In an implementation, a reversible terminator is attached to the reagent to allow a single nucleotide to be incorporated onto a growing DNA strand. In some such implementations, one or more of the nucleotides has a unique fluorescent label that emits a color when excited. The color (or absence thereof) is used to detect the corresponding nucleotide. In the implementation shown, the imaging system 908 excites one or more of the identifiable labels (e.g., a fluorescent label) and thereafter obtains image data for the identifiable labels. The labels may be excited by incident light and/or a laser and the image data may include one or more colors emitted by the respective labels in response to the excitation. The image data (e.g., detection data) may be analyzed by the system 900. The imaging system 908 may be a fluorescence spectrophotometer including an objective lens and/or a solid-state imaging device. The solid-state imaging device may include a charge coupled device (CCD) and/or a complementary metal oxide semiconductor (CMOS). However, other types of imaging systems and/or optical instruments may be used. For example, the imaging system 908 may be or be associated with a scanning electron microscope, a transmission electron microscope, an imaging flow cytometer, high-resolution optical microscopy, confocal microscopy, epifluorescence microscopy, two photon microscopy, differential interference contrast microscopy, etc.

After the image data is obtained, the drive assembly 940 interfaces with the sipper assembly 934 and the pump manifold assembly 938 to flow another reaction component (e.g., a reagent) through the flow cell 906 that is thereafter received by the waste reservoir 942 via a primary waste fluidic line 966 and/or otherwise exhausted by the system 900. Some reaction components perform a flushing operation that chemically cleaves the fluorescent label and the reversible terminator from the sstDNA. The sstDNA is then ready for another cycle.

The primary waste fluidic line 966 is coupled between the pump manifold assembly 938 and the waste reservoir 942. In some implementations, the pumps 956 and/or the pump valves 958 of the pump manifold assembly 938 selectively flow the reaction components from the flow cell assembly 902, 904, through the fluidic line 964 and the sample manifold assembly 936 to the primary waste fluidic line 966.

The flow cell assembly 902, 904 is coupled to a central valve 968 via the flow cell interface 910. An auxiliary waste fluidic line 970 is coupled to the central valve 968 and to the waste reservoir 942. In some implementations, the auxiliary waste fluidic line 970 receives excess fluid of a sample of interest from the flow cell assembly 902, 904, via the central valve 968, and flows the excess fluid of the sample of interest to the waste reservoir 942 when back loading the sample of interest into the flow cell 906, as described herein. That is, the sample of interest may be loaded from the rear of the flow cell 906 and any excess fluid for the sample of interest may exit from the front of the flow cell 906. By back loading samples of interest into the flow cell 906, different samples can be separately loaded to corresponding channels 944 of the corresponding flow cell 906 and the single flow cell manifold 948 can couple the front of the flow cell 906 to the central valve 968 to direct excess fluid of each sample of interest to the auxiliary waste fluidic line 970. Once the samples of interest are loaded into the flow cell 906, the flow cell manifold 948 can be used to deliver common reagents from the front of the flow cell 906 (e.g., upstream) for each channel 944 of the flow cell 906 that exit from the rear of the flow cell 906 (e.g., downstream). Put another way, the sample of interest and the reagents may flow in opposite directions through the channels 944 of the flow cell 906.

Referring to the sipper assembly 934, in the implementation shown, the sipper assembly 934 includes a shared line valve 972 and a bypass valve 974. The shared line valve 972 may be referred to as a reagent selector valve. The valves 922 of the reagent selector valve assemblies 918, 920, the central valve 968, and/or the valves 972, 974 of the sipper assembly 934 may be selectively actuated to control the flow of fluid through fluidic lines 976, 977, 978, 979, 980. One or more of the valves 922, 958, 968, 972, 974 may be implemented by a rotary valve, a pinch valve, a flat valve, a solenoid valve, a check valve, a piezo valve, etc. Other fluid control devices may prove suitable.

The sipper assembly 934 may be coupled to a corresponding number of reagents reservoirs 982 via reagent sippers 984. The reagent reservoirs 982 may contain fluid (e.g., reagent and/or another reaction component). In some implementations, the sipper assembly 934 includes a plurality of ports. Each port of the sipper assembly 934 may receive one of the reagent sippers 984. The reagent sippers 984 may be referred to as fluidic lines.

The shared line valve 972 of the sipper assembly 934 is coupled to the central valve 968 via the shared reagent fluidic line 976. Different reagents may flow through the shared reagent fluidic line 976 at different times. In an implementation, when performing a flushing operation before changing between one reagent and another, the pump manifold assembly 938 may draw wash buffer through the shared reagent fluidic line 976, the central valve 968, and the corresponding flow cell assembly 902, 904. The shared reagent fluidic line 976 may thus be involved in the flushing operation. While one shared reagent fluidic line 976 is shown, any number of shared fluidic lines may be included in the system 900.

The bypass valve 974 of the sipper assembly 934 is coupled to the central valve 968 via the reagent fluidic lines 977, 978. The central valve 968 may have one or more ports that correspond to the reagent fluidic lines 977, 978.

The dedicated fluidic lines 979, 980 are coupled between the sipper assembly 934 and the reagent selector valve assemblies 918, 920. Each of the dedicated reagent fluidic lines 979, 980 may be associated with a single reagent. The fluids that may flow through the dedicated reagent fluidic lines 979, 980 may be used during sequencing operations and may include a cleave reagent, an incorporation reagent, a scan reagent, a cleave wash, and/or a wash buffer. Because only a single reagent may flow through each of the dedicated reagent fluidic lines 979, 980 the dedicated reagent fluidic lines 979 980 themselves thus may not be flushed when performing a flushing operation before changing between one reagent and another. The approach of including dedicated reagent fluidic lines 979, 980 may be advantageous when the system 900 uses reagents that may have adverse reactions with other reagents. Moreover, reducing a number of fluidic lines or length of the fluidic lines that are flushed when changing between different reagents reduces reagent consumption and flush volume and may decrease cycle times of the system 900. While four dedicated reagent fluidic lines 979, 980 are shown, any number of dedicated fluidic lines may be included in the system 900.

The bypass valve 974 is also coupled to the cache 960 of the pump manifold assembly 938 via the bypass fluidic line 962. One or more reagent priming operations, hydration operations, mixing operations, and/or transfer operations may be performed using the bypass fluidic line 962. The priming operations, the hydration operations, the mixing operations, and/or the transfer operations may be performed independent of the flow cell assembly 902, 904. Thus, the operations using the bypass fluidic line 962 may occur during, for example, incubation of one or more samples of interest within the flow cell assembly 902, 904. That is, the shared line valve 972 can be utilized independently of the bypass valve 974 such that the bypass valve 974 can utilize the bypass fluidic line 962 and/or the cache 960 to perform one or more operations while the shared line valve 972 and/or the central valve 968 simultaneously, substantially simultaneously, or offset synchronously perform other operations. Thus, the system 900 can perform multiple operations at once, thereby reducing run time.

Referring now to the drive assembly 940, in the implementation shown, the drive assembly 940 includes a pump drive assembly 986 and a valve drive assembly 988. The pump drive assembly 986 may be adapted to interface with the one or more pumps 956 to pump fluid through the flow cell 906 and/or to load one or more samples of interest into the flow cell 906. The valve drive assembly 988 may be adapted to interface with one or more of the valves 954, 958, 968, 972, 974 to control the position of the corresponding valves 954, 958, 968, 972, 974.

Referring to the controller 926, in the implementation shown, the controller 926 includes a user interface 990, a communication interface 992, one or more processors 994, and a memory 996 storing instructions executable by the one or more processors 994 to perform various functions including the disclosed implementations. The user interface 990, the communication interface 133, and the memory 996 are electrically and/or communicatively coupled to the one or more processors 994.

In an implementation, the user interface 990 is adapted to receive input from a user and to provide information to the user associated with the operation of the system 900 and/or an analysis taking place. The user interface 990 may include a touch screen, a display, a key board, a speaker(s), a mouse, a track ball, and/or a voice recognition system. The touch screen and/or the display may display a graphical user interface (GUI).

In an implementation, the communication interface 992 is adapted to enable communication between the system 900 and a remote system(s) (e.g., computers, a library preparation system) via a network(s). The network(s) may include the Internet, an intranet, a local-area network (LAN), a wide-area network (WAN), a coaxial-cable network, a wireless network, a wired network (e.g., Ethernet), a satellite network, a digital subscriber line (DSL) network, a cellular network, a Bluetooth connection, a near field communication (NFC) connection, etc. Some of the communications provided to the remote system may be associated with analysis results, imaging data, etc. generated or otherwise obtained by the system 900. Some of the communications provided to the system 900 may be associated with a fluidics analysis operation, patient records, and/or a protocol(s) to be executed by the system 900.

The one or more processors 994 and/or the system 900 may include one or more of a processor-based system(s) or a microprocessor-based system(s). In some implementations, the one or more processors 994 and/or the system 900 includes one or more of a programmable processor, a programmable controller, a microprocessor, a microcontroller, a graphics processing unit (GPU), a digital signal processor (DSP), a reduced-instruction set computer (RISC), an application specific integrated circuit (ASIC), a field programmable gate array (FPGA), a field programmable logic device (FPLD), a logic circuit, and/or another logic-based device executing various functions including the ones described herein.

The memory 996 can include one or more of a semiconductor memory, a magnetically readable memory, an optical memory, a hard disk drive (HDD), an optical storage drive, a solid-state storage device, a solid-state drive (SSD), a flash memory, a read-only memory (ROM), erasable programmable read-only memory (EPROM), electrically erasable programmable read-only memory (EEPROM), a random-access memory (RAM), a non-volatile RAM (NVRAM) memory, a compact disc (CD), a compact disc read-only memory (CD-ROM), a digital versatile disk (DVD), a Blu-ray disk, a redundant array of independent disks (RAID) system, a cache and/or any other storage device or storage disk in which information is stored for any duration (e.g., permanently, temporarily, for extended periods of time, for buffering, for caching).

The memory 996 may store instructions executable on the processors 994 that make up a sequencer communication module. The sequencer communication module may transmit and receive communications, via the communication interface 992 for example using an Ethernet network, to a library preparation system. These communications may include information related to the library of samples. For example, the sequencer communication module may receive an indication of a preparation status of the library of samples from the library preparation system. The preparation status may be that library preparation has been completed, and the library of samples is ready to load into the sequencer. The preparation status may also include an estimated duration until library preparation is complete.

In another example, the sequencer communication module may receive identification information for each sample (e.g., a sample ID) in the library of samples from the library preparation system. Moreover, for each sample, the sequencer communication module may receive, from the library preparation system, an indication of an index attached to the sample. Additionally, the sequencer communication module may receive instructions from the library preparation system indicating a particular lane of a flow cell for each sample to be sequenced in. The sequencer communication module may also receive one or more run parameters from the library preparation system, so that the sequencer can sequence the library of samples using the received run parameters. The run parameters may include the number of cycles for each sample, or any other suitable run parameters for sequencing.

Additionally, the sequencer communication module may transmit communications to the library preparation system. For example, the sequencer communication module may transmit status information for the sequencer to the library preparation system, such as the sequencer is ready to receive the library of samples, an estimated duration until the sequencer can reach a safe pause point in the sequencing recipe that would not cause data quality issues to any runs that are already occurring and can receive the library of samples, indications of the sequencing setup steps that have been completed, the sequencing setup steps that remain, the expected durations of the remaining sequencing steps, etc. The sequencer communication module may also transmit acknowledgements to the library preparation system of the communications transmitted by the library preparation system. Still further, the sequencer communication module may transmit and receive any suitable communications to and from the library preparation system related to the library of samples.

FIG. 24B is a schematic illustration of an implementation of a portion of the pump manifold assembly 986 for use with the system 900 of FIG. 24A. In the implementation shown, the pump manifold assembly 986 includes a body 832 carrying the pump valves 822, a cache valve 834, and the pumps 821. The pumps 821 may be syringe pumps and may be adapted to receive a volume of approximately 500 microliters (μL). Other volumes may prove suitable.

The sample loading assembly 938 defines pump ports 1005 (see, FIG. 24A). Each pump port 1005 is coupled to a corresponding port 1007 of the pump manifold assembly 938 via separate pump-channel fluidic lines 830.

The pump valves 958, the cache valve 834, and/or the pumps 821 are operable to individually control fluid flow to each channel 826 of the plurality of channels 826 of the flow cell 825. In the implementation shown, two pump drive assemblies 847 are provided. The pump drive assemblies 847 may be adapted to individually actuate one or more of the pumps 821 to perform one or more of the operations disclosed. In an implementation, one of the pump drive assemblies 847 may operate two of the pumps 821 and the other of the pump drive assemblies 847 may operate six of the pumps 821. Other arrangements may prove suitable.

The pump valves 958, the cache valve 834, and/or the pumps 821 may be operable to flow one or more reagents through the bypass fluidic line 962 and/or to the primary waste fluidic line 966. The body 832 of the pump manifold assembly 986 may also carry a plurality of sensors 836, 837. The sensors 836, 837 may include pressure sensors or flow rate sensors. Other types of sensors may prove suitable. In another implementation, one or more of the sensors 836, 837 and/or the cache valve 834 may be excluded. In some such implementations, the bypass fluidic line 962 may also be excluded. Other arrangements may prove suitable.

The pump manifold assembly 986 includes the cache 960, the pump-channel fluidic lines 830, a plurality of pump fluidic lines 838, a shared fluidic line 840, a cache fluidic line 842, and the primary waste fluidic line 966. The cache fluidic line 842 is coupled to and between the cache 960 and the cache valve 834. The pump-channel fluidic line 830 and the pump fluidic line 838 may be collectively referred to as a pump-channel fluidic line. In the implementation shown, each pump valve 822 is coupled to a corresponding pump-channel fluidic line 830, a corresponding pump fluidic line 838, and the shared fluidic line 840. Each pump 956 is coupled to a corresponding pump fluidic line 838. The pumps 821 are operable to individually control fluid flow to the pump-channel fluidic line 830 and to one of the channels 826 of the flow cell.

The cache valve 834 is coupled to the cache fluidic line 842, the primary waste fluidic line 966, and the shared fluidic line 840. The sensors 836, 837 may be adapted to determine one or more of a pressure value or a flow rate value of one or more of: at least one of the pump-channel fluidic lines 830 or the shared fluidic line 840. Five sensors 836 are coupled to the pump-channel fluidic lines 830. The sensors 836 may be differently positioned. Additional or less sensors including zero sensors may prove suitable.

The pump valves 958, the cache valve 834, and/or the pumps 956 may be operable to flow one or more reagents through the bypass fluidic line 962 and/or to the primary waste fluidic line 966. The body 832 of the pump manifold assembly 938 may also carry a plurality of sensors 836, 837. The sensors 836, 837 may include pressure sensors or flow rate sensors. Other types of sensors may prove suitable. In another implementation, one or more of the sensors 836, 837 and/or the cache valve 834 may be excluded. In some such implementations, the bypass fluidic line 962 may also be excluded. Other arrangements may prove suitable.

The pump manifold assembly 938 includes the cache 960, the pump-channel fluidic lines 830, a plurality of pump fluidic lines 838, a shared fluidic line 840, a cache fluidic line 8424, and the primary waste fluidic line 966. The cache fluidic line 8424 is coupled to and between the cache 960 and the cache valve 834. The pump-channel fluidic line 830 and the pump fluidic line 838 may be collectively referred to as a pump-channel fluidic line. In the implementation shown, each pump valve 958 is coupled to a corresponding pump-channel fluidic line 830, a corresponding pump fluidic line 838, and the shared fluidic line 840. Each pump 956 is coupled to a corresponding pump fluidic line 838. The pumps 956 are operable to individually control fluid flow to the pump-channel fluidic line 830 and to one of the channels 944 of the flow cell 906.

The cache valve 834 is coupled to the cache fluidic line 8424, the primary waste fluidic line 966, and the shared fluidic line 840. The sensors 836, 837 may be adapted to determine one or more of a pressure value or a flow rate value of one or more of: at least one of the pump-channel fluidic lines 830 or the shared fluidic line 840. Five sensors 836 are coupled to the pump-channel fluidic lines 830. The sensors 836 may be differently positioned. Additional or less sensors including zero sensors may prove suitable.

To draw fluid from or to urge fluid toward the flow cell 906 using one or more of the pumps 821, one or more of the pump valves 958 may be actuated into a first position that fluidly communicates the pump-channel fluidic lines 830 and the pump fluidic lines 838 and one or more of the pumps 821 may be actuated to move the fluid.

To move reaction components toward the waste reservoir 817 using one or more of the pumps 821, one or more of the pump valves 958 may be actuated to a second position that fluidly communicates the pump fluidic lines 838 and the shared fluidic line 840, the cache valve 834 may be actuated to a first position that fluidly communicates the shared fluidic line 840 and the primary waste fluidic line 966, and one or more of the pumps 821 may be actuated to move the fluid.

To perform a mixing operation using one or more reaction components received through the bypass fluidic line 962, the pump valves 958 may be actuated to a second position that fluidly communicates the pump fluidic lines 838 and the shared fluidic line 840, the cache valve 834 may be actuated to a second position that fluidly couples the cache fluidic line 842 and the shared fluidic line 840, and one or more of the pumps 821 may be actuated to move the fluid. In some implementations, a larger volume of the reaction component(s) may be transferred through the bypass fluidic line 962 to prime the shared fluidic line 840 using all of the pumps 821. Then, to increase precision on a subsequent fluid transfer, two of the pumps 821 may be used while the remaining pumps 821 are idle, for example. A different number of pumps 821 including using one pump 956 may be used instead.

FIGS. 24C-24F show the process of loading one or more samples of interest from a library preparation system and loading those samples of interest into a flow cell of a sequencing instrument. The library preparation system of FIGS. 24C-24F may be implemented by any of the examples disclosed such as the system 300 of FIG. 1. The sequencing instrument of FIGS. 24C-24F may be implemented by any of the examples disclosed such as the system 900 of FIG. 24A.

FIG. 24C shows the process of the pump manifold assembly 936 priming the fluidic lines 188 and the sample sipper assembly 174 of the library preparation system with a liquid in a direction generally indicated by arrow 950. The liquid may be buffer and the buffer used to prime the fluidic lines 188 may be referred to as the lead or lead buffer.

FIG. 24D shows the process of the sample sipper assembly 174 drawing the sample of interest into the sequencing instrument in a direction generally indicated by arrow 952. An air bubble may be positioned between the buffer and the sample of interest in some examples.

FIG. 24E shows the process of the sample sipper assembly 174 drawing additional buffer behind the sample of interest into the sequencing instrument. The buffer positioned behind the sample of interest may be referred to as the lag or lag buffer. An air bubble may be positioned on either side of the sample of interest and positioned between the buffer and the sample of interest in some examples.

FIG. 24F shows the process of the pump manifold assembly 936 urging the lag buffer, the sample of interest, and/or the lead buffer into the flow cell 906. The lag buffer enters the flow cell 906 first in the implementation shown.

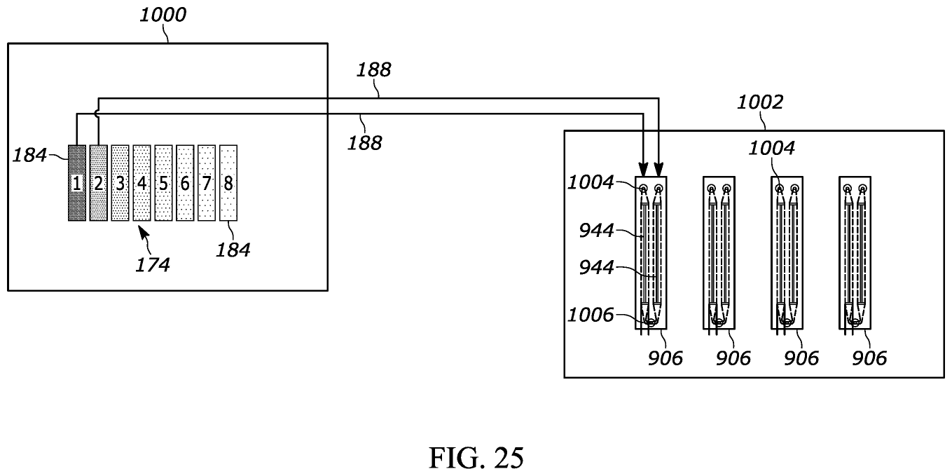

FIG. 25 illustrates a schematic implementation of a sipper assembly 174 of a first system 1000 and a second system 1002 carrying a plurality of flow cells 906. Each sipper 184 of the sipper assembly 174 is fluidly coupled to a corresponding channel 944 of a flow cell 906 by one of the fluidic lines 188.

The flow cells 906 each have a pair of channels 944, a first channel opening 1004 positioned at a first end of each channel 944, and a second channel opening 1006 positioned at a second end of each channel 944. Each pair of channels 944 have a common second channel opening 1006 in the implementation shown. The first system 1000 may be a library preparation system such as the system 300, 400, 500, 600, 700, 800 of FIGS. 1-23 and the second system 1002 may be a sequencing system such as the system 900 of FIG. 24A. Thus, for example, the samples prepared by the first system 1000 may automatically flow from the first system 1000 to the second system 1002.

In other implementations, the samples prepared by the first system 1000 can be automatically directed from the first system 1000 to the second system 1002 in a different manner. For example, the second system 1002 may be integrated into the first system 1000. As another example, the samples prepared by the first system 1000 can be automatically loaded into a consumable (e.g., a cartridge, a flow cell) and the consumable can be automatically moved to and disposed within the second system 1002. In another example, a pipettor from either the first system 1000 or the second system 1002 can extend into the other system to aspirate and dispense sample. In another example, a shuttle may be placed between the first system 1000 or the second system 1002 to carry sample between the two systems. In this example, a sample may be loaded into a sample carrying container such as a sample tube or sample well plate, and placed on the shuttle to be transferred. The shuttle could also be used to transfer a cartridge or a flow cell. In another example laboratory automation, such as via a track, may be used to transfer sample, such as in a sample carrying container, a cartridge or a flow cell, from the first system 1000 to the second system 1002.

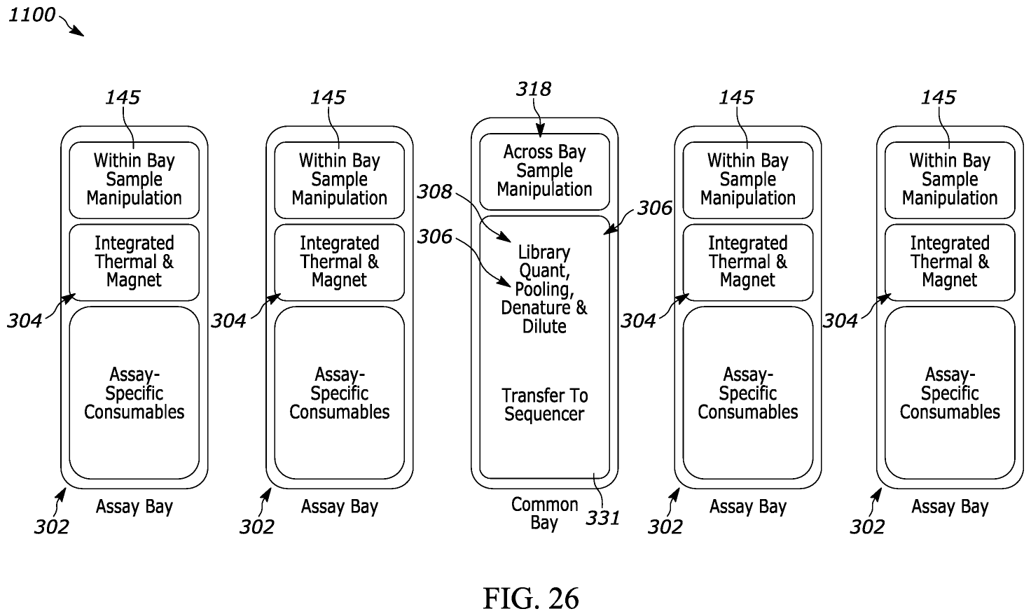

FIG. 26 is a schematic implementation of another system 1000 that can be used to implement the system of FIG. 1.

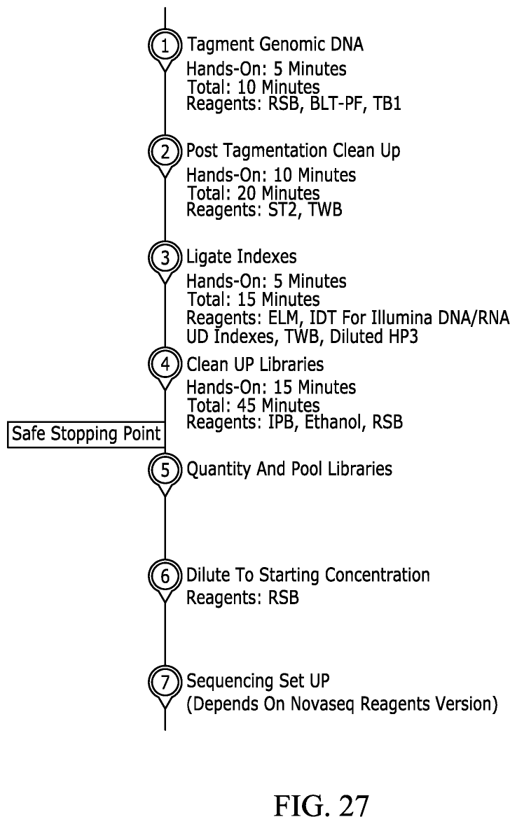

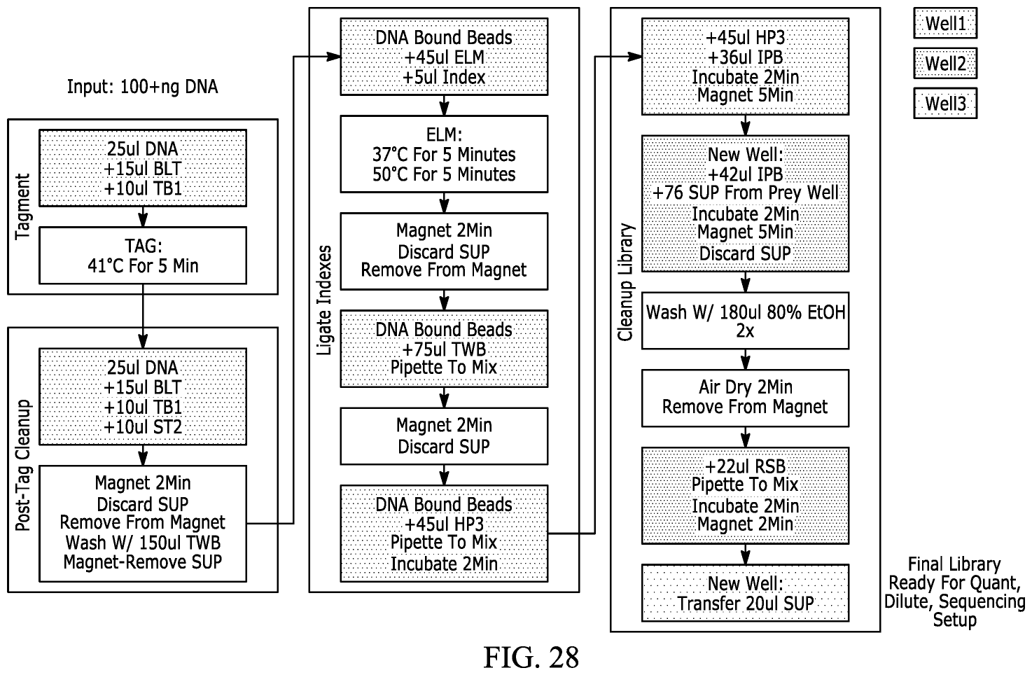

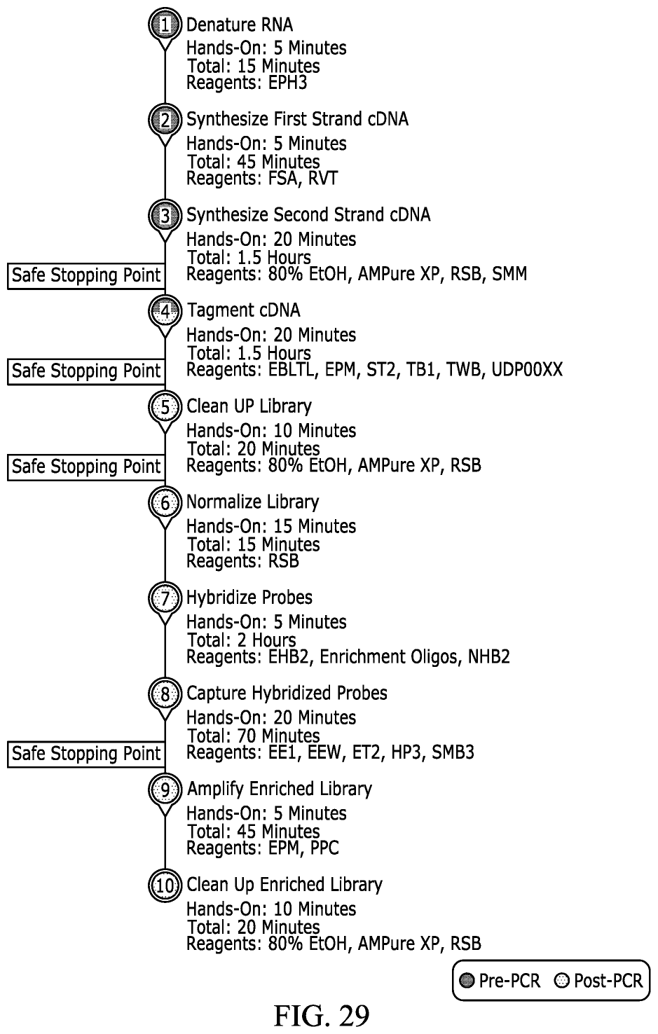

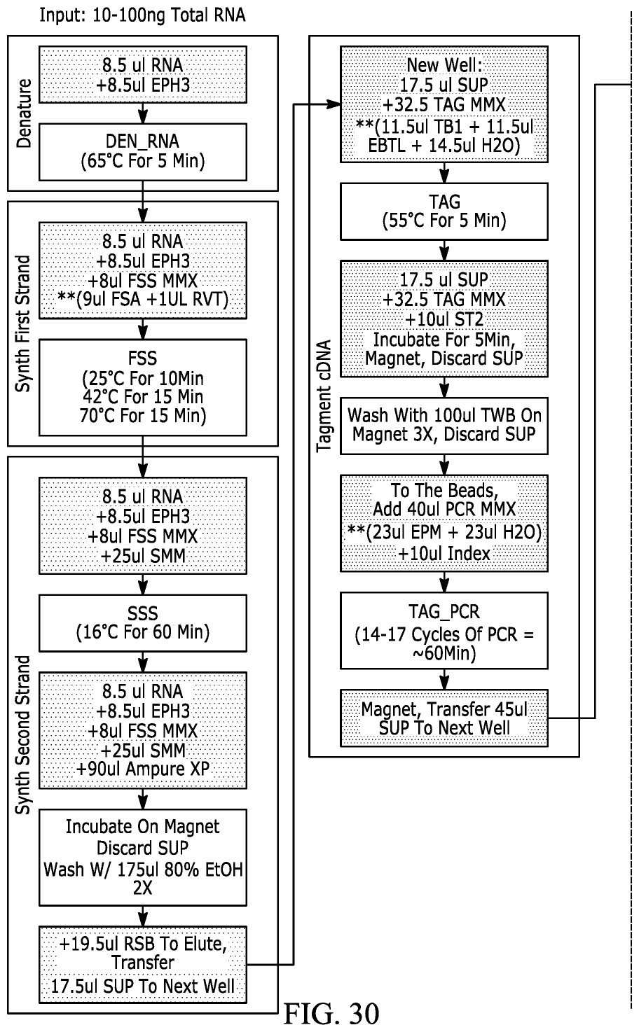

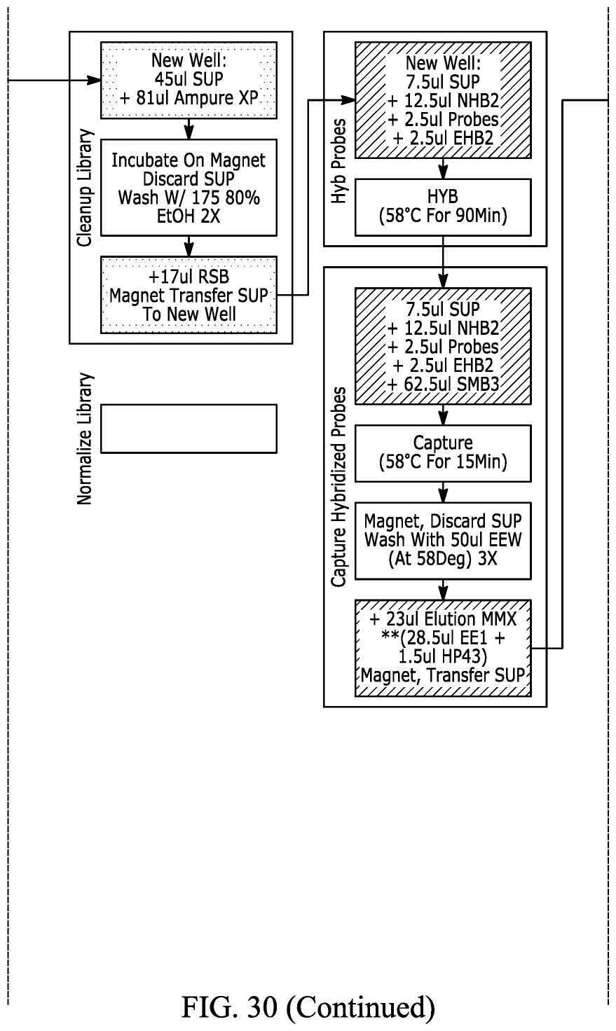

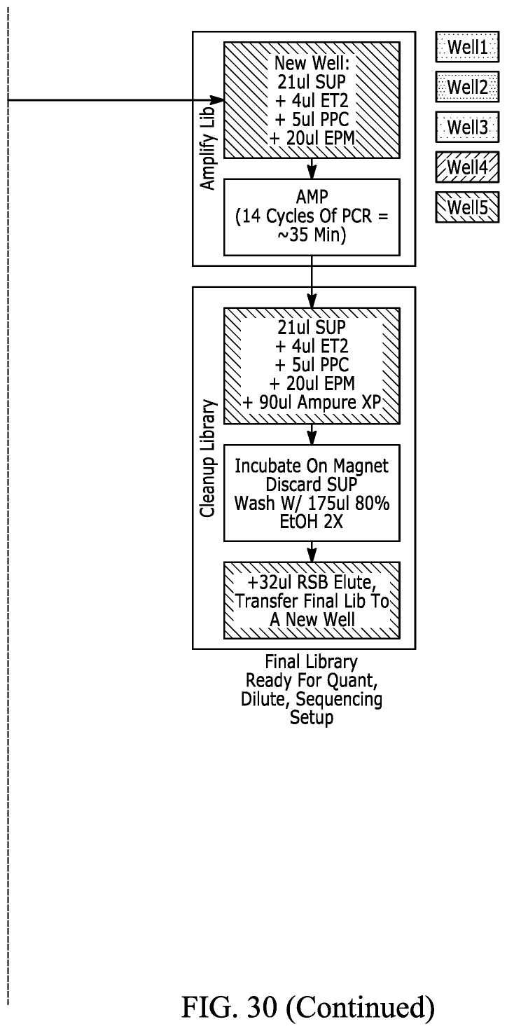

FIGS. 27-30 are workflows that can be performed using the teachings of this disclosure.

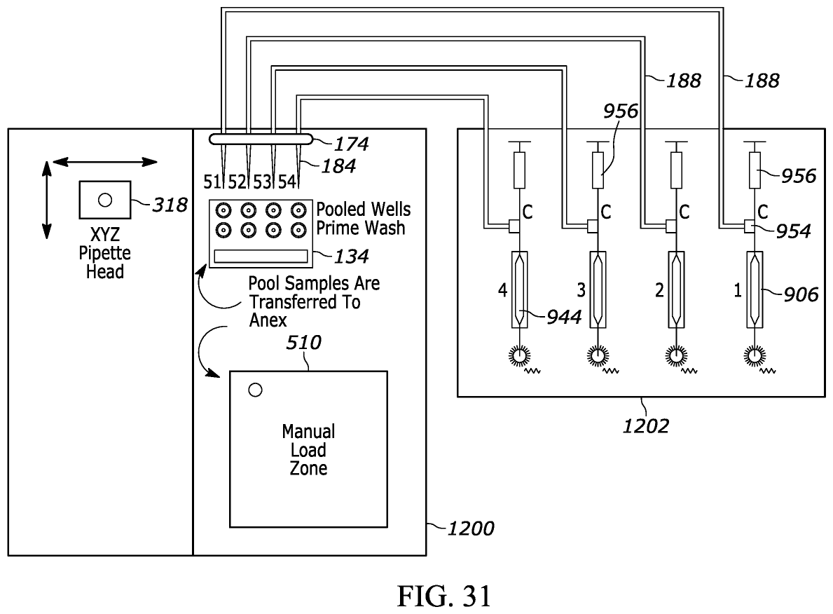

FIG. 31 illustrates a schematic implementation of a sipper assembly 174 of a first system 1200 and a second system 1202 carrying a plurality of flow cells 906. Each sipper 184 of the sipper assembly 174 is fluidly coupled to a corresponding channel 944 of a flow cell 906 by one of the fluidic lines 188.

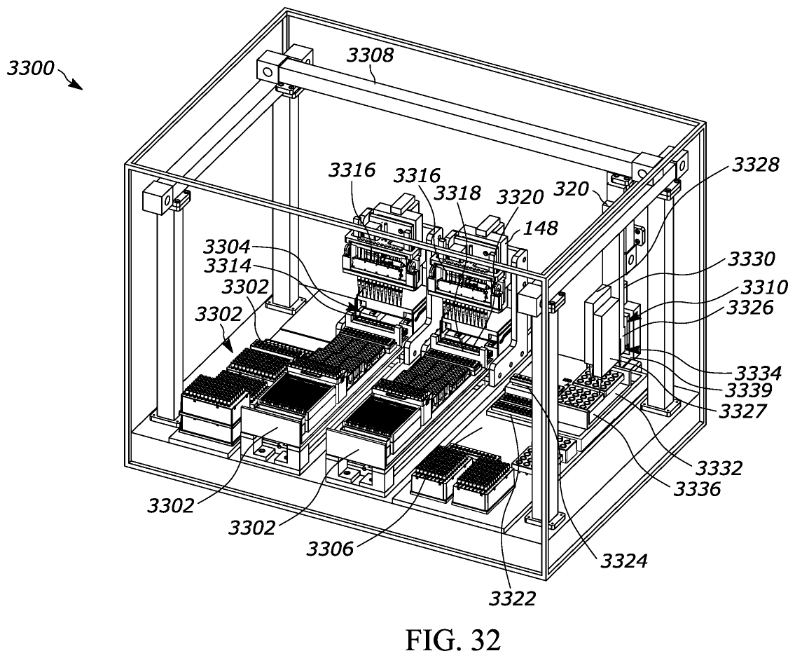

FIG. 32 is an of another system 3300 that can be used to implement the system of FIG. 1. The system 3200 shown is a library prep system and includes a consumables area 3302, assay bays 3304, a common bay 3306, a cross-bay gantry 3308, and a sample sipper assembly 3310. The assay bay 3304 may be referred to as the first working area and/or the consumables area 3302 may be referred to as a consumables bay and/or the second working area. The sipper assembly 3310 may be omitted in other implementations.

The consumables area 3302 is shown carrying a plurality of well plates 3312 and the assay bays 3304 each including an assay bay plate receptacle 3314 and a pipette assembly 3316, a thermocycler 3318 and a magnet 3320 to perform amplification processes and cleanup processes associated with preparing a library of samples for sequencing. The well plates 3312 may be referred to as working plates. The assay bay plate receptacle 3314 may be referred to as a plate receptacle. The common bay 3306 has a common bay plate receptacle 3322 and an imaging system 3324 to perform quantification processes associated with preparing a library of samples for sequencing. The cross-bay gantry 3308 has a gripper 3326 movable between the consumables area 3302, the assay bays 3304, and the common bay 3306 in operation. The cross-bay gantry 3308 is also shown including a first contact dispense 3327 and a second contact dispense 3328. The sample sipper assembly 3310 has a plurality of sippers 3329 and an actuator 3330 to move the sippers 3329 relative to a plate receptacle 3332. The sample sipper assembly 3310 is associated with transferring the library of samples to a sequencing system such as the sequencing system 900 of FIG. 24A. The fluidic line 188 of FIGS. 1 and/or 24 may be used to fluidly couple the sippers 3329 of the sample sipper assembly 3310 and the sequencing instrument.

A loading area 3334 is shown including the sample sipper assembly 3310 and the plate receptacle 3332. The loading area 3334 may include a stage 3336 to move the plate receptacle 3332 relative to the sample sipper assembly 3310.

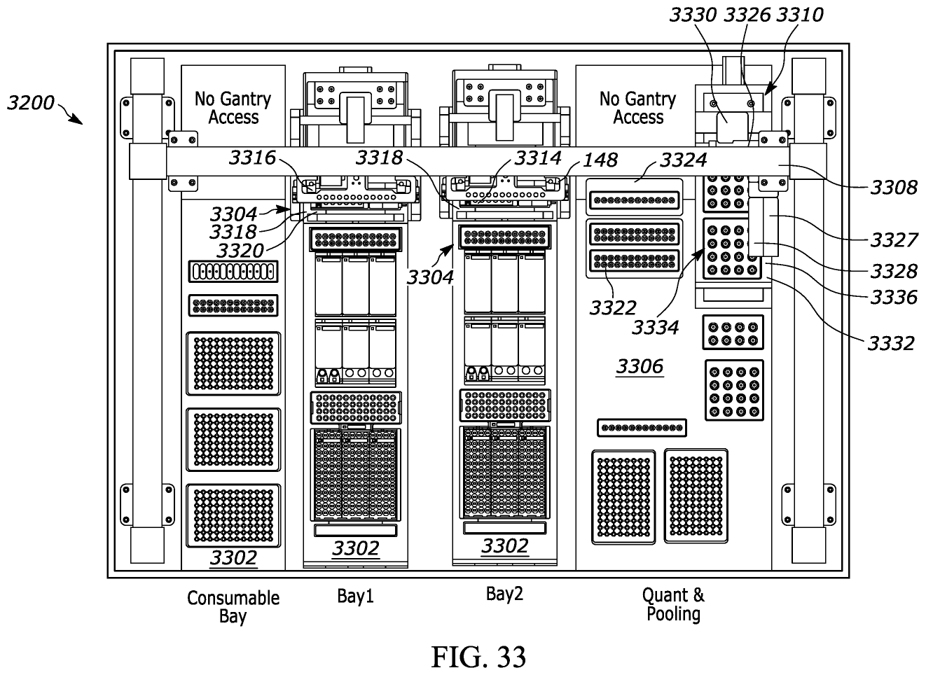

FIG. 33 is a plan view of the system 3200 of FIG. 32.

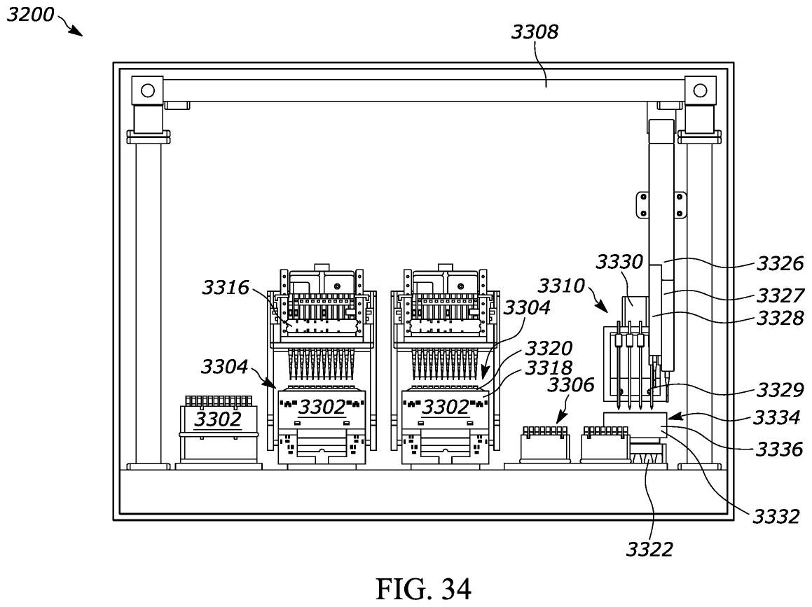

FIG. 34 is a front view of the system 3200 of FIG. 32.

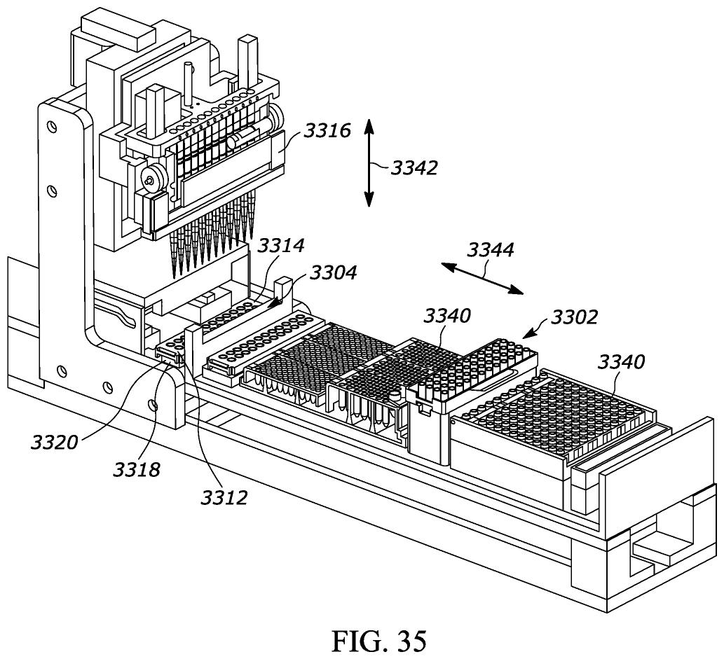

FIG. 35 is an isometric view of one of the assay bays 3304 of the system 3300 of FIG. 32. The assay bay 3304 includes the pipette assembly 3316, the thermocycler 3318, the magnet 3320, and a drawer 3338 that is shown carrying different consumables 3340. The consumables 3340 may include liquid reagents, dry reagents, indexes, beads, well plates, waste, and/or pipette tips. The well plates may be referred to as working plates or plates. The waste may include liquid waste and/or tip waste. The drawer 3338 may carry additional or other consumables, however.

The pipette assembly 3316 is movable relative to the assay bay plate receptacle 3314 in a direction generally indicated by arrow 3342 to allow the pipette assembly 3316 to dispense and/or aspirate liquid from the well plate 3312 shown positioned on the assay bay plate receptacle 3314. The pipette assembly 3316 is movable relative to the assay bay plate receptacle 3314 in a direction generally indicated by arrow 3344 to allow the pipette assembly 3316 dispense and/or aspirate liquid from the consumables 3340.

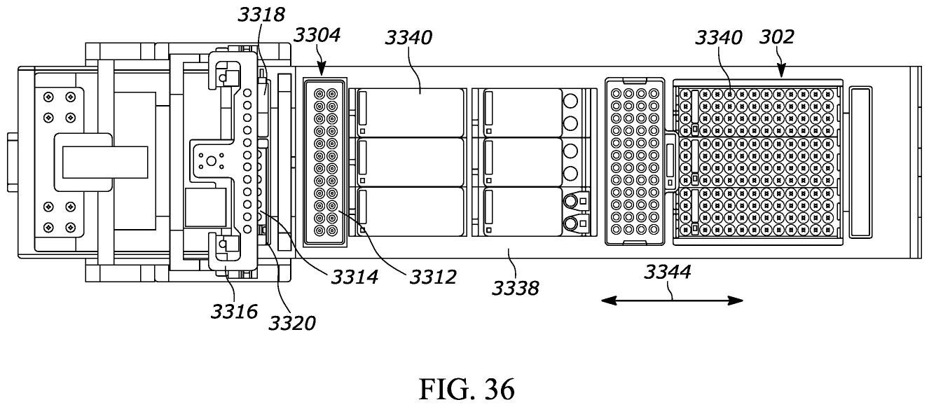

FIG. 36 is a top view of the assay bay 3304 of FIG. 35 including the pipette assembly 3316, the thermocycler 3318, the magnet 3320, and the drawer 3338 that are used to perform amplification processes and cleanup processes associated with preparing a library of samples for sequencing.

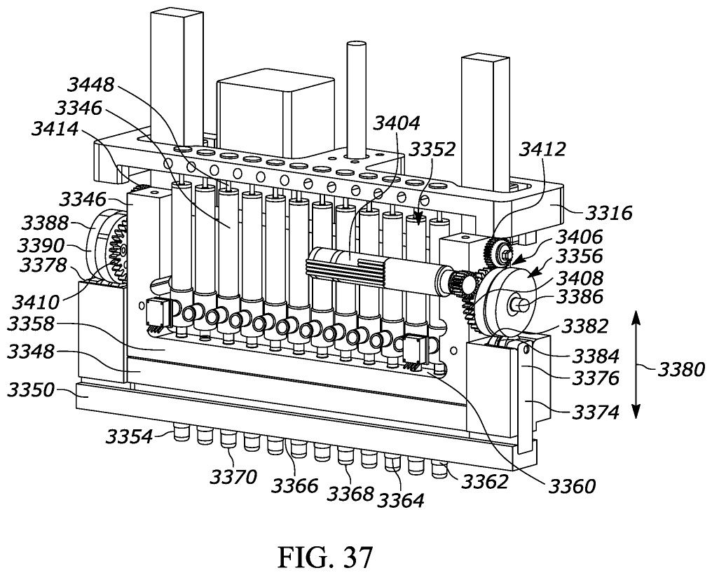

FIG. 37 is an isometric view of a portion of the example pipette assembly 3316 of FIG. 36. The pipette assembly 3316 includes a body 3346, a guide 3348, a bar 3350, a plurality of pipettes 3352, a plurality of gaskets 3354, and a pipette cam assembly 3356. The body 3346 includes a base 3358 that defines pipette apertures 3360 and the guide 3348 includes protrusions 3362 that define pipette apertures 3364 that align with the pipette apertures 3360 of the base 3358. The bar 3350 includes a plurality of apertures 3366 through which the protrusions 3362 of the guide 3348 extend in the implementation shown.

The pipettes 3352 are shown coupled to the body 3346 and extend through the pipette apertures 3360, 3364 of the body 3346 and the guide 3348. The pipettes 3352 each have an end 3368 including a flange 3370 and the gaskets 3354 are positioned between corresponding flanges 3370 of the pipettes 3352 and the protrusions 3362.

The pipette cam assembly 3356 moves the body 3346 away from the guide 3348 and moves the flanges 3370 of the pipettes 3352 toward the protrusions 3362 in operation to compress the gaskets 3354 and moves the body 3346 toward the guide 3348 and moves the flanges 3370 of the pipettes 3352 away from the protrusions 3362 to relax the gaskets 3354. The gaskets 3354 being compressed when the end 3368 of the pipettes 3352 are positioned within a pipette tip 3372 enables the gasket to form a coupling with the pipette tip 3372. The gaskets 3354 being relaxed when the end 3368 of the pipettes 3352 are positioned within the pipette tip 3372 enables the pipette tip 3372 to not be coupled with the pipette 3352.

The guide 3348 includes outward facing channels 3374 and the bar 3350 includes a first arm 3376 and a second arm 3378 that extend within the outward facing channels 3374 of the guide 3348. The first arm 3376 and the second arm 3378 may interact with surfaces of the guide 3348 to guide the movement of the first arm 3376 and the second arm 3378 in direction generally indicated by arrow 3380.

The pipette assembly 3316 is shown including guide bearings 3382, bar bearings 3384, and a cam shaft 3386. The guide bearings 3382 are coupled to the guide 3348 the bar bearings 3384 are coupled to the corresponding first arm 3376 and the second arm 3378 of the bar 3350. The cam shaft 3386 includes inner lobes 3388 and outer lobes 3390 where the inner lobes 3388 engage the guide bearings 3382 and the outer lobes 3390 engage the bar bearings 3384. The inner lobes 3388 of the cam shaft 3386 engaging the guide bearings 3382 move the body 3346 away from the guide 3348 and moves the flanges 3370 of the pipettes 3352 toward the protrusions 3362 to compress the gaskets 3354 in operation. The inner lobes 3388 of the cam shaft 3386 engaging the guide bearings 3382 in a second position enables the body 3346 to move toward from the guide 3348 and moves the flanges 3370 of the pipettes 3352 away the protrusions 3362 to relax the gaskets 3354 in operation. The gaskets 3354 being compressed enables the gasket to form a coupling with the pipette tip 3372 and the gaskets 3354 being relaxed enables the pipette tip 3372 to not be coupled with the pipette 3352

The outer lobes 3390 of the cam shaft 3386 engaging the bar bearings 3384 moves the bar 3350 toward the flanges 3370 of the pipettes 3352 to engage the pipette tips 3372 with the bar 3350 and urge the pipette tips 3372 to be released from the pipette assembly 3316. One of the bar bearings 3384 face one of the guide bearings 3382 in the implementation shown. The bearings 3382, 3384 may be differently positioned, however.

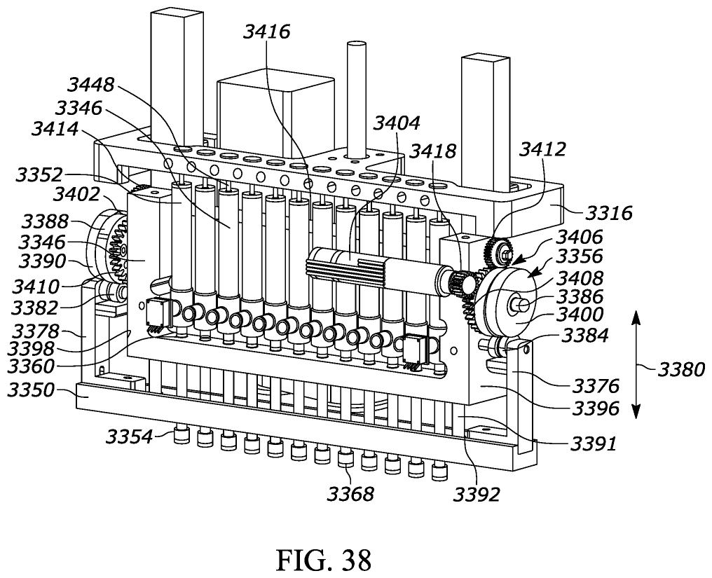

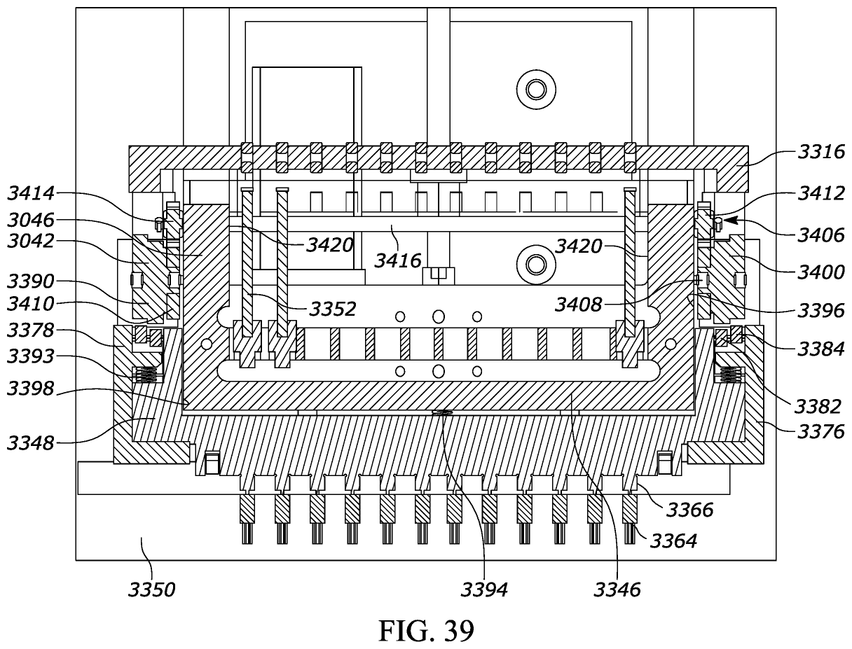

FIG. 38 is an isometric view of the pipette assembly 3316 of FIG. 37 with the guide 3348 removed. The pipette assembly 3316 may include vertical guides 3391 that are coupled to the body 3346 and include a stop 3392. The vertical guides 3391 may threadably engage the body 3346 of the pipette assembly 3316 and the stop 3392 may limit the movement of the bar 3350 toward the body 3346, for example. The guide 3348 may define guide apertures through which the vertical guides 3391 pass. The vertical guides 3391 and surfaces of the guide 3348 defining the guide apertures may guide the movement of the guide 3348 in the direction generally indicated by the arrow 3380. FIG. 39 is a cross-sectional front view of the pipette assembly 3316 of FIG. 38. Bar springs 3393 are shown positioned between the bar 3350 and the guide 3348 and a guide spring 3394 is shown positioned between the body 3346 and the guide 3348. The bar springs 3393 urge the bar bearings 3384 toward the corresponding outer lobe 3390 and the guide spring 3394 urges the body 3346 away from the guide 3348.

Referring back to FIG. 38, the body 3346 has a first side 3396 and a second side 3398 and the cam shaft 3386 has a first cam shaft portion 3400 and a second cam shaft portion 3402. The first cam shaft portion 3400 is coupled to the first side 3396 of the body 3346 and the second cam shaft portion 3402 is coupled to the second side 3398 of the body 3346. The first cam shaft portion 3400 is spaced from the second cam shaft portion 3402 in the implementation shown. For example, the pipettes 3352 are shown positioned between the first and second cam shaft portions 3400, 3402.

The pipette assembly 3316 is shown including a motor 3404 and a gear set 3406. The motor 3404 is carried by the pipette assembly 3316 and may be coupled to the body 3346, for example. The gear set 3406 is coupled to the cam shaft 3386. Movement of the motor 3404 rotates the cam shaft 3386 in operation. The motor 3404 is shown positioned to rotate the gear set 3406 which rotates the cam shaft 3386.

The gear set 3406 includes first and second gears 3408, 3410, first and second pinons 3412, 3414, and a shaft 3416 that couples the first and second pinons 3412, 3414. The shaft 3416 rotating thus rotates both the first and second pinions 3412, 3414. A motor gear 3418 is coupled to the motor 3404 and meshes with the first gear 3408. The first gear 3408 meshes with the first pinion 3412 and the second pinion 3414 meshes with the second gear 3410. The first gear 3408 is coupled to the first side 3396 of the body 3346 and the inner lobe 3388 and the outer lobe 3390 on the first side 3396 of the body 3346 and the second gear 3410 is coupled to the second side 3398 of the body 3346 and the inner lobe 3388 and the outer lobe 3390 on the second side 3398 of the body 3346.

The motor 3404 rotates the motor gear 3418 in operation and the engagement between the motor gear 3418 and the first gear 3408 rotates the first pinion 3412 and also rotates the first cam shaft portion 3400. The first pinion 3412 rotating rotates the shaft 3416 and rotates the second pinion 3414. The second pinion 3414 rotating rotates the second gear 3410 and the second cam shaft portion 3402.

The first side 3396 and the second side 3398 of the body 3346 define shaft apertures 3420 and the shaft 3416 is rotationally coupled within the shaft apertures 3420. The shaft 3416 is spaced from the pipettes 3352.

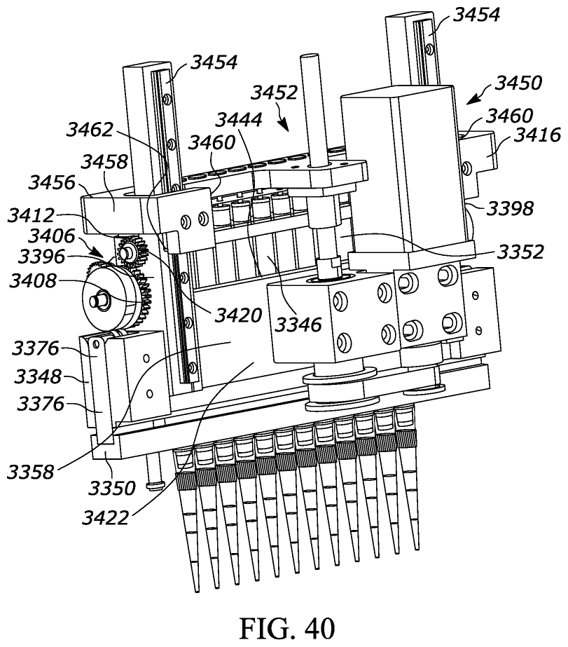

FIG. 40 is a rear isometric view of the pipette assembly 3316 of FIG. 38. The body 3346 has the first side 3396, the second side 3398, and a wall 3442 that define a receptacle 3444. The pipettes 3352 are positioned within the receptacle 3444. The pipettes 3352 each have a barrel 3446 and pistons 3448 are positioned and movable within the corresponding barrels 3446.

An actuator 3450 is coupled to the pistons 3448 to move the pistons 3448 between a retracted position and an extended position within the barrels 3446. The actuator 3450 includes a ball screw 3452 in the implementation shown. The actuator 3450 may also include a pair of linear rails 3454 and a lift 3456. The lift 3456 is coupled to the pistons 3448 and the linear rails 3454 and movable by the ball screw 3452 in the implementation shown. The lift 3456 is C-shaped 3458 and has ends 3460. Carriages 3462 are coupled to the corresponding ends 3460 of the lift 3456 and coupled to the linear rails 3454.

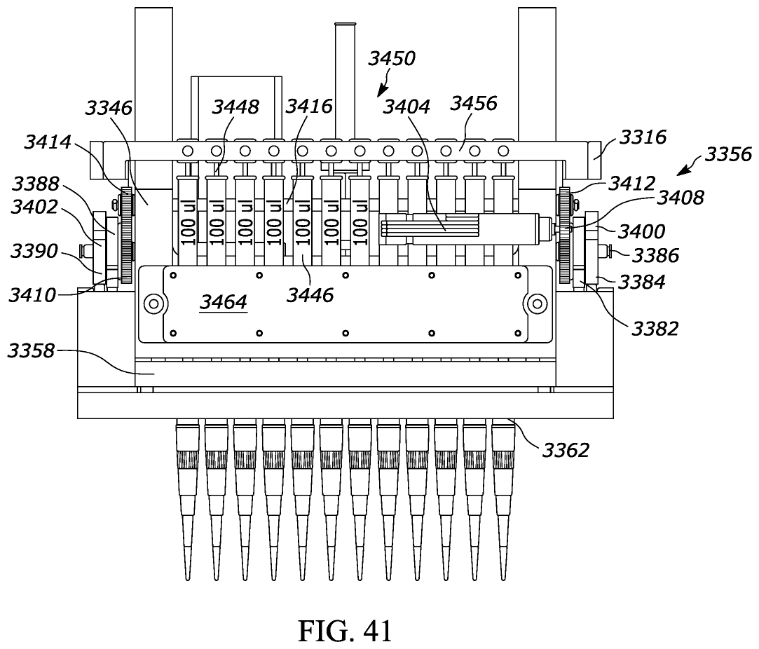

FIG. 41 is a front view of the pipette assembly 3316 of FIG. 38 including a printed circuit board assembly 3464.

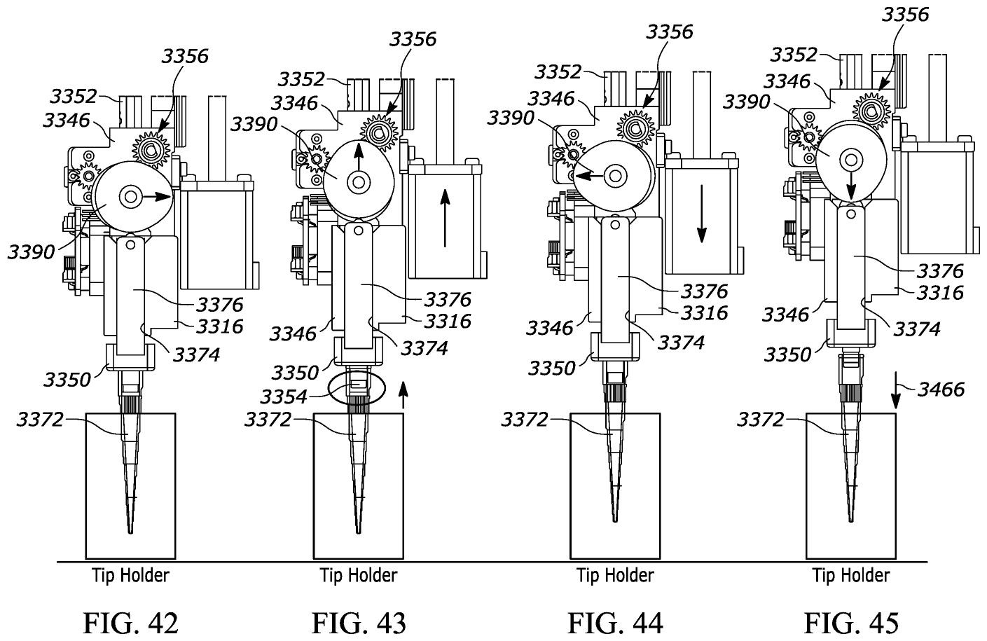

FIG. 42 is a side view of the pipette assembly 3316 of FIG. 38 showing the end 3368 of the pipette 3352 inserted into a pipette tip 3372.

FIG. 43 is a side view of the pipette assembly 3316 of FIG. 38 showing the cam shaft 3386 rotated 90 relative to the position of the cam shaft 3386 of FIG. 42. The gasket 3354 is compressed to form a coupling with the pipette tip 3372 in the position shown. The pipette assembly 3316 may compress the gasket 3354 in the implementation shown in a repeatable manner to ensure that the pipette tips 3371 are secured to the pipette assembly 3316 in the same or a substantially similar position. The pipette tip 3371 being in a known and repeatable position enables more reagent to be drawn from reagent wells and/or for less dead volume.

FIG. 44 is a side view of the pipette assembly 3316 of FIG. 38 showing the cam shaft 3386 rotated 90 relative to the position of the cam shaft 3386 of FIG. 43. The gasket 3354 is relaxed to allow the allow the pipette tip 3372 to be uncoupled from the end 3368 of the pipette 3352.

FIG. 45 is a side view of the pipette assembly 3316 of FIG. 38 showing the cam shaft 3386 rotated 90 relative to the position of the cam shaft 3386 of FIG. 44. The gasket 3354 is relaxed and the bar 3350 is moved in a direction generally indicated by arrow 3466 to push the pipette tip 3372 off of the pipette 3352.

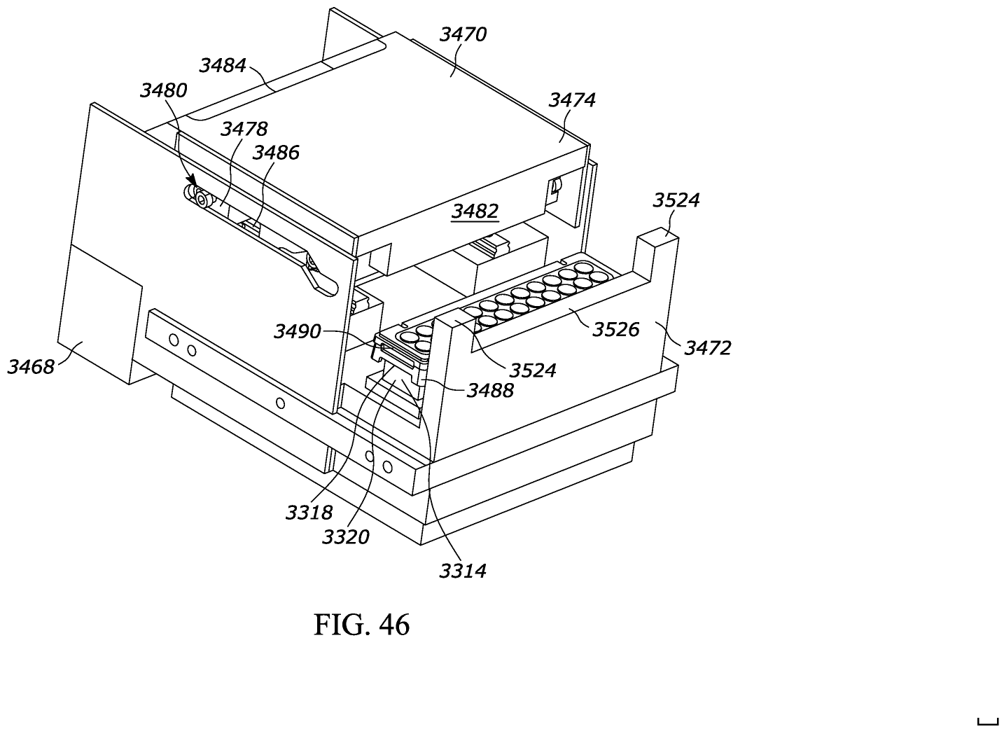

FIG. 46 is an isometric view of the thermocycler 3318 of one of the assay bays 3304 of FIG. 32. The thermocycler 3318 includes a base 3468, the plate receptacle 3314, and a cover assembly 3470. The base 3468 includes a stop wall 3472, the plate receptacle 3314 is positioned on the base 3468, and the cover assembly 3470 is movably coupled to the base 3468.

The cover assembly 3470 includes a sled 3474, a cover 3476, a cover follower 3478, and a cam assembly 3480. The sled 3474 has a front wall 3482 and a rear wall 3484 and a receptacle 3486 is defined between the front wall 3482 and the rear wall 3484. The cover 3476 is positioned within the receptacle 3486 of the sled 3474 and the cover follower is positioned within the receptacle 3486 of the sled 3474 and movably coupled to the cover 3476.

The cam assembly 3480 causes the cover 3476 to move toward the base 3468 to cover the plate receptacle 3314 and causes the cover follower 3478 to move toward the base 3468 in operation. The plate receptacle 3314 is shown receiving a plate 3488 having a well 3490 and the thermocycler 3318 may adjust a temperature of a sample within the well 3490 of the plate 3488 in operation.

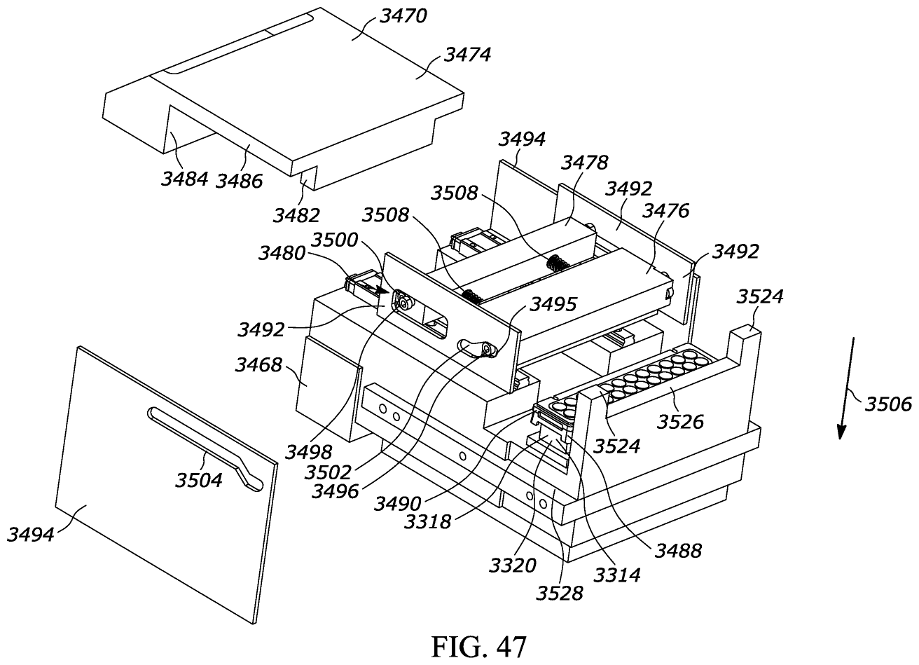

FIG. 47 is an expanded isometric view of the thermocycler 3318 of FIG. 46. The cam assembly 3480 is shown including inner cam plates 3492, outer cam plates 3494, cover bearings 3495, inner slot bearings 3496, cover follower bearings 3498, and outer slot bearings 3500. The inner cam plates 3492 are coupled to the sled 3474 and each define an inner cam slot 3502 and the outer cam plates 3494 are coupled to the base 3468 and each define an outer cam slot 3504.

The cover bearings 3495 are coupled to the cover 3476 and are positioned to engage the stop wall 3472 and the inner slot bearings 3496 are coupled to the cover 3476 and movably positioned within the inner cam slot 3502. The cover follower bearings 3498 are coupled to the cover 3476 and positioned to engage the rear wall 3484 of the sled 3474 and the outer slot bearings 350 are coupled to the cover follower 3478 and movably positioned within the outer cam slot 3504.

The cover bearings 3495 engage the stop wall 3472 in operation and cause the inner slot bearings 3496 to move within the inner cam slot 3502 and the cover bearings 3495 to move along the stop wall 3472 such that the cover 3476 moves toward the base 3468 in a direction generally indicated by arrow 3506 to cover the plate receptacle 3314. The cover follower bearings 3498 engage the rear wall 3484 in operation and cause the outer slot bearings 3500 to move within the outer cam slot 3504 and the cover follower bearings 3498 to move along the rear wall 3484 to move the cover follower 3478 in the direction generally indicated by the arrow 3506 toward the base 3468. Springs 3508 are included that bias the cover 3476 away from the cover follower 3478. The spring 3508 may be a coil spring or another biasing element.

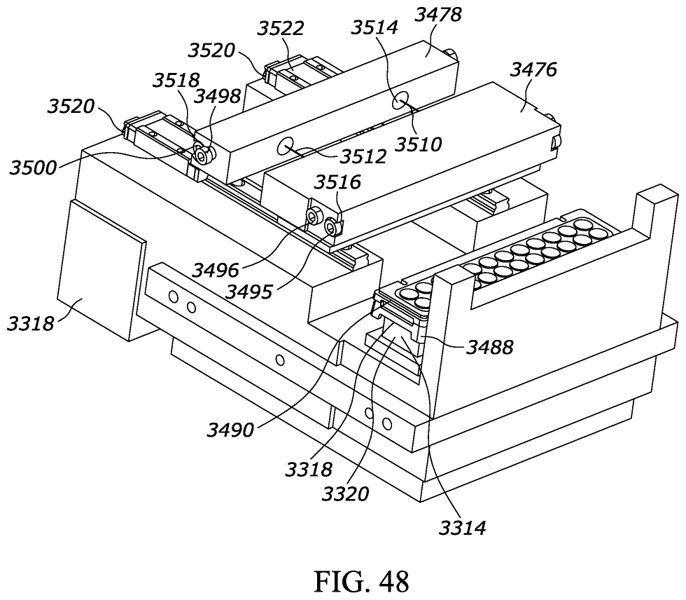

FIG. 48 is an isometric view of the thermocycler 3318 of FIG. 46 with the inner cam plates 3492, the outer cam plates 3494, the sled 3474, and the springs 3508 omitted. Guide rods 3510 are shown movably coupling the cover 3476 and the cover follower 3478. The cover 3476 has blind bores 3512 and the cover follower 3478 has through bores 3514. The guide rods 3510 are positioned within the corresponding blind bores 3512 of the cover 3476 and through bores 3514 of the cover follower 3478. The springs 3508 surround the corresponding guide rods 3510.

The cover 3476 defines cover bearing receptacles 3516 in which the cover bearings 3495 are positioned and the cover follower 3478 defines cover follower bearing receptacles 3518 in which the cover follower bearings 3498 are positioned. Linear rails 3520 coupled to the base 3468 and carriages 3522 are coupled to the rear wall 3484 and coupled to the linear rails 3520.

Referring back to FIG. 46, the stop wall 3472 has extensions 3524 between which an opening 3526 is defined. The front wall 3482 is sized to pass between the extensions 3524 and the cover bearings 3495 are to engage the extension. An actuator 3528 and the magnet 3320 may be included where the actuator 3528 moves the magnet 3320 relative to the plate receptacle 3314. The thermocycler 3318 may be used during amplification processes and the magnet 3320 and/or the actuator 3528 may be used during cleanup processes.

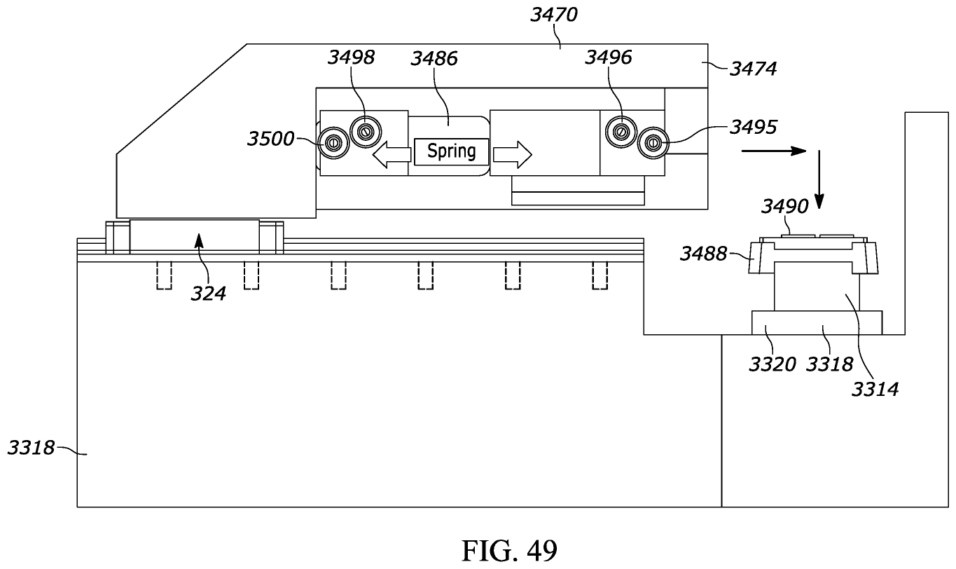

FIG. 49 is a side view of the thermocycler 3318 with the cover assembly 3470 in the rear position and not covering the plate 3488. The spring 3508 is shown in the expanded position.

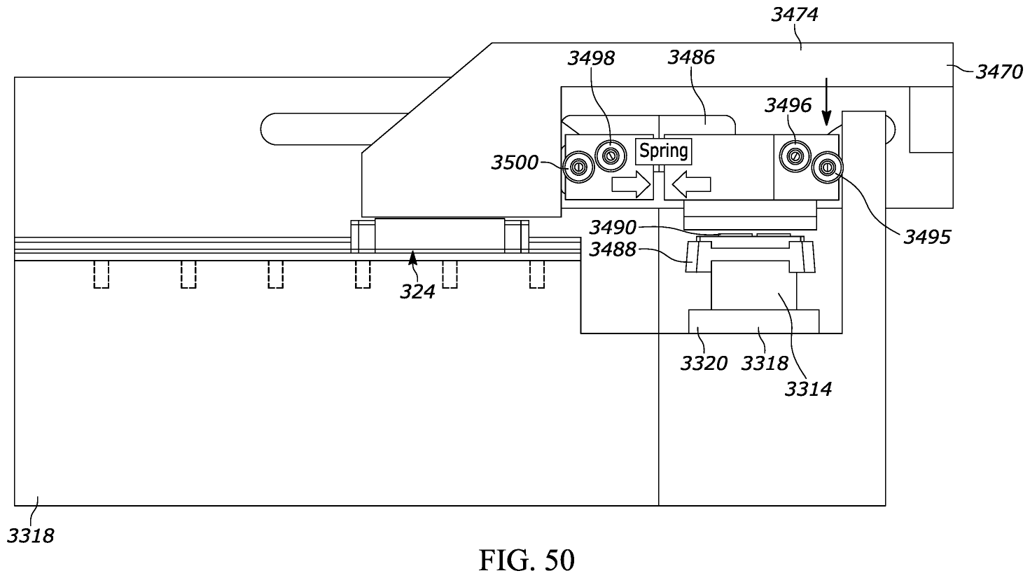

FIG. 50 is a side view of the thermocycler 3318 with the cover assembly 3470 in the forward and lowered position covering the plate 3488. The spring 3508 is shown in the compressed position.

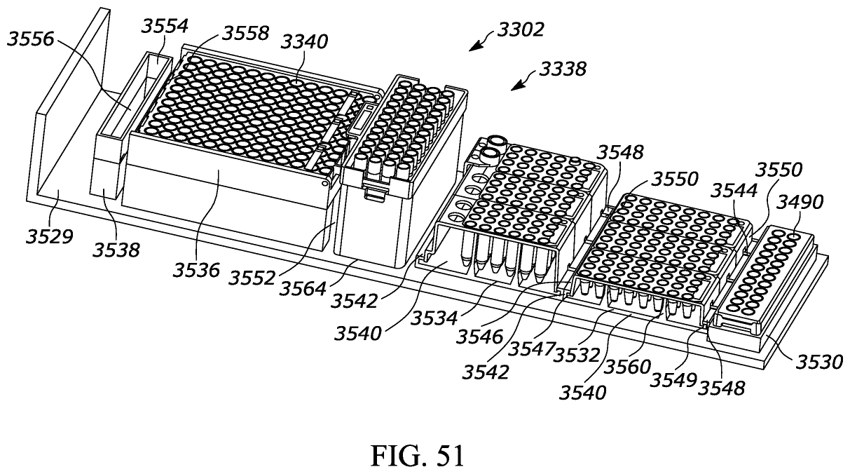

FIG. 51 is an isometric view of the drawer 3338 of one of the assay bays 3304 of FIG. 32. The drawer 3338 includes a platform 3529, a plate receptacle 3530, a small liquid reagent well plate receptacle 3532, a large liquid reagent well plate receptacle 3534, a dry well plate receptable 3536, and a waste reservoir 3538. The plate receptacle 3530, the small liquid reagent well plate receptacle 3532 and the large liquid reagent well plate receptacle 3534 are coupled to the platform 3529 and the dry well plate receptable 3536 is positioned on the platform 3529.

The small liquid reagent well plate receptacle 3532 has a base 3540, a first end wall 3542, and a second end wall 3544. The first end wall 3542 is coupled to the base 3540 and has an inward extending lip 3546 that forms a first groove 3547 with the base 3540 and the second end wall 3544 is coupled to the base 3540 that has an inward extending lip 3548 that forms a second groove 3549 and includes a key 3550. The large liquid reagent well plate receptacle 3534 is also coupled to the platform 3529 and includes a base 3540, a first end wall 3542, and a second end wall 3544. The first end wall 3542 has an inward extending lip 3546 that forms a first groove 3547 with the base of the large liquid reagent well plate receptacle 3534 and the second end wall 3544 has an inward extending lip 3548 that forms a second groove 3549 with the base of the large liquid reagent well plate receptacle 3534 and comprising a key 3550. The consumables 3340 form a snap-fit connection with the first groove 3547 and the second groove 3549 and the key 3550 provides a poka-yoka mechanism to enable the consumables 3340 to be coupled with the small liquid reagent well plate receptacle 3532 and the large liquid reagent well plate receptacle 3534 in a particular orientation.

The dry well plate receptable 3536 is positioned on the platform 3529 and defines a waste reservoir compartment 3552. The waste reservoir 3538 has a wider portion 3554 including an inlet 3556 and a narrow portion 3558 that extends from the wider portion 3554 and is shown positioned within the waste reservoir compartment 3552. The dry well plate receptacle 3536 and/or the waste reservoir compartment 3552 may be differently configured, however. The waste reservoir 3538 may receive pipette tips 3372 and/or liquid waste. The inlet 3556 is a rectangular inlet 3557 to receive waste associated with a multiple-tip pipette such as the pipette assembly 3316, for example. The inlet 3556 may be a different shape, however.

A central well plate support wall 3560 extends from the base 3540 and is positioned between the first end wall 3542 and the second end wall 3544 of the small liquid reagent well plate receptacle 3532. The small liquid reagent well receptacle 3532 includes two central well plate support wall 3560 in the implementation shown. A central well plate support wall 3562 extends from the base 3540 and is positioned between the first end wall 3542 and the second end wall 3544 of the large liquid reagent well plate receptacle 3534. The large liquid reagent well receptacle 3534 includes two central well plate support walls 3562 in the implementation shown. The height of the support walls 3560, 3562 corresponds to the height of the corresponding consumable 3340/well plate. The support walls 3560, 3562 may support the corresponding consumable 3340 and/or encourage a top surface of the consumable 3340 to be substantially flat and/or to deter the top surface of the consumable 3340 from being concave, for example.

The small liquid reagent well plate receptacle 3532 is positioned between the plate receptacle 3530 and the large liquid reagent well plate receptacle 353 in the implementation shown. The large liquid reagent well plate receptacle 3534 is positioned between the small liquid reagent well plate receptacle 3532 and the dry well plate receptacle 3536 in the implementation shown. The dry well plate receptacle 3536 is positioned between the large liquid reagent well plate receptacle 3534 and the wider portion 3554 of the waste reservoir 3538 including the inlet 3556 in the implementation shown. The reagent well plate receptacles 3532 and/or 3534 and/or the dry well plate receptacle 3536 may be in different positions and/or may be omitted.

The drawer 3338 also includes a tip receptacle 3564 in the implementation shown. The tip receptacle 3564 is positioned between the large liquid reagent well plate receptacle 3534 and the dry well plate receptacle 3536. The tip receptacle 3564 may be in another position, however.

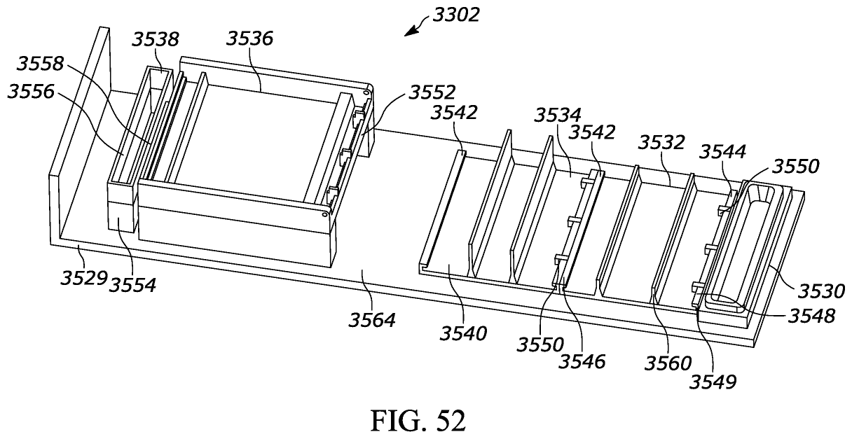

FIG. 52 is an isometric view of the drawer 3338 of FIG. 51 with the consumables 3340 removed but including the dry well plate receptacle 3536 and the waste reservoir 3538.

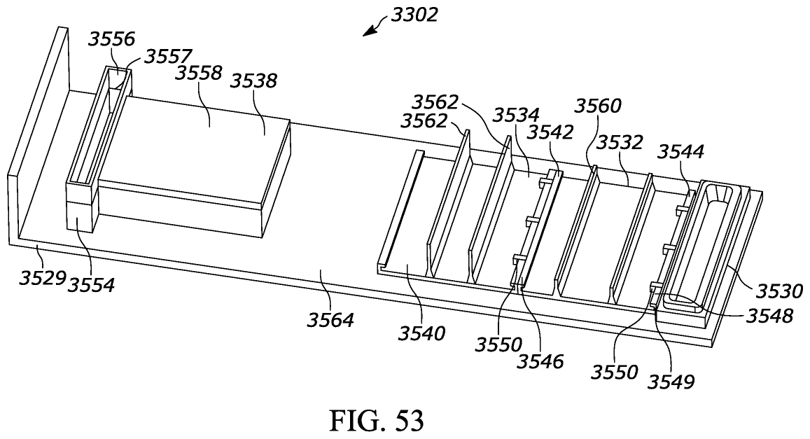

FIG. 53 is an isometric view of the drawer 3338 of FIG. 51 with the consumables 3340 and the dry well plate receptacle 3536 removed.

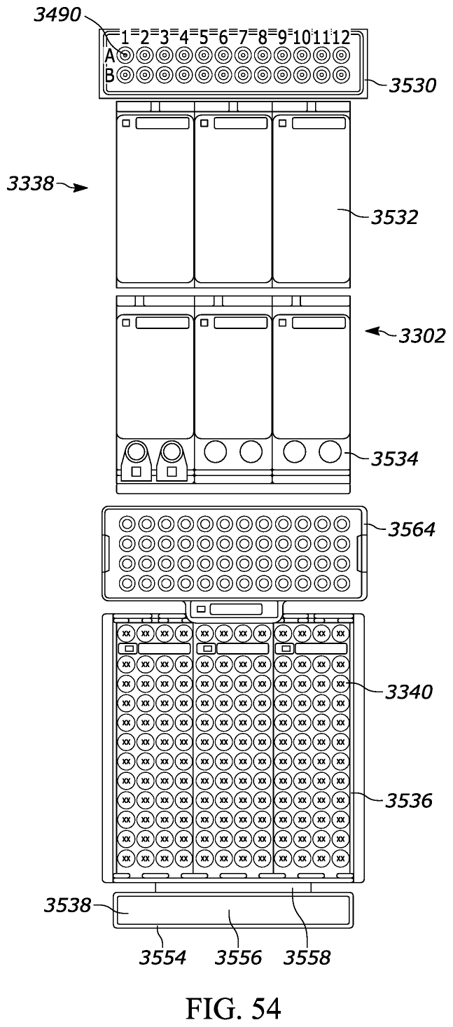

FIG. 54 is a top view of the drawer 3338 of FIG. 51 with the consumables 3340, the dry well plate receptacle 3536, and the waste reservoir 3538 included.

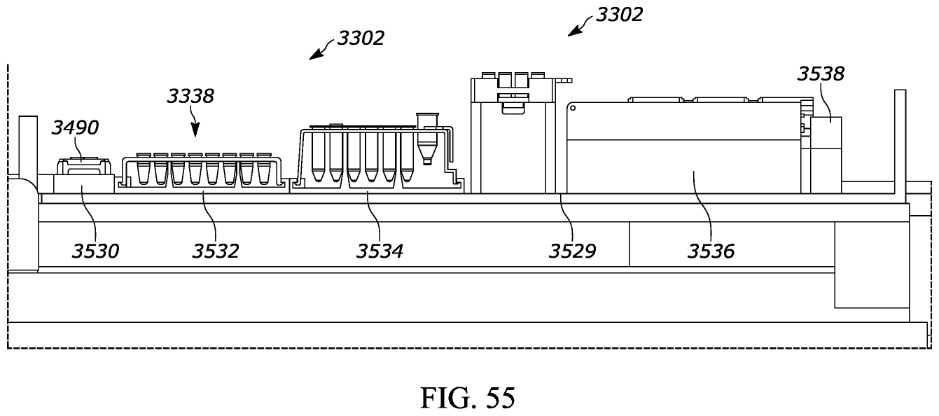

FIG. 55 is a side view of the drawer 3338 of FIG. 51 with the consumables 3340, the dry well plate receptacle 3536, and the waste reservoir 3538 included.

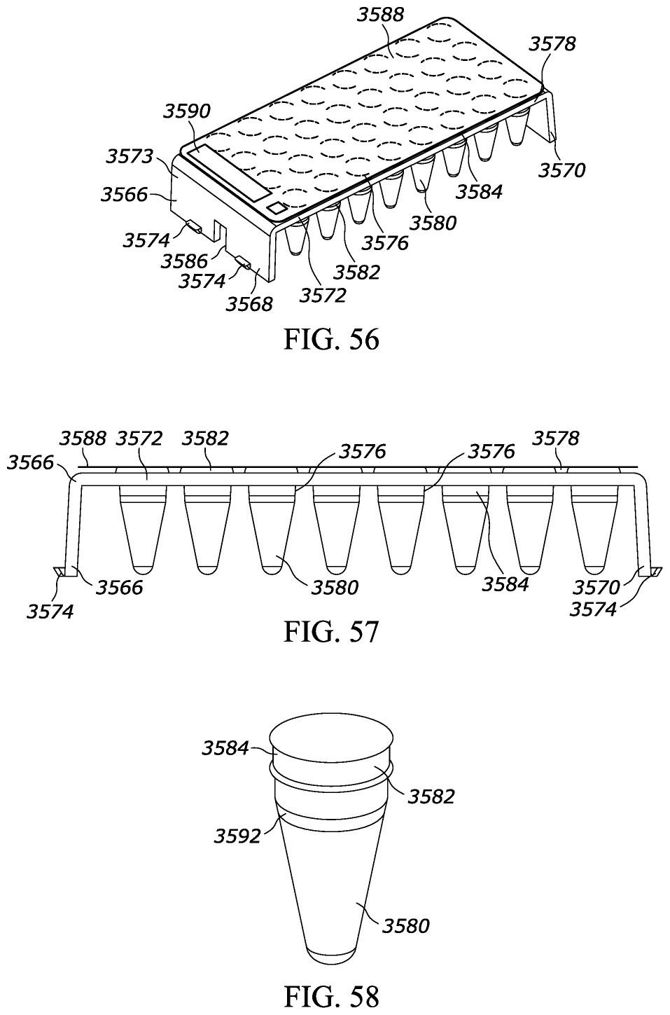

FIG. 56 is an isometric view of a reagent well plate 3566 including a first end wall 3568, a second end wall 3570, and a panel 3572 that can be used with the system 3300 of FIG. 32. The reagent well plate 3566 of FIG. 56 is a small liquid reagent well plate 3573 in the implementation shown. The first end wall 3568 has a male snap-fit component 3574 and the second end wall 3570 has a male snap-fit component 3574. The first end wall 3568 and the second end wall 3570 each have two male snap-fit components in the implementation shown. The panel 3572 is coupled to and extends between the first end wall 3568 and the second end wall 3570 and defines reagent well receptacles 3576 and includes a top surface 3578. Reagent wells 3580 are positioned within the reagent well receptacles 3576. The reagent wells 3580 have an end 3582 having an annular collar 3584 that are shown engaging the top surface 3578 of the panel 3572.

The first end wall 3568 has a keying notch 3586 in the implementation shown. The keying notch 3586 may be receive the key 3550 of the small liquid reagent well plate receptacle 3532 and the large liquid reagent well plate receptacle 3534 to provide a poka-yoka mechanism to enable the consumables 3340 to be coupled with the small liquid reagent well plate receptacle 3532 and the large liquid reagent well plate receptacle 3534 in a particular orientation.

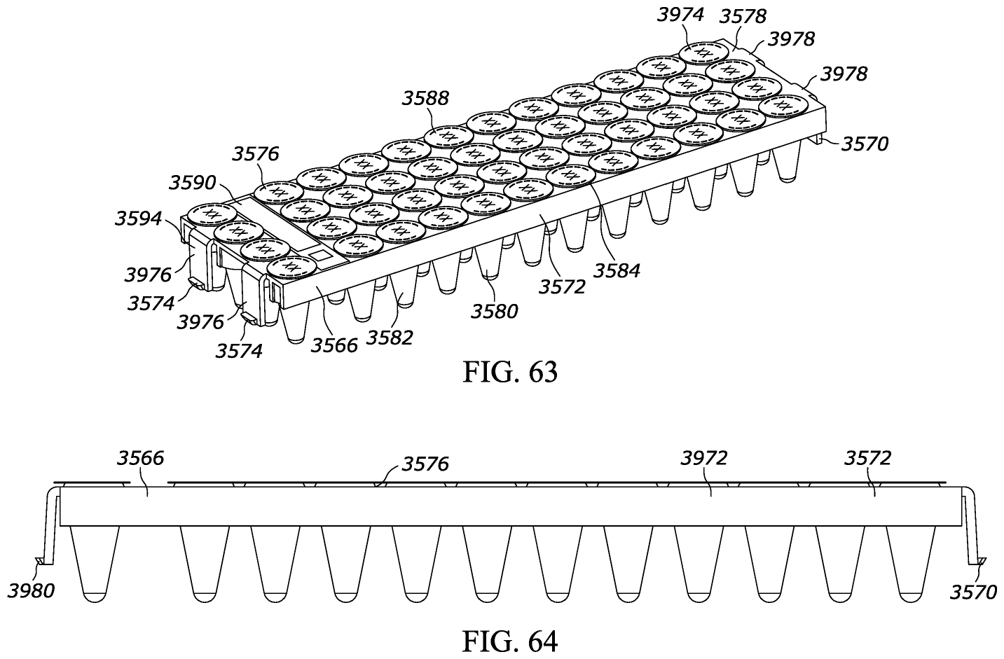

An impermeable barrier 3588 is coupled to the ends 3582 of the reagent wells 3580. The impermeable barrier 3588 may include foil and/or plastic. The impermeable barrier 3588 is shown as a single sheet that is coupled to the ends 3582 of the reagent wells 3580. Individual impermeable barriers 3588 may alternatively be coupled to each of the reagent wells 3580 as shown in FIG. 63, for example. A machine-readable code 3590 is also included. The machine-readable code 3590 may be coupled to the impermeable barrier 3588 as shown in the implementation of FIG. 56. The machine-readable code 3590 may additionally or alternatively be coupled to the panel 3572 and/or to the end walls 3568 and/or 3570.

FIG. 57 is a side view of the reagent well plate 3566 of FIG. 56. The first end wall 3568 and the second end wall 3570 extend outwardly from the panel 3572 in the implementation shown and the panel 3572 may be concave. The panel 3572 may alternatively have a different shape and/or not be concave. The panel 3572 may be substantially flat when the first end wall 3568 and the second end wall 3570 are coupled to a reagent well plate receptacle 3532 and/or 3534. The first end wall 3568 and the second end wall 3570 may be substantially parallel to one another and/or less outwardly tapered when the first end wall 3568 and the second end wall 3570 are coupled to a reagent well plate receptacle 3532 and/or 3534, for example.

FIG. 58 is an isometric view of one of the reagent wells 3580 of the reagent well plate 3566 of FIG. 56. The reagent well 3580 has a second annular collar 3592 longitudinally spaced from the annular collar 3584 in the implementation shown. The panel 3572 is to be positioned between the annular collar 3584 and the second annular collar 3592 of the reagent well 3580 when the reagent well 3580 is received within the reagent well receptacle 3576 and the reagent well 3580 is coupled to the panel 3572.

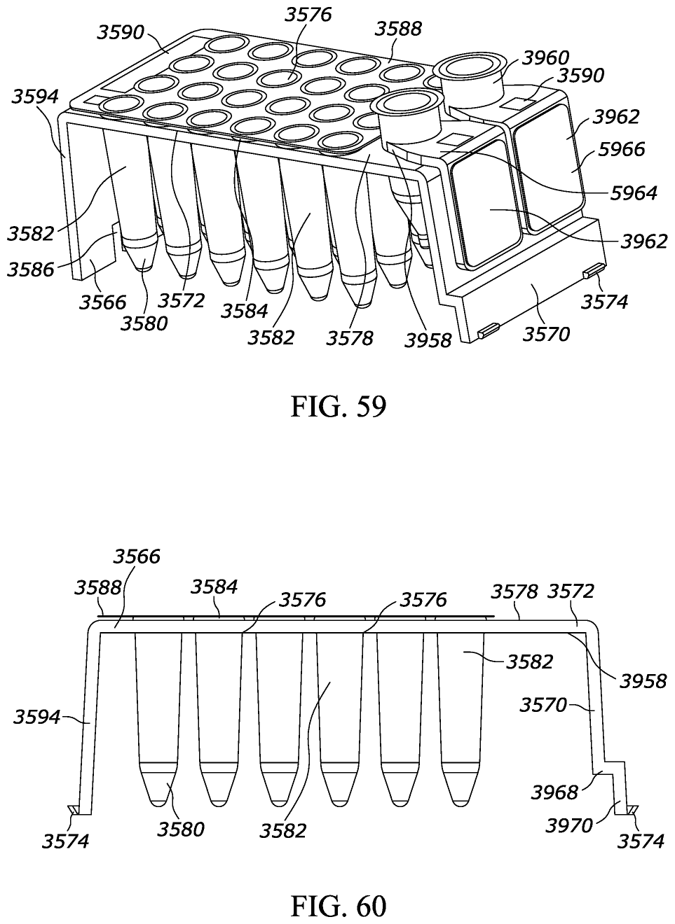

FIG. 59 is an isometric view of another reagent well plate 3594 including a first end wall 3568, a second end wall 3570, and a panel 3572. The reagent well plate 3594 is a large liquid reagent well plate 3956 in the implementation shown. The reagent wells 3580 of FIG. 59 are shown longer and/or larger than the reagent wells 3580 of FIG. 58. The panel 3572 of the reagent well plate 3594 of FIG. 56 has a plurality of second reagent well receptacles 3958 having a different size than the reagent well receptacles 3576. The second reagent well receptacles 3958 are positioned between the second end wall 3570 and the reagent well receptacles 3576 in the implementation shown.

Bulk reagent wells 3960 are positioned within the second reagent well receptacles 3958. An L-tab 3962 is coupled to each of the bulk reagent wells 3960. The L-tab 3962 has a first leg 3964 coupled to the bulk reagent well 3960 and a second leg 3966 extending from the first leg 3964 at an angle that corresponds to an angle of the second end wall 3570. The second leg 3966 is shown extending along the second end wall 3570. The L-tab 3962 is positioned within a dimensional envelope of the reagent well plate 3566 in the implementation shown.

FIG. 60 is a side view of the reagent well plate 3594 of FIG. 59. The second end wall 3570 includes a first wall section 3968 and a second wall section 3970 that are coupled to form a recess 3971. The L-tab 3962 is positioned within a dimensional envelope of the recess 3971 as shown in FIG. 59.

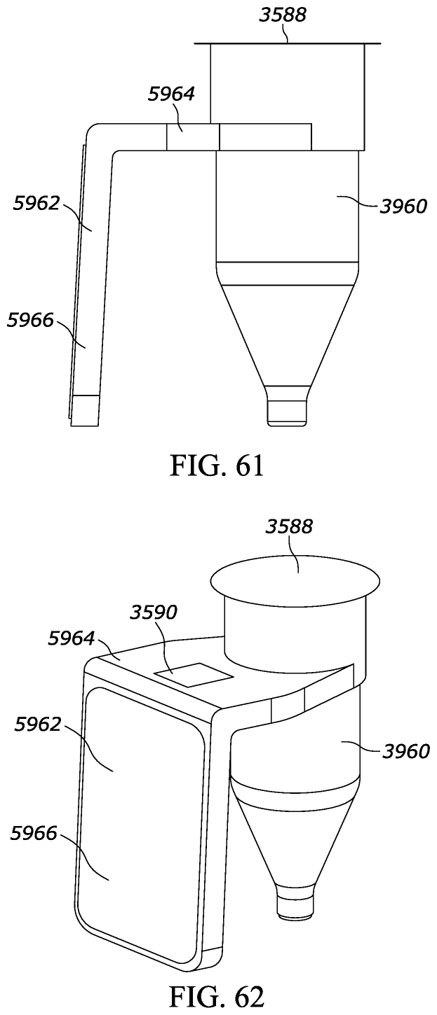

FIG. 61 is a side view of the bulk reagent well 3960 of FIG. 60.

FIG. 62 is an isometric view of the bulk reagent well 3960 of FIG. 60. A machine-readable code 3590 is shown on the first leg 3964 of the L-tab 3962. The machine-readable code 3590 may include information about the reagent contained within the bulk reagent well 3960 such the type of reagent, the date of manufacture, etc.

FIG. 63 is an isometric view of another reagent well plate 3972 including a first end wall 3568, a second end wall 3570, and a panel 3572 that be used with the system of FIG. 32. The reagent well plate 3972 is a dry reagent well plate 3974 in the implementation shown.

The first end wall 3568 has a pair of first end wall portions 3976 and a pair of the male snap-fit components 3574 and the second end wall 3570 includes a pair of second end wall portions 3978 and a pair of the male snap-fit components 3574 in the implementation shown. Each of the first end wall portions 3976 includes one of the male snap-fit components 3574 and each of the second end wall portions 3978 includes one of the male snap-fit components 3574. The male snap fit components 3574 each have a tapered tab 3980. The male snap fit components 3574 may be alternatively configured, however.

The male snap-fit connections 3574 may form a snap-fit connection with the dry well plate receptacle 3536, for example. A plurality of the impermeable barriers 3588 is shown where, each reagent well 3580 covered by one of the impermeable barriers 3588.

FIG. 64 is a side view of the reagent well plate 3594 of FIG. 63.

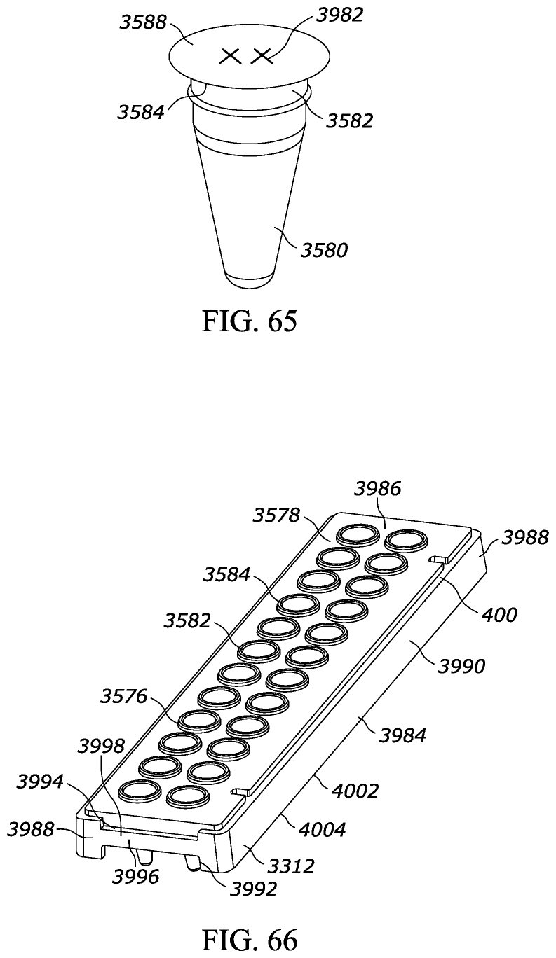

FIG. 65 is an isometric view of the reagent well 3580 of the reagent well plate 3594 of FIG. 63. The impermeable barrier 3588 includes indicia 3982. The indicia 3982 may include a machine-readable code and/or an indicator of the reagent contained within the reagent well 3580.

FIG. 66 is an isometric view of a well plate 3312 that can be used with the system 3300 of FIG. 32. The well plate 3312 includes a rectangular wall 3984 and a panel 3986 that is coupled to the rectangular wall 3984. The rectangular wall 3984 has end walls 3988 and side walls 3990. The end wall 3988 and the side wall 3990 extending outwardly from the panel 3986. The end walls 3988 each have a cutout 3992 and a recess 3994 that form a handle 3996 that extends between the side walls 3990. The gripper 3326 may interact with the handle 3996 to move the well plate 3312 between the consumables area 3302, the assay bay 3304, and/or the common bay 3306. For example, the gripper 3326 may have extensions that may be inserted into the cutout 3992 to lift and/or hold the well plate 3312. The extensions may inwardly extend.

The panel 3986 defines reagent well receptacles 3576 and includes a top surface 3578. The panel 3986 has a plurality of rows of the reagent well receptacles 3576 in the implementation shown. The panel 3986 may alternatively have a single row of the reagent well receptacles 3576 or more than two rows of the reagent well receptacles 3576.

The reagent wells 3580 include an end 3582 having an annular collar 3584. The reagent wells 3580 are positioned within the reagent well receptacles 3576 and the annular collars 3584 are shown engaging the top surface 3578.

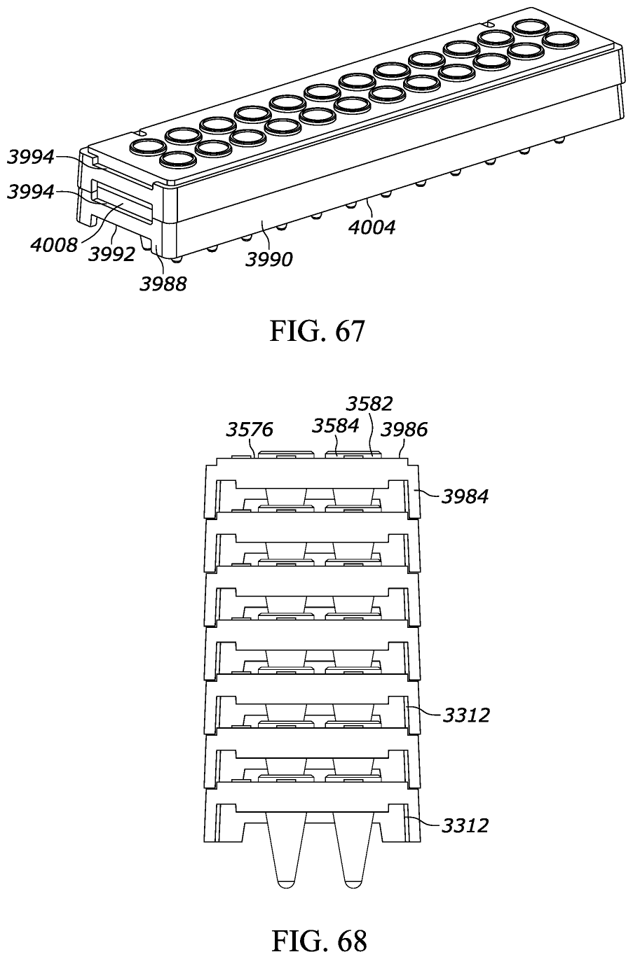

The end walls 3988 that include the handle 3996 form a dog-bone shape 3998 in the implementation shown. The rectangular wall 3984 and the panel 3986 form a step 4000 and the rectangular wall 3984 has an end 4002 that forms an opening 4004 that is sized to receive the step 4000 of an adjacent well plate 3312 as shown in FIG. 67.

FIG. 67 is an isometric view of a stack of well plates 3312 of FIG. 66. The cutout 3992 and the recess 3994 of an adjacent well plate are shown forming form an opening 4008. The opening 4008 may be sized to allow the gripper 3326 to access the opening 4008 and lift off one or more of the top well plate(s) 3312 from a stack of well plates 3312, for example.

FIG. 68 is a cross-sectional end view of a stack of the well plates 3312 of FIG. 66.

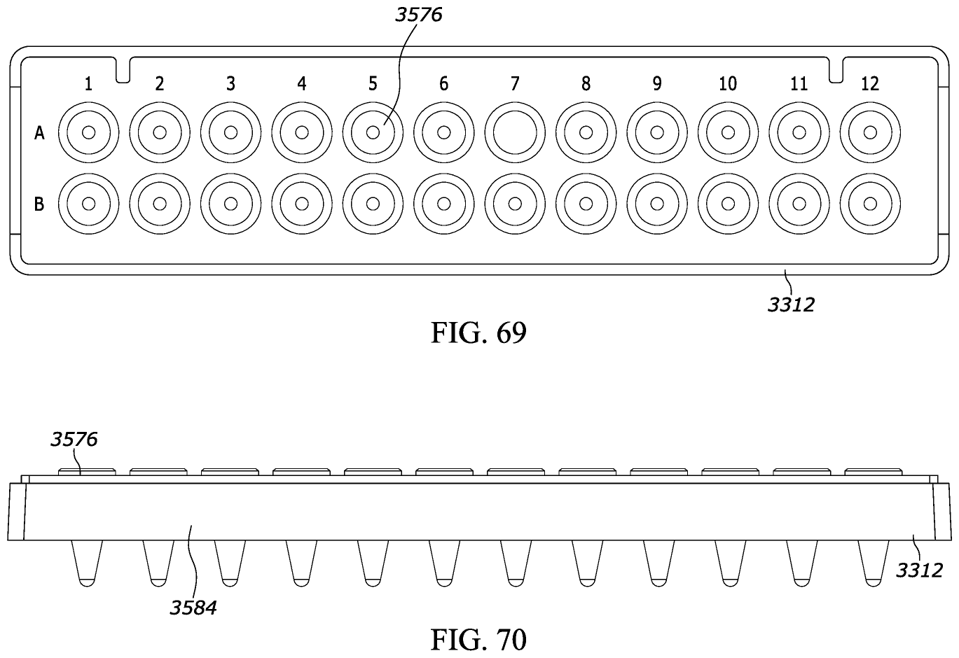

FIG. 69 is a top view of the well plate 3312 of FIG. 66.

FIG. 70 is a side view of the well plate of FIG. 66.

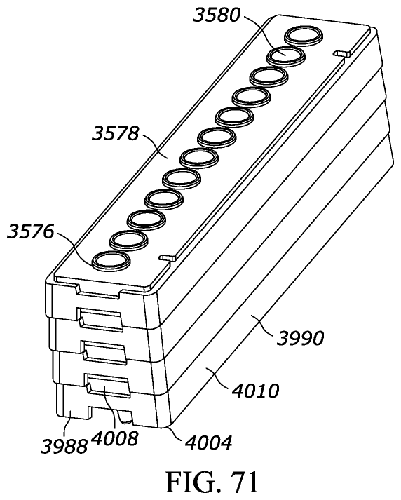

FIG. 71 is an isometric view of a stack of other well plates 4010 that can be used with the system 3300 of FIG. 32. The well plates 4010 are similar to the well plates 3312 of FIG. 66 but include a single row of the reagent well receptacles 3576.

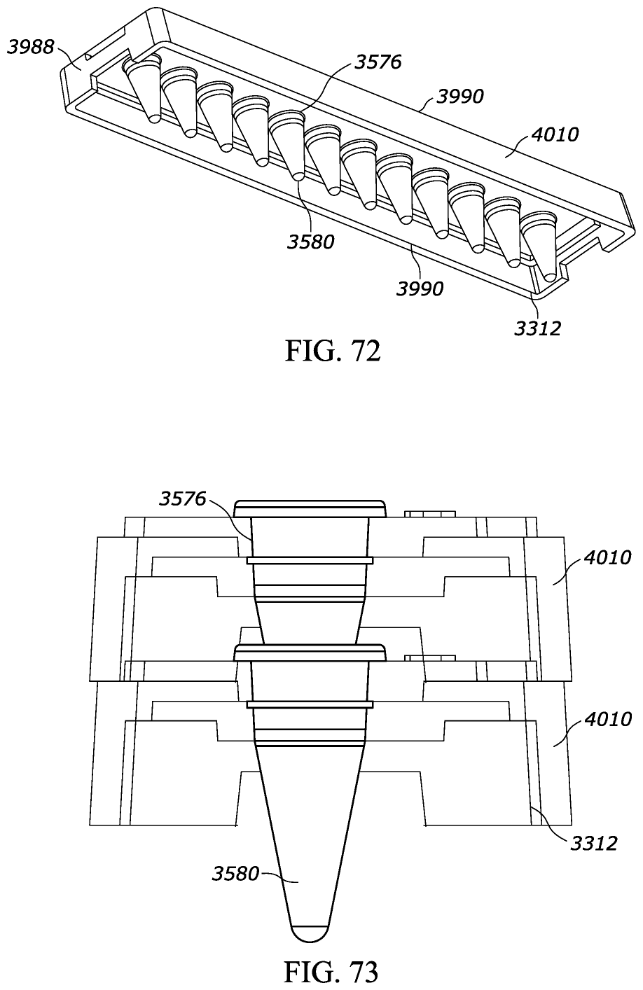

FIG. 72 is a bottom isometric view of the well plate 4010 of FIG. 71.

FIG. 73 is a cross-sectional end view of a stack of the well plates 4010 of FIG. 71.

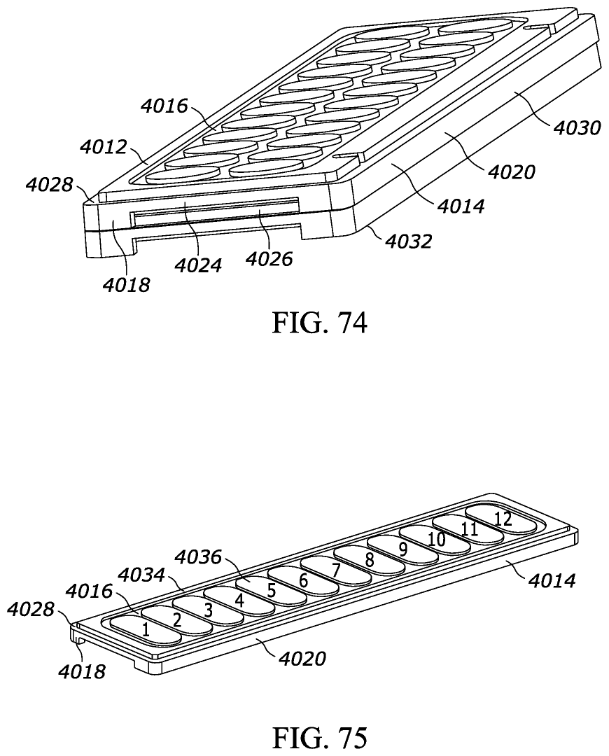

FIG. 74 is an isometric view of a stack of lids 4012 that can be used to cover the well plates 3312 of FIG. 66. The lid 4012 has a lid rectangular wall 4014 and a lid panel 4016. The lid panel 4016 has two rows of receptacles 4017 that are configured to cover the two rows of the reagent wells 3580.

The lid rectangular wall 4014 has lid end walls 4018 and lid side walls 4020. The lid end walls 4018 each comprising a lid cutout 4022 that forms a handle 4024 that extend between the lid side walls 4020. The lid cutout 4022 of an adjacent lid 4012 form an opening 4026.

The lid rectangular wall 4014 and the lid panel 4016 form a lid step 4028 and the lid rectangular wall 4014 has an end 4030 that forms a lid opening 4032 that is sized to receive the lid step 4028 of an adjacent lid 4012.

FIG. 75 is an isometric view of a lid 4034 that can be used to cover the well plates 4010 of FIG. 71. The lid 4034 is similar to the lid 4012 of FIG. 74 but the panel 4016 has receptacles 4036 that are configured to cover the single row of the reagent wells 3580.

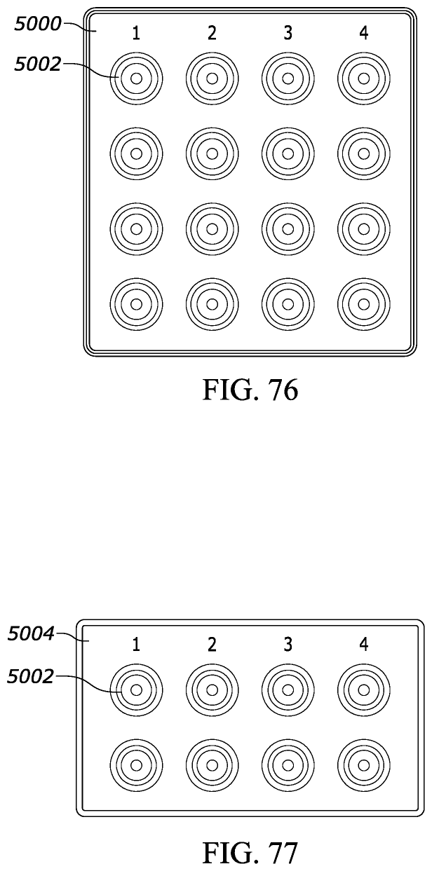

FIG. 76 is top plan view of an example sample cartridge 5000 including a plurality of wells 5002 that can be used with any of the disclosed implementations. The sample cartridge 5000 can be used with the sample sipper assembly 3310 of the system 3300 of FIG. 32 as an example. The wells 5002 may include pooled wells, prime wells, and/or wash wells in some implementations.

FIG. 77 is top plan view of an example reagent cartridge 5004 including a plurality of wells 5002 that can be used with any of the disclosed implementations. The reagent cartridge 5004 can be used with the common bay 3306 of the system 3300 of FIG. 32 as an example. The reagent cartridge 5004 may include reagent such as reagents used during quantification processes, denaturing processes, and/or diluting processes.

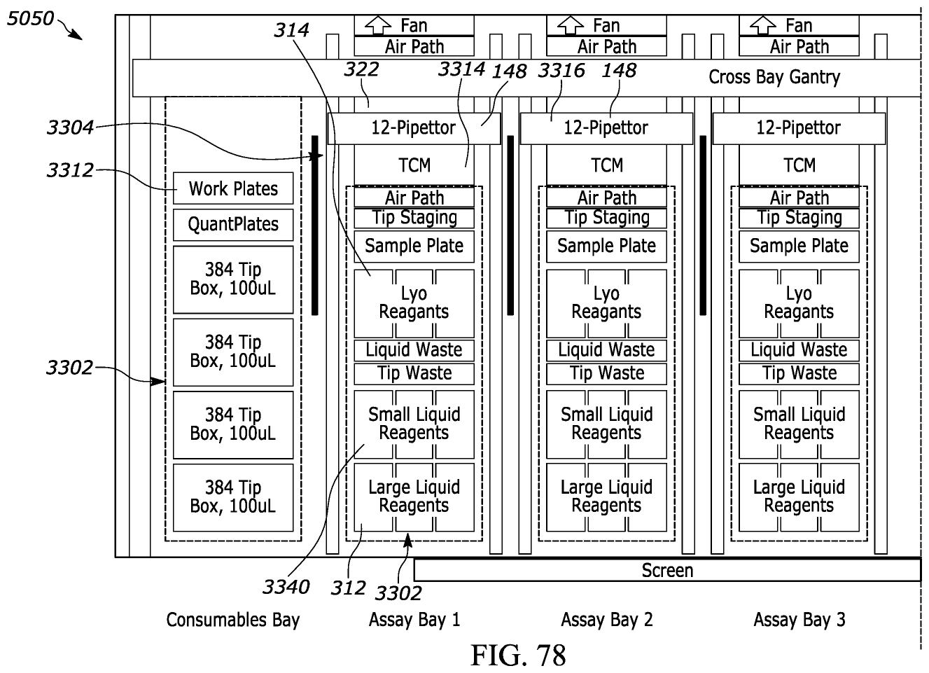

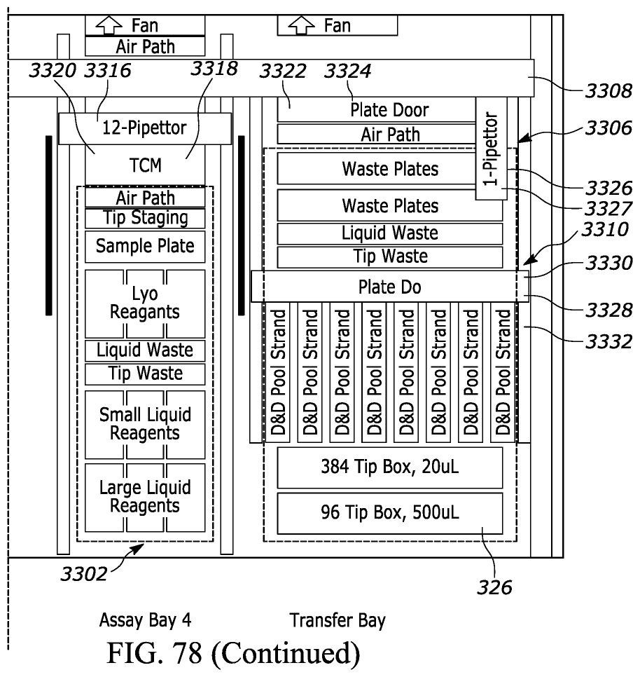

FIG. 78 is a plan view of an example system 5050 that be used to implement the system 300 of FIG. 1. The system 5050 of FIG. 78 includes four assay bays 3304, a common bay 3306, and the sipper assembly 3310.

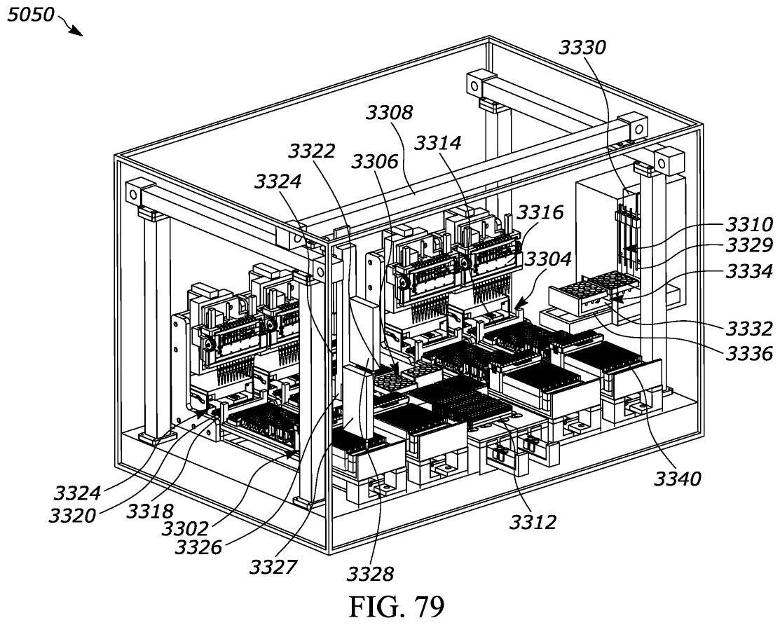

FIG. 79 is a front isometric view of an implementation of the system 5050 of FIG. 78.

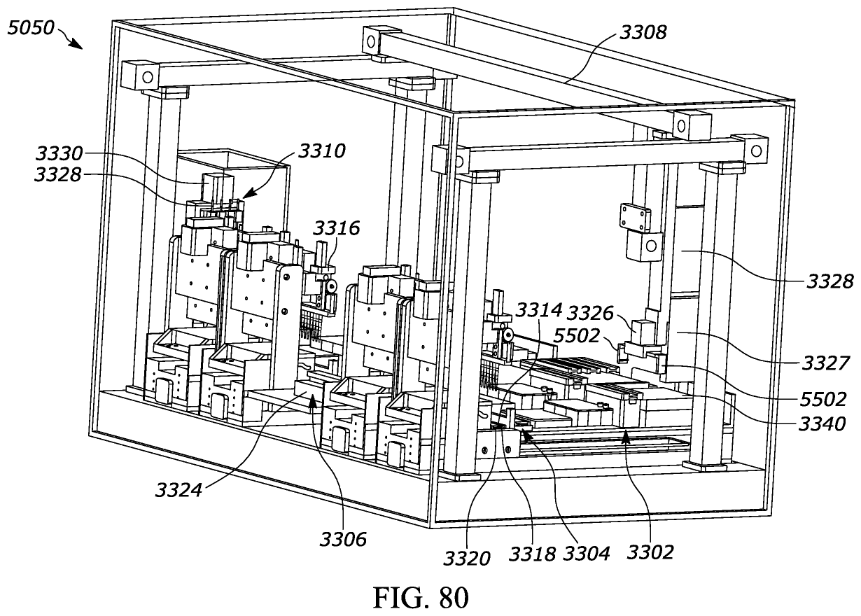

FIG. 80 is a rear isometric view of an implementation of the system 5050 of FIG. 78. The gripper 3326 of the cross-bay gantry 3308 is shown including extensions 5052. The extensions 5052 are movable toward and/or away from one another to move consumables 3340 between the consumables area 3302, the assay bays 3304, and/or the common bay 3306 as an example. The gripper 3326, the first contact dispenser 3327, and the second contact dispenser 3328 are shown carried by the cross-bay gantry 3308.

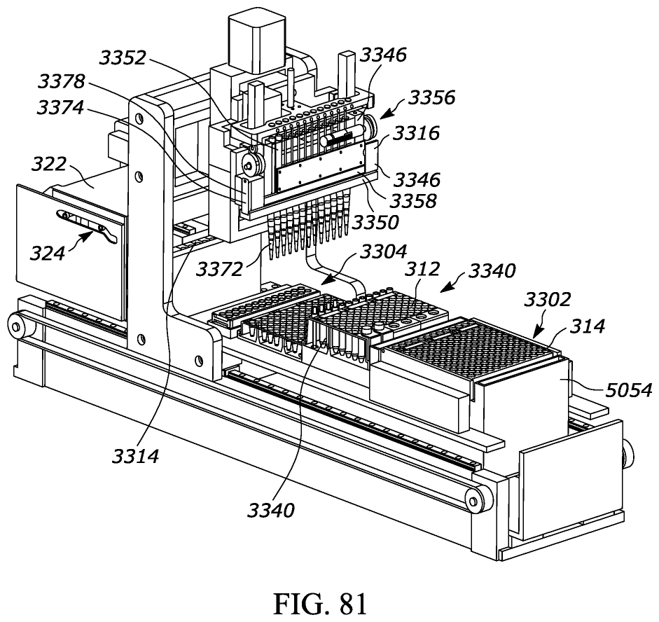

FIG. 81 is an isometric view of one of the assay bays 3304 of the system 5050 of FIG. 79. The assay bay 3304 of FIG. 81 is similar to the assay bay 3304 of FIG. 35. The assay bay 3304 of FIG. 81 includes a drawer 5054 with the consumables 3340 differently arranged, however.

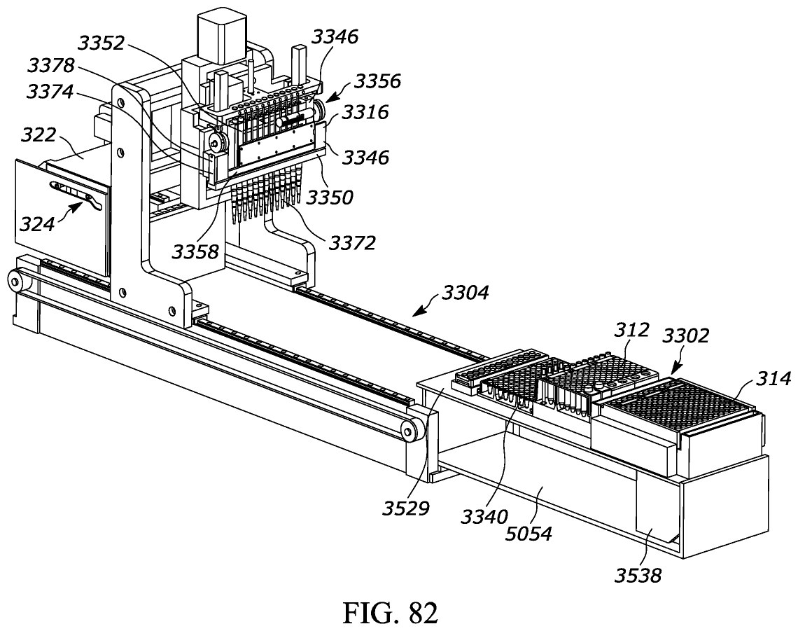

FIG. 82 is an isometric view of the assay bay 3304 of FIG. 81 with the drawer 5054 partially removed. The waste reservoir 3538 is shown extending through the platform 3529 of the drawer 5054.

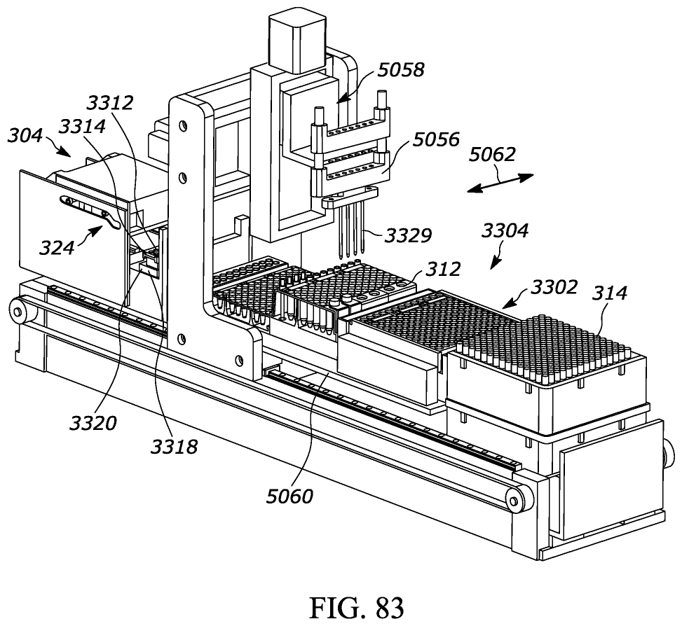

FIG. 83 is an isometric view of an example implementation of an assay bay 3304 including an alternative pipette assembly 5056 that can be used to implement system 300 of FIG. 1 and/or the system 3300 of FIG. 32. The pipette assembly 5056 may include an x-y-z stage 5058 that moves the sippers 3329 relative to the well plate 3312 positioned on the assay bay plate receptacle 3314 and/or relative to the consumables 3340 positioned on a drawer 5060 of the assay bay 3304. The pipette 5056 is shown including four tips and each row of the well plate 3312 includes twelve wells in the implementation shown.

The pipette assembly 5056 may perform processes associated with a first group of wells of the well plate 3312 using a first set of tips and the pipette assembly 5056 may perform processes associated with a second group of wells of the well plate 3312 using a second of tips. The stage 5058 may move the tips of the pipette assembly 5056 in a direction generally indicated by arrow 5062 between the first group of wells and the second group of wells. The pipette assembly 5056 may temporarily store the first set of tips when performing processes associated with the second group of wells and/or the pipette assembly 5056 may temporarily store the second set of tips when performing processes associated with the first group of wells.

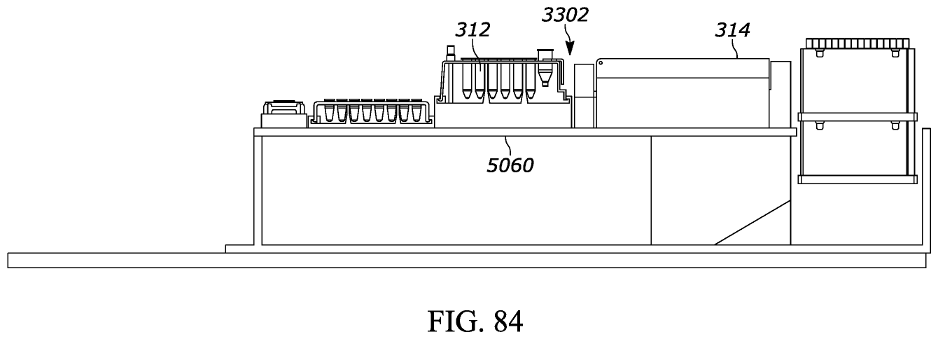

FIG. 84 is a side view of the drawer 5060 of the assay bay of 3304 of FIG. 83.

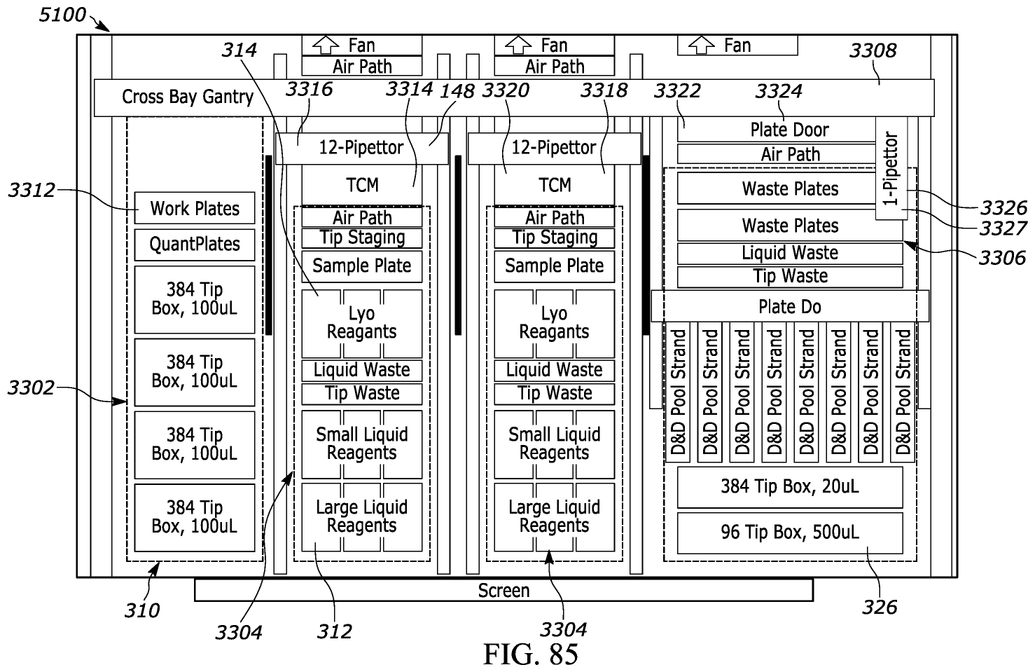

FIG. 85 is a plan view of an example system 5100 that be used to implement the system 300 of FIG. 1. The system 5100 of FIG. 83 includes a consumables area 3302, two assay bays 3304, a common bay 3306, and the sipper assembly 3310.

Example 1. A modular system for preparing a library of samples for sequencing, the modular system comprising: a first assay bay for performing a first assay, comprising: a first contact dispenser; a first working area comprising: a working plate receptacle; a thermocycler; and a magnet; and a first drawer, the first drawer comprising a consumables area adapted to receive a working plate adapted to contain a sample, and a plurality of consumables for interacting with the sample; a common bay, the common bay comprising an analyzer area including an imaging system; and a mover operatively coupled to the first assay bay and the common bay, wherein the mover is movable in a first direction along the first assay bay and a second direction perpendicular to the first direction between the first assay bay and the common bay, such that the mover is configured to move the working plate from the consumables area to the working plate receptacle in the first working area, wherein the first contact dispenser is linearly movable in the first direction between the consumables area and the first working area, such that the first contact dispenser is configured to move the plurality of consumables between the consumables area and the working plate in the working plate receptacle in the first working area, and wherein the mover is further configured to move the sample from the first working area to the analyzer area for analysis by the imaging system.

Example 2. The modular system of example 1, wherein the first contact dispenser is not movable in the second direction.

Example 3. The modular system of example 1 or 2, wherein the first contact dispenser and the mover are both movable in a third direction perpendicular to the first and second directions.

Example 4. The modular system of any one of the preceding examples, wherein the common bay further comprises a pooling area, and wherein the mover is configured to move between the analyzer area and the pooling area.

Example 5. The modular system of any one of the preceding examples, wherein the first contact dispenser comprises a first contact head configured to hold tips.

Example 6. The modular system of any one of the preceding examples, wherein the first drawer is linearly movable in the first direction relative to the first working area between a loading position and an operating position, wherein when the first drawer is in the loading position, the first drawer is spaced a first distance from the first working area, and when the first drawer is in the operating position, the first drawer is spaced a second distance from the first working area, the second distance being less than the first distance.

Example 7. The modular system of any one of the preceding examples, further comprising a movable stage coupled to the first contact dispenser for linearly moving the first contact dispenser in the first direction and the third direction.

Example 8. The modular system of example 7, further comprising a first actuator operatively coupled to the movable stage and to the magnet, the first actuator configured to actuate movement of the movable stage and to move the magnet relative to the working plate receptacle.

Example 9. The modular system of any one of the preceding examples, further comprising a gantry system, wherein the mover is movable along the gantry system.

Example 10. The modular system of any one of the preceding examples, further comprising a second actuator configured to actuate movement of the mover in the first and second directions.

Example 11. The modular system of any one of the preceding examples, further comprising a door that is movable to enclose the thermocycler and the working plate receptacle.

Example 12. The modular system of any one of the preceding examples, wherein the thermocycler is aligned with the working plate receptacle in the first direction.

Example 13. The modular system of any one of the preceding examples, wherein the thermocycler is configured to amplify the sample within the working plate.

Example 14. The modular system of any one of the preceding examples, wherein the consumables area is adapted to receive a lid for the working plate, and wherein the first contact dispenser is configured to move the lid from the consumables area and to place the lid on the working plate.

Example 15. The modular system of any one of the preceding examples, wherein the plurality of consumables comprise a tip tray comprising a first reusable tip and a second reusable tip, one or more additional working plates, an index tray adapted to contain indexes, a bead tray adapted to contain beads, and a reagent reservoir adapted to contain a reagent.

Example 16. The modular system of example 15, wherein the first contact dispenser is configured to move the beads in the consumables area to the working plate in the first working area using the first reusable tip and to move one or more reagents from the reagent reservoir in the consumables area to the working plate using the second reusable tip.

Example 17. The modular system of example 15, wherein the first contact dispenser is configured to move the beads in the consumables area to the working plate in the first working area using the first reusable tip and to move one or more reagents from the reagent reservoir in the consumables area to the working plate using the first reusable tip.

Example 18. The modular system of any one of examples 15-17, wherein the first contact dispenser is configured to aspirate the indexes from the index tray in the consumables area and to dispense the indexes in the working plate in the first working area.

Example 19. The modular system of any one of examples 15-18, wherein the first contact dispenser is configured to aspirate the beads from the bead tray in the consumables area and to dispense the beads in the working plate in the first working area.

Example 20. The modular system of example 19, wherein the magnet is movable toward the working plate receptacle to draw the beads in the working plate toward the magnet, and wherein the first contact dispenser is configured to aspirate a first reagent from the reagent reservoir in the consumables area and to dispense the first reagent in the working plate.

Example 21. The modular system of example 20, wherein the first contact dispenser is configured to aspirate the sample and the first reagent from the working plate, the mover is configured to move the working plate to the consumables area and to move a second working plate of the one or more additional working plates in the consumables area to the working plate receptacle in the first working area, the first contact dispenser is configured to dispense the sample and the first reagent in the second working plate, and the mover is configured to move the second working plate from the first working area to the analyzer area.

Example 22. The modular system of any one of the preceding examples, wherein the imaging system is configured to obtain image data of the portion of the first reagent and the sample to determine a concentration of the sample.

Example 23. The modular system of any one of the preceding examples, further comprising a second assay bay disposed in parallel with the first assay bay, the second assay bay for performing a second assay and comprising: a second contact dispenser; a second working area comprising: a working plate receptacle; a thermocycler; and a magnet; and a second drawer, the second drawer comprising a second consumables area adapted to receive a working plate adapted to contain a sample, and a plurality of consumables for interacting with the sample, wherein the mover is operatively coupled to the second assay bay and is configured to move the working plate from the second consumables area to the working plate receptacle in the second working area, wherein the second contact dispenser is linearly movable in the first direction between the consumables area and the second working area, such that the second contact dispenser is configured to move the plurality of consumables between the second consumables area and the working plate in the working plate receptacle in the second working area, and wherein the mover is movable in the second direction between the second assay bay and the common bay, such that the mover is configured to move the sample from the second working area to the analyzer area for analysis by the imaging system.

Example 24. The modular system of example 23, wherein the second assay is performed simultaneously with the first assay.

Example 25. A system, comprising: the modular system of any one of the preceding examples; a sequencer; and a plurality of fluidic lines fluidly coupling the common bay and the sequencer, such that the prepared samples automatically flow from the common bay to the sequencer.

Example 26. The system of example 25, wherein the common bay comprises a sipper assembly having a plurality of sippers, wherein the sequencer comprises a plurality of flowcells, and wherein the plurality of fluidic lines fluidly couple the plurality of sippers with the plurality of flowcells.

Example 27. A modular bay for preparing a library of samples for sequencing, the modular bay comprising: a contact dispenser; a working area comprising: a working plate receptacle; a thermocycler; and a magnet; and a drawer, the drawer comprising a consumables area adapted to receive a working plate adapted to contain a sample, and a plurality of consumables for interacting with the sample, wherein the contact dispenser is linearly movable in a longitudinal direction between the consumables area and the working area, such that the contact dispenser is configured to move the plurality of consumables between the consumables area and the working plate in the working area.

Example 28. The modular bay of example 26 or 27, wherein the contact dispenser comprises a contact head configured to hold tips.

Example 29. The modular bay of example 27 or 28, wherein the drawer is linearly movable in the longitudinal direction relative to the working area between a loading position and an operating position, wherein when the drawer is in the loading position, the drawer is spaced a first distance from the working area, and when the drawer is in the operating position, the drawer is spaced a second distance from the working area, the second distance being less than the first distance.

Example 30. The modular bay of any one of examples 27-29, wherein the contact dispenser is not movable in a lateral direction perpendicular to the longitudinal direction.

Example 31. The modular bay of any one of examples 27-30, further comprising a movable stage operatively coupled to the contact dispenser for linearly moving the contact dispenser in the longitudinal direction.

Example 32. The modular bay of example 31, further comprising an actuator configured to actuate movement of the movable stage in the longitudinal direction.

Example 33. The modular bay of any one of examples 27-32, further comprising a door that is movable to enclose the thermocycler and the working plate receptacle.

Example 34. The modular bay of any one of examples 27-33, wherein the thermocycler is aligned with the working plate receptacle in the longitudinal direction.

Example 35. The modular bay of any one of examples 27-34, wherein the thermocycler is configured to amplify the sample within the working plate.

Example 36. The modular bay of any one of examples 27-35, wherein the consumables area is adapted to receive a lid for the working plate, and wherein the contact dispenser is configured to move the lid from the consumables area and to place the lid on the working plate.

Example 37. The modular bay of any one of examples 27-36, wherein the plurality of consumables comprise a tip tray comprising a first reusable tip and a second reusable tip, one or more additional working plates, an index tray adapted to contain indexes, a bead tray adapted to contain beads, and a reagent reservoir adapted to contain a reagent.

Example 38. The modular bay of example 37, wherein the contact dispenser is configured to move the beads from the bead tray in the consumables area to the working plate in the working area using the first reusable tip and to move one or more reagents from the reagent reservoir in the consumables area to the working plate using the second reusable tip.

Example 39. The modular bay of example 37 or 38, wherein the contact dispenser is configured to aspirate the indexes from the index tray in the consumables area and to dispense the indexes in the working plate in the working area.

Example 40. The modular bay of any one of examples 37-39, wherein the contact dispenser is configured to aspirate the beads from the bead tray in the consumables area and to dispense the beads in the working plate in the working area.

Example 41. The modular bay of example 40, wherein the magnet is movable toward the working plate receptacle to draw the beads in the working plate toward the magnet, and wherein the contact dispenser is configured to aspirate a first reagent from the reagent reservoir in the consumables area and to dispense the first reagent in the working plate.

Example 42. The modular bay of example 41, wherein the contact dispenser is configured to aspirate the sample and the first reagent from the working plate, and the contact dispenser is configured to dispense the sample and the first reagent in a second working plate of the one or more additional working plates in the consumables area.

Example 43. An apparatus, comprising: a system, comprising: a consumables area comprising: a consumables receptacle to receive a tip tray comprising a first tip and a second tip, a first plate having a well containing a sample, a second plate having a well, an index tray having a well containing indexes, and a bead tray having a well containing beads; a mover; a contact dispenser; a stage to move the contact dispenser; a plate receptacle; a magnet; a thermocycler; and an analyzer area comprising an imaging system.

Example 44. The apparatus of example 43, further comprising an actuator to move the magnet relative to the plate receptacle.

Example 45. The apparatus of any one of examples 43-44, wherein the thermocycler is aligned with the plate receptacle.

Example 46. The apparatus of any one of examples 43-45, wherein the mover is to move the first plate from the consumables area to the plate receptacle.

Example 47. The apparatus of example 46, wherein the stage is to align the contact dispenser with the tip tray and the contact dispenser is to couple with the first tip from the tip tray, wherein the stage is to align the contact dispenser with the index tray and the contact dispenser is to aspirate the indexes from the index tray, wherein the stage is to align the contact dispenser with the first plate, and wherein the contact dispenser is to dispense the indexes into the well of the first plate.

Example 48. The apparatus of example 47, wherein the thermocycler is to amplify the sample within the well of the first plate.

Example 49. The apparatus of any one of examples 43-48, wherein the consumables area further comprises a lid and wherein the mover is to move the lid from the consumables area and to place the lid on the first plate to cover the well of the first plate with the lid.

Example 50. The apparatus of any one of examples 43-49, wherein the mover is to move the lid from the first plate to the consumables area.

Example 51. The apparatus any one of examples 49-50, wherein the stage is to align the contact dispenser with the bead tray and the contact dispenser is to aspirate the beads from the bead tray, wherein the stage is to align the contact dispenser with the first plate, and wherein the contact dispenser is to dispense the beads into the well of the first plate.

Example 52. The apparatus of example 16, wherein the stage is to align the contact dispenser with the first plate and the contact dispenser is to dispense a first reagent into the well of the first plate.

Example 53. The apparatus of example 52, wherein the stage is to align the contact dispenser with the tip tray and the contact dispenser is to place the first tip in the tip tray and the contact dispenser to couple with the second tip from the tip tray.

Example 54. The apparatus of any one of examples 52-53, wherein the actuator is to move the magnet toward the plate receptacle to draw the beads toward the magnet and wherein the stage is to align the contact dispenser with the first plate to allow the contact dispenser to aspirate the first reagent from the well.

Example 55. The apparatus of example 54, wherein the system comprises a waste and wherein the contact dispenser is to dispense the first reagent into the waste.

Example 56. The apparatus of example 55, wherein the stage is to align the contact dispenser with the first plate and the contact dispenser is to dispense a second reagent into the well of the first plate.

Example 57. The apparatus of example 56, wherein the actuator is to move the magnet toward the plate receptacle to draw the beads toward the magnet, wherein the stage is to align the contact dispenser with the first plate to allow the contact dispenser to aspirate the second reagent and the sample from the well of the first plate, and wherein the stage is to align the contact dispenser with the second plate to allow the contact dispenser to dispense the second reagent and the sample into the well of the second plate.

Example 58. The apparatus of example 57, wherein the imaging system is to obtain image data of a portion of the second reagent and the sample and the system to determine a concentration of the sample.

Example 59. The apparatus of example 58, further comprising a second contact dispenser.

Example 60. The apparatus of example 59, wherein the stage is to align the second plate with the second contact dispenser and wherein the second contact dispenser is to dispense a diluent into the well of the second plate to dilute the sample based on the concentration of the sample determined.

Example 61. The apparatus of any one of examples 43-60, wherein the system comprises a first working area comprising the contact dispenser, the stage to move the contact dispenser, the plate receptacle, the magnet, and the thermocycler.

Example 62. The apparatus of any one of examples 43-61, wherein the system comprises a second working area comprising the mover, a second contact dispenser, a plate receptacle, and the analyzer comprising the imaging system.

Example 63. The apparatus of any one of example 43-62, wherein the system comprises a loading area.

Example 64. The apparatus of example 63, wherein the loading area comprises a sipper assembly.

Example 65. The apparatus of example 64, wherein the sipper assembly comprises a sample sipper assembly.

Example 66. The apparatus of any one of examples 63-64, wherein the loading area comprises a plate receptacle.

Example 67. The apparatus of example 66, wherein the loading area comprises a stage to move the plate receptacle relative to the sipper assembly.

Example 68. The apparatus of any one of examples 43-67, further comprising a second system.

Example 69. The apparatus of example 68, wherein the second system is fluidly coupled to the system.

Example 70. The apparatus of any one of examples 68-69, wherein the second system comprises a sequencing instrument.

Example 71. The apparatus of any one of examples 43-70, wherein the thermocycler is positioned beneath the plate receptacle.

Example 72. The apparatus of any one of examples 43-71, wherein the plate receptacle includes a thermal block defining well receptacles and the thermocycler is positioned beneath the well receptacles.

Example 73. The apparatus of any one of examples 43-72, further including a heat sink coupled to the thermocycler.

Example 74. The apparatus of any one of examples 43-72, further comprising a lid and an actuator, the actuator to move the lid relative to the plate receptacle to cover the plate receptacle.

Example 75. An apparatus, comprising: a library preparation system; and a sequencing system fluidly coupled to the library preparation system.

Example 76. An apparatus, comprising: a system, comprising: a bay, comprising; a consumables area comprising a consumables receptacle; a first contact dispenser; a stage to move the first contact dispenser; a working area, comprising: a first plate receptacle; a magnet; and a thermocycler; a second working area comprising a second plate receptacle and an analyzer area comprising an imaging system; a second contact dispenser; a second stage to move the second contact dispenser relative to first bay and the second working area; and a mover.

Example 77. A modular system for preparing a library of samples for sequencing, the modular system comprising: a first assay bay, comprising: a first contact dispenser; a first working area comprising: a working plate receptacle; a thermocycler; and a magnet; and a first drawer, the first drawer comprising a consumables area adapted to receive a sample plate adapted to contain a sample, and a plurality of consumables for interacting with the sample; a common bay, the common bay comprising an analyzer area including an imaging system; and a mover operatively coupled to the first assay bay and the common bay, wherein the first contact dispenser is linearly movable in a first direction between the consumables area and the first working area, such that the first contact dispenser is configured to (i) move the sample plate in the consumables area to the working plate in the first working area, and (ii) move the plurality of consumables between the consumables area and the sample plate in the first working area, and wherein the mover is movable in the first direction and a second direction perpendicular to the first direction between the first assay bay and the common bay, such that the mover is configured to move the sample plate from the first working area to the analyzer area for analysis by the imaging system.

Example 78. A modular bay for preparing a library of samples for sequencing, the modular bay comprising: a contact dispenser; a working area comprising: a working plate receptacle; a thermocycler; and a magnet; and a drawer, the drawer comprising a consumables area adapted to receive a sample plate adapted to contain a sample, and a plurality of consumables for interacting with the sample, wherein the contact dispenser is linearly movable in a longitudinal direction between the consumables area and the working area, such that the contact dispenser is configured to (i) move the sample plate in the consumables area to the working plate in the working area, and (ii) move the plurality of consumables.

Example 79. An apparatus, comprising: a pipette assembly, comprising: a body comprising a base defining a plurality of pipette apertures, a guide comprising a plurality of protrusions that define pipette apertures that align with the pipette apertures of the base; a bar comprising a plurality of apertures through which the protrusions of the guide extend; a plurality of pipettes coupled to the body and extending through the pipette apertures of the body and the guide, the pipettes each having an end comprising a flange; a plurality of gaskets positioned between corresponding flanges of the pipettes and the protrusions; and a pipette cam assembly to move the body away from the guide and move the flanges of the pipettes toward the protrusions to compress the gaskets and to move the body toward the guide and move the flanges of the pipettes away from the protrusions to relax the gaskets.

Example 80. The apparatus of example 79, wherein the gaskets being compressed when the end of the pipettes are positioned within a pipette tip enables the gasket to form a coupling with the pipette tip.

Example 81. The apparatus of any one of examples 79-80, wherein the gaskets being relaxed when the end of the pipettes are positioned within a pipette tip enables the pipette tip to not be coupled with the pipette.

Example 82. The apparatus of any one of examples 79-81, wherein the guide comprises outward facing channels and wherein the bar comprises a first arm and a second arm that extend within the outward facing channels of the guide.

Example 83. The apparatus of any one of examples 79-81, wherein the bar comprises a first arm and a second arm and wherein the pipette cam assembly comprises: guide bearings coupled to the guide; bar bearings coupled to the corresponding first arm and the second arm; a cam shaft comprising inner lobes and outer lobes, the inner lobes to engage the guide bearings and the outer lobes to engage the bar bearings.

Example 84. The apparatus of example 83, wherein the inner lobes of the cam shaft engaging the guide bearings move the body away from the guide and move the flanges of the pipettes toward the protrusions to compress the gaskets.

Example 85. The apparatus of any one of examples 83-84, wherein the inner lobes of the cam shaft engaging the guide bearings is a second position enable the guide to move toward the bar and move the flanges of the pipettes away from the protrusions to relax the gaskets.

Example 86. The apparatus of any one of examples 83-85, wherein the outer lobes of the cam shaft engaging the bar bearings move the bar toward the flanges of the pipettes to enable the bar to engage pipette tips carried by the pipettes and urge the pipette tips to be released from the pipette assembly.

Example 87. The apparatus of any one of examples 83-86, wherein one of the bar bearings face one of the guide bearings.

Example 88. The apparatus of any one of examples 83-87, further comprising bar springs positioned between the bar and the guide to urge the bar bearings toward the corresponding outer lobe.

Example 89. The apparatus of any one of examples 79-88, further comprising a guide spring positioned between the body and the guide to urge the body away from the guide.

Example 90. The apparatus of any one of examples 83-89, wherein the body comprises a first side and a second side and wherein the cam shaft comprises a first cam shaft portion and a second cam shaft portion, the first cam shaft portion coupled to the first side of the body and the second cam shaft portion coupled to the second side of the body.

Example 91. The apparatus of example 90, wherein the first cam shaft portion is spaced from the second cam shaft portion.

Example 92. The apparatus of any one of examples 83-91, further comprising a motor and a gear set, the gear set coupled to the cam shaft.

Example 93. The apparatus of example 92, wherein movement of the motor rotates the cam shaft.

Example 94. The apparatus of any one of examples 92-93, wherein the gear set comprises first and second gears, first and second pinions, and a shaft coupling the first and second pinions.

Example 95. The apparatus of example 94, wherein the body comprises a first side and a second side and wherein the first gear is coupled to the first side of the body and the inner lobe and the outer lobe on the first side of the body and the second gear coupled to the second side of the body and the inner lobe and the outer lobe on the second side of the body.

Example 96. The apparatus of example 95, wherein the first side and the second side of the body define shaft apertures and the shaft is rotationally coupled within the shaft apertures.

Example 97. The apparatus of example 96, wherein the shaft is spaced from the pipettes.

Example 98. The apparatus of any one of examples 79-97, wherein the body comprises a first side and a second side, and a wall defining a receptacle, the pipettes positioned within the receptacle.

Example 99. The apparatus of any one of examples 79-98, wherein the pipettes each comprise a barrel, further comprising a plurality of pistons positioned and movable within the corresponding barrels.

Example 100. The apparatus of example 99, further comprising an actuator coupled to the pistons to move the pistons between a retracted position and an extended position within the barrels.

Example 101. The apparatus of example 100, wherein the actuator comprises a ball screw.

Example 102. The apparatus of example 101, wherein the actuator comprises a pair of linear rails and a lift, the lift coupled to the pistons and the linear rails and movable by the ball screw.

Example 103. The apparatus of example 102, wherein the lift is C-shaped and has ends, further comprising carriages coupled to the corresponding ends of the lift and coupled to the linear rails.

Example 104. An apparatus, comprising: a thermocycler, comprising: a base comprising a stop wall; a plate receptacle on the base; and a cover assembly movably coupled to the base, the cover assembly, comprising: a sled comprising a front wall and a rear wall and a receptacle defined between the front wall and the rear wall; a cover positioned within the receptacle of the sled; a cover follower positioned within the receptacle of the sled and movably coupled to the cover; and a cam assembly to cause the cover to move toward the base to cover the plate receptacle and to cause the cover follower to move toward the base, wherein the plate receptacle is to receive a plate having a well and the thermocycler is to adjust a temperature of a sample within the well of the plate.

Example 105. The apparatus of example 104, wherein the cam assembly, comprising: inner cam plates coupled to the sled and each defining an inner cam slot; outer cam plates coupled to the base and each defining an outer cam slot; cover bearings coupled to the cover and positioned to engage the stop wall; inner slot bearings coupled to the cover and movably positioned within the inner cam slot; cover follower bearings coupled to the cover follower and positioned to engage the rear wall of the sled; and outer slot bearings coupled to the cover follower and movably positioned within the outer cam slot.

Example 106. The apparatus of example 105, wherein the cover bearings are to engage the stop wall and cause the inner slot bearings to move within the inner cam slot and the cover bearings to move along the stop wall to move the cover toward the base to cover the plate receptacle.

Example 107. The apparatus of any one of examples 105-106, wherein the cover follower bearings are to engage the rear wall and cause the outer slot bearings to move within the outer cam slot and the cover follower bearings to move along the rear wall to move the cover follower toward the base.

Example 108. The apparatus of any one of examples 104-107, further comprising springs biasing the cover away from the cover follower.

Example 109. The apparatus of any one of examples 104-108, further comprising guide rods movably coupling the cover and the cover follower.

Example 110. The apparatus of example 109, wherein the cover comprises blind bores and the cover follower comprises through bores, the guide rods positioned within the corresponding blind bores of the cover and through bores of the cover follower.

Example 111. The apparatus of any one of examples 109-110, wherein the springs surround corresponding guide rods.

Example 112. The apparatus of any one of examples 105-111, wherein the cover defines cover bearing receptacles in which the cover bearings are positioned.

Example 113. The apparatus of any one of examples 105-112, wherein the cover follower defines cover follower bearing receptacles in which the cover follower bearings are positioned.

Example 114. The apparatus of any one of examples 104-113, further comprising linear rails coupled to the base and carriages coupled to the rear wall and coupled to the linear rails.

Example 115. The apparatus of any one of examples 104-114, wherein the stop wall comprises extensions between which an opening is defined, the front wall sized to pass between the extensions.

Example 116. The apparatus of example 115, wherein the cover bearings are to engage the extension.

Example 117. The apparatus of any one of examples 104-116, further comprising a magnet and an actuator, wherein the actuator is to move the magnet relative to the plate receptacle.

Example 118. An apparatus, comprising: a drawer comprising a platform, the drawer comprising: a plate receptacle coupled to the platform; a small liquid reagent well plate receptacle coupled to the platform and comprising: a base; a first end wall and having an inward extending lip that forms a first groove with the base; a second end wall coupled to the base and having an inward extending lip that forms a second groove with the base and comprising a key; and a large liquid reagent well plate receptacle coupled to the platform and comprising: a base; a first end wall having an inward extending lip that forms a first groove with the base of the large liquid reagent well plate receptacle; a second end wall coupled to the base and having an inward extending lip that forms a second groove with the base of the large liquid reagent well plate receptacle and comprising a key; and a dry well plate receptable positioned on the platform and defining a waste reservoir compartment; a waste reservoir having a wider portion including an inlet and a narrow portion that extends from the wider portion and is positioned within the waste reservoir compartment.

Example 119. The apparatus of example 118, further comprising a central well plate support wall extending from the base and positioned between the first end wall and the second end wall of the small liquid reagent well plate receptacle.

Example 120. The apparatus of any one of examples 118-119, further comprising a central well plate support wall extending from the base and positioned between the first end wall and the second end wall of the large liquid reagent well plate receptacle.

Example 121. The apparatus of any one of examples 118-120, wherein the small liquid reagent well plate receptacle is positioned between the plate receptacle and the large liquid reagent well plate receptacle.

Example 122. The apparatus of any one of examples 118-121, wherein the large liquid reagent well plate receptacle is positioned between the small liquid reagent well plate receptacle and the dry well plate receptacle.

Example 123. The apparatus of any one of examples 118-122, wherein the dry well plate receptacle is positioned between the large liquid reagent well plate receptacle and the wider portion of the waste reservoir including the inlet.

Example 124. The apparatus of any one of examples 118-123, wherein the inlet comprises a rectangular inlet to receive waste associated with a multiple-tip pipette.

Example 125. The apparatus of any one of examples 118-124, wherein the drawer further comprises a tip receptacle.

Example 126. The apparatus of example 125, wherein the tip receptacle is positioned between the large liquid reagent well plate receptacle and the dry well plate receptacle.

Example 127. An apparatus, comprising: a reagent well plate, comprising: a first end wall comprising a male snap-fit component; a second end wall comprising a male snap-fit component; and a panel coupled to and extending between the first end wall and the second end wall, the panel defining a plurality of reagent well receptacles and including a top surface; and a plurality of reagent wells comprising an end having an annular collar, the reagent wells positioned within the reagent well receptacles and the annular collars engaging the top surface.

Example 128. The apparatus of example 127, wherein the first end wall comprises a keying notch.

Example 129. The apparatus of any one of examples 127-128, further comprising an impermeable barrier coupled to the ends of the reagent wells.

Example 130. The apparatus of any one of examples 127-129, further comprising a machine-readable code coupled to the panel.

Example 131. The apparatus of any one of examples 127-130, wherein the reagent well plate comprises a small liquid reagent well plate.

Example 132. The apparatus of any one of examples 127-131, wherein the first end wall and the second end wall extend outwardly from the panel.

Example 133. The apparatus of any one of examples 127-132, wherein the panel is concave.

Example 134. The apparatus of example 133, wherein the panel is substantially flat when the first end wall and the second end wall are coupled to a reagent well plate receptacle.

Example 135. The apparatus of anyone of examples 127-134, wherein each of the reagent wells comprise a second annular collar longitudinally spaced from the annular collar, the panel positioned between the annular collar and the second annular collar of the corresponding reagent wells.

Example 136. The apparatus of any one of examples 127-135, wherein the panel comprises a plurality of second reagent well receptacles having a different size than the reagent well receptacles, the second reagent well receptacles positioned between the second end wall and the reagent well receptacles.

Example 137. The apparatus of example 136, further comprising bulk reagent wells to be positioned within the second reagent well receptacles.

Example 138. The apparatus of example 137, further comprising an L-tab coupled to each of the bulk reagent wells.

Example 139. The apparatus of example 138, wherein the L-tab comprise a first leg coupled to the bulk reagent well and a second leg extending from the first leg at an angle that corresponds to an angle of the second end wall.

Example 140. The apparatus of any one of examples 138-139, wherein the L-tab is positioned within a dimensional envelope of the reagent well plate.

Example 141. The apparatus of any one of examples 127-140, wherein the second end wall comprises first wall section and a second wall section that are coupled to form a recess.

Example 142. The apparatus of example 141, wherein the L-tab is positioned within a dimensional envelope of the recess.

Example 143. The apparatus of any one of examples 127-142, wherein the first end wall comprises a pair of first end wall portions and a pair of the male snap-fit components, each first end wall portion including one of the male snap-fit components.

Example 144. The apparatus of any one of examples 127-143, wherein the second end wall comprises a pair of second end wall portions and a pair of the male snap-fit components, each second end wall portion including one of the male snap-fit components.

Example 145. The apparatus of any one of examples 127-128, 130-144, further comprising plurality of impermeable barriers, each reagent well covered by one of the impermeable barriers.

Example 146. The apparatus of any one of examples 127-145, wherein each of the reagent wells comprise a second annular collar longitudinally spaced from the annular collar and wherein a snap-fit connection is formed between the plate, the annular collar, and the second annular collar.

Example 147. The apparatus of any one of examples 127-146, wherein the male snap fit components comprise a tapered tab.

Example 148. An apparatus, comprising: a well plate, comprising: a rectangular wall comprising end walls and side walls, the end walls each comprising a cutout and a recess that form a handle that extend between the side walls; a panel coupled to the rectangular wall, the panel defining a plurality of reagent well receptacles and including a top surface, the end walls and the side walls extending outwardly from the panel; a plurality of reagent wells comprising an end having an annular collar, the reagent wells positioned within the reagent well receptacles and the annular collars engaging the top surface.

Example 149. The apparatus of example 148, wherein the cutout and the recess of an adjacent plate form an opening.

Example 150. The apparatus of any one of examples 148-149, wherein the end walls comprising the handle form a dog-bone shape.

Example 151. The apparatus of any one of examples 148-150, wherein the rectangular wall and the panel form a step.

Example 152. The apparatus of example 151, wherein the rectangular wall comprises an end that forms an opening that is sized to receive the step of an adjacent well plate.

Example 153. The apparatus of any one of claims 148-152, wherein the panel comprises a plurality of rows of the reagent well receptacles.

Example 154. The apparatus of any one of examples 148-153, wherein the panel comprises a row of the reagent well receptacles.

Example 155. The apparatus of any one of examples 148-154, further comprising a lid comprising a lid rectangular wall and a lid panel, the lid rectangular wall comprising lid end walls and lid side walls, the lid end walls each comprising a lid cutout that forms a lid handle that extend between the lid side walls.

Example 156. The apparatus of example 155, wherein the lid cutout and an adjacent plate form an opening.

Example 157. The apparatus of any one of examples 155-156, wherein the lid rectangular wall and the lid panel form a lid step.

Example 158. The apparatus of example 157, wherein the lid rectangular wall comprises an end that forms a lid opening that is sized to receive the lid step of an adjacent lid.

Example 159. The apparatus of example 158, wherein the rectangular wall and the panel form a step and wherein the lid opening is sized to receive the step of an adjacent well plate.

Example 160. An apparatus, comprising: a consumables area to carry a plurality of well plates, each of the well plates comprising a rectangular wall comprising a cutout; a plurality of assay bays each comprising an assay bay plate receptacle, a pipette assembly, a thermocycler and a magnet to perform amplification processes and cleanup processes associated with preparing a library of samples for sequencing; a common bay comprising a common bay plate receptacle and an imaging system to perform quantification processes associated with preparing the library of samples for sequencing; and a cross-bay gantry comprising a gripper movable between the consumables area, the assay bays, and the common bay, the gripper comprising arms including inward extending extensions that are movable toward or away from one another, wherein the extensions of the gripper are positionable in the cutout of a corresponding well plate to move the well plate between any of the consumables bay, the assay bay plate receptacle, and the common bay plate receptacle.

Example 161. An apparatus, comprising: a library preparation system, comprising: a consumables area to carry a plurality of well plates; a plurality of assay bays each comprising an assay bay plate receptacle, a pipette assembly, a thermocycler and a magnet to perform amplification processes and cleanup processes associated with preparing a library of samples for sequencing; a common bay comprising a common bay plate receptacle and an imaging system to perform quantification processes associated with preparing the library of samples for sequencing; and a cross-bay gantry comprising a gripper movable between the consumables area, the assay bays, and the common bay; a sample sipper assembly comprising a plurality of sippers and an actuator to move the sippers relative to a plate receptacle of the library preparation system, the sample sipper assembly associated with transferring the library of samples to a sequencing system.

Example 162. The apparatus of example 161, further comprising fluidic lines fluidly coupling the sippers of the sample sipper assembly and the sequencing instrument.

Example 163. The apparatus of any one of examples 161-162, further comprising a loading area comprising the sample sipper assembly and the plate receptacle.

Example 164. The apparatus of example 163, wherein the loading area comprises a stage to move the plate receptacle relative to the sample sipper assembly.

Example 165. An apparatus, comprising: a plurality of assay bays each comprising an assay bay plate receptacle, a pipette assembly, a thermocycler and a magnet to perform amplification processes and cleanup processes associated with preparing a library of samples for sequencing, the pipette assembly, comprising: a body comprising a base defining a plurality of pipette apertures, a guide comprising a plurality of protrusions that define pipette apertures that align with the pipette apertures of the base; a bar comprising a plurality of apertures through which the protrusions of the guide extend; a plurality of pipettes coupled to the body and extending through the pipette apertures of the body and the guide, the pipettes each having an end comprising a flange; a plurality of gaskets positioned between corresponding flanges of the pipettes and the protrusions; and a pipette cam assembly to move the body away from the guide and move the flanges of the pipettes toward the protrusions to compress the gaskets and to move the body toward the guide and move the flanges of the pipettes away from the protrusions to relax the gaskets; and a common bay comprising a common bay plate receptacle and an imaging system to perform quantification processes associated with preparing the library of samples for sequencing; and a cross-bay gantry comprising a gripper movable between the assay bays and the common bay.

Example 166. The apparatus of example 165, wherein the gaskets being compressed when the end of the pipettes are positioned within a pipette tip enables the gasket to form a coupling with the pipette tip.

Example 167. The apparatus of any one of examples 165-166, wherein the gaskets being relaxed when the end of the pipettes are positioned within a pipette tip enables the pipette tip to not be coupled with the pipette.

Example 168. The apparatus of any one of examples 165-167, wherein the guide comprises outward facing channels and wherein the bar comprises a first arm and a second arm that extend within the outward facing channels of the guide.

Example 169. The apparatus of any one of examples 165-168, wherein the bar comprises a first arm and a second arm and wherein the pipette cam assembly comprises: guide bearings coupled to the guide; bar bearings coupled to the corresponding first arm and the second arm; a cam shaft comprising inner lobes and outer lobes, the inner lobes to engage the guide bearings and the outer lobes to engage the bar bearings.

Example 170. The apparatus of example 169, wherein the inner lobes of the cam shaft engaging the guide bearings move the body away from the guide and move the flanges of the pipettes toward the protrusions to compress the gaskets.

Example 171. The apparatus of any one of examples 169-170, wherein the inner lobes of the cam shaft engaging the guide bearings in a second position enable the guide to move toward the bar and move the flanges of the pipettes away from the protrusions to relax the gaskets.

Example 172. The apparatus of any one of examples 168-170, wherein the outer lobes of the cam shaft engaging the bar bearings move the bar toward the flanges of the pipettes to enable the bar to engage pipette tips carried by the pipettes and urge the pipette tips to be released from the pipette assembly.

Example 173. The apparatus of any one of examples 169-172, wherein one of the bar bearings face one of the guide bearings.

Example 174. The apparatus of any one of examples 169-173, further comprising bar springs positioned between the bar and the guide to urge the bar bearings toward the corresponding outer lobe.

Example 175. The apparatus of any one of examples 165-174, further comprising a guide spring positioned between the body and the guide to urge the body away from the guide.

Example 176. The apparatus of any one of 169-175, wherein the body comprises a first side and a second side and wherein the cam shaft comprises a first cam shaft portion and a second cam shaft portion, the first cam shaft portion coupled to the first side of the body and the second cam shaft portion coupled to the second side of the body.

Example 177. The apparatus of example 176, wherein the first cam shaft portion is spaced from the second cam shaft portion.

Example 178. The apparatus of any one of examples 169-177, further comprising a motor and a gear set, the gear set coupled to the cam shaft.

Example 179. The apparatus of example 178, wherein movement of the motor rotates the cam shaft.

Example 180. The apparatus of any one of examples 178-179, wherein the gear set comprises first and second gears, first and second pinions, and a shaft coupling the first and second pinions.

Example 181. The apparatus of example 180, wherein the body comprises a first side and a second side and wherein the first gear is coupled to the first side of the body and the inner lobe and the outer lobe on the first side of the body and the second gear is coupled to the second side of the body and the inner lobe and the outer lobe on the second side of the body.

Example 182. The apparatus of any one of examples 180-181, wherein the first side and the second side of the body define shaft apertures and the shaft is rotationally coupled within the shaft apertures.

Example 183. The apparatus of any one of examples 180-182, wherein the shaft is spaced from the pipettes.

Example 184. The apparatus of any one of examples 165-183, wherein the body comprises a first side and a second side, and a wall defining a receptacle, the pipettes positioned within the receptacle.

Example 185. The apparatus of any one of examples 165-184, wherein the pipettes each comprise a barrel, further comprising a plurality of pistons positioned and movable within the corresponding barrels.

Example 186. The apparatus of example 185, further comprising an actuator coupled to the pistons to move the pistons between a retracted position and an extended position within the barrels.

Example 187. The apparatus of example 186, wherein the actuator comprises a ball screw.

Example 188. The apparatus of example 187, wherein the actuator comprises a pair of linear rails and a lift, the lift coupled to the pistons and the linear rails and movable by the ball screw.

Example 189. The apparatus of example 188, wherein the lift is C-shaped and has ends, further comprising carriages coupled to the corresponding ends of the lift and coupled to the linear rails.

Example 190. The apparatus of any one of examples 165-189, further comprising a sample sipper assembly comprising a plurality of sippers and an actuator to move the sippers relative to a well plate, the sample sipper assembly associated with transferring the library of samples to a sequencing system.

Example 191. An apparatus, comprising: a plurality of assay bays each comprising an assay bay plate receptacle, a pipette assembly, a thermocycler and a magnet to perform amplification processes and cleanup processes associated with preparing a library of samples for sequencing, the thermocycler comprising: a base comprising a stop wall; a plate receptacle on the base; and a cover assembly movably coupled to the base, the cover assembly, comprising: a sled comprising a front wall and a rear wall and a receptacle defined between the front wall and the rear wall; a cover positioned within the receptacle of the sled; a cover follower positioned within the receptacle of the sled and movably coupled to the cover; and a cam assembly to cause the cover to move toward the base to cover the plate receptacle and to cause the cover follower to move toward the base, wherein the plate receptacle is to receive a plate having a well and the thermocycler is to adjust a temperature of a sample within the well of the plate, a common bay comprising a common bay plate receptacle and an imaging system to perform quantification processes associated with preparing the library of samples for sequencing; and a cross-bay gantry comprising a gripper movable between the assay bays and the common bay.

Example 192. The apparatus of example 191, wherein the cam assembly, comprising: inner cam plates coupled to the sled and each defining an inner cam slot; outer cam plates coupled to the base and each defining an outer cam slot; cover bearings coupled to the cover and positioned to engage the stop wall; inner slot bearings coupled to the cover and movably positioned within the inner cam slot; cover follower bearings coupled to the cover follower and positioned to engage the rear wall of the sled; and outer slot bearings coupled to the cover follower and movably positioned within the outer cam slot.

Example 193. The apparatus of example 192, wherein the cover bearings are to engage the stop wall and cause the inner slot bearings to move within the inner cam slot and the cover bearings to move along the stop wall to move the cover toward the base to cover the plate receptacle.

Example 194. The apparatus of any one of examples 192-193, wherein the cover follower bearings are to engage the rear wall and cause the outer slot bearings to move within the outer cam slot and the cover follower bearings to move along the rear wall to move the cover follower toward the base.

Example 195. The apparatus of any one of examples 191-194, further comprising springs biasing the cover away from the cover follower.

Example 196. The apparatus of any one of examples 191-195, further comprising guide rods movably coupling the cover and the cover follower.

Example 197. The apparatus of example 196, wherein the cover comprises blind bores and the cover follower comprises through bores, the guide rods positioned within the corresponding blind bores of the cover and through bores of the cover follower.

Example 198. The apparatus of any one of examples 196-197, wherein the springs surround corresponding guide rods.

Example 199. The apparatus of any one of examples 191-198, wherein the cover defines cover bearing receptacles in which the cover bearings are positioned.

Example 200. The apparatus of any one of examples 191-199, wherein the cover follower defines cover follower bearing receptacles in which the cover follower bearings are positioned.

Example 201. The apparatus of any one of examples 191-200, further comprising linear rails coupled to the base and carriages coupled to the rear wall and coupled to the linear rails.

Example 202. The apparatus of any one of examples 191-201, wherein the stop wall comprises extensions between which an opening is defined, the front wall sized to pass between the extensions.

Example 203. The apparatus of example 202, wherein the cover bearings are to engage the extension.

Example 204. The apparatus of any one of examples 191-203, further comprising an actuator, wherein the actuator is to move the magnet relative to the plate receptacle.

Example 205. An apparatus, comprising: a plurality of assay bays each comprising a drawer comprising a platform, an assay bay plate receptacle, a pipette assembly, a thermocycler and a magnet to perform amplification processes and cleanup processes associated with preparing a library of samples for sequencing, the drawer comprising: a plate receptacle coupled to the platform; a small liquid reagent well plate receptacle coupled to the platform and comprising: a base; a first end wall having an inward extending lip that forms a first groove with the base; a second end wall coupled to the base and having an inward extending lip that forms a second groove and comprising a key; and a large liquid reagent well plate receptacle coupled to the platform and comprising: a base; a first end wall having an inward extending lip that forms a first groove with the base of the large liquid reagent well plate receptacle; a second end wall coupled to the base and having an inward extending lip that forms a second groove with the base of the large liquid reagent well plate receptacle and comprising a key; and a dry well plate receptable positioned on the platform and defining a waste reservoir compartment; a waste reservoir having a wider portion including an inlet and a narrow portion that extends from the wider portion and is positioned within the waste reservoir compartment; a common bay comprising a common bay plate receptacle and an imaging system to perform quantification processes associated with preparing the library of samples for sequencing; and a cross-bay gantry comprising a gripper movable between the assay bays and the common bay.

Example 206. The apparatus of example 205, further comprising a central well plate support wall extending from the base and positioned between the first end wall and the second end wall of the small liquid reagent well plate receptacle.

Example 207. The apparatus of any one of examples 205-206, further comprising a central well plate support wall extending from the base and positioned between the first end wall and the second end wall of the large liquid reagent well plate receptacle

Example 208. The apparatus of any one of examples 205-207, wherein the small liquid reagent well plate receptacle is positioned between the plate receptacle and the large liquid reagent well plate receptacle.

Example 209. The apparatus of any one of examples 205-208, wherein the large liquid reagent well plate receptacle is positioned between the small liquid reagent well plate receptacle and the dry well plate receptacle.

Example 210. The apparatus of any one of examples 205-209, wherein the dry well plate receptacle is positioned between the large liquid reagent well plate receptacle and the wider portion of the waste reservoir including the inlet.

Example 211. The apparatus of any one of examples 205-210, wherein the inlet comprises a rectangular inlet to receive waste associated with a multiple-tip pipette.

Example 212. The apparatus of any one of examples 205-211, wherein the drawer further comprises a tip receptacle.

Example 213. The apparatus of example 212, wherein the tip receptacle is positioned between the large liquid reagent well plate receptacle and the dry well plate receptacle.

Example 214. A library preparation system for preparing a library of samples for sequencing, the system comprising: a working area for preparing a library of samples for genomic sequencing; a communication interface; and one or more processors communicatively coupled to the communication interface, and configured to communicate, via the communication interface, with a sequencer by transmitting or receiving information related to the library of samples.

Example 215. The library preparation system of example 214, wherein to communicate with the sequencer, the one or more processors are configured to: transmit, to the sequencer via the communication interface, identification information for the library of samples.

Example 216. The library preparation system of any one of examples 214-215, wherein to communicate with the sequencer, the one or more processors are configured to: transmit, to the sequencer via the communication interface, one or more run parameters for sequencing the library of samples.

Example 217. The library preparation system of any one of examples 214-216, wherein to communicate with the sequencer, the one or more processors are configured to: transmit, to the sequencer via the communication interface, an indication of a preparation status of the library of samples.

Example 218. The library preparation system of any one of examples 214-217, wherein to communicate with the sequencer, the one or more processors are configured to: transmit, to the sequencer via the communication interface, an instruction indicating a particular lane of a flow cell for the sequencer to sequence a particular sample of the library of samples.

Example 219. The library preparation system of any one of examples 214-218, wherein to communicate with the sequencer, the one or more processors are configured to: receive, via the communication interface, status information from the sequencer.

Example 220. The library preparation system of any one of examples 214-219, wherein to communicate with the sequencer, the one or more processors are configured to: receive, via the communication interface, an indication from the sequencer that the sequency is ready to receive the library of samples.

Example 221. The library preparation system of example 220, further comprising: a fluidic line configured to be coupled to the library preparation system and the sequencer, wherein the library preparation system transmits the library of samples to the sequencer via the fluidic line in response to receiving the indication that the sequencer is ready to receive the library of samples.

Example 222. The library preparation system of any one of examples 214-221, wherein the working area includes: a working plate; and a thermocycler.

Example 223. The library preparation system of any one of 214-222, further comprising: a contact dispenser; and a drawer comprising a consumables area adapted to receive a sample plate adapted to contain a sample, and a plurality of consumables for interacting with the sample, wherein the contact dispenser is configured to (i) move the sample plate in the consumables area to the working plate in the working area, and (ii) move the plurality of consumables.

Example 224. The library preparation system of any one of examples 214-223, wherein the communication interface includes a wired communication link attached to the library preparation system and the sequencer.

Example 225. A method for communicating between a library preparation system and a sequencer, the method comprising: preparing, by a library preparation system having a contact dispenser and a working area for causing one or more consumables to interact with a sample, a library of samples for genomic sequencing; and communicating, by one or more processors in the library preparation system via a communication interface, with a sequencer by transmitting or receiving information related to the library of samples.

Example 226. The method of example 225, wherein communicating with the sequencer includes: transmitting, by the one or more processors to the sequencer via the communication interface, identification information for the library of samples.

Example 227. The method of any one of examples 225-226, wherein communicating with the sequencer includes: transmitting, by the one or more processors to the sequencer via the communication interface, one or more run parameters for sequencing the library of samples.

Example 228. The method of any one of examples 225-227, wherein communicating with the sequencer includes: transmitting, by the one or more processors to the sequencer via the communication interface, an indication of a preparation status of the library of samples.

Example 229. The method of any one of examples 225-228, wherein communicating with the sequencer includes: transmitting, by the one or more processors to the sequencer via the communication interface, an instruction indicating a particular lane of a flow cell for the sequencer to sequence a particular sample of the library of samples.

Example 230. The method of any one of examples 225-229, wherein communicating with the sequencer includes: receiving, at the one or more processors via the communication interface, status information from the sequencer.

Example 231. The method of any one of examples 225-230, wherein communicating with the sequencer includes: receiving, at the one or more processors via the communication interface, an indication from the sequencer that the sequency is ready to receive the library of samples.

Example 232. The method of any one of examples 225-231, further comprising: transmitting, by the library preparation system via a fluidic line coupled to the library preparation system and the sequencer, the library of samples to the sequencer in response to receiving the indication that the sequency is ready to receive the library of samples.

Example 233. The method of any one of examples 225-232, wherein the communication interface includes a wired communication link attached to the library preparation system and the sequencer.

Example 234. A method comprising, comprising: receiving a small liquid reagent well plate by a small liquid reagent well plate receptacle on a platform of a drawer, wherein a snap-fit connection is formed when the small liquid reagent well plate receptacle receives the small liquid reagent well plate; receiving a large liquid reagent well plate by a large liquid reagent well plate receptacle coupled to the platform of the drawer, wherein a snap-fit connection is formed when the large liquid reagent well plate receptacle receives the large liquid reagent well plate; receiving a dry reagent well plate by a dry well plate receptable positioned on the platform and defining a waste reservoir compartment, wherein a waste reservoir is positioned within the waste reservoir compartment; and receiving the drawer in one of a plurality of assay bays of a library preparation system, the library preparation system comprising the assay bays and a common bay, the assay bays to each perform amplification processes and cleanup processes associated with preparing a library of samples for sequencing, the common bay to perform library quantification processes, library pooling processes, denaturing processes, and diluting processes associated with preparing the library of samples for sequencing.

Example 235. The method of example 234, wherein each assay bay comprises an assay bay plate receptacle, a pipette assembly, a thermocycler and a magnet to perform the amplification processes and cleanup processes associated with preparing the library of samples for sequencing.

Example 236. The method of any one of example 234-235, wherein the small liquid reagent well plate comprises a first end wall comprising a male snap-fit component, a second end wall comprising a male snap-fit component, a panel coupled to and extending between the first end wall and the second end wall, and a plurality of reagent wells positioned within the reagent well receptacles.

Example 237. The method of any one of examples 234-236, wherein the small liquid reagent well plate receptacle comprises a base, a first end wall coupled to the base and having an inward extending lip that forms a first groove with the base, and a second end wall coupled to the base and having an inward extending lip that forms a second groove with the base and comprising a key.

Example 238. The method of any one of examples 234-237, wherein receiving the small liquid reagent well plate by the small liquid reagent well plate receptacle on the platform of the drawer comprises a keying notch of the small liquid reagent well plate receiving a key of the small liquid reagent well plate receptacle.

Example 239. The method of any one of example 234-238, wherein the large liquid reagent well plate comprises a first end wall comprising a male snap-fit component, a second end wall comprising a male snap-fit component, a panel coupled to and extending between the first end wall and the second end wall, and a plurality of reagent wells positioned within the reagent well receptacles.

Example 240. The method of any one of examples 234-239, wherein the large liquid reagent well plate receptacle comprises a base, a first end wall coupled to the base and having an inward extending lip that forms a first groove with the base, and a second end wall coupled to the base and having an inward extending lip that forms a second groove with the base and comprising a key.

Example 241. The method of any one of examples 234-240, wherein receiving the large liquid reagent well plate by the large liquid reagent well plate receptacle on the platform of the drawer comprises a keying notch of the large liquid reagent well plate receiving a key of the large liquid reagent well plate receptacle.

Example 242. The method of any one of examples 234-241, wherein receiving the small liquid reagent well plate by the small liquid reagent well plate receptacle on the platform of the drawer comprises supporting a panel of the small liquid reagent well plate using a central well plate support wall extending from the base of the small liquid reagent well plate receptacle and positioned between a first end wall and a second end wall of the small liquid reagent well plate receptacle.

Example 243. The method of example 242, wherein supporting the panel of the small liquid reagent well plate comprises supporting the panel using a plurality of the central well plate support walls of the small liquid reagent well plate receptacle.

Example 244. The method of any one of examples 242-243, wherein the panel of the small liquid reagent well plate being supported by the central well plate of the small liquid reagent well plate receptacle enables the panel of the small liquid reagent well plate to be substantially flat.

Example 245. The method of anyone of examples 234-244, wherein a coupling between the small liquid reagent well plate and the small liquid reagent well plate receptacle and enables the panel of the small liquid reagent well plate to be substantially flat.

Example 246. The method of any one of examples 234-245, wherein receiving the large liquid reagent well plate by the large liquid reagent well plate receptacle on the platform of the drawer comprises supporting a panel of the large liquid reagent well plate using a central well plate support wall extending from the base of the small liquid reagent well plate receptacle and positioned between a first end wall and a second end wall of the small liquid reagent well plate receptacle.

Example 247. The method of example 246, wherein supporting the panel of the large liquid reagent well plate comprises supporting the panel using a plurality of the central well plate support walls of the large liquid reagent well plate receptacle.

Example 248. The method of any one of examples 245-247, wherein the panel of the large liquid reagent well plate being supported by the central well plate support of the large liquid reagent well plate receptacle enables the panel of the large liquid reagent well plate to be substantially flat.

Example 249. The method of anyone of examples 234-248, wherein a coupling between the large liquid reagent well plate and the large liquid reagent well plate receptacle enables the panel of the large liquid reagent well plate to be substantially flat.

Example 250. The method of any one of examples 234-249, wherein the large liquid reagent well plate comprises reagent well receptacles and second reagent well receptacles, the second reagent well receptacles having a different size than the reagent well receptacles, bulk reagent wells positioned within the second reagent well receptacles.

Example 251. A method, comprising: performing amplification processes and cleanup processes on a sample contained in a well plate positioned on a plate receptacle of a thermocycler on an assay bay of a library preparation system; removing the well plate from the plate receptacle of the thermocycler using a gripper by positioning the gripper within a cutout of the well plate and moving gripper away from the plate receptacle; moving the well plate using the gripper to an common bay of the library preparation system; performing library quantification processes at the common bay.

Example 252. The method of example 251, further comprising performing library pooling processes at the common bay.

Example 253. The method of any one of examples 251-252, further comprising performing at least one of denaturing processes or diluting processes at the common bay.

Example 254. The method of any one of examples 251-253, wherein the well plate comprises: a rectangular wall comprising end walls and side walls, the end walls each comprising the cutout and a recess that form a handle that extends between the side walls; a panel coupled to and extending between the rectangular wall, the panel defining a plurality of reagent well receptacles and including a top surface, the end walls and the side walls extending outwardly from the panel; and a plurality of reagent wells comprising an end having an annular collar, the reagent wells positioned within the reagent well receptacles and the annular collars engaging the top surface;

Example 255. The method of any one of examples 251-254, further removing a lid from the well plate at the plate receptacle using the gripper.

Example 256. The method of example 255, wherein removing the lid comprises positioning the gripper in a lid cutout that that forms a lid handle that extends between lid side walls of the lid and moving the lid away from the well plate.

Example 257. The method of any one of examples 251-256, further comprising moving a well plate from a consumables area to the assay bay using the gripper.

Example 258. The method of example 257, wherein moving the well plate comprises positioning the gripper in a cutout of the well plate that forms a handle of the well plate and moving the well plate away from the consumables area.

Example 259. The method of any one of examples 257-258, wherein moving the well plate from the consumables area comprises moving the well plate from a stack of well plate at the consumables area.

Example 260. A method, comprising: inserting ends of pipettes of a pipette assembly of an assay bay of a library preparation system and gaskets carried by the pipettes into pipette tips on a drawer of the assay bay; compressing the gaskets of the pipette assembly to couple the pipette tips and the pipette assembly; dispensing reagent into a well plate on a plate receptacle of the assay bay using the pipette assembly and the pipette tips; and performing amplification processes and cleanup processes associated on a sample contained within the well plate at the plate receptacle.

Example 261. The method of example 260, wherein compressing the gaskets comprises moving a body of the pipette assembly away from a guide of the pipette assembly and moving flanges of the pipettes toward protrusions of the guide to compress the gaskets.

Example 262. The method of example 261, wherein moving the body of the pipette assembly away from the guide of the pipette assembly and moving the flanges of the pipettes toward protrusions of the guide to compress the gaskets comprises using a pipette cam assembly.

Example 263. The method of any one of examples 260-262, further moving a body of the pipette assembly away from a guide of the pipette assembly and moving flanges of the pipettes toward protrusions of the guide to compress the gaskets by engaging inner lobes of a cam shaft of a pipette cam assembly of the pipette assembly with guide bearings of the pipette assembly.

Example 264. The method of any one of examples 260-263, further comprising relaxing the gaskets to enable the pipette tips to be uncoupled from the pipette assembly.

Example 265. The method of example 264, wherein relaxing the gaskets comprises moving a body of the pipette assembly toward a guide of the pipette assembly and moving flanges of the pipettes to relax the gaskets.

Example 266. The method of example 265, wherein moving the guide of the pipette assembly toward the bar of the pipette assembly and moving the flanges of the pipettes away from the protrusions of the guide to relax the gasket comprises using a pipette cam assembly.

Example 267. The method of any one of examples 260-266, further comprising enabling flanges of the pipettes to move away from protrusions of a guide of the pipette assembly to relax the gaskets by positioning inner lobes of a cam shaft of a pipette cam assembly of the pipette assembly in a second position relative to guide bearings of the pipette assembly to enable the body to move toward the guide of the pipette assembly and enable the flanges of the pipettes to move away from the protrusions of the guide to relax the gaskets.

Example 268. The method of example 267, further comprising releasing pipette tips from the pipette assembly.

Example 269. The method of example 268, wherein releasing the pipette tips from the pipette assemblies comprises moving the bar toward the flanges of the pipettes away from the protrusions of the guide and engaging the pipette tips with the bar and urging the pipette tips to be released from the pipette assembly.

Example 270. The method of any one of examples 258-267, further comprising moving pistons within barrels of the pipettes between a retracted position and an extended position using an actuator.

Example 271. A method, comprising: moving a sled of a cover assembly of a thermocycler toward a stop wall of a base of the thermocycler, a cover positioned within a receptacle of the sled and a cover follower positioned within the receptacle of the sled and movably coupled to the cover; engaging the stop wall with cover bearings coupled to the cover; engaging a rear wall of the sled with cover follower bearings coupled to the cover follower; moving inner slot bearings within inner cam slots of inner cam plates to move the cover toward covering a plate receptacle, the inner slot bearings coupled to the cover and the inner cam plates coupled to the sled; and moving outer slot bearings within outer cam slots of outer cam plates to move the cover follower toward the base, the outer slot bearings coupled to the cover follower and the outer cam plates coupled to the plate.

Example 272. The method of example 271, further comprising performing amplification processes on a sample in a well plate positioned on the plate receptacle.

Example 273. The method of any one of examples 271-272, further comprising performing cleanup processes on the sample in the well plate positioned on the plate receptacle.

Example 274. The method of example 273, wherein performing clean up processes comprises moving a magnet toward the well plate on the plate receptacle.

Example 275. The method of any one of examples 271-274, wherein the cover bearings engaging the stop wall causes the inner slot bearings to move within the inner cam slot and the cover bearings to move along the stop wall to move the cover toward the base to cover the plate receptacle.

Example 276. The method of any one of examples 271-275, wherein the cover follower bearings engaging the rear wall causes the outer slot bearings to move within the outer cam slot and the cover follower bearings to move along the rear wall to move the cover follower toward the base.

Example 277. The method of any one of examples 271-276, further comprising biasing the cover away from the cover follower.

Example 278. A method, comprising: performing amplification processes and cleanup processes on samples in well plates at a plurality of assay bays, each assay bay comprising an assay bay plate receptacle, a pipette assembly, a thermocycler and a magnet; moving the samples from the assay bays to a common bay using a cross-bay gantry; and performing library quantification processes on the samples, the library quantification processes at the common bay; and archiving the library of samples.

Example 279. The method of example 278, wherein archiving the library of samples comprises sealing the samples.

Example 280. The method of any one of examples 278-279, wherein archiving the library of samples comprises freezing the samples.

Example 281. A method, comprising: performing amplification processes and cleanup processes on samples in well plates at a plurality of assay bays, each assay bay comprising an assay bay plate receptacle, a pipette assembly, a thermocycler and a magnet; moving the samples from the assay bays to a common bay using a cross-bay gantry; and performing library quantification processes and library pooling processes on the samples at the common bay.

Example 282. The method of example 281, wherein the library pooling processes comprise preparing a equimolar pool using the samples based on the quantification values determined by the library quantification processes.

Example 283. The method of any one of examples 281-282, further comprising archiving a remaining portion the library of samples.

Example 284. The method of example 283, wherein archiving the library of samples comprises sealing the samples.

Example 285. The method of any one of examples 283-284, wherein archiving the library of samples comprises freezing the samples.

Example 286. A method, comprising: performing amplification processes and cleanup processes on samples in well plates at a plurality of assay bays, each assay bay comprising an assay bay plate receptacle, a pipette assembly, a thermocycler and a magnet; moving the samples from the assay bays to a common bay using a cross-bay gantry; and preparing a sequence ready pool of samples at the common bay.

Example 287. The method of example 286, wherein the preparing the sequence ready pool of samples at the common bay comprising performing library quantification processes and library pooling processes on the samples.

Example 288. The method of any one of examples 286-287, wherein the preparing the sequence ready pool of samples at the common bay comprising performing denaturing processes on the samples.

Example 289. The method of any one of examples 286-288, wherein the preparing the sequence ready pool of samples at the common bay comprising performing diluting processes on the samples.

Example 290. A method, comprising: performing amplification processes and cleanup processes on samples in well plates at a plurality of assay bays, each assay bay comprising an assay bay plate receptacle, a pipette assembly, a thermocycler and a magnet; moving the samples from the assay bays to a common bay using a cross-bay gantry; and preparing a sequence ready pool of samples at the common bay; and transferring the sequence ready pool of the samples to a sequencer.

Example 291. The method of example 290, wherein transferring the sequence ready pool of samples to the sequencer comprises transferring the library of samples to a sequencing system using a sample sipper assembly.

Example 292. The method of any one of examples 290-291, wherein the preparing the sequence ready pool of samples at the common bay comprising performing library quantification processes and library pooling processes on the samples.

Example 293. The method of any one of examples 290-292, wherein the preparing the sequence ready pool of samples at the common bay comprising performing denaturing processes on the samples.

Example 294. The method of any one of examples 290-293, wherein the preparing the sequence ready pool of samples at the common bay comprising performing diluting processes on the samples.

Example 295. An apparatus, comprising: a library preparation system comprising: an assay bay to perform amplification processes and cleanup processes; and a common bay to perform quantification process.

Example 296. An apparatus, comprising: a library preparation system, comprising: a sample sipper assembly comprising sippers to be coupled to a sample cartridge; and a sequencing instrument, comprising; a flow cell interface to be coupled to a flow cell having a plurality of channels; a central valve and an auxiliary waste fluidic line coupled to the central valve and to be coupled to a waste reservoir, the central valve coupled to the flow cell interface and movable between a first position fluidically connecting an inlet of the plurality of channels to the auxiliary waste fluidic line and a second position fluidically connecting a reagent reservoir and the plurality of channels; and a sample loading assembly positioned between the flow cell interface and the sample sipper assembly of the library preparation system, the sample loading assembly comprising: a body carrying a plurality of sample valves and defining a plurality of sample ports and a plurality of flow cell ports, each sample port coupled to a corresponding sipper of the sample sipper assembly via a sample fluidic line, wherein sample sipper assembly of the library preparation system is positioned downstream of the flow cell interface of the sequencing instrument.

Example 297. The apparatus of example 296, wherein each flow cell port is coupled to a corresponding port of the flow cell interface and associated with one of the plurality of channels of the flow cell via a flow cell fluidic line.

Example 298. The apparatus of any one of examples 296-297, wherein the sample valves are movable to fluidically couple a sipper of the library preparation system and sample port of the sequencing instrument and a corresponding outlet of a channel of the plurality of channels of the flow cell.

Example 299. The apparatus of any one of examples 296-298, wherein the sample valves are movable to fluidically decouple a sipper of the library preparation system and sample port of the sequencing instrument and a corresponding outlet of a channel of the plurality of channels of the flow cell.

Example 300. The apparatus of any one of examples 296-299, wherein the sample valves are operable to individually load each channel of the plurality of channels of the flow cell.

Example 301. The apparatus of any one of examples 296-300, wherein the sequencing instrument further comprises a plurality of pumps and wherein the body of the sample loading assembly further defines a plurality of pump ports, each pump port being coupled to one of the pumps of the plurality of pumps via a pump-channel fluidic line.

Example 302. The apparatus of example 301, wherein each sample valve is operable to fluidly couple a sipper of the sample sipper assembly of the library preparation system and a corresponding pump of the plurality of pumps of the sequencing instrument and to fluidly couple a pump of the plurality of pumps and a corresponding channel of the plurality of channels of the flow cell.

Example 303. The apparatus of any one of examples 301-302, wherein the pumps are operable to individually control fluid flow for each channel of the plurality of channels of the flow cell.

Example 304. The apparatus of any one of examples 296-303, wherein the outlets of the plurality of channels are fluidly couplable to a waste reservoir.

Example 305. The apparatus of example 304, wherein the sequencing instrument comprises a pump manifold assembly comprising a plurality of pumps and wherein the pump manifold assembly is to fluidly couple the outlets of the plurality of channels to the waste reservoir.

Example 306. The apparatus of any one of examples 301-305, wherein the sequencing instrument comprises a pump manifold assembly comprising the pumps and a cache, the sequencing instrument further comprising a bypass valve and a bypass fluidic line coupling the bypass valve and the cache.

Example 307. The apparatus of example 306, wherein the sequencing instrument further comprises a shared line valve, a plurality of dedicated reagent fluidic lines, and a shared reagent fluidic line, the shared reagent fluidic line coupling the shared line valve and the central valve and adapted to flow one or more reagents to the flow cell, each dedicated reagent fluidic line coupling the bypass fluidic line and the central valve and adapted to flow one or more reagents toward the flow cell.

Example 308. The apparatus of any one of examples 306-307, wherein the pump manifold assembly carries a plurality of pump valves and a cache valve and includes a plurality of pump-channel fluidic lines, a plurality of pump fluidic lines, a shared fluidic line, a cache fluidic line, and a primary waste fluidic line, the cache fluidic line being coupled to and between the cache and the cache valve, each pump valve being coupled to a corresponding pump-channel fluidic line, a corresponding pump fluidic line, and the shared fluidic line, the cache valve being coupled to the cache fluidic line, the primary waste fluidic line, and the shared fluidic line.

Example 309. The apparatus of example 308, wherein the pump valves and the pumps are operable to individually control fluid flow for each channel of the plurality of channels of the flow cell and the pump valves, the cache valve, and the pumps are operable to control fluid flow between the bypass fluidic line and the shared fluidic line.

Example 310. The apparatus of example 310, wherein the pump valves, the cache valve, and the pumps are operable to control fluid flow between the shared fluidic line and the primary waste fluidic line.

Example 311. An apparatus, comprising: a library preparation system, comprising: a sample sipper assembly comprising sippers to be coupled to a sample cartridge a sequencing instrument, comprising: one or more valves adapted to be coupled to corresponding reagent reservoirs; a flow cell interface adapted to be coupled to a flow cell; a pump adapted to load a channel of the flow cell with the sample of interest via the flow cell interface associated with an outlet of the flow cell and a corresponding sipper of the sample sipper assembly.

Example 312. The apparatus of example 311, wherein the sequencing instrument further comprising a pump manifold assembly having a plurality of pumps including the pump and a plurality of pump valves, wherein each pump and a corresponding pump valve are operable to individually control the flow of the sample of interest between each sipper of the sample sipper assembly of the library preparation system and a corresponding channel of the flow cell.

Example 313. The apparatus of any one of examples 311-312, wherein the sequencing instrument further comprises a sample loading assembly having a plurality of sample valves, wherein each sample valve is operable to individually load each channel of the plurality of channels of the flow cell with the sample of interest.

Example 314. The apparatus of any one of examples 311-313, further comprising a flow cell assembly including the flow cell having a plurality of channels and a flow cell manifold, wherein the flow cell manifold includes an inlet, a plurality of fluidic lines, and a plurality of outlets, wherein each outlet of the flow cell manifold is coupled to a corresponding channel of the flow cell.

Example 315. A method, comprising: moving a first sample valve of one or more sample valves to a first position to fluidically couple a first sipper of a sipper manifold assembly of a library preparation system with a first pump of a sequencing instrument; drawing a first sample of interest through the first sipper of the sipper manifold assembly from the library preparation system toward the first pump of the sequencing instrument; moving the first sample valve to a second position to fluidically couple the first pump and a channel of a flow cell coupled to a flow cell interface of the sequencing instrument; and pumping the first sample of interest into the first channel of the flow cell through an outlet of the first channel.

Example 316. The method of example 315, further comprising; moving a second sample valve of the one or more sample valves to a first position to fluidically couple a second sipper of the sipper manifold assembly of the library preparation system with a second pump of the sequencing instrument; drawing a second sample of interest through the second sipper of the sipper manifold assembly from the library preparation system toward the second pump of the sequencing instrument; moving the second sample valve to a second position to fluidically couple the second pump and a second channel of the flow cell coupled to the flow cell interface of the sequencing instrument; and pumping the second sample of interest into the second channel of the flow cell through an outlet of the second channel.

Example 317. The method of any one of examples 315-316, further comprising fluidically coupling a reagent reservoir with an inlet of the channel of the flow cell.

Example 318. The method of any one of examples 315-317, wherein pumping the first sample of interest from the first sample reservoir into the channel of the flow cell includes moving the first sample of interest from a sample cartridge at the library preparation system using the sipper of the sipper manifold assembly to a corresponding sample port of a sample loading assembly of the sequencing instrument, out of an associated pump port of the sample loading assembly, and into a pump-channel fluidic line of a pump manifold assembly of the sequencing instrument, and moving the first sample of interest from the pump-channel fluidic line, through the associated pump port, and through a corresponding flow cell port of the sample loading assembly, each flow cell port being coupled to a corresponding port of the flow cell interface and associated with one of the channels of the plurality of channels of the flow cell.

Example 319. The method of any one of examples 315-318, wherein moving the first sample valve of the one or more sample valves to the first position includes fluidically coupling a sample port of a sample loading assembly of the sequencing instrument and the sipper of the sample sipper assembly and a corresponding pump of the sequencing instrument and wherein moving the first sample valve of the one or more sample valves to the second position includes fluidically coupling the corresponding pump and the channel of the plurality of channels of the flow cell.

Example 320. The method of any one of examples 315-319, further comprising operating one or more of a plurality of pumps of the sequencing instrument to individually control fluid flow for each channel of the plurality of channels of the flow cell.

Example 321. The method of any one of examples 315-320, further comprising flowing the first sample of interest out of the first channel of the flow cell and into an auxiliary waste fluidic line of the sequencing instrument, the auxiliary waste fluidic line being upstream of the flow cell and fluidically coupled to a central valve and a waste reservoir of the sequencing instrument.

Example 322. The method of any one of examples 315-321, wherein an inlet of the first channel is fluidically connected to a waste reservoir via a central valve when the central valve is in a first position, the sequencing instrument comprising the waste reservoir and the central valve.

Example 323. The method of example 321, further comprising: moving the central valve to a second position to fluidically couple a reagent reservoir with the channel and a second channel of the flow cell; and pumping a first volume of reagent through the first channel and into the waste reservoir.

Example 324. A method, comprising: priming fluidic lines and a sample sipper assembly of a library preparation system with lead buffer using a pump manifold assembly of a sequencing instrument; drawing samples of interest into the fluidic lines and the sequencing instrument from the library preparation system using the sample sipper assembly and the pump manifold assembly; drawing lag buffer into the fluidic lines and behind the samples of interest and the sequencing instrument from the library preparation system using the sample sipper assembly and the pump manifold assembly; and urging the lag buffer, the samples of interest, and the lead buffer toward channels of a flow cell positioned on a flow cell interface of the sequencing instrument.

Example 325. The method of example 324, further comprising flowing the lag buffer into the channels of the flow cell before the samples of interest.

Example 326. The method of any one of examples 324-325, further comprising drawing an air bubble into the fluidic lines between the lead buffer and the samples of interest.

Example 327. The method of any one of examples 324-326, further comprising drawing an air bubble into the fluidic lines between the lag buffer and the samples of interest.

Example 328. The method of any one of examples 324-327, further comprising flowing a disinfectant through the fluidic lines.

Example 329. The method of example 328, wherein the disinfectant comprises bleach.

Example 330. The method of any one of examples 324-329, wherein the lead buffer and the lag buffer comprise buffer.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

As used herein, an element or step recited in the singular and proceeded with the word “a” or “an” should be understood as not excluding plural of said elements or steps, unless such exclusion is explicitly stated. Furthermore, references to “one implementation” are not intended to be interpreted as excluding the existence of additional implementations that also incorporate the recited features. Moreover, unless explicitly stated to the contrary, implementations “comprising,” “including,” or “having” an element or a plurality of elements having a particular property may include additional elements whether or not they have that property. Moreover, the terms “comprising,” including, “having,” or the like are interchangeably used herein.

The terms “substantially,” “approximately,” and “about” used throughout this Specification are used to describe and account for small fluctuations, such as due to variations in processing. For example, they can refer to less than or equal to +5%, such as less than or equal to ±2%, such as less than or equal to 1%, such as less than or equal to ±0.5%, such as less than or equal to +0.2%, such as less than or equal to 0.1%, such as less than or equal to ±0.05%. In one example, these terms include situation where there is no variation—0%.

There may be many other ways to implement the subject technology. Various functions and elements described herein may be partitioned differently from those shown without departing from the scope of the subject technology. Various modifications to these implementations may be readily apparent to those skilled in the art, and generic principles defined herein may be applied to other implementations. Thus, many changes and modifications may be made to the subject technology, by one having ordinary skill in the art, without departing from the scope of the subject technology. For instance, different numbers of a given module or unit may be employed, a different type or types of a given module or unit may be employed, a given module or unit may be added, or a given module or unit may be omitted.

Underlined and/or italicized headings and subheadings are used for convenience only, do not limit the subject technology, and are not referred to in connection with the interpretation of the description of the subject technology. All structural and functional equivalents to the elements of the various implementations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

It should be appreciated that all combinations of the foregoing concepts and additional concepts discussed in greater detail below (provided such concepts are not mutually inconsistent) are contemplated as being part of the subject matter disclosed herein. In particular, all combinations of claimed subject matter appearing at the end of this disclosure are contemplated as being part of the subject matter disclosed herein.

1. A modular system for preparing a library of samples for sequencing, the modular system comprising:
a first assay bay for performing a first assay, comprising:
a first contact dispenser;
a first working area comprising:
a working plate receptacle;
a thermocycler; and
a magnet; and
a first drawer, the first drawer comprising a consumables area adapted to receive a working plate adapted to contain a sample, and a plurality of consumables for interacting with the sample;
a common bay, the common bay comprising an analyzer area including an imaging system; and
a mover operatively coupled to the first assay bay and the common bay,
wherein the mover is movable in a first direction along the first assay bay and a second direction perpendicular to the first direction between the first assay bay and the common bay, such that the mover is configured to move the working plate from the consumables area to the working plate receptacle in the first working area,
wherein the first contact dispenser is linearly movable in the first direction between the consumables area and the first working area, such that the first contact dispenser is configured to move the plurality of consumables between the consumables area and the working plate in the working plate receptacle in the first working area, and
wherein the mover is further configured to move the sample from the first working area to the analyzer area for analysis by the imaging system.

2. The modular system of claim 1, wherein the first contact dispenser is not movable in the second direction, wherein the first contact dispenser and the mover are both movable in a third direction perpendicular to the first and second directions, wherein the common bay further comprises a poolinq area, and wherein the mover is configured to move between the analyzer area and the pooling area, and wherein the first contact dispenser comprises a first contact head configured to hold tips, wherein the first drawer is linearly movable in the first direction relative to the first working area between a loading position and an operating position, wherein when the first drawer is in the loading position, the first drawer is spaced a first distance from the first working area, and when the first drawer is in the operating position, the first drawer is spaced a second distance from the first working area, the second distance being less than the first distance

3-6. (canceled)

7. The modular system of claim 1, further comprising a movable stage coupled to the first contact dispenser for linearly moving the first contact dispenser in the first direction and the third direction, further comprising:
a first actuator operatively coupled to the movable stage and to the magnet, the first actuator configured to actuate movement of the movable stage and to move the magnet relative to the working plate receptacle;
a gantry system, wherein the mover is movable along the gantry system;
a second actuator configured to actuate movement of the mover in the first and second directions; and
a door that is movable to enclose the thermocycler and the working plate receptacle,
wherein the thermocycler is aligned with the working plate receptacle in the first direction and configured to amplify the sample within the working plate.

8-13. (canceled)

14. The modular system of claim 1, wherein the consumables area is adapted to receive a lid for the working plate, and wherein the first contact dispenser is configured to move the lid from the consumables area and to place the lid on the working plate. wherein the plurality of consumables comprise a tip tray comprising a first reusable tip and a second reusable tip, one or more additional working plates, an index tray adapted to contain indexes, a bead tray adapted to contain beads, and a reagent reservoir adapted to contain a reagent, wherein the first contact dispenser is configured to aspirate the indexes from the index tray in the consumables area and to dispense the indexes in the working plate in the first working area, wherein the first contact dispenser is configured to aspirate the beads from the bead tray in the consumables area and to dispense the beads in the working plate in the first working area, wherein the magnet is movable toward the working plate receptacle to draw the beads in the working plate toward the magnet, and wherein the first contact dispenser is configured to aspirate a first reagent from the reagent reservoir in the consumables area and to dispense the first reagent in the working plate, and wherein the first contact dispenser is configured to aspirate the sample and the first reagent from the working plate, the first contact dispenser is configured to dispense the sample and the first reagent in a second working plate, and the mover is configured to move the second working plate from the first working area to the analyzer area.

15-20. (canceled)

22. The modular system of claim 1, wherein the imaging system is configured to obtain image data of the portion of the first reagent and the sample to determine a concentration of the sample.

23. The modular system of of claim 1, further comprising a second assay bay disposed in parallel with the first assay bay, the second assay bay for performing a second assay and comprising:
a second contact dispenser;
a second working area comprising:
a working plate receptacle;
a thermocycler; and
a magnet; and
a second drawer, the second drawer comprising a second consumables area adapted to receive a working plate adapted to contain a sample, and a plurality of consumables for interacting with the sample,
wherein the mover is operatively coupled to the second assay bay and is configured to move the working plate from the second consumables area to the working plate receptacle in the second working area,
wherein the second contact dispenser is linearly movable in the first direction between the consumables area and the second working area, such that the second contact dispenser is configured to move the plurality of consumables between the second consumables area and the working plate in the working plate receptacle in the second working area, and
wherein the mover is movable in the second direction between the second assay bay and the common bay, such that the mover is configured to move the sample from the second working area to the analyzer area for analysis by the imaging system.

24. The modular system of claim 23, wherein the second assay is performed simultaneously with the first assay.

25-26. (canceled)

27. A modular bay for preparing a library of samples for sequencing, the modular bay comprising:
a contact dispenser;
a working area comprising:
a working plate receptacle;
a thermocycler; and
a magnet; and
a drawer, the drawer comprising a consumables area adapted to receive a working plate adapted to contain a sample, and a plurality of consumables for interacting with the sample,
wherein the contact dispenser is linearly movable in a longitudinal direction between the consumables area and the working area, such that the contact dispenser is configured to move the plurality of consumables between the consumables area and the working plate in the working area.

28. The modular bay of claim 27, wherein the contact dispenser comprises a contact head configured to hold tips and wherein the contact dispenser is not movable in a lateral direction perpendicular to the longitudinal direction, further comprising:
a movable stage operatively coupled to the contact dispenser for linearly moving the contact dispenser in the longitudinal direction;
an actuator configured to actuate movement of the movable stage in the longitudinal direction; and
a door that is movable to enclose the thermocycler and the working plate receptacle,
wherein the thermocycler is aligned with the working plate receptacle in the longitudinal direction and configured to amplify the sample within the working plate

29. The modular bay of claim 27, wherein the drawer is linearly movable in the longitudinal direction relative to the working area between a loading position and an operating position, wherein when the drawer is in the loading position, the drawer is spaced a first distance from the working area, and when the drawer is in the operating position, the drawer is spaced a second distance from the working area, the second distance being less than the first distance.

30-35. (canceled)

36. The modular bay of claim 27, wherein
the consumables area is adapted to receive a lid for the working plate, and wherein the contact dispenser is configured to move the lid from the consumables area and to place the lid on the working plate.

37. The modular bay of claim 27, wherein the plurality of consumables comprise a tip tray comprising a first reusable tip and a second reusable tip, one or more additional working plates, an index tray adapted to contain indexes, a bead tray adapted to contain beads, and a reagent reservoir adapted to contain a reagent.

38. The modular bay of claim 37, wherein the contact dispenser is configured to aspirate the indexes from the index tray in the consumables area and to dispense the indexes in the working plate in the working area, wherein the contact dispenser is configured to aspirate the beads from the bead tray in the consumables area and to dispense the beads in the working plate in the working area, wherein the magnet is movable toward the working plate receptacle to draw the beads in the working plate toward the magnet, and wherein the contact dispenser is configured to aspirate a first reagent from the reagent reservoir in the consumables area and to dispense the first reagent in the working plate, and wherein the contact dispenser is configured to aspirate the sample and the first reagent from the working plate, and the contact dispenser is configured to dispense the sample and the first reagent in a second working plate of the one or more additional working plates in the consumables area.

39-42. (canceled)

43. An apparatus, comprising:
a system, comprising:
a consumables area comprising:
a consumables receptacle to receive a tip tray comprising a first tip and a second tip, a first plate having a well containing a sample, a second plate having a well, an index tray having a well containing indexes, and a bead tray having a well containing beads;
a mover;
a contact dispenser;
a stage to move the contact dispenser;
a plate receptacle;
a magnet;
a thermocycler; and
an analyzer area comprising an imaging system.

44. The apparatus of claim 43, further comprising an actuator to move the magnet relative to the plate receptacle and wherein the thermocycler is aligned with the plate receptacle.

45. (canceled)

46. The apparatus of claim 43, wherein the mover is to move the first plate from the consumables area to the plate receptacle, wherein the stage is to align the contact dispenser with the tip tray and the contact dispenser is to couple with the first tip from the tip tray, wherein the stage is to align the contact dispenser with the index tray and the contact dispenser is to aspirate the indexes from the index tray, wherein the stage is to align the contact dispenser with the first plate, and wherein the contact dispenser is to dispense the indexes into the well of the first plate, wherein the thermocycler is to amplify the sample within the well of the first plate.

47-48. (canceled)

49. The apparatus of claim 43, wherein the consumables area further comprises a lid and wherein the mover is to move the lid from the consumables area and to place the lid on the first plate to cover the well of the first plate with the lid, wherein the mover is to move the lid from the first plate to the consumables area, wherein the stage is to align the contact dispenser with the bead tray and the contact dispenser is to aspirate the beads from the bead tray, wherein the stage is to align the contact dispenser with the first plate, and wherein the contact dispenser is to dispense the beads into the well of the first plate.

50-51. (canceled)

52. The apparatus of claim 49, wherein the stage is to align the contact dispenser with the first plate and the contact dispenser is to dispense a first reagent into the well of the first plate, wherein the stage is to align the contact dispenser with the tip tray and the contact dispenser is to place the first tip in the tip tray and the contact dispenser to couple with the second tip from the tip tray, wherein the actuator is to move the magnet toward the plate receptacle to draw the beads toward the magnet and wherein the stage is to align the contact dispenser with the first plate to allow the contact dispenser to aspirate the first reagent from the well.

53-54. (canceled)

55. The apparatus of claim 52, wherein the system comprises a waste and wherein the contact dispenser is to dispense the first reagent into the waste, wherein the stage is to align the contact dispenser with the first plate and the contact dispenser is to dispense a second reagent into the well of the first plate, wherein the actuator is to move the magnet toward the plate receptacle to draw the beads toward the magnet, wherein the stage is to align the contact dispenser with the first plate to allow the contact dispenser to aspirate the second reagent and the sample from the well of the first plate, and wherein the stage is to align the contact dispenser with the second plate to allow the contact dispenser to dispense the second reagent and the sample into the well of the second plate.

56-57. (canceled)

58. The apparatus of claim 55, wherein the imaging system is to obtain image data of a portion of the second reagent and the sample and the system to determine a concentration of the sample, further comprising a second contact dispenser, and wherein the stage is to align the second plate with the second contact dispenser and wherein the second contact dispenser is to dispense a diluent into the well of the second plate to dilute the sample based on the concentration of the sample determined.

59-60. (canceled)

61-67. (canceled)

68. The apparatus of claim 43, further comprising a second system fluidly coupled to the system, wherein the second system comprises a sequencing instrument.

69-330. (canceled)
